(19) United States
(12) Patent Application Publication
(10) Pub. No.: US 2021/0318274 A1
(43) Pub. Date: Oct. 14, 2021

(54) METHODS FOR EVALUATING LIVER FUNCTION

(71) Applicant: HepQuant, LLC, Denver, CO (US)

(72) Inventors: Gregory Thomas Everson, Englewood, CO (US); Steve Mark Helmke, Denver, CO (US)

(21) Appl. No.: 17/227,042

(22) Filed: Apr. 9, 2021

Related U.S. Application Data
(60) Provisional application No. 63/007,810, filed on Apr. 9, 2020.

(51) Int. Cl. G01N 30/72; H01J 49/42; H01J 49/00; G01N 30/86; G16H 10/40

(52) U.S. Cl. CPC G01N 30/7233; H01J 49/42; G01N 2030/027; G01N 30/8624; G16H 10/40; H01J 49/0031

(57) ABSTRACT

Improved methods and kits are provided for non-invasively evaluating liver function of a patient including rapidly and efficiently processing, detecting, and quantifying distinguishable compounds from patient blood or serum samples. A method is provided for estimating risk of experiencing a clinical event in 1 year for an individual patient having a chronic liver disease. Methods for determining hepatic reserve in a subject are provided.

### CROSS REFERENCES TO RELATED APPLICATIONS

This application claims priority to and the benefit of U.S. provisional application No. 63/007,810, filed Apr. 9, 2020, the entire contents of which are incorporated herein by reference.

### BACKGROUND OF THE DISCLOSURE

Chronic liver disease progression is slow and silent and tests of liver function and disease severity to monitor patients and to predict outcomes are of great clinical value. Several such tests have been proposed and developed. The HALT-C trial was designed to investigate and directly compare the clinical utility of a number of experimental tests that attempted to quantify liver function. Everson et al., HALT-C Trial Group. Aliment Pharmacol Ther. 2009; 29: 589-601. In HALT-C, the dual cholate test for evaluating liver function showed particular strength in monitoring patients and predicting clinical outcomes. Everson et al. Hepatology 2012; 55: 1019-29.

The dual cholate clearance method relies on the natural clearance by the liver of the endogenous bile acid cholate (cholic acid, CA). In the dual cholate clearance test two distinguishable cholic acids are given to the patient, each labeled with stable isotopes to distinguish them from the endogenous cholic acid naturally present. For example, the patient receives a 20 mg dose of cholic acid-24-¹³C (13C-CA) in an intravenous bolus. At the same time, the patient drinks a dose of 40 mg cholic-acid-2,2,4,4-d4 (4D-CA) dissolved in NaHCO₃ and mixed with juice. In the HALT-C trial, peripheral blood samples were drawn before administration of the two doses (time 0) and at 5, 10, 15, 20, 30, 40, 45, 60, 75, 80, 90, 105, 120, 150, and 180 minutes. The serum concentrations of the labeled cholates at all of these time points were used to generate oral D₄-CA and IV ¹³C-CA clearance curves. This resulted in measures of the oral cholate clearance normalized to body weight (Cholate Clₒᵣₐₗ), the IV cholate clearance normalized to body weight (Cholate Cl_{IV}), and their ratio (Cholate Shunt). Each of the tests had some ability to identify patients who would have negative outcomes. Of the various measures, the Cholate Clₒᵣₐₗ was found to be an excellent predictor of future clinical outcomes and index of liver disease. Everson et al. Hepatology 2012; 55: 1019-29. A minimal model based on spline functions was developed and validated. Everson et al. Aliment Pharmacol Ther. 2007; 26: 401-10. The minimal model requires serum collection at time 0 and the 5, 20, 45, 60, and 90-minute samples to accurately reproduce the oral D₄-CA and IV ¹³C-CA clearance curves. This is the basis for the HepQuant-SHUNT™ test.

Previously, the present inventors developed and partially validated a liquid chromatography-mass spectrometry (LC-MS) assay to quantify D₄-CA and ¹³C-CA that included a multi-step extraction procedure of the two analytes from human serum, with separation and detection of the analytes using single ion monitoring LC-MS. However, prior art sample preparation and analysis is laborious, and previous methods employing GC-MS or LC-MS analysis suffer from certain drawbacks.

U.S. Pat. No. 8,613,904, Everson et al., discloses methods for evaluating liver function in a patient comprising obtaining patient serum samples following administration of two distinguishable stable isotope labeled cholate compounds, laborious sample processing and analysis of patient serum samples utilizing GC-MS. The cholate compounds are isolated from serum samples by a method including isolation and derivitization of the analyte. Sample analyte derivitization is employed because analyte volatilization is required for GC analysis. Sample preparation included the steps of adding unlabeled cholic acid internal standard to 0.5 mL of patient serum samples, diluting sample with aqueous sodium hydroxide, applying diluted sample to solid phase extraction (SPE) cartridge (e.g., Waters™ Sep-pak C18), eluting sample from the SPE cartridge, drying and acidifying sample eluate with dilute HCl, extracting acidified sample with diethyl ether, and evaporating the ether layer to form an evaporated sample, treating the evaporated sample with 2,2-dimethoxypropane (DMP) in methanol and HCl in the dark for 30 minutes, and derivitizing the treated sample with hexamethyldisilazane (HMDS) catalyzed with pyridine and trimethylchlorosilane (TMCS) with heating to 55-60° C. for 2 hours, evaporating solvents from derivitized sample, and reconstituting sample by repeated addition and evaporation of hexane to form a reconstituted sample. The reconstituted sample is injected to capillary GC-MS system for ratiometry. Sample derivitization may cause problems associated with chemical degradation and formation of new products which may occur under high heat conditions. In addition, laborious manual sample preparation methods for GC-MS require extensive sample preparation times, resulting in low throughput, and may result in reduced recovery of analyte from sample.

U.S. Pat. No. 8,778,299, Everson, discloses methods for evaluating liver function comprising obtaining patient serum samples following administration of two distinguishable stable isotope labeled cholate compounds, processing and analysis of patient serum samples utilizing HPLC-MS. U.S. Pat. No. 8,778,299 discloses a manual method of sample extraction for HPLC-MS comprising the steps of adding unlabeled cholic acid internal standard to at least 0.5 mL of patient serum samples, diluting samples with aqueous sodium hydroxide, applying diluted sample to solid phase extraction (SPE) cartridge (e.g., Waters™ reverse phase Sep-pak C18), eluting sample from the SPE cartridge, drying and acidifying sample eluate with dilute HCl, extracting acidified sample with diethyl ether, separating and evaporating the ether layer, and dissolving the evaporated sample in mobile phase buffer to form a reconstituted sample. The reconstituted sample is injected to an HPLC-MS system, for example, using multimode electrospray (MM-ES) ionization with atmospheric pressure chemical ionization (APCI). Selected ion monitoring (SIM) is performed for unlabeled and stable isotope labeled cholates at, e.g., 407.30, 408.30 and 411.30 m/z for cholic acid, 13C-cholic acid and 4-D cholic acid, respectively. The HPLC-MS method eliminated the need for sample derivitization required in the GC-MS method. Unlike GC analysis, sample volatilization is not required for LC, which shortens analysis times and avoids problems associated with chemical degradation and formation of new products which may occur under high heat conditions. However, there is still a number of manual steps required for analyte extraction, and a need for correction of ion overlap between administered isotopes using simultaneous equations for solving minor ion bleed-over and contamination of ion peaks in the LC-MS method.

Improved sample processing and quantification methods are desirable to enhance analytical assay performance and optimize assay efficiencies, for example, in support of clinical assay development and pre-market device approval by the FDA.

### SUMMARY OF THE INVENTION

Improved methods for evaluating liver function in a patient are provided herein including rapidly and efficiently processing, detecting and quantifying distinguishable compounds from patient blood or serum samples.

Improved methods are provided herein for blood or serum sample processing and analyte detection and quantification for use in liver function tests. Simplified methods for blood or serum sample processing suitable for automation have been developed.

A method is provided for quantifying one or more distinguishable compounds in a blood or serum sample from a subject, the method comprising receiving a blood or serum sample obtained from a subject having or suspected of having or developing a chronic liver disease or hepatic disorder, wherein the sample was collected from the subject less than 3 hours after oral and/or intravenous administration of the one or more distinguishable compounds to the subject; processing the blood or serum sample to form a processed sample; injecting the processed sample onto a mass detection system; measuring the concentration of the one or more distinguishable compounds in the processed sample comprising mass detection; and quantifying the concentration of the one or more distinguishable compounds in the blood or serum sample. The processed sample may be a supernatant or an eluate. The processing of the blood or serum sample may comprise forming a supernatant. Optionally, the supernatant may be injected onto a separation system comprising a preparative component, and/or an analytical component to form an eluate which may be injected to a mass detection system. In some embodiments, the method does not include a separation system. The optional separation system may comprise a chromatography system. In some embodiments, the method does not include chromatography.

A method is provided for quantifying one or more distinguishable compounds in a blood or serum sample from a subject, the method comprising receiving a blood or serum sample obtained from a subject having or suspected of having or developing a chronic liver disease or hepatic disorder, wherein the sample was collected from the subject less than 3 hours after oral and/or intravenous administration of the one or more distinguishable compounds to the subject; processing the blood or serum sample to form a supernatant; injecting the supernatant onto a separation system comprising a preparative component, and an analytical component to form an eluate; and measuring the concentration of the one or more distinguishable compounds in the eluate, wherein the measuring comprises quantifying the concentration of the one or more distinguishable compounds in the sample using a mass detection system. Optionally, the separation system may include a mobile phase component.

The optional separation system may comprise a chromatography system. The chromatography system may include a liquid chromatography (LC) system, optionally wherein the LC system is selected from the group consisting of an HPLC and a UPLC system.

In some embodiments, the mass detection system may comprise a mass spectrometer. The mass spectrometer comprises an ion source system and a mass resolution/detection system. The ion source system may be selected from the group consisting of electrospray ionization (ES), matrix-assisted laser desorption/ionization (MALDI), fast atom bombardment (FAB), chemical ionization (CI), atmospheric pressure chemical ionization (APCI), liquid secondary ionization (LSI), laser diode thermal desorption (LDTD), and surface-enhanced laser desorption/ionization (SELDI). The mass resolution/detection system may be selected from the group consisting of triple quadrupole mass spectrometer (MS/MS); single quadrupole mass spectrometer (MS); Fourier-transform mass spectrometer (FT-MS); and time-of-flight mass spectrometer (TOF-MS). The triple quadrupole mass spectrometer (MS/MS) may be run in a multiple reaction mode (MRM), optionally a negative ion multiple reaction mode.

In some embodiments, the injecting includes injecting the supernatant to the preparative component; and eluting the preparative component onto the analytical component. In some embodiments, the preparative component and the analytical component are in line. In some embodiments, the supernatant is injected to a preparative column, and the flow is reversed to elute the preparative column onto the analytical column in line. The preparative component may comprise a solid phase resin and the analytical component may comprise a solid phase resin. The solid phase resin of the preparative and analytical components may each independently be selected from the group consisting of a normal phase resin, reverse phase resin, hydrophobic interaction solid phase resin, hydrophilic interaction solid phase resin, ion-exchange solid phase resin, size-exclusion solid phase resin, and affinity-based solid phase resin. In some embodiments, the preparative and analytical components each include a reverse phase resin, optionally wherein the reverse phase resin is independently a C8 or a C18 resin.

Methods for analysis of blood or serum samples have been developed employing LC-MS/MS using multiple reaction monitoring (MRM). Differences of present methods from previous methods include: (i) unlabeled cholic acid is now quantified in each individual sample rather than only in the baseline samples, (ii) the previous multi-step extraction procedure including a combination of solid phase extraction, liquid-liquid extraction, evaporation and reconstitution has been replaced by an automated online extraction procedure, and (iii) analyte detection and quantification is now based on analyte ion transitions in multiple reaction mode (MS/MS versus MS). Advantages of the improved methods provided herein include improved recovery of analyte from the sample, increased analyte selectivity, increased sample throughput, decreased time of processing, reduced patient sample volume, and improve lower limit of quantitation (LOQ).

In some embodiments, a method is provided for quantifying one or more distinguishable compounds in a blood or serum sample from a subject, the method comprising receiving a blood or serum sample obtained from a subject having or suspected of having or developing a chronic liver disease or hepatic disorder, wherein the sample was collected from the subject less than 3 hours after oral administration of a first distinguishable compounds to the subject; adding a protein precipitation solution to the sample to form a precipitated sample and a supernatant; injecting the supernatant onto an analytical column; and measuring the concentration of the first distinguishable compounds in the analytical column eluate, wherein the measuring comprises quantifying the concentration of the first distinguishable compound in the sample by multiple reaction mode (MRM) liquid chromatography-quadrapole mass spectroscopy (LC-MS/MS). Optionally the method comprises centrifuging the precipitated sample to form the supernatant. Optionally the method comprises injecting comprises injecting the supernatant to an extraction column; and eluting the extraction column onto the analytical column. In some embodiments, after injecting supernatant onto extraction column, the method comprises reversing the flow to apply the extracted supernatant onto the analytical column.

In some embodiments, a second distinguishable compound was also administered to the subject by parenteral, optionally intravenous, administration less than 3 hours prior to collecting the sample from the subject. The first and second distinguishable compounds may be administered within 15 minutes, 10 minutes, 5 minutes, two minutes, or simultaneously to the subject.

In some embodiments, the protein precipitation solution may comprise a miscible organic solvent. The protein precipitation solution may be an aqueous solution comprising at least 50% by volume of a miscible organic solvent. The protein precipitation solution may include a water miscible organic solvent elected from the group consisting of methanol, ethanol, isopropanol, acetonitrile and acetone. In some embodiments, the miscible organic solvent is an organic alcohol. The organic alcohol may be a C₁-C₆ organic alcohol. The organic alcohol may be selected from the group consisting of methanol, ethanol, and isopropanol. In another aspect, the protein precipitation solution may include dimethoxyacetone, which when exposed to an aqueous acidic solution may decompose to acetone and methanol.

In some embodiments, the protein precipitation solution further comprises an internal standard distinguishable compound.

The volume of the blood or serum sample may be 10 μL or more, 20 μL or more, 30 μL or more, 40 μL or more, 50 μL or more, or preferably from 50-500 μL. The blood sample may be whole blood. The blood sample may be venous blood or capillary blood.

In some embodiments, the distinguishable compound analyte extraction recovery from the blood or serum sample is >80%, >85%, >90%, >95%, or >97%.

In some embodiments, the first and/or second distinguishable compound(s) is capable of exhibiting high hepatic extraction of at least 50%, 60%, 70%, 75%, or at least 80%, in first pass through the liver of a healthy subject following oral administration.

In some embodiments, the first and/or second distinguishable compound(s) is a distinguishable bile acid, bile acid conjugate, bile acid analog, or FXR agonist. The distinguishable bile acid, bile acid conjugate, or bile acid analog may be selected from the group consisting of a distinguishable selected from the group consisting of a distinguishable cholic acid (CA), dehydrolithocholic acid (dehydroLCA), lithocholic acid (LCA), isodeoxycholic acid (isoDCA), isolithocholic acid (isoLCA), allolithocholic acid (alloLCA), glycolithocholic acid (GLCA), deoxycholic acid (DCA), chenodeoxycholic acid (CDCA), taurolithocholic acid (TLCA), apocholic acid (apoCA), 23-nordeoxycholic acid (nor-DCA), 12-ketolithocholic acid (12-ketoLCA), 7-ketolithocholic acid (7-ketoLCA), 6,7-diketolithocholic acid (6,7-diketoLCA), glycodeoxycholic acid (GDCA), 6-keto-lithocholic acid (6-ketoLCA), glycochenodeoxycholic acid (GCDCA), hyodeoxycholic acid (HDCA), ursodeoxycholic acid (UDCA), cholic acid (CA), taurodeoxycholic acid (TDCA), allocholic acid (ACA), beta-hyodeoxycholic acid (beta-HDCA), murocholic acid (muroCA), hyocholic acid (HCA), 12-dehydrocholic acid (12-DHCA), beta-muricholic acid (beta-MCA), norcholic acid (norCA), 7-ketodeoxycholic acid (7-ketoDCA), glycocholic acid (GCA), alpha-muricholic acid (alpha-MCA), glycohyodeoxycholic acid (GHDCA), 3beta-cholic acid (betaCA), glycoursodeoxycholic acid (GHCA), omega-muricholic acid (omegaMCA), taurocholic acid (TCA), glycohyocholic acid (GHCA), taurohyodeoxycholic acid (THDCA), 7,12-diketolithocholic acid (7,12-diketoLCA), dehydrocholic acid (DHCA), ursocholic acid (UCA), taurohyocholic acid (THCA), tauro beta-muricholic acid (TbetaMCA), tauro alpha-muricholic acid (TalphaMCA), glycodehydrocholic acid (GDHCA), tauro omega-murichlic acid (TomegaMCA), taurohydrocholic acid (TDHCA), ursodeoxycholic acid (UDCA), hyodeoxycholic acid (HDCA), (3α,6α-dihydroxy-5β-cholan-24-oic acid), deoxycholic acid, all beta cholic acid, lithocholic acid 3-hemisuccinate, epideoxycholic acid, ursodeoxycholic acid methyl ester, ursocholanic acid, obeticholic acid (2alpha-ethyl-chenodeoxycholic acid), cholic acid methyl ester, cholic alcohol, epilithocholic acid, or an isotopically labeled derivative, or analog or epimer thereof. In some embodiments, the stable isotope labeled bile acid, bile acid conjugate, or bile acid analog is selected from the group consisting of 2,2,4,4-d4-cholic acid (D4-CA; CA-D4), 24-¹³C-cholic acid (¹³C-CA), 2,2,3,4,4-d₅ cholic acid (D₅-CA), 3,6,6,7,8,11,11,12-d8 cholic acid (D8-CA), lithocholic acid-2,2,4,4-D4 (LCA-D4), ursodeoxycholic acid-2,2,4,4-D4 (UDCA-D4), ursodeoxycholic acid (24-13C-UDCA), deoxycholic acid-2,2,4,4-D4 (DCA-D4), glycochenodeoxycholic acid-2,2,4,4-D4 (GCDCA-D4), glycochenodeoxycholic acid (glycine-2,2,3,4,4,6,6,7,8-D9-CDCA), glycodeoxycholic acid-2,2,4,4-D4 (GDCA-D4), glycocholic acid-2,2,4,4-D4 (GCA-D4), glycocholic acid (glycine-1-13C-CA), deoxycholic acid-24-13C (DCA-24-13C), deoxycholic acid (2,2,4,4,11,11-D6-DCA), alpha-muricholic acid (2,2,3,4,4-D5-αMCA), beta-muricholic acid (2,2,3,4,4-D5-βMCA), chenodeoxycholic acid (2,2,3,4,4,6,6,7,8-D9-CDCA), chenodeoxycholic acid (2,2,3,4,4-D5-CDCA), chenodeoxycholic acid (2,2,4,4-D4-CDCA), chenodeoxycholic acid (24-13C-CDCA), gamma-muricholic acid (2,2,3,4,4-D5-γMCA), omega-muricholic acid (2,2,3,4,4-D5-ωMCA), taurochenodeoxycholic acid, sodium salt (taurine-2,2,3,4,4,6,6,7,8-D9-CDCA); taurochenodeoxycholic acid, sodium salt (taurine-2,2,4,4-D4-CDCA); taurocholic acid, sodium salt (taurine-13C2-CA); taurocholic acid, sodium salt (taurine-2,2,4,4-D4-CA); taurodeoxycholic acid, sodium salt (taurine-2,2,4,4,11,11-D6-DCA); taurodeoxycholic acid, sodium salt (taurine-2,2,4,4-D4-DCA); tauroursodeoxycholic acid, sodium salt (taurine-2,2,4,4-D4-UDCA); tauroursodeoxycholic acid, sodium salt (taurine-13C2-UDCA), glycolithocholic acid (glycine-2,2,4,4-D4-LCA), 11,12-dideuterated chenodeoxycholic acid (D2-chenodeoxycholic acid, D2-CA), glycoursodeoxycholic acid (glycine-2,2,4,4-D4-UDCA), and glycoursodeoxycholic acid (glycine-13C2-UDCA).

In some embodiments, the distinguishable compound is a distinguishable bile acid, bile acid conjugate, or bile acid analog. In some embodiments, the distinguishable bile acid is an isotopically labeled bile acid, preferably a stable isotope labeled bile acid. In some embodiments, the stable isotope labeled cholic acid is cholic acid-2,2,4,4-D4 (D4-CA; CA-D4), 24-¹³C-cholic acid (¹³C-CA), 2,2,3,4,4-d₅ cholic acid (D₅-CA).

A method for screening for or monitoring of liver function, liver disease, or a hepatic disorder in a subject is provided comprising obtaining a blood or serum sample from a subject having or suspected of having or at risk of a chronic liver disease, following oral administration of a composition comprising a distinguishable compound to the subject, wherein the blood or serum sample was collected from the subject less than 3 hours after oral administration of the distinguishable compound to the subject; measuring the concentration of the orally administered distinguishable compound in the blood or serum sample from the subject, wherein the measuring comprises quantifying the concentration of the distinguishable compound in the sample by LC-MS/MS according to claim 1; and comparing the concentration of distinguishable compound in the blood sample to (i) a distinguishable compound concentration cutoff value or cutoffs of values established from a known patient population, and/or to (ii) the concentration of the distinguishable compound in one or more earlier samples from the same subject over time. In some embodiments, the blood or serum sample had been collected from the subject no more than 180 minutes, no more than 150 minutes, no more than 120 minutes no more than 90 minutes, no more than 75 minutes, no more than 65 minutes, no more than 60 minutes, no more than 55 minutes, no more than 45 minutes, no more than 35 minutes, no more than 30 minutes, no more than 25 minutes, no more than 15 minutes after administration of the distinguishable compound(s). In some embodiments, the blood or serum sample consists of a single blood or serum sample. In some embodiments, the blood or serum samples consist of a plurality of samples. In some embodiments, the blood or serum samples consist of 2 or more, 3 or more, 4 or more, 5 or more, 6 or more, 7 or more, 8 or more, 9 or more, 10 or more samples. In some embodiments, the blood or serum samples consist of 2, 3, 4, 5, 6, 7, 8, 9, 10, 11, or 12 samples. In some embodiments, the blood or serum samples consist of from 2 to 7 samples.

In some embodiments, the blood or serum sample consists of a single blood or serum sample, collected at one time point selected from about 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, or 180 minutes, or any time point in between, after oral only administration of the distinguishable compound.

In some embodiments, the single blood or serum sample is collected at one time point selected from about 30 to 180 minutes, 45 to 120 minutes, about 50 to 80 minutes, about 45 minutes, about 60 minutes, or about 90 minutes after oral administration of the distinguishable compound(s).

In some embodiments, the blood or serum sample consists of a single blood or serum sample, collected at one time point selected from about 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, or 180 minutes, or any time point in between, after intravenous only administration of the distinguishable compound.

In some embodiments, the blood or serum sample consists of a plurality of blood or serum samples, optionally collected at 2 or more time point selected from baseline, about 5, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, or 180 minutes, or any time point in between, after administration of the distinguishable compound(s).

In some embodiments, following oral only administration, the concentration of distinguishable compound in the single blood or serum sample compared to distinguishable compound concentration cutoff value or cutoffs of values in the known patient population is an estimation of portal hepatic filtration rate (portal HFR) in the subject.

In some embodiments, the method for estimation of portal HFR in the subject further comprises converting the concentration of the distinguishable compound in the sample by using an equation into an estimated portal HFR (mL/min/kg) in the subject; and comparing the estimated portal HFR in the subject to a portal HFR (FLOW) cutoff value or cutoffs of values established from a known patient population or within the subject over time.

In some aspects, a method is provided for converting a STAT value in a subject into an estimated portal HFR (FLOW)(mL/min/kg) value in the subject, comprising the equation:

y=A(x)+C, wherein;

x=LOG estimated portal HFR (FLOW) value (mL/min/kg) in the subject;
y=LOG STAT value (μM adjusted to 75 kg bodyweight) in the subject;
A=slope coefficient from 0.9 to 1.1; and
C=a constant from −0.05 to 0.05.

In some aspects, a method is provided for converting a STAT value in a subject into an estimated portal HFR (FLOW)(mL/min/kg) value in the subject, comprising the equation y=0.9702x+0.0206, wherein x=LOG estimated portal HFR value (mL/min/kg) in the subject; and y=LOG STAT value (μM adjusted to 75 kg bodyweight) in the subject.

In some aspects, a method is provided for converting a STAT value in a subject into an estimated portal HFR (FLOW)(mL/min/kg) value in the subject, comprising the equation Ln(x)=1.031×Ln (y)−0.0212, wherein x=estimated portal HFR value (mL/min/kg) in the subject; and y=STAT value (μM adjusted to 75 kg bodyweight) in the subject.

In some embodiments, the concentration of distinguishable compound in the single sample following oral only administration is compared to distinguishable compound concentration cutoff value or cutoffs of values in the known patient population is an estimation of a DSI value in the subject.

In some aspects, the method for estimation of a DSI value in the subject further comprises converting the concentration of the distinguishable compound in the sample by using an equation into a DSI value in the subject; and comparing the estimated DSI value in the subject to a DSI value cutoff value or cutoffs of values established from a known patient population or within the subject over time.

In some aspects, an equation is provided for converting the concentration of the distinguishable compound in the single specific sample (STAT value) into an estimated DSI value in the subject comprising y=A ln(x)+C, wherein A=slope value from 8.5 to 10.5; C=constant from 18 to 22; x=STAT value (in μM adjusted to 75 kg bodyweight); and y=DSI value in the subject.

In some aspects an equation is provided for converting a STAT value into an estimated DSI value in the subject, optionally wherein the equation is:

y=9.4514 ln(x)+21.12, wherein;

x=STAT value (in μM adjusted to 75 kg bodyweight); and

y=DSI value in the subject. As shown in FIG. 12B, this equation exhibited R²=0.8499 with n=1736 tests.

In another embodiment, an equation is provided for converting the concentration of the distinguishable compound in the single specific sample (STAT value) into an estimated DSI value in the subject is:

y=A(Ln x)²+B(Ln x)+C, wherein;

y=estimated DSI value in the subject;

x=STAT value in the subject;

A=coefficient from 1 to 1.5;

B=coefficient from 9 to 10; and

C=constant from 19.5 to 22.

In some aspects an equation is provided for converting a STAT value into an estimated DSI value in the subject, comprising the equation:

y=1.3816(Ln x)²+9.2339(Ln x)+20.196, wherein: and

y=estimated DSI value, and x=STAT value in the subject. As shown in FIG. 12C, this equation exhibited R²=0.8684 with n=1783 tests.

In some embodiments, the hepatic disorder or liver disease in the subject is selected from the group consisting of chronic hepatitis C, chronic hepatitis B, cytomegalovirus, Epstein Barr virus, alcoholic liver disease, amiodarone toxicity, methotrexate toxicity, nitrofurantoin toxicity, non-alcoholic steatohepatitis (NASH), non-alcoholic fatty liver disease (NAFLD), haemochromatosis, Wilson's disease, autoimmune chronic hepatitis, primary biliary cirrhosis, primary sclerosing cholangitis (PSC), and hepatocellular carcinoma (HCC).

In some embodiments, the estimated portal HFR value or estimated DSI value in the subject is used to screen patients for liver function or liver disease; monitor liver disease patients undergoing antiviral therapy; monitor disease progression in patients with chronic liver disease; determine stage of disease in a patient diagnosed with HCV or PSC; prioritize liver disease patients for liver transplant; determine selection of patients with chronic hepatitis B who should receive antiviral therapy; assessing the risk of hepatic decompensation in patients with hepatocellular carcinoma (HCC) being evaluated for hepatic resection; identifying a subgroup of patients on waiting list with low MELD (Model for End-stage Liver Disease score) who are at-risk for dying while waiting for an organ donor; as an endpoint in a clinical trial; replacing liver biopsy in pediatric populations; tracking of allograft function; measuring return of liver function in living donors; measuring functional impairment in cholestatic liver disease in a subject; for instituting a treatment or intervention in a patient; or, used in combination with ALT to identify early stage F0-F2 HCV patients.

In some embodiments, a method is provided for assessment of hepatic shunt and/or relative hepatic function in a subject having or suspected of having or at risk of a hepatic disorder or chronic liver disease, comprising the steps of: (a) obtaining a multiplicity of blood or serum samples collected from a subject over intervals for a period of less than 3 hours after the subject had been orally administered a first distinguishable compound and simultaneously intravenously administered a second distinguishable compound; (b) quantifying the first and the second distinguishable compounds in the samples by the method comprising LC-MS/MS with MRM; (c) calculating the hepatic shunt in the subject using the formula: AUCₒᵣₐₗ/AUCᵢᵥ×Doseᵢᵥ/Doseₒᵣₐₗ×100%, wherein AUCₒᵣₐₗ is the area under the curve of the serum concentrations of the first distinguishable compound and AUCᵢᵥ is the area under the curve of the second distinguishable compound; and (d) comparing the hepatic shunt in the subject to a shunt cutoff value or cutoffs of values established from a known patient population wherein the hepatic shunt in the subject compared to shunt cutoff value or cutoffs of values is an indicator of relative hepatic function of the subject.

In some embodiments, the samples comprise blood or serum samples collected from the subject at 2 or more, 3 or more, 3 or more, 4 or more, 5 or more, 6 or more, 7 or more, 8 or more, 9 or more, 10 or more, 11 or more, 12 or more, 13 or more or 15 or more time points, preferably collected over intervals spanning a period of time of about 90 minutes or less after administration, preferably collected at about 5, 20, 45, 60 and 90 minutes after the administration of the distinguishable compounds.

In some embodiments, a method is provided for determining a portal HFR value in a patient having or suspected of having or at risk of a chronic liver disease, comprising:

(i) receiving a plurality of blood or serum samples collected from a patient having or at risk of a chronic liver disease, following oral administration of a dose of a distinguishable compound (doseₒᵣₐₗ) to the patient, wherein the samples have been collected from the patient over intervals spanning a period of time of less than 3 hours after administration;
(ii) measuring concentration of the distinguishable compound in each sample, optionally wherein the measuring comprises processing the sample, and analyzing the processed sample by LC-MS/MS with MRM to obtain the concentration of distinguishable compound;
(iii) generating an individualized oral clearance curve from the concentration of the distinguishable compound in each sample comprising using a computer algorithm curve fitting to a model distinguishable compound clearance curve;
(iv) computing the area under the individualized oral clearance curve (AUC) (mg/mL/min) and dividing the dose (in mg) by AUC of the orally administered distinguishable compound to obtain the oral distinguishable compound clearance in the patient; and
(v) dividing the oral distinguishable compound clearance by the weight of the patient in kg to obtain the portal HFR value in the patient (mL/min/kg).

In some embodiments, the concentration of the distinguishable compound in each sample comprises analyzing a sample comprising any appropriate technique known in the art. Any appropriate known chromatography technique, spectrometry technique, or combination of techniques may be employed For example, the concentration of distinguishable compound in the sample may include analysis comprising a chromatographic technique, for example, employing gas chromatography (GC) or liquid chromatography (LC). Any appropriate known chromatography technique, or combination of techniques, may be employed such as partition chromatography, normal-phase chromatography, displacement chromatography, reversed-phase chromatography, size-exclusion chromatography, ion-exchange chromatography, bioaffinity chromatography, aqueous normal-phase chromatography, and so forth. In specific embodiments, reverse-phase chromatography such as C8 or C18 reverse-phase chromatography may be employed in extraction and analytical columns. The detection and quantification of the distinguishable compound in the sample may comprise high performance liquid chromatography (HPLC), HPLC-diode-array detection (HPLC-DAD), HPLC-fluorescence, ultra-performance liquid chromatography (UPLC), GC-MS, LC-MS, LC-MS/MS may be employed. Mass spectrometry (MS) may be employed alone or in combination with a chromatography technique to quantify the distinguishable compound in the sample. In some embodiments, LC-MS may be employed to quantify the distinguishable compound in the sample. The LC-MS may employ selected-ion monitoring (SIM), for example over a specific mass range of atomic mass units (amu) encompassing the exact mass of the distinguishable compound(s). In other embodiments, the distinguishable compound may be a radiolabeled compound, for example, radiolabeled using ³H or ¹⁴C. The analytical technique may involve liquid scintillation counting (LSC). The distinguishable compound may be a stable isotope labeled compound, for example, labeled with ²H or ¹³C. In some embodiments, LC-MS/MS is employed. In specific embodiments, LC-MS/MS is employed using multiple reaction monitoring (MRM) to quantify the distinguishable compound in the sample. In specific embodiments, MS/MS is employed using multiple reaction monitoring (MRM) to quantify the distinguishable compound in the sample. In some embodiments, MS/MS is used without LC or GC. In some embodiments, MS/MS is used with LC or GC.

A method is provided for determining an systemic HFR value in a patient having or suspected of having or at risk of a chronic liver disease, comprising

(i) receiving a plurality of blood or serum samples collected from a patient having or at risk of a chronic liver disease, following intravenous administration of a dose of a distinguishable compound (doseᵢᵥ) to the patient, wherein the samples have been collected from the patient over intervals spanning a period of time of less than 3 hours after administration;
(ii) measuring concentration of the distinguishable compound in each sample comprising LC-MS/MS with MRM;
(iii) generating an individualized intravenous clearance curve from the concentration of the distinguishable compound in each sample comprising using a computer algorithm curve fitting to a model distinguishable compound clearance curve; and
(iv) computing the area under the individualized intravenous clearance curve (AUC) (mg/mL/min) and dividing the dose (in mg) by AUC of the intravenously administered distinguishable compound to obtain the intravenous distinguishable compound clearance in the patient; and (v) dividing the intravenous distinguishable compound clearance by the weight of the patient in kg to obtain the systemic HFR value in the patient (mL/min/kg).

In other embodiments, a method for determining a disease severity index (DSI) value in a patient is provided, the method comprising:

(a) obtaining one or more liver function test values in a patient having or at risk of a chronic liver disease, wherein the one or more liver function test values are obtained from one or more liver function tests selected from the group consisting of SHUNT, portal hepatic filtration rate (portal HFR), and systemic hepatic filtration rate (systemic HFR), wherein the liver function tests comprise measuring a distinguishable compound in a blood or serum sample from the subject by a method comprising LC-MS/MS with MRM according to claim 1; and
(b) employing a disease severity index equation (DSI equation) to obtain a DSI value in the patient, wherein the DSI equation comprises one or more terms and a constant to obtain the DSI value, wherein at least one term of the DSI equation independently represents a liver function test value in the patient from step (a) or a mathematically transformed liver function test value in the patient from step (a); and the at least one term of the DSI equation is multiplied by a coefficient specific to the liver function test.

The method for estimating a DSI value in a subject may optionally include (c) comparing the DSI value in the patient to one or more DSI cut-off values, one or more normal healthy controls, or one or more DSI values within the patient over time.

In some embodiments, the mathematically transformed liver function test value in the patient is selected from a log, antilog, natural log, natural antilog, or inverse of the liver function test value in the patient.

In some embodiments, each term of the DSI equation independently represents a liver function test value in the patient from step (a) or a mathematically transformed liver function test value in the patient from step (a).

In some embodiments, the DSI equation is selected from the group consisting of:

DSI=f(Shunt, Portal HFR, Systemic HFR), wherein Shunt is a shunt value in the subject, portal HFR is a portal HFR value in the subject, and systemic HFR is systemic HFR value in the subject;

DSI=A (Shunt)+B (Log Portal HFR)+C (log Systemic HFR)+D, wherein A=a number from 5 to 6, or 5.2 to 5.8; B=a number from 6 to 8, or 6.5 to 7.5; C=a number from 8 to 10, or 8.5 to 9.5; D=a number from 40 to 60, or 44 to 55; and

DSI=A.

$$\sqrt{{( B - Log Portal HFR )}^{2} + {( C - Log Systemic HFR )}^{2} ,}$$

wherein A=a number from 8 to 12, or 10 to 11; B=a number from 3 to 5, or 3.5 to 4.5; C=a number from 1.5 to 3.5, or 2 to 3.

In some embodiments, the comparing of the DSI value in the patient to one or more DSI cut-off values is indicative of at least one clinical outcome. In some embodiments, the clinical outcome or clinical event is selected from the group consisting of Child-Turcotte-Pugh (CTP) increase, varices, encephalopathy, ascites, and liver related death.

In some embodiments, the comparing the DSI value within the patient over time is used to monitor the effectiveness of a treatment of chronic liver disease in the patient, wherein a decrease in the DSI value within the patient over time is indicative of treatment effectiveness.

In some embodiments, the comparing the DSI value within the patient over time is used to monitor the need for treatment of chronic liver disease in the patient, wherein an increase in the DSI value within the patient over time is indicative of a need for treatment in the patient.

In some embodiments, the treatment of chronic liver disease in the patient is selected from the group consisting of antiviral treatment, antifibrotic treatment, antibiotics, immunosuppressive treatments, anti-cancer treatments, FXR agonist, ursodeoxycholic acid, insulin sensitizing agents, interventional treatment, liver transplant, lifestyle changes, and dietary restrictions, low glycemic index diet, antioxidants, vitamin supplements, transjugular intrahepatic portosystemic shunt (TIPS), catheter-directed thrombolysis, balloon dilation and stent placement, balloon-dilation and drainage, weight loss, exercise, and avoidance of alcohol.

In some embodiments, the comparing the DSI value within the patient over time is used to monitor status of chronic liver disease in the patient, wherein change in DSI value within the patient over time is used to inform the patient of status of the disease and risk for future clinical outcomes, wherein an increase in the DSI value within the patient over time is indicative of a worse prognosis, and a decrease in the DSI value within the patient over time is indicative of a better prognosis.

In some embodiments, a method is provided for estimating a clinical event rate for a patient having a chronic liver disease, the method comprising obtaining a baseline DSI value (dsi0) for the patient; optionally, obtaining a repeat DSI value (dsi_{T}) for the patient, wherein T=months between collection of baseline and repeat DSI samples; and calculating estimated events per person-year of observation as a function of baseline DSI value, and optionally the repeat DSI value. In some embodiments, the calculating comprises a Poisson regression model equation.

In some embodiments, the Poisson regression model equation is:

Y=β0+β1X1+β2X2+β3X3, wherein;

Y=log of the event rat (ln(rate));
X1, X2, and X3 are explanatory variables selected from the group consisting of dsi0, dsiT, (dsi_{T}−dsi0), and (dsi_{T}*dsi0); and

β0(intercept), β1, β2, β3 are regression coefficients.

In some embodiments, the regression coefficients are obtained from a clinical study of a multiplicity of patients having a chronic liver disease, and having a defined rate of clinical events over time. In some embodiments, the clinical events are selected from the group consisting of Childs-Turcotte-Pugh 2 point score progression (CTP+2), variceal hemorrhage, ascites, encephalopathy, or death.

In some embodiments, calculating estimated events per person-year of observation as a function of baseline DSI value, and optionally a repeat DSI value, comprises a Poisson regression model equation selected from the group consisting of:

Y=β0+β1dsi0;

Y=β0+β1dsi0+β2dsi_{T};

Y=β0+β1dsi0+β2dsi24;

Y=β0+β1dsi0+β2dsi_{T}+β3(dsi0*dsi_{T});

Y=β0+β1dsi0+β2dsi24+β3(dsi0*dsi24);

Y=β0+β1dsi0+β2dltaDSI;

Y=β0+β1dsi0+β2(Δdsi);

Y=β0+β1dsi0+β2(dsi_{T}−dsi0); and

Y=β0+β1dsi0+β2(dsi24−dsi0), wherein;

dsi0 is the DSI value at baseline in the subject;
dsi_{T} is the DSI value at T months in the subject;
dsi24 is the DSI value at 24 months in the subject;
dltaDSI=Δdsi=(dsi_{T}−dsi0);
Y=log of the event rat (ln(rate)); and
β0(intercept), β1, β2, β3 are regression coefficients.

In some embodiments, a method is provided for estimating a baseline or repeat DSI value in a subject comprising obtaining a blood or serum sample from the subject, following oral administration of a composition comprising a distinguishable compound to the subject, wherein the blood or serum sample was collected from the subject less than 3 hours after oral administration of the distinguishable compound to the subject; and measuring the concentration of the orally administered distinguishable compound in the blood or serum sample from the subject, wherein the measuring comprises quantifying the concentration of the distinguishable compound in the sample comprising LC-MS/MS. Optionally the blood or serum sample may consists of a single blood or serum sample.

In some embodiments, a method is provided for estimating a clinical event rate for a patient having a chronic liver disease, the method comprising obtaining a baseline DSI value (dsi0) for the patient; and calculating estimated events per person-year of observation as a function of baseline DSI value. In some embodiments, the calculating comprises the Poisson regression model equation Y=β0+β1dsi0, wherein β0 is a coefficient within the range of from −7.359 to −5.279, optionally wherein β0=−6.2997; β1 is a coefficient within the range of from 0.107 to 0.191, optionally wherein β1=0.1498; and dsi0=baseline DSI value from the patient.

In some embodiments, a method is provided for estimating a clinical event rate for a patient having a chronic liver disease, the method comprising obtaining a baseline DSI value (dsi0) for the patient; obtaining a repeat DSI value (dsi_{T}) for the patient, wherein T=months between collection of baseline and repeat DSI samples; and calculating estimated events per person-year of observation as a function of baseline DSI value. In some embodiments, the calculating comprises the Poisson regression model equation;

Y=β0+β1dsi0+β2(dsi24−dsi0), wherein;

β0 is a coefficient within the range of from −8.417 to −6.057, optionally wherein; β0=−7.2008; β1 is a coefficient within the range of from 0.127 to 0.217, optionally wherein;

β1=0.1726; β2 is a coefficient within the range of from 0.092 to 0.185, optionally wherein; and

β2=0.1395; dsi0=baseline DSI value for the patient; and dsi24=repeat DSI value for the patient wherein the patient sample was obtained 24 months after baseline.

In some embodiments, a method for providing baseline or subsequent DSI values in the subject is provided, comprising obtaining a blood or serum sample from the subject, following simultaneous oral administration and intravenous administration of first and second compositions comprising distinguishable compounds to the subject, wherein blood or serum sample(s) had been collected from the subject less than 3 hours after oral administration of the distinguishable compounds to the subject; and measuring the concentration of the orally and intravenously administered distinguishable compounds in the blood or serum sample(s) from the subject, wherein the measuring comprises quantifying the concentration of the distinguishable compound in the sample by LC-MS/MS.

A method is provided for monitoring the effectiveness of a treatment of a chronic liver disease in a patient in need thereof, comprising determining a baseline Disease Severity Index (DSI) value in the patient prior to the treatment; determining at least one subsequent DSI value in the patient over time after initiating the treatment; and comparing the at least one subsequent DSI value to the baseline DSI value, wherein a decrease in the at least one subsequent DSI value over time compared to the baseline DSI value in the patient is indicative of treatment effectiveness in the patient. The determining the DSI value may comprise measuring the concentration of one or more distinguishable compounds in a blood or serum sample from the patient comprising LC-MS/MS or MS/MS without LC.

In some aspects, a decrease in the at least one subsequent DSI value over time compared to baseline DSI value is indicative of improved liver function, improved portal circulation, decreased portal-systemic shunting, decreased liver fibrosis, decreased Ishak fibrosis score, decreased disease severity, and/or decreased risk of clinical outcome in the patient. The decrease in the at least one subsequent DSI value over time compared to baseline DSI value in the patient may be at least about −1.5 points, at least about −2 points, or at least about −3 points. In another aspect, an increase or no change in the at least one subsequent DSI value over time compared to baseline DSI value is indicative the patient is a non-responder to the treatment.

In some aspects, DSI may be used as an endpoint in a clinical trial. For example, when using DSI as an endpoint, a significant treatment response in a given patient may be defined as a 2 point or greater decrease in DSI value over time, for example, during and or after treatment. The percentage of responders may be compared between treatment and placebo arms. The percentage of responders using DSI as an endpoint may also be compared to the percentage of responders using other tests as endpoints. Other tests such as standard laboratory tests, clinical models (e.g., MED and CTP scores), liver biopsy, hepatic venous pressure gradient (HVPG), magnetic resonance imaging (MRI), computed tomography perfusion imaging, and other imaging tests may be insensitive or nonspecific. They may not adequately assess the liver's improvement after suppression of necro-inflammation by treatment. In contrast, the present methods for determining including systemic hepatic filtration rate (HFR), portal HFR, SHUNT, and DSI specifically target the uptake of cholate and use a single non-invasive test of 90 minutes duration to quantify systemic circulation, portal circulation, and portal-systemic shunt and to derive a DSI value in intact human subjects. The present methods can measure the improvement in hepatic function that occurs after successful therapy in realtime.

In another aspect, an increase in the at least one subsequent DSI value over time compared to baseline DSI value may be used as an indication of worsening liver function, worsening portal circulation, increased portal-systemic shunting, increased liver fibrosis, increased Ishak fibrosis score, increased disease severity, and/or increased risk of clinical outcome in the patient, optionally wherein the increase in at least one subsequent DSI value over time compared to baseline is at least about 1 point.

A method of determining a DSI value in a patient is provided comprising obtaining one or more distinguishable compound test results in the patient comprising a SHUNT value, a STAT value, portal hepatic filtration rate (portal HFR) value and/or a systemic hepatic filtration rate (systemic HFR) value from the patient; and deriving a disease severity index (DSI) value from the SHUNT value, STAT value, portal HFR, systemic HFR and/or SHUNT values. The obtaining of the distinguishable compound test results may comprise quantifying the one or more distinguishable compounds by LC-MS/MS.

In some embodiments, a kit of components is provided for determining one or more of STAT, portal HFR, systemic HFR, SHUNT, cholate elimination rate, RCA20, DSI values, algebraic HR values, and/or indexed HR in a subject having, or suspected of having or developing, a hepatic disorder; the kit comprising a first component comprising one or more vials, each vial comprising a first composition comprising a single oral dose of a first distinguishable compound.

The kit may further comprise a microsampling device, optionally wherein the microsampling device includes a component selected from the group consisting of a dried blood spot filter paper, capillary tube, and a volumetric microsampling device.

The kit may further comprise a second component comprising one or more vials, each vial comprising a second composition including single intravenous dose of a second distinguishable compound.

The kit may further comprise a third component comprising one or more vials, each vial comprising a quantity of human albumin for mixing with the single intravenous dose of the second distinguishable compound prior to intravenous administration. The human albumin may be human serum albumin. The second composition may optionally further comprise human albumin pre-mixed with the second distinguishable compound.

The kit may further comprise a fourth component comprising one or a plurality of sample collection tubes and/or transport vials; and a fifth component comprising a suitable container means. The kit may include sample collection tubes comprising one or more sets of sterile blood-serum sample collection tubes, wherein each set consists of enough tubes for collection of a plurality of samples from the subject over a period of no more than 180, 90 minutes, 60 minutes, or 45 minutes after administration of the first and second distinguishable compounds.

The kit may include first and second distinguishable compounds independently selected from the group consisting of distinguishable bile acids, bile acid conjugates, and bile acid analogs. The first and second distinguishable compounds may be stable isotope labeled distinguishable bile acids. The first and second stable isotope labeled distinguishable bile acids may be selected from 2,2,4,4-²H-cholic acid and 24-¹³C cholic acid.

The first composition and/or the second composition may further independently further comprise one or more components selected from the group consisting of pharmaceutically acceptable excipients, diluents, colorings, flavorings, buffer compounds, pH adjusting agents, and vehicles. In some embodiments, the diluent may be selected from water, sodium bicarbonate solution, non-citrus juice, or normal saline (NS).

The first and/or second composition may comprise sodium bicarbonate. The first composition and the second composition independently may be in a form selected from a powder form or a solution form. The first and second compositions may both be in a solution form. In some embodiments, the first composition may comprise a first distinguishable bile acid and sodium bicarbonate, optionally, wherein the first distinguishable bile acid is 2,2,4,4-²H-cholic acid. In some embodiments, the second composition comprises a second distinguishable bile acid and sodium bicarbonate, optionally, wherein the second distinguishable bile acid is 24-²³C-cholic acid.

The kit may include container means selected from one or more of the group consisting of plastic containers, reagent containers, vials, tubes, flasks, and bottles.

The kit may include shipping box, labels, instructions, package inserts, lancets, capillary tubes, syringes, indwelling catheter, 3-way stopcock, timer, and transfer pipets.

For example, the kit may include a the shipping box comprising a single box for both shipping the vials to a health care practitioner and shipping the samples from the health care practitioner to a reference lab for analysis.

### BRIEF DESCRIPTION OF THE DRAWINGS

FIG. 1 shows a schematic representation of the Portal HFR and SHUNT tests. Healthy control subjects (upper panel) generally exhibit low SHUNT, high Portal HFR and high Systemic HFR; whereas subjects with liver disease (lower panel) exhibit higher SHUNT, lower Portal HFR and lower Systemic HFR.

FIG. 2A shows chemical structures and ring numbering systems for C24 bile acids and C27 bile acids, and a representative C24 bile acid:cholic acid, also known as 3α, 7α, 12α-trihydroxy-5β-cholan-24-oic acid. Salts of cholic acid are called cholates.

FIG. 2B shows a graph with a representative cholic acid calibration curve for 0.1 uM to 10 uM cholic acid vs. response using LC-MS/MS, wherein y=2.2467x+0.68, and R2=0.9995.

FIG. 3 shows a representative ion chromatogram of time vs. peak intensity, cps, of cholic acid in a Lower Limit of Quantification QC (LLOQC at 0.10 μmon in serum lot pool #4) monitored at m/z=407.3→343.1 at retention time 4.04 min (left panel), and internal standard D₅-CA at m/z=412.3→290.2 at retention time 4.03 min (right panel). The ion chromatograms on the left hand side show the MS/MS signals of cholic acid and the ion chromatograms on the right hand side show the MS/MS signals of the internal standard cholic acid-D₅. Please note that the scales of the x-axes are different as the Analyst software always adjusts the x-axis scale based on the highest peak.

FIG. 4 shows a representative ion chromatogram of cholic acid in an Upper Limit of Quantification QC (ULOQC at 10.0 μmon in serum lot pool #4) monitored at m/z=407.3→343.1 at retention time 4.04 min (left panel), and internal standard D₅-CA at m/z=412.3→290.2 at retention time 4.03 min (right panel).

FIG. 5 shows representative ¹³C-Cholic Acid Calibration Curve for 0.1 uM to 10 uM 13C-cholic acid vs. response using LC-MS/MS, wherein y=2.4233x−0.038, and R2=0.9981.

FIG. 6 shows a representative ion chromatogram of ¹³C-cholate (m/z=408.25 [M-H]−→343.1) in a Lower Limit of Quantification QC (LLOQ at 0.10 μmon in serum lot pool #4) (left panel), and internal standard D₅-CA at m/z=412.3→290.2 at retention time 4.03 min (right panel).

FIG. 7 shows a representative ion chromatogram of ¹³C-cholate (m/z=408.25 [M-H]⁻→343.1) in an Upper Limit of Quantification QC (ULOQC at 10.0 μmon in serum lot pool #4) (left panel), and internal standard D₅-CA at m/z=412.3→290.2 at retention time 4.03 min (right panel).

FIG. 8 shows a representative Cholic Acid-D₄ Calibration Curves for 0.1 uM to 10 uM cholic acid-d4 vs. response using LC-MS/MS, wherein y=1.7102x−0.0357, and R2=0.999.

FIG. 9 shows a representative ion chromatogram of cholate-D₄ (411.25 [M-H]⁻→347.100 Da) in a Lower Limit of Quantification QC (LLOQ at 0.10 μmon in serum lot pool #4) (left panel), and internal standard D₅-CA at m/z=412.3→290.2 at retention time 4.03 min (right panel).

FIG. 10 shows a representative ion chromatogram of cholate-D4 (411.25 [M-H]⁻→347.100 Da) in a Upper Limit of Quantification QC (ULOQC at 10.0 μmon in serum lot pool #4) (left panel), and internal standard D₅-CA at m/z=412.3→290.2 at retention time 4.03 min (right panel).

FIG. 11A-B show connections and positions of the chromatography column switching valve between preparative extraction column and analytical column in the LC-MS/MS system.

FIG. 11A shows valve position 1, where the HPLC Pump I flows through the injector so the sample is injected to the extraction column.

FIG. 11B shows valve position 2, where the HPLC Pump II back flushes the extraction column onto the analytical column which is eluted to the API 4000 MS/MS system where MRM monitoring is employed.

FIG. 12A shows the accuracy and correlation (R²=0.8965) of the 60 minute STAT test relative to the FLOW test from early CHC patients and the equation for interconverting the log STAT and log FLOW values to obtain an estimated flow rate.

FIG. 12B shows a graph of one exemplary relationship of DSI to STAT values in n=1363 subjects, and n=1736 tests. The relationship of DSI to STAT equation was derived where y=9.4514 ln(x)+21.12, where x=STAT value (04 adjusted to 75 kg bodyweight), and y=DSI value. R²=0.8499.

FIG. 12C shows a graph of another exemplary relationship of DSI to STAT values in n=1783 tests. The relationship of DSI to STAT equation was derived where y=1.3816(Ln x)²+9.2339 (Ln x)+20.196, where x=STAT value (04 adjusted to 75 kg bodyweight), and y=DSI value. R²=0.8684.

FIG. 13A-D show correlation between scoring systems for FLOW and Ishak scoring, SHUNT and Ishak scoring, FLOW and Metavir scoring, and SHUNT and Metavir scoring, respectively.

FIG. 13A shows results for the previously disclosed FLOW test in healthy controls and all stages of CHC. Data from HALT-C (later stage CHC, stably compensated, Ishak F2-6) was combined with data from the Early CHC Study (healthy controls (C) and early stage CHC, Ishak F1-2) and a study of healthy donors for living donor liver transplantation (healthy controls (C)). The F2 patient data was not different between studies and was combined. The portal blood flow (mean+/−SEM) for healthy controls and patients with all stages of CHC was graphed as a continuous function demonstrating the ability to assess the entire spectrum of disease. The n for each group is indicated above its symbol. HepQuant FLOW testing could increase early detection of liver disease when it is most treatable.

FIG. 13B shows data for the previously disclosed SHUNT test in Healthy Controls and All Stages of CHC. Data from HALT-C was combined with data from the Early CHC Study (healthy controls (C) and early stage CHC, Ishak F1-2) and a study of healthy donors for living donor liver transplantation (healthy controls (C)). The F2 patient data was not different between studies and was combined. The portal-systemic shunt fraction (mean+/−SEM) for healthy controls and patients with all stages of CHC was graphed as a continuous function demonstrating the ability to assess the entire spectrum of disease. The n for each group is indicated above its symbol. Increased variability at F1 is due to the small number of patients that were diagnosed at this early stage. HepQuant SHUNT testing could increase early detection of liver disease when it is most treatable.

FIG. 13C shows data for the previously disclosed FLOW test in Healthy Controls and All Stages of CHC. Data from HALT-C (later stage CHC, stably compensated, METAVIR F1-4) was combined with data from the Early CHC Study (healthy controls (C) and early stage CHC, METAVIR F1) and a study of healthy donors for living donor liver transplantation (healthy controls (C)). The F1 patient data was not different between studies and was combined. The portal blood flow (mean+/−SEM) for healthy controls and patients with all stages of CHC was graphed as a continuous function demonstrating the ability to assess the entire spectrum of disease. The n for each group is indicated above its symbol.

FIG. 13D shows data for the previously disclosed SHUNT test in Healthy Controls and All Stages of CHC. Data from HALT-C (later stage CHC, stably compensated, METAVIR F1-4) was combined with data from the Early CHC Study (healthy controls (C) and early stage CHC, METAVIR F1) and a study of healthy donors for living donor liver transplantation (healthy controls (C)). The F1 patient data was not different between studies and was combined. The portal-systemic shunt fraction (mean+/−SEM) for healthy controls and patients with all stages of CHC was graphed as a continuous function demonstrating the ability to assess the entire spectrum of disease. The n for each group is indicated above its symbol.

FIG. 14A shows HFR (Portal HFR, FLOW) for PSC patients in various stages of disease compared to healthy controls.

FIG. 14B shows SHUNT for PSC patients in various stages of disease compared to healthy controls.

FIG. 14C shows STAT for PSC patients in various stages of disease compared to healthy controls.

FIG. 15 shows FLOW and SHUNT test results with FLOW cutoff values (5, 10 and 20 mL/min/kg for marked severe, moderate, and mild disease, respectively) and SHUNT cutoff values (26%, 43%, and 60% for mild, moderate and marked sever disease, respectively) for individual healthy controls and in PSC patients.

FIG. 16 shows FLOW and SHUNT test results with FLOW cutoff values (5, 10 and 20 mL/min/kg for marked severe, moderate, and mild disease, respectively) and SHUNT cutoff values (26%, 43%, and 60% for mild, moderate and marked sever disease, respectively) for individual healthy controls and HCV patients.

FIG. 17 shows a graph of the relationship of a DSI value in a patient to % of maximum hepatic capacity. A higher DSI value is indicative of a lower % of maximum hepatic capacity.

FIG. 18 shows DSI linearly correlates with Ishak fibrosis score (liver biopsy, left panel) but is not influenced by steatosis (biopsy fat score, right panel), as provided in Example 9 of U.S. Pat. No. 9,091,701. n for each data point is shown in the graph.

FIG. 19 shows performance of DSI in identifying the patients with future clinical outcomes as compared to that of Ishak fibrosis score (liver biopsy), platelet count (CBC), and MELD (Model for End-stage Liver Disease score). At the optimum cutoffs, DSI surprisingly outperformed other standard test methods including liver biopsy and MELD for prediction of future clinical outcomes. Specifically, DSI exhibited the highest sensitivity, specificity, PPV, and NPV when compared to liver biopsy, platelet count and MELD

FIG. 20 shows a plot of cholate test results for non-cirrhotic chronic hepatitis C patients (Ishak F2,3,4; n=19, 63, 45) with DSI value cut-offs of 15, 25 and 35 for mild disease, moderate disease and severe disease and test results of cholate based tests SHUNT (%), systemic HFR (mL/min/kg), portal HFR (mL/min/kg) and DSI. Portal HFR is plotted on the X axis and systemic HFR on the Y axis, SHUNT, the ratio of systemic to portal HFR is represented by the diagonal lines, DSI is displayed in shaded regions. Surprisingly, non-cirrhotic patients with high DSI have greater risk of outcomes as discussed in Example 10 of U.S. Pat. No. 9,091,701, where portal HFR, systemic HFR and SHUNT were measured by methods comprising HPLC-MS with SIM.

FIG. 21 shows a plot of cholate test results for cirrhotic chronic hepatitis C patients (Ishak F5, 6; n=48,49) with DSI value cut-offs of 15, 25 and 35 for mild disease, moderate disease and severe disease test results of cholate based tests SHUNT (%), systemic HFR (mL/min/kg), portal HFR (mL/min/kg) and DSI. Portal HFR is plotted on the X axis and systemic HFR on the Y axis, SHUNT, the ratio of systemic to portal HFR is represented by the diagonal lines, DSI is displayed in shaded regions. Surprisingly, cirrhotic patients with low DSI have lower risk of outcomes as discussed in Example 10 of U.S. Pat. No. 9,091,701, where portal HFR, systemic HFR and SHUNT were measured by methods comprising HPLC-MS with SIM.

FIG. 22 shows a plot of cholate test results for primary sclerosing cholangitis (PSC) patients and healthy controls. Portal HFR is plotted on the X axis and systemic HFR on the Y axis, SHUNT, the ratio of systemic to portal HFR is represented by the diagonal lines, DSI is displayed in shaded regions. Predictive DSI cut-offs of 14, 18, and 36 for mild disease, moderate disease, and severe PSC disease, including varices, and decompensation are shown at the interfaces between zones, as disclosed in U.S. Pat. No. 9,091,701, where portal HFR, systemic HFR and SHUNT were measured by methods comprising HPLC-MS with SIM.

FIG. 23 shows a plot of DSI versus MELD scores in PSC patients on the waiting list for liver transplantation. DSI was superior to MELD in assessing risk for complications and priority for liver transplant in PSC patients. Despite low MELD scores, PSC patients with DSI>20 developed portal hypertension-related complications, and PSC patients with DSI>40 required liver transplantation, as disclosed in U.S. Pat. No. 9,091,701, where portal HFR, systemic HFR and SHUNT were measured by methods comprising HPLC-MS with SIM.

FIG. 24 shows a graph of patients achieving SVR compared to quartiles for hepatic function. The probability of SVR correlated best with DSI, as discussed in Example 13 of U.S. Pat. No. 9,091,701, where portal HFR, systemic HFR and SHUNT were measured by methods comprising HPLC-MS with SIM. 230 chronic HCV patients (Ishak F2-6) enrolled in the HALT-C Trial, characterized by advanced fibrosis and failure of prior treatment with interferon-based treatment, were tested at baseline and then retreated with PEG/RBV. Patients achieving sustained virological response SVR (n=32, including 5 cirrhotics) and non-responders (NR) were retested at 2 yrs. Testing could predict sustained virological response (SVR) to peginterferon/ribavirin (PEG/RBV) and to measure the improvement in hepatic function in those achieving SVR.

FIG. 25 shows a graph of DSI baseline and serial DSI values in 13 patients eventually diagnosed with HCC from HALT-C ancillary study. The Dashed Line near bottom of the graph is DSI 18.3 cutoff value. 12/13 HCC cases had baseline DSI>18.3. Relative Risk of HCC for DSI>18.3 is 11.4.

FIG. 26 shows a graph of estimated DSI baseline and serial estimated DSI values in 13 patients eventually diagnosed with HCC from HALT-C ancillary study. Estimated DSI values were obtained from STAT values by use of an equation. 12/13 HCC cases had baseline estimated DSI>18.3. Relative Risk of HCC for Est DSI>18.3 is 11.4.

FIG. 27 shows a graph of survival probability for patients divided into baseline DSI tertiles vs. study years for 220 HALT-C patients. Patients in tertile (A) had baseline DSI value <15.395, (B) DSI value from 15.395-19.898, and (C) DSI value >19.898. The number of subjects in each DSI tertile per study year is shown under the graph. Shaded area of graph indicates 95% confidence limits for each tertile.

FIG. 28 shows the predicted event rate for each of the 4 Poisson regression models for all 188 subjects as a function of their baseline DSI. Notice that models B and D have the same predicted values. This is expected as shown in the equations in the description of Model D.

FIG. 29 shows an agreement plot for relationship between baseline and 24 month DSI values for 188 HALT-C patients. The plot shows difference (dsi24-dsi0) vs. (dsi0+dsi24)/2. The agreement plot shows regression to mean, but with positive slope. Those with lower baseline DSI tended to have smaller DSI at 24-months and those with higher baseline DSI tended to have higher DSI 24-months.

FIG. 30 shows LUXON® MS/MS method parameters including Ionization mode: Positive. Flow: 6 L/min. Gaz: Air.

FIG. 31 shows an exemplary desorption peak for C 0.1 d4-CA (d4-cholic acid) standard 413.4/359.4 (large peak) with internal standard d5-CA-245 (414.4/245.1) (inset peak).

FIG. 32 shows two example mass spectra of intensity, positive mode, cps vs. m/z (mass to charge ratio) for product ion (MS2) 12CA at 355.40 m/z, Da (left panel) and product ion (MS2) for d5-DA at 360.40 m/z, Da (right panel).

FIG. 33A shows a graph of concentration ratio vs. area ratio from MS/MS without LC of standard samples of 12C-CA at 0.1 uM, 0.2 uM, 0.6 uM, 1 uM, 2 uM, 6 uM, and 10 um (n=3 each). Linearity was greater than 0.99 for 12C-CA.

FIG. 33B shows a graph of concentration ratio vs. area ratio from MS/MS without LC of standard samples of 13C-CA at 0.1 uM, 0.2 uM, 0.6 uM, 1 uM, 2 uM, 6 uM, and 10 um (n=3 each). Linearity was greater than 0.99 for 13C-CA.

FIG. 33C shows a graph of concentration ratio vs. area ratio from MS/MS without LC of standard samples of d4-CA at 0.1 uM, 0.2 uM, 0.6 uM, 1 uM, 2 uM, 6 uM, and 10 um (n=3 each). Linearity was greater than 0.99 for d4-CA.

FIG. 34A shows dose-normalized plasma unconjugated OCA AUC(0-24 h)(h*ng/mL/mg) at baseline (day 1) in a clinical study. The circles are observed data; red lines are linear regression fit. R²=0.394. OCA=Obeticholic acid.

FIG. 34B shows dose-normalized plasma unconjugated OCA AUC(0-24 h)(h*ng/mL/mg) at end of treatment (day 85), in a clinical study. The circles are observed data; red lines are linear regression fit. R²=0.424. OCA=Obeticholic acid. Obeticholic acid plasma exposure was related to DSI measurement.

FIG. 35A shows a graph of average antipyrine clearance for 3 groups of patients divided into Child-Pugh CP A5, CP A6, and CP B class. Patients in the CP B class exhibited decreased average antipyrine clearance compared to CP A5 and CP A6 patient groups.

FIG. 35B shows a graph of average antipyrine clearance for CP A5 subjects (N=85) from FIG. 35A that were sub-divided into 4 groups based on DSI score of 5-15, 15-25, 25-35, and 35-45. Patients exhibiting higher DSI scores exhibited lower average antipyrine clearance.

FIG. 35C shows a graph of average antipyrine clearance for CP A6 subjects (N=53) from FIG. 35A that were sub-divided into 4 groups based on DSI score of 5-15, 15-25, 25-35, and 35-45. Patients exhibiting higher DSI scores exhibited lower average antipyrine clearance.

FIG. 35D shows a graph of average antipyrine clearance for CP B subjects (N=12) from FIG. 35A that were sub-divided into 4 groups based on DSI score of 5-15, 15-25, 25-35, and 35-45. Patients exhibiting higher DSI scores generally exhibited lower average antipyrine clearance.

FIG. 36A shows a graph of average Methionine breath test results for groups of patients divided by Child-Pugh scores A5, A6 and B. Patients in the CP B class exhibited decreased average methionine breath test values compared to CP A5 and CP A6 patient groups.

FIG. 36B shows a graph of average Methionine breath test results for Child-Pugh A5 patients (N=105) that were sub-divided into 4 groups based on DSI score within ranges of 5-15, 15-25, 25-35, and 35-45. In general, patients in higher DSI score groups exhibited reduced average ¹³CO₂ exhalation score, regardless of CP class.

FIG. 36C shows a graph of average Methionine breath test results for Child-Pugh A6 patients (N=63) that were sub-divided into 4 groups based on DSI score within ranges of 5-15, 15-25, 25-35, and 35-45. In general, patients in higher DSI score groups exhibited reduced average ¹³CO₂ exhalation score, regardless of CP class.

FIG. 36D shows a graph of average methionine breath test results for Child-Pugh class B patients (N=15) that were sub-divided into 4 groups based on DSI score within ranges of 5-15, 15-25, 25-35, and 35-45. In general, patients in higher DSI score groups exhibited reduced average ¹³CO₂ exhalation score, regardless of CP class.

FIG. 37A shows a graph of average caffeine elimination rate for groups of patients divided by Child-Pugh scores A5, A6 and B. Patients in the CP B class exhibited decreased average caffeine elimination rate compared to CP A5 and CP A6 patient groups.

FIG. 37B shows a graph of average caffeine elimination rate for groups of patients (N=97) from Child-Pugh score A5 that were further sub-divided into 4 groups based on DSI score within ranges of 5-15, 15-25, 25-35, and 35-45. In general, patients in higher DSI score groups exhibited reduced caffeine elimination rate, regardless of CP class.

FIG. 37C shows a graph of average caffeine elimination rate for groups of patients (N=58) from Child-Pugh score A6 that were further sub-divided into 4 groups based on DSI score within ranges of 5-15, 15-25, 25-35, and 35-45. In general, patients in higher DSI score groups exhibited reduced caffeine elimination rate, regardless of CP class.

FIG. 37D shows a graph of average caffeine elimination rate for groups of patients (N=13) from Child-Pugh class B that were further sub-divided into 4 groups based on DSI score within ranges of 5-15, 15-25, 25-35, and 35-45. In general, patients in higher DSI score groups exhibited reduced caffeine elimination rate, regardless of CP class.

FIG. 38A shows a graph of average MEGX 15 min concentration following administration of lidocaine for three Child-Pugh score groups CP A5, CP A6, and CP B. Patients in the CP B class exhibited decreased average MEGX 15 min concentration compared to CP A5 and CP A6 patient groups.

FIG. 38B shows a graph of average MEGX 15 min concentration for Child-Pugh A5 patients (N=98) that were sub-divided into 4 groups based on DSI score within ranges of 5-15, 15-25, 25-35, and 35-45. In general, patients in different DSI score groups exhibited different avg. MEGX 15 minute concentrations.

FIG. 38C shows a graph of average MEGX 15 min concentration for Child-Pugh A6 patients (N=60) that were sub-divided into 4 groups based on DSI score within ranges of 5-15, 15-25, 25-35, and 35-45. In general, patients in different DSI score groups exhibited different avg. MEGX 15 minute concentrations

FIG. 38D shows a graph of average MEGX 15 min concentration for Child-Pugh class B patients (N=13) that were sub-divided into 4 groups based on DSI score within ranges of 5-15, 15-25, 25-35, and 35-45. In general, patients in different DSI score groups exhibited different avg. MEGX 15 minute concentrations.

FIG. 39A shows a graph of avg. galactose elimination capacity v Child-Pugh score for three groups: CP A5, CP A6, and CP B. Patients in the CP B class exhibited decreased average galactose elimination capacity compared to CP A5 and CP A6 patient groups.

FIG. 39B shows a graph of avg. galactose elimination capacity for Child-Pugh A5 patients (N=104) were sub-divided into 4 DSI score groups based on DSI score within ranges of 5-15, 15-25, 25-35, and 35-45. In general, patients in the higher DSI score groups exhibited decreased average galactose elimination capacity.

FIG. 39C shows a graph of avg. galactose elimination capacity for Child-Pugh A6 patients (N=64) that were sub-divided into 4 groups based on DSI score within ranges of 5-15, 15-25, 25-35, and 35-45. In general, patients in the higher DSI score groups exhibited decreased average galactose elimination capacity.

FIG. 39D shows a graph of average galactose elimination capacity for Child-Pugh class B patients (N=15) that were sub-divided into 4 groups based on DSI score within ranges of 5-15, 15-25, 25-35, and 35-45. In general, patients in higher DSI score groups exhibited reduced galactose elimination capacities, regardless of CP class and score.

FIG. 40A-E show a summary of the changes in PK of 5 diverse drugs divided by DSI score into groups of DSI 5-15, DSI 15-25, DSI 25-35, DSI 35-45 in each of antipyrine clearance, methionine breath test, caffeine elimination rate, lidocaine MEGX15 min concentration, and Galactose elimination. In general, patients exhibiting the highest DSI scores (35-45), exhibited the lowest PK average values for each of antipyrine clearance (FIG. 40A), methionine breath test (FIG. 40B), caffeine elimination (FIG. 40C), lidocaine MEGX15 min concentration (FIG. 40D), and galactose elimination capacity (FIG. 40E).

FIG. 41 shows a functional map of Indexed Hepatic Reserve in Fontan patients versus lean controls. Solid circles indicate HR values in individual Fontan patients (n=18), open circles show values in individual lean controls. Fontan patients 17 and 20 exhibit indexed HR of only about 50% and 51% respectively compared to lean healthy controls indicating poor liver function. Fontan patient 16 exhibits an indexed HR of about 100% compared to lean controls.

### DETAILED DESCRIPTION OF THE INVENTION

Improved methods for evaluating liver function in a patient are provided herein including rapidly and efficiently processing, detecting and quantifying distinguishable compounds from patient blood or serum samples.

A method is provided for estimating risk of experiencing a clinical event in 1 year for an individual patient having a chronic liver disease.

U.S. Pat. No. 8,613,904, Everson et al., discloses methods for evaluating liver function in a patient comprising obtaining patient serum samples following administration of two distinguishable stable isotope labeled cholate compounds, laborious sample processing and analysis of patient serum samples utilizing GC-MS.

U.S. Pat. No. 8,778,299, Everson, discloses methods for evaluating liver function comprising obtaining patient serum samples following administration of two distinguishable stable isotope labeled cholate compounds, processing and analysis of patient serum samples utilizing HPLC-MS.

U.S. Pat. No. 9,091,701, Everson, discloses methods for determining liver function and obtaining a Disease Severity Index (DSI) value in a patient comprising obtaining patient serum samples following administration of two distinguishable stable isotope labeled cholate compounds, processing and analysis of patient serum samples utilizing HPLC-MS.

Improved methods are provided herein for rapidly processing and efficiently processing patient samples, extracting and quantifying distinguishable compounds in patient blood or serum samples. For example, compared to the prior art procedures, methods are provided wherein the unlabeled endogenous cholic acid is now quantified in each individual sample rather than only the baseline samples. The original multi-step extraction procedure including a combination of solid phase extraction, liquid-liquid extraction, evaporation and reconstitution is replaced herein by an automated online extraction procedure. Detection and quantification is based on analyte ion transitions in multiple reaction mode and MS/MS is employed rather than MS using selected ion monitoring. The improvements in analysis allow for utilization of a much smaller blood or serum sample amount at each time point. Therefore, additional types of blood sample collection methods may be successfully utilized.

In the present disclosure, mass spectrometry (MS or MS/MS) may be employed alone or optionally in combination with a chromatography technique to quantify the distinguishable compound in the sample.

Advantages of the methods of the present disclosure for analysis allow for a much smaller blood or serum sample collection volume than previously required for use in liver function tests such as the dual cholate SHUNT test, FLOW test, portal HFR, systemic HFR, STAT test, DSI test, RCA20, cholate elimination rate, algebraic Hepatic Reserve, or indexed Hepatic Reserve tests. As low as 5 microliters of blood per sample may be utilized. Each of these liver function tests requires administration of one or more distinguishable compounds to a subject and blood or serum sample collection at one or more, two or more, three or more, four or more, or five or more time points following oral and/or intravenous administration of the one or more distinguishable compounds.

Distinguishable Compounds. In some embodiments, one or more distinguishable compounds may be administered to a subject by oral and/or intravenous administration. One or more blood or serum samples is obtained from the subject less than 3 hours after administration. The blood or serum sample(s) are processed as provided herein, and the one or more distinguishable compounds are quantified in each sample, as provided herein. Any safely orally administered distinguishable compound may be employed having the following characteristics: about 100% absorption following oral administration, high hepatic extraction (>50%, >60%, >70%, >80%, or >90% in first pass through the liver of a healthy subject), and removal from the blood or plasma exclusively by the liver. The distinguishable compound for measurement of portal flow can be an endogenous compound or a xenobiotic.

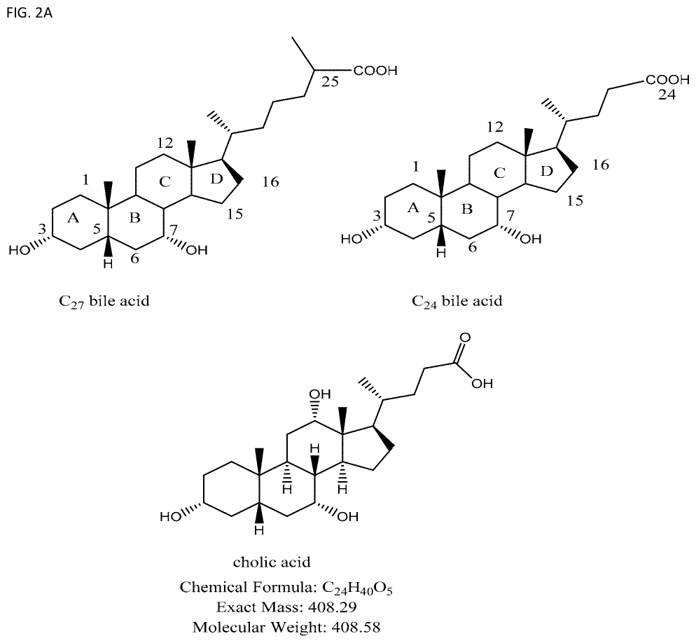

In some embodiments, the distinguishable compound may be a distinguishable bile acid. Human bile acids are generally C24 molecules comprised of a C19 cyclopentanophenanthrene (steroid) nucleus and a carboxylate side-chain. C27 molecules also exist. Basic structures and ring numbering systems for C24 bile acids and C27 bile acids are shown in FIG. 2A. A representative C24 bile acid:cholic acid is also shown. The structural diversities of human bile acids come from several factors: (1) AB ring fusion stereochemistry, cis/5β-H or trans/5α-H; (2) sites of hydroxylation which can occur at C3, C6, C7 and C12; (3) conjugation of glycine or taurine at the C24-carboxyl group; and (4) dehydrogenation and epimerization of hydroxyl groups. The first three factors are mainly derived from host metabolism and the last one may be attributed to gut microbial biotransformation. Considering only the host synthesis and metabolism of bile acids, 48 possible BA species with oxidation sites at C3, C6, C7 and/or C12 can be produced. This number increases to 384 if the entire host-gut microbial co-metabolism is considered. Lan et al., 2016, Anal Chem 88(14):7041-7048.

The distinguishable bile acid may be an endogenous bile acid, a bile acid conjugate, labeled bile acid, isotopically labeled bile acid, or a bile acid analog. The distinguishable bile acid may be, for example, dehydrolithocholic acid (dehydroLCA), lithocholic acid (LCA), isodeoxycholic acid (isoDCA), isolithocholic acid (isoLCA), allolithocholic acid (alloLCA), glycolithocholic acid (GLCA), deoxycholic acid (DCA), chenodeoxycholic acid (CDCA), taurolithocholic acid (TLCA), apocholic acid (apoCA), 23-nordeoxycholic acid (nor-DCA), 12-ketolithocholic acid (12-ketoLCA), 7-ketolithocholic acid (7-ketoLCA), 6,7-diketolithocholic acid (6,7-diketoLCA), glycodeoxycholic acid (GDCA), 6-keto-lithocholic acid (6-ketoLCA), glycochenodeoxycholic acid (GCDCA), hyodeoxycholic acid (HDCA), ursodeoxycholic acid (UDCA), cholic acid (CA), taurodeoxycholic acid (TDCA), allocholic acid (ACA), beta-hyodeoxycholic acid (beta-HDCA), murocholic acid (muroCA), hyocholic acid (HCA), 12-dehydrocholic acid (12-DHCA), beta-muricholic acid (beta-MCA), norcholic acid (norCA), 7-ketodeoxycholic acid (7-ketoDCA), glycocholic acid (GCA), alpha-muricholic acid (alpha-MCA), glycohyodeoxycholic acid (GHDCA), 3beta-cholic acid (betaCA), glycoursodeoxycholic acid (GHCA), omega-muricholic acid (omegaMCA), taurocholic acid (TCA), glycohyocholic acid (GHCA), taurohyodeoxycholic acid (THDCA), 7,12-diketolithocholic acid (7,12-diketoLCA), dehydrocholic acid (DHCA), ursocholic acid (UCA), taurohyocholic acid (THCA), tauro beta-muricholic acid (TbetaMCA), tauro alpha-muricholic acid (TalphaMCA), glycodehydrocholic acid (GDHCA), tauro omega-murichlic acid (TomegaMCA), taurohydrocholic acid (TDHCA), or an isotopically labeled derivative, or analog or epimer thereof.

For example, the distinguishable bile acid may be an endogenous bile acid or bile acid conjugate. The distinguishable compound may be a distinguishable cholate compound. Cholate compounds may be selected from any of the following labeled compounds: cholic acid, any glycine conjugate of cholic acid, any taurine conjugate of cholic acid; chenodeoxycholic acid, any glycine conjugate of chenodeoxycholic acid, any taurine conjugate of chenodeoxycholic acid; deoxycholic acid, any glycine conjugate of deoxycholic acid, any taurine conjugate of deoxycholic acid; or lithocholic acid, or any glycine conjugate or taurine conjugate thereof.

Cholates occur naturally and are not known to have any deleterious or adverse effects when given intravenously or orally in the doses used in HQ tests. The serum cholate concentrations that are achieved by either the intravenous or oral doses are similar to the serum concentrations of bile acids that occur after the ingestion of a fatty meal. Because cholates are naturally occurring with a pool size in humans of 1 to 5 g, the 20 and 40 mg doses of labeled cholates used herein are unlikely to be harmful.

The distinguishable bile acid may be a labeled bile acid. Labeled bile acids may include, for example, radiolabeled bile acids, non-radiolabeled stable isotope labeled bile acids, or fluorescent-labeled bile acids. Labeled bile acids may include fluorescein lisicol trisodium salt (NRL-972 trisodium salt), fluorescein lisicol (NRL-972), (18)F-chenodeoxycholic acid, cholyl-Lys-fluorescein (CLF), fluorescein isothiocyanate glycocholate (FITC-GC), lithocholyl-lysyl-fluorescein (LLF), and dansyl-labeled cholic acid.

The distinguishable bile acid may be a stable isotope labeled bile acid taurine conjugate, for example, selected from taurochenodeoxycholic acid, sodium salt (taurine-2,2,3,4,4,6,6,7,8-D9-CDCA); taurochenodeoxycholic acid, sodium salt (taurine-2,2,4,4-D4-CDCA); taurocholic acid, sodium salt (taurine-13C2-CA); taurocholic acid, sodium salt (taurine-2,2,4,4-D4-CA); taurodeoxycholic acid, sodium salt (taurine-2,2,4,4,11,11-D6-DCA); taurodeoxycholic acid, sodium salt (taurine-2,2,4,4-D4-DCA); tauroursodeoxycholic acid, sodium salt (taurine-2,2,4,4-D4-UDCA); tauroursodeoxycholic acid, sodium salt (taurine-12C2-UDCA).

The distinguishable bile acid may be a stable isotope labeled bile acid glycine conjugate, for example, selected from glycochenodeoxycholic acid (glycine-2,2,3,4,4,6,6,7,8-D9-CDCA), glycochenodeoxycholic acid (glycine-2,2,4,4-D4-CDCA), glycocholic acid (glycine-2,2,4,4-D4-CA), glycocholic acid (glycine-1-13C-CA), glycodeoxycholic acid (glycine-2,2,4,4,11,11-D6-DCA), glycodeoxycholic acid (glycine-2,2,4,4-D4-DCA), glycolithocholic acid (glycine-2,2,4,4-D4-LCA), glycoursodeoxycholic acid (glycine-2,2,4,4-D4-UDCA), and glycoursodeoxycholic acid (glycine-13C2-UDCA).

The distinguishable bile acid may be a stable isotope labeled primary bile acid, for example, selected from alpha-muricholic acid (e.g., 2,2,3,4,4-D5-αMCA), beta-muricholic acid (e.g., 2,2,3,4,4-D5-βMCA), chenodeoxycholic acid (e.g., 2,2,3,4,4,6,6,7,8-D9-CDCA), chenodeoxycholic acid (e.g., 2,2,3,4,4-D5-CDCA), chenodeoxycholic acid (e.g., 2,2,4,4-D4-CDCA), chenodeoxycholic acid (e.g., 24-13C-CDCA), gamma-muricholic acid (e.g., 2,2,3,4,4-D5-γMCA), omega-muricholic acid (e.g., 2,2,3,4,4-D5-ωMCA), and cholic acid (e.g., 2,2,3,4,4-D5-CA; 2,2,4,4-D4-CA; 3,6,6,7,8,11,11,12-D8-CA; and 24-13C-CA).

The distinguishable bile acid may be a stable isotope labeled secondary bile acid, for example, selected from deoxycholic acid (2,2,4,4,11,11-D6-DCA), deoxycholic acid (2,2,4,4-D4-DCA), deoxycholic acid (24-13C-DCA), glycoursodeoxycholic acid (glycine-13C2-UDCA), lithocholic acid (2,2,4,4-D4-LCA), tauroursodeoxycholic acid, sodium salt (taurine-13C2-UDCA), ursodeoxycholic acid (2,2,4,4-D4-UDCA), and ursodeoxycholic acid (24-13C-UDCA).

The distinguishable compound may be a bile acid analog or epimer. The term “analog”, refers to a structural analog, also known as a chemical analog, having a structure similar to that of another one, but differing from it in respect of one or more components. For example, a bile acid analog may be a synthetic or semi-synthetic bile acid analog. The bile acid analog may be obeticholic acid, also known as 6alpha-ethyl-chenodeoxycholic acid, or 3alpha,5beta,6alpha,7alpha)-6-ethyl-3,7-dihydroxy-cholan-24-oic acid. Obeticholic acid (OCA) is an analog of chenodeoxycholic acid differing by addition of an ethyl moiety rather than a hydrogen residue in the 6alpha position. Chenodeoxycholic acid is an active physiological ligand for the Farnesoid X receptor (FXR) which is involved in many physiological and pathophysiological processes. OCA is known to be an FXR agonist.

The distinguishable compound may be an FXR agonist, for example, obeticholic acid (OCA), chenodeoxycholic acid, or ethyl-3,7,23-trihydroxy-24-nor-5-cholan-23-sulfate sodium salt).

The bile acid analog may be ursodeoxycholic acid (UDCA), also known as ursodiol. Ursodeoxycholic acid is an epimer of chenodeoxycholic acid. Ursodeoxycholic acid is considered to be a secondary bile acid, which are metabolic products of intestinal bacteria. UDCA is known to be useful in the treatment of primary biliary cholangitis, reduction in gallstone formation, to improve bile flow, and after bariatric surgery to prevent cholelithiasis due to rapid weight loss with biliary cholesterol oversaturation.

The bile acid analog may be hyodeoxycholic acid (HDCA), also known as 3a, 6α-dihydroxy-5β-cholan-24-oic acid. Hyodeoxycholic acid differs from deoxycholic acid in that the position of a hydroxyl group. 6a-hydroxyl is in the 12-position in the former. HDCA is known as a secondary bile acid, a metabolic by product of intestinal bacteria.

In various aspects, any bile acid or bile acid conjugate may be in the form of a physiologically acceptable salt, e.g., the sodium salt of cholic acid. In one aspect, the term cholic acid refers to the sodium salt of cholic acid. Cholic acid (cholate) is the distinguishable cholate compound in some preferred embodiments. As used herein, the terms cholate compound, cholate and cholic acid are used interchangeably.

Xenobiotics that could be administered orally and also have high first pass hepatic elimination could include, but are not limited to, propanolol, nitroglycerin or derivative of nitroglycerin, or galactose and related compounds.

In some embodiments, the distinguishable compound is propranolol. Propranolol is a nonselective β blocker and has been shown to be effective for the prevention of variceal bleeding and rebleeding and is widely used as the pharmacotherapy for the treatment of portal hypertension in patients with cirrhosis. (Suk et al. 2007, Effect of propranolol on portal pressure and systemic hemodynamics in patients with liver cirrhosis and portal hypertension: a prospective study. Gut and Liver 1 (2): 159-164). Propranolol is almost entirely cleared by the liver. It has been demonstrated that total (+)-propranolol plasma clearance constitutes a good estimate of hepatic blood flow in patients with normal liver function. (Weiss et al., 1978 (+)-Propranolol clearance, an estimation of hepatic blood flow in man, Br. J. Clin. Pharmacol. 5: 457-460).

In other embodiments, the distinguishable compound is isosorbide 5-mononitrate. This compound can be administered orally and detected in plasma, for example, by HPLC-EIMS. (Sun et al., High performance liquid chromatography-electrospray ionization mass spectrometric determination of isosorbide 5-mononitrate in human plasma, J. Chromatogr. B Analyt. Technol. Biomed. Sci. 2007 Feb. 1; 846(1-2):323-8).

In some embodiments, the distinguishable compound is galactose. Galactose elimination capacity (GEC) has been used as an index of residual hepatic function. Galactose in the GEC test typically is administered intravenously at a dose of 0.5 mg/kg and venous samples taken every 5 min between 20 and 60 minutes. The clearance of galactose is decreased in individuals with chronic liver disease and cirrhosis. The fact that this carbohydrate has a high extraction ratio, however, makes the metabolism of galactose dependent on liver blood flow and hepatic functional mass. (Tygstrup N, Determination of the hepatic elimination capacity (Lm) of galactose by a single injection, Scand J Lab Clin invest, 18 Suppl 92, 1966, 118-126). The carbohydrate galactose is metabolized almost exclusively in the liver, and the elimination rate at blood concentrations high enough to yield near-saturated enzymatic conversion, the GEC is used as a quantitative measure of the metabolic capacity of the liver. One study has shown that among patients with a newly-diagnosed cirrhosis and a decreased GEC, the GEC was a strong predictor of mortality. (Jepsen et al, 2009, The galactose elimination capacity and mortality in 781 Danish patients with newly-diagnosed liver cirrhosis: a cohort study. BMC Gastroenterol. 2009, 9:50).

In certain embodiments, one or more differentiable isotopes are incorporated into the selected distinguishable compound in order to be utilized to assess hepatic function. The differentiable isotope can be either a radioactive or a stable isotope incorporated into the distinguishable compound. Stable (¹³C, ²H, ¹⁵N, ¹⁸O) or radioactive isotopes (¹⁴C, ³H, Tc-99m) can be used. Advantages of stable isotopes are the lack of exposure to radioactivity, natural abundance, and the specificity of the analyses used for test compound identification (mass determination by mass spectrometry). Stable isotopically labeled compounds are commercially available. The distinguishable compound may be a stable isotope labeled bile acid.

Stable isotope labeled bile acids may be selected from, for example, lithocholic acid-2,2,4,4-D4 (LCA-D4), ursodeoxycholic acid-2,2,4,4-D4 (UDCA-D4), deoxycholic acid-2,2,4,4-D4 (DCA-D4), cholic acid-2,2,4,4-D4 (D4-CA; CA-D4), 24-¹³C-cholic acid (^{D}C-CA), 2,2,3,4,4-d₅ cholic acid (D₅-CA), glycochenodeoxycholic acid-2,2,4,4-D4 (GCDCA-D4), glycodeoxycholic acid-2,2,4,4-D4 (GDCA-D4), glycocholic acid-2,2,4,4-D4 (GCA-D4), deoxycholic acid-24-13C (DCA-24-13C), which are commercially available from, e.g., Sigma-Aldrich, IsoSciences (King of Prussia, Pa.), CDN Isotopes, and Cambridge Isotope Laboratories, Inc.

In some embodiments, the distinguishable compound for oral administration can be any distinguishable cholate compound that is distinguishable analytically from an endogenous cholic acid. In one aspect, the distinguishable cholate compound is selected from any isotopically labeled cholic acid compound known in the art. Distinguishable cholate compounds used in any one of these assays might be labeled with either stable (¹³C, ²H, ¹⁸O) or radioactive (¹⁴C, ³H) isotopes. Distinguishable cholate compounds can be purchased (for example CDN Isotopes Inc., Quebec, CA). In a preferred aspect, the distinguishable cholate is selected from any known safe, non-radioactive stable isotope of cholic acid. In one specific aspect, the distinguishable cholate compound is 2,2,4,4-²H cholic acid, also known as cholic-acid-2,2,4,4-d₄ (D4-CA). In another specific aspect, the distinguishable cholate compound is 24-¹³C cholic acid, also known as cholic acid-24-¹³C (¹³C-CA). In another specific aspect, the distinguishable compound is 2,2,3,4,4-²H cholic acid, also known as cholic acid-2,2,3,4,4-d₅ (D₅-CA).

In other embodiments, the distinguishable compound may be an unlabeled endogenous compound, such as unlabeled cholate. In the aspect using an unlabeled endogenous compound, the oral test dose is sufficiently great, for example 2.5-7.5 mg/kg cholate, for the resulting serum concentration to be distinguishable above the baseline serum concentration of that endogenous compound.

The platform for detecting and measuring the distinguishable compound in the blood sample from the subject may be dependent on the type of administered distinguishable compound. For stable isotopes, the concentration of the distinguishable compound in a blood sample can be measured by any known method, e.g., previously gas chromatography/mass spectroscopy (GC-MS) or liquid chromatography/mass spectroscopy (LC-MS) have been employed. As provided herein, the distinguishable compound may be detected and quantified from a blood or serum sample using MS, MS-MS, or LC-MS/MS using multiple reaction monitoring (MRM). For radiolabeled test compounds, e.g., scintillation spectroscopy can be employed. For analysis of unlabeled compounds, e.g., autoanalyzers, luminescence, or ELISA can be employed. It is further contemplated that strip tests with a color developer sensitive directly or indirectly to the presence and quantity of test compound can be employed for use in a home test or a point of care test.

Portal Blood Flow

Portal blood flow has been found to be a key parameter for liver assessment. The liver receives ˜75% of its blood through the portal vein which brings in the nutrients for processing and deleterious compounds for detoxification. This low blood pressure system is sensitive to the earliest disruption of the microvasculature so that the early stages of CLD can be detected by decreased portal flow and increased shunting before any other physiological impacts. The high pressure hepatic systemic blood flow is decreased less and only later in the disease process. Unlike biopsy, which samples only 1/50,000^{th} of the liver, the portal flow is a measure of the entire organ. As disease progresses there is increasing disruption of the microvasculature architecture and increasing impairment of portal flow which causes the major manifestations of advanced chronic liver disease (CLD). Impaired flow causes ascites, portal hypertension, and esophageal varices. Impaired flow causes increased shunting of toxins which leads to hepatic encephalopathy.

Cholate is a unique probe of the portal blood flow and the hepatic systemic flow. Many liver tests have attempted to use the clearance of oral or IV compounds but only cholate has succeeded in assessing early and late stage CLD. Other oral compounds are absorbed at various sites along the GI tract and do not target the portal circulation. Other compounds are taken up by nonspecific transporters. Oral cholate is specifically absorbed by the terminal ileum epithelial cells via the high affinity ileal Na⁺-dependent bile salt transporter (ISBT) and is effluxed by MRP3 transporters directly into the portal blood flow (Trauner and Boyer, 2003, Bile salt transporters: Molecular characterization, function, and regulation. Physiol Rev. 83: 633-671). A different set of high affinity transporters including the Na⁺/taurocholate cotransporter (NTCP) and organic anion transporting proteins (OATPs) then takes it up into hepatocytes with highly efficient first pass extraction (Trauner and Boyer, 2003, infra) so that any cholate that escapes extraction is a direct measure of the portal flow. Once intracellular, it is rapidly conjugated to glycine and taurine so that the unconjugated form does not then re-appear in the intrahepatic circulation, which would confuse the pharmacokinetics. Other unconjugated bile salts such as deoxycholate and chenodeoxycholate would behave similarly but they are much stronger solubilizing agents and would not be as safe to administer. Patient safety may be ensured by using a stable isotope labeled endogenous compound avoiding the risks of xenobiotic or radiation exposure. All the proteins and systems involved are highly conserved and essential so that the pharmacokinetics of cholate are consistent between individuals and not affected by gender, age, or genetic makeup, or by diet or concomitant medications.

The portal blood flow can be non-invasively and accurately quantified by exploiting the unique physiology of the endogenous bile acid, cholate, which can be labeled, for example, with safe non-radioactive stable isotopes. Highly conserved enteric transporters (ISBT, MRP3) specifically target oral cholate to the portal circulation. Highly conserved hepatic transporters (NTCP, OATPs) clear cholate from the portal and systemic circulation. Therefore, noninvasive quantitative assessment of the portal circulation can be performed by administration to a patient of a distinguishable cholate compound and assessment of a level of the distinguishable cholate compound in blood samples drawn at various multiple time points to determine an oral clearance curve. The FLOW (portal HFR) test accurately measures the portal blood flow from a minimum of 5 blood samples taken over a period of 90 minutes after an oral dose of deuterated-cholate.

A major study of almost 300 CHC patients, portal flow measured by cholate testing was superior in predicting clinical outcomes to the current gold standard of fibrosis measured by biopsy (Everson et al., 2011). In the Early CHC study impairment of the portal flow and increased shunting measured by cholate testing was the earliest detectable pathophysiology. These results have led to a new understanding of CLD that it is the disruption of hepatic microvasculature and not fibrosis per se that is deleterious. This microvasculature disruption impairs the portal blood flow which can be non-invasively and accurately quantified by exploiting the unique physiology of the endogenous bile acid, cholate.

Portal-Systemic Shunting

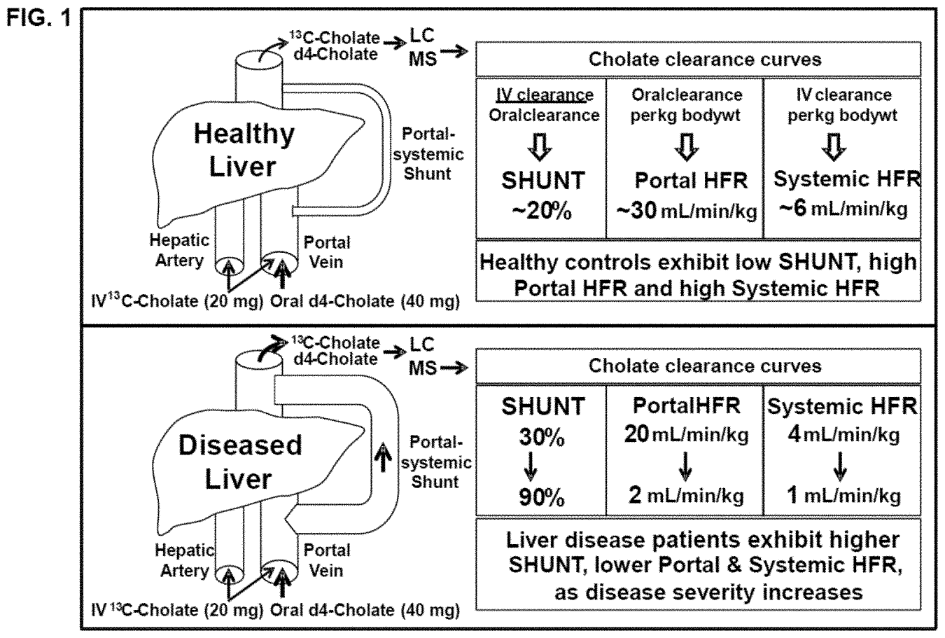

As shown in FIG. 1, oral cholate is taken up by specific enteric transporters directly into the portal vein and removed by hepatic transporters in its first-pass through the liver. IV cholate distributes systemically and is extracted by both the hepatic artery and portal vein.

In health, the orally administered deuterated cholate is delivered to the liver via the portal circulation. Its clearance is a measure of the portal circulation—hence the designation Portal HFR. The intravenously administered ¹³C-cholate is delivered to the liver via both hepatic arterial and portal venous circulations—hence the designation Systemic HFR. SHUNT is a ratio of Systemic HFR to Portal HFR. The normal ranges for these tests are shown in the top panels.

With disease—SHUNT increases and both portal and systemic HFR decrease—as shown in the bottom panels.

For example, normal healthy controls typically exhibit SHUNT (IV cholate clearance/oral cholate clearance) of about 20%, portal HFR (oral cholate clearance per kg body weight) of about 30 mL/min/kg, and systemic HFR (intravenous cholate clearance per kg body weight) of about 6 mL/min/kg. Liver disease patients typically exhibit higher SHUNT values of between from about 30% to 90%. Liver disease patients typically exhibit lower portal HFR of from about 20 mL/min/kg to about 2 mL/min/kg. Liver disease patients typically exhibit lower systemic HFR of from about 4 mL/min/kg to about 1 mL/min/kg.

In the diseased liver, as more blood escapes extraction by intra- and extra-hepatic shunting to the systemic circulation, the SHUNT increases, HFR or portal flow decreases, and STAT increases. In a normal control subject, the effective portal blood flow (portal HFR, FLOW) is high in a healthy liver due to low vascular resistance. Portal-systemic shunting (SHUNT) is minimal. Oral cholate at 60 min (STAT) is low. For example, in a healthy control FLOW=37 mL min⁻¹ kg⁻¹, SHUNT=18% and STAT=0.2 μM. However, in a subject with liver disease, inflammation, fibrosis, and increased vascular resistance reduce the effective portal blood flow (FLOW). Portal-systemic shunting (SHUNT) is increased. Oral cholate at 60 min (STAT) is high. For example in a CHC F2 patient, FLOW=9 mL min⁻¹ kg⁻¹, SHUNT=35% and STAT=1.6 μM.

Portal HFR (FLOW) and SHUNT tests may be used to determine portal blood flow and liver function, for example, in healthy controls and patients with a chronic liver disease, such as chronic hepatitis C. SHUNT and FLOW tests employing measuring an orally and/or intravenously administered distinguishable compounds and measuring the distinguishable compounds in a multiplicity of blood or serum samples by GC-MS or HPLC-MS are disclosed in U.S. Pat. Nos. 8,613,904 and 8,778,299, which are each incorporated herein by reference.

The STAT test was developed as a screening test and is utilized to estimate portal blood flow and screen large populations for detection of patients with chronic liver disease, including chronic hepatitis C, PSC and NAFLD. The STAT test was developed to estimate portal blood flow and screen large populations for detection of patients with chronic liver disease, including chronic hepatitis C, PSC and NAFLD. The relationship of STAT to prior art methods of determining clearance of cholate from the portal circulation, specifically the FLOW and SHUNT tests, has been validated using a large cohort of patients with chronic hepatitis C. Methods for preforming the STAT test comprising measuring the distinguishable compound in a single blood or serum sample using HLPC-MS are disclosed in U.S. Pat. No. 8,961,925, which is incorporated herein by reference.

In some embodiments, the STAT test value in a subject may be obtained by a method comprising (a) receiving a single blood or serum sample collected from the subject having following oral administration of a dose of a distinguishable compound (doseₒᵣₐₗ) to the subject, wherein the sample has been collected from the subject at a specific time point within about 20-180 minutes after administration; and (b) measuring concentration of the distinguishable cholate compound in the sample by MS, MS/MS, or LC-MS/MS with MRM.

In some embodiments, the single blood or serum sample in the STAT test is collected at one single time point selected from about 20, 25, 30, 35, 40, 45, 50, 55, 50, 65, 70, 75, 80, 85, 90, 95, 100, 105, 110, 115, 120, 125, 130, 135, 140, 145, 150, 155, 160, 165, 170, 175, or 180 minutes, or any time point in between, after oral administration of the distinguishable cholate compound.

In some embodiments, the single blood or serum sample in the STAT test is collected at one time point selected from about 45, about 60 or about 90 minutes after oral administration of the distinguishable cholate compound.

In some embodiments, the single blood or serum sample is collected at about 60 minutes after oral administration of the distinguishable cholate compound.

In some embodiments, the single blood or serum sample is collected at about 45 minutes after oral administration of the distinguishable cholate compound.

In some embodiments, the single blood or serum sample is collected at about 90 minutes after oral administration of the distinguishable cholate compound.

In some embodiments, a method is provided for determining a STAT test value in a subject having or suspected of having or developing a chronic liver disease, comprising (a) receiving a single blood or serum sample collected from the subject following oral administration of a dose of a distinguishable compound (doseₒᵣₐₗ) to the subject, wherein the sample has been collected from the subject at a specific time point within about 20-180 minutes after administration; and (b) measuring concentration of the distinguishable cholate compound in the sample by MS, MS-MS with MRM, or LC-MS/MS with MRM.

In some embodiments, a method is provided for determining a portal HFR value in a patient comprising (a) receiving a plurality of blood or serum samples collected from a patient having or at risk of a chronic liver disease, following oral administration of a dose of a distinguishable cholate (doseₒᵣₐₗ) to the patient, wherein the samples have been collected from the patient over intervals spanning a period of time after administration; (b) measuring concentration of the distinguishable cholate in each sample by a method comprising MS, MS-MS with MRM, or LC-MS/MS with MRM; (c) generating an individualized oral clearance curve from the concentration of the distinguishable cholate in each sample comprising using a computer algorithm curve fitting to a model distinguishable cholate clearance curve; (d) computing the area under the individualized oral clearance curve (AUC) (mg/mL/min) and dividing the dose (in mg) by AUC of the orally administered stable isotope labeled cholic acid to obtain the oral cholate clearance in the patient; and (e) dividing the oral cholate clearance by the weight of the patient in kg to obtain the portal HFR value in the patient (mL/min/kg).

Test Outputs. Cholate concentrations (endogenous unlabeled CA, 13C-CA, and d4-CA) may be measured from the timed serum samples (collected 0, 5, 20, 45, 60, and 90 minutes after oral and i.v. coadministration) and concentrations of each labeled cholate as a function of time may be modeled as a spline curve in order to calculate the area under curve (AUC). The cholate SHUNT test parameters may include: DSI, indexed Hepatic Reserve, algebraic Hepatic Reserve, RISK-ACE, SHUNT %, RCA20, Systemic HFR, Portal HFR, cholate elimination rate, and volume of distribution.

Briefly, DSI is a score without units representing a quantitative measurement of liver function. DSI (Disease Severity Index) is a score that is a function of the sum of cholate clearances from systemic and portal circulations adjusted to disease severity ranging from healthy persons to end-stage liver disease. Hepatic Reserve represents a percentage of maximum hepatic functional capacity measured by DSI normalized to the DSI range in persons of lean body mass. Individual Risk Score for Annual Clinical Events (RISK-ACE) may be based upon baseline DSI (Model A) and also baseline DSI plus the ΔDSI that occurred over 2 years (Model D) in an HCV population with approximately 25% experiencing clinical event over a maximum of 8.7 years of followup. SHUNT % represents a quantitative measurement of portal-systemic shunting. SHUNT % is a measurement of the percentage of spillover of the orally administered d4-cholate. The first-pass hepatic elimination of cholate in percent of orally administered cholate is defined as (100%−SHUNT). Systemic HFR, mL min⁻¹ kg⁻¹ represents a model independent clearance of intravenously injected 13C-cholate, adjusted for body weight, and calculated from dose/AUC. Portal HFR, mL min⁻¹ kg⁻¹ represents a model independent apparent clearance of orally administered d4-cholate, adjusted for body weight, and calculated from dose/AUC. Cholate Elimination Rate, kₑₗᵢₘ min⁻¹ may be expressed as the first phase of elimination of the intravenously administered 13C-cholate, calculation from Ln/linear regression of [13C-cholate] versus time (using only the 5- and 20-minute time points). Intravenously administered 13C-cholate is rapidly delivered to the liver via the hepatic artery. In contrast, the same 13C-cholate slowly transits to the liver via the portal vein due to the capacitance of the splanchnic vascular bed. Thus, the first phase of cholate elimination is more dependent upon clearance from the hepatic artery than from portal vein. The Volume of distribution, V_{d}, L kg⁻¹: The body's volume into which cholate is distributed. This is calculated from the intercept on the Y axis of the Ln/linear regression of [13C-cholate] versus time (using only the 5- and 20-min time points).

In some embodiments, a method is provided for determining systemic HFR value in a patient may be determined by a method comprising (a) receiving a plurality of blood or serum samples collected from the patient having or at risk of a chronic liver disease, following intravenous administration of a dose of a distinguishable cholate (doseₒᵣₐₗ) to the patient, wherein the samples have been collected from the patient over intervals spanning a period of time after administration; (b) measuring concentration of the distinguishable cholate in each sample by a method comprising MS, MS-MS with MRM, or LC-MS/MS with MRM; (c) generating an individualized intravenous clearance curve from the concentration of the distinguishable cholate in each sample comprising using a computer algorithm curve fitting to a model distinguishable cholate clearance curve; (d) computing the area under the individualized systemic clearance curve (AUC) (mg/mL/min) and dividing the dose (in mg) by AUC of the intravenously administered stable isotope labeled cholic acid to obtain the intravenous cholate clearance in the patient; and (e) dividing the intravenous cholate clearance by the weight of the patient in kg to obtain the systemic HFR value in the patient (mL/min/kg).

In some embodiments, a method is provided for determining a hepatic SHUNT value in a subject having or suspected of having or at risk of a hepatic disorder or chronic liver disease, comprising: (a) obtaining a multiplicity of blood or serum samples collected from the subject over intervals for a period of less than 3 hours after the subject had been orally administered a first distinguishable compound and simultaneously intravenously administered a second distinguishable compound; (b) quantifying the first and the second distinguishable compounds in the samples by the method comprising MS, MS-MS with MRM, or LC-MS/MS with MRM; (c) calculating the hepatic shunt in the subject using the formula:

AUCₒᵣₐₗ/AUCᵢᵥ×Doseᵢᵥ/Doseₒᵣₐₗ×100%;

wherein AUCₒᵣₐₗ is the area under the curve of the serum concentrations of the first distinguishable compound and AUC is the area under the curve of the second distinguishable compound; and (d) comparing the hepatic shunt in the subject to a shunt cutoff value or cutoffs of values established from a known patient population or within the subject over time. The hepatic shunt in the subject compared to shunt cutoff value or cutoffs of values, or in the subject over time, is an indicator of relative hepatic function of the subject.

In some embodiments, the estimated hepatic blood flow (HBF) in a patient may be calculated with the following equation:

HBF=(Cholate clearance after intravenous administration)/[1−(SHUNT/100))×(1−(Hematocrit %/100))].

Previously, human studies demonstrated the clinical utility of FLOW and SHUNT testing in chronic hepatitis C (CHC). A number of new liver tests have been proposed over the years but there have been few studies to directly compare their efficacy and actual clinical utility. A very large multicenter HALT-C trial was conducted whose main objective was to determine the efficacy of long term hepatitis C virus suppression but which also included an ancillary study to evaluate a battery of new quantitative liver function tests. (Everson et al., 2009. Quantitative tests of liver function measure hepatic improvement after sustained virological response: Results from the HALT-C trial. Aliment Pharmacol Ther. 29: 589-601). Nearly 300 patients with advanced (Ishak F2-6) but compensated CLD were tested. Another Early CHC study compared these tests in 25 healthy controls and 23 early stage (Ishak F1-2) CHC patients in order to examine the entire spectrum of this CLD. The liver's metabolic capacity was assessed using caffeine, antipyrine, lidocaine, and galactose tests. All these activities were reduced in patients with cirrhosis, but none were different in early stage CHC patients compared to healthy controls. (Everson et al., 2008. The spectrum of hepatic functional impairment in compensated chronic hepatitis c: Results from the hepatitis c anti-viral long-term treatment against cirrhosis trial. Aliment Pharmacol Ther. 27: 798-809). These results suggest that metabolic capacity is maintained until there is significant loss of functional parenchyma in later stage CLD. In HALT-C the patients were tested serially every 2 years and followed to monitor outcomes. FLOW, using a cutoff of <9.5 ml/min/kg, was superior to the other tests in predicting clinical outcomes with the highest sensitivity, specificity, positive predictive value (PPV), negative predictive value (NPV) and the best performance by ROC analysis (Quantitative liver function tests improve the prediction of clinical outcomes in chronic hepatitis C: results from the Hepatitis C Antiviral Long-term Treatment Against Cirrhosis Trial, Everson et al., Hepatology, 2012 April; 55(4):1019-29). FLOW had a higher ROC c statistic (0.84) relative to SHUNT (0.79). The improvement after SVR was more significant for FLOW (p=0.0002) than for SHUNT (p=0.0003) (Everson et al., 2009, infra). In the Early CHC study, FLOW decreased from 34±14 ml/min/kg (mean±SD) in controls to 23±10 ml/min/kg in early CHC (p<0.002) but the increase in SHUNT (20±6% in controls vs, 31±14% in early CHC patients p<0.0002) was more statistically significant. None of the other tests could distinguish early stage CHC patients from healthy controls. These results suggest that SHUNT and FLOW outperform other functional tests in detecting early liver disease, tracking patients, and predicting clinical outcomes.

In typical embodiments, concentrations of both oral and IV cholates are measured at 5 different times within 90 minutes of administration and clearances are calculated. The IV clearance over the oral clearance is the portal-systemic SHUNT fraction. The oral clearance per kilogram of body weight represents the Portal Hepatic Filtration Rate (Portal HFR, FLOW), or amount of portal blood delivery. STAT is the concentration of oral cholate at 60 minutes, and may be used to accurately estimate the portal HFR.

The SHUNT test non-invasively and accurately measures the portal blood flow following oral administration of a distinguishable cholate compound and also measures the systemic hepatic blood flow following intravenous co-administration of a second distinguishable cholate compound. Therefore the SHUNT test can be used to determine the amount of portal-systemic shunting. In some embodiments, an IV dose of ¹³C-cholate is administered concurrently with an oral dose of deuterated-cholate and a minimum of 5 blood samples taken over a period of 90 minutes after administration.

The dual cholate clearance SHUNT method yields 3 test results: Portal-systemic shunt fraction (SHUNT (%)); Portal Hepatic Filtration Rate (Portal HFR, which is also defined as FLOW in above discussions and examples, (mL/min/kg)) based on orally administered distinguishable cholate compound in the blood; and Systemic Hepatic Filtration rate (Systemic HFR, (mL/min/kg)), based on intravenously administered distinguishable cholate compound in the blood. Cholate-2,2,4,4-d₄ (40 mg) is given orally and taken up into the portal vein by specific enteric transporters. Cholate-24-¹³C (20 mg) is given IV and is taken up primarily through the hepatic artery from the systemic circulation. Specific hepatic transporters clear cholate from the portal and systemic circulation. For example, highly conserved hepatic transporters (NTCP, OATPs) clear cholate from the portal and systemic circulation.

The SHUNT equation may be expressed as:

$$SHUNT Fraction = F = \frac{{HFR}_{z}}{{HFR}_{p}}$$

SHUNT Fraction (F) is not indexed against controls, it is the ratio of clearances within the individual. Since the expression for SHUNT is a ratio, the units of clearance drop from the value for SHUNT—in this equation SHUNT is a fraction. It may also be expressed as percent shunt by multiplying by 100%.

Cholate absorption is assumed to be approximately 100%. Absorption of unconjugated cholate is rapid following oral administration-d4-cholate is detected in peripheral venous blood samples within 5 to 10 minutes of its administration, and peak absorption is between 20 to 60 minutes. Although cholate is a ligand for OATP transporters, most of its absorption is passive by flip-flop through the intestinal epithelial cell membrane due to its detergent-like properties. Literature indicates that absorption is 100%.

Each of the liver function tests of the present are based on peripheral venous blood sampling from the systemic compartment. In the cholate SHUNT test 13C-cholate is administered intravenously to measure cholate clearance since this dose is 100% available to the systemic compartment. Clearance of 13C-cholate may be defined by:

Cl_{iv 13C-cholate}=doseᵢᵥ/AUCᵢᵥ.

In contrast, d4-cholate is orally administered, absorbed from the intestine, delivered to the liver via the portal vein. Given its rapid and efficient intestinal absorption, d4-cholate that is not extracted in the first pass through the liver spills into the systemic compartment and is then cleared similarly to 13C-cholate. The clearance of d4-cholate may be defined by:

Cl_{po d4-cholate}=F×(doseₚₒ/AUCₚₒ),

where F is the fraction of the oral dose escaping first-pass extraction. Setting Cl_{iv 13C-cholate} equal to Cl_{po d4-cholate} allows calculation of F from the peripheral venous blood concentrations. With disease progression, F increases when HFRₚ decreases to a relatively greater extent than the decline in HFRₛ—usually in association with development of a collateral circulation.

F is only measured in the cholate SHUNT test. F is indirectly assessed in both the FLOW and STAT tests—the latter tests measure the peripheral venous concentrations of d4-cholate, which increase as F increases, by either AUC (FLOW) or a single time point (STAT).

It is assumed that the Volume of distribution and hepatic extraction of [13C-CA] is identical to [d4-CA], i.e., either could be given IV or PO in same subject under same condition and yield the same clearances. Evidence in support of this assumption is the finding that 13C-labeled and deuterium-labeled bile acids are handled similarly within the human body. (Everson G T. Steady-state kinetics of serum bile acids in healthy human subjects: single and dual isotope techniques using stable isotopes and mass spectrometry. J Lipid Res 1987; 28: 238-252.)

Three key outputs from the cholate Shunt test, DSI, SHUNT, and HR are unitless. DSI and HR may be indexed against HFRs of healthy persons—HFRs are defined by clearance divided by weight and expressed per kg body weight. Although clearance values are normalized to subject body weight, other methods of indexing may be utilized, for example, to subject lean body mass, ideal body weight, body surface area, blood volume, estimated blood volume may be utilized.

The unit of kilogram of body weight (kg) is in both numerator and denominator and cancels. Thus, normalizing HFRs to a given body weight or size for calculation of DSI or HR is not necessary.

For example, if HFRs are normalized to 75 kg body weight:

HFR=Cl/Body Wt_{kg}=dose/(AUC×Body Wt_{kg})

HFR_{adjustd 75 kg}=dose/[(AUC×Body Wk_{kg})/75 kg]=HFR×75

the multiplier, 75, appears in both numerator and denominator for DSI, HR, and SHUNT and cancels.

SHUNT, a ratio of clearances within an individual, is not indexed against a control group but is also unitless since the expression of clearance by body weight (or size) is in both numerator and denominator and cancels. SHUNT, likewise, does not require normalization to body weight or size.

Estimation of Portal Hepatic Filtration Rate

The STAT test is a simplified, non-invasive convenient test intended for screening purposes can reasonably estimate the portal blood flow from a single blood sample taken at a single time point, e.g., 60 minutes after oral administration of a distinguishable cholate compound, e.g., a deuterated cholate.

Comparison of Portal HFR (FLOW), SHUNT, STAT and DSI Liver Function Tests.

A comparison of typical embodiments of SHUNT, FLOW, STAT, and DSI tests is shown in Table 1.

TABLE 1
Liver Function Tests.

| | Exemplary | | | |
|---|---|---|---|---|
| | Distin- | Route of | | What is |
| Test | guishable | Adminis- | | Measured |
| Name | Compound | tration | Samples | or Defined |
| SHUNT | ¹³C-cholate | Intravenous | n = 5 | Clearances and |
| | 4D-²H-cholate | Oral | over 90 min | Shunt- |
| | | | | comprehensive |
| | | | | assessment of |
| | | | | hepatic blood |
| | | | | flow and hepatic |
| | | | | function |
| FLOW | 4D-²H-cholate | Oral | n = 5 | Portal |
| | | | over 90 min | circulation |
| | | | | (portal hepatic |
| | | | | filtration rate; |
| | | | | Portal HFR); |
| | | | | portal HFR may |
| | | | | be estimated |
| | | | | from STAT |
| STAT | 4D-²H-cholate | Oral | n = 1 | Estimates |
| | | | at 60 min | FLOW and |
| | | | | correlates |
| | | | | with SHUNT |
| | | | | Estimates DSI |
| DSI | ¹³C-cholate | Intravenous | n = 5 | Clearances and |
| | 4D-²H-cholate | Oral | over 90 min | Shunt- |
| | | | | comprehensive |
| | | | | assessment of |
| | | | | hepatic blood |
| | | | | flow and |
| | | | | hepatic |
| | | | | function; |
| | | | | DSI may also |
| | | | | be estimated |
| | | | | from STAT. |

Values for normal liver function were established in healthy controls in previous studies: the average SHUNT is 20%, average HFR (FLOW) is 30, and average STAT is 0.4.

In the diseased liver, as more blood escapes extraction by intra- and extra-hepatic shunting to the systemic circulation, the SHUNT increases (˜30-90%), HFR (FLOW) or portal flow decreases (˜20 to 2 mL/min/kg), and STAT increases (0.6 to 5 uM).

Improved methods for blood or serum sample preparation, analyte detection and quantification are provided herein allowing for improved recovery of analyte from the sample, increased analyte selectivity, increased sample throughput, significantly decreased time of processing, 10-fold reduction in required patient sample volume, and improved lower limit of quantitation (LOQ). These methods and advantages may be applied to one or more of the SHUNT, FLOW, STAT, and DSI liver function tests.

### Definitions

As used herein, “a” or “an” may mean one or more than one of an item.

The term “about” when referring to any numerical parameter means +/−10% of the numerical value. For example, the phrase “about 60 minutes” refers to 60 minutes+/−6 minutes.

The term “accuracy” (measurement) when used herein refers to closeness of agreement between a measured quantity value and a true quantity value of a measurand.

The term “acceptability” as used herein is based on individual criteria that set minimal operational characteristics for a measurement procedure.

The term “precision” as used herein refers to closeness of agreement between independent test/measurement results obtained under stipulated conditions.

The term “trueness” as used herein refers to the closeness of agreement between the expectation of a test result or a measurement result and a true value.

The term “measureand” is used when referring to the quantity intended to be measured instead of analyte (component represented in the name of a measurable quantity).

The term “verification” as used herein focuses on whether specifications of a measurement procedure can be achieved, whereas the term “validation” verifies that the procedure is fit for an intended purpose.

The term “measurement procedure” refers to a detailed description of a measurement according to one or more measurement principles and to a given measurement method, based on a measurement model and including any calculation to obtain a measurement result.

As used herein “clearance” may mean the removing of a substance from one place to another.

As used herein, the term “simultaneously” when referring to 2 or more events refers to occurring within 20 minutes or less, within 15 minutes, 10 minutes, 5 minutes, or within 3 minutes of each other.

As used herein the terms, “patient”, “subject” or “subjects” include but are not limited to humans, the term may also encompass other mammals, or domestic or exotic animals, for example, dogs, cats, ferrets, rabbits, pigs, horses, cattle, birds, or reptiles.

The acronym “HALT-C” refers to the Hepatitis C Antiviral Long-term Treatment against Cirrhosis trial. The HALT-C trial was a large, prospective, randomized, controlled trial of long-term low dose peg interferon therapy in patients with advanced hepatitis C who had not had a sustained virologic response to a previous course of interferon-based therapy. An NIH-sponsored Hepatitis C Antiviral Long-Term Treatment against Cirrhosis (HALT-C) Trial examined whether long-term use of antiviral therapy (maintenance treatment) would slow the progression of liver disease. In noncirrhotic patients who exhibited significant fibrosis, effective maintenance therapy was expected to slow or stop histological progression to cirrhosis as assessed by serial liver biopsies. However, tracking disease progression with biopsy carries risk of complication, possibly death. In addition, sampling error and variation of pathologic interpretation of liver biopsy limits the accuracy of histologic assessment and endpoints. The histologic endpoint is less reliable because advanced fibrosis already exists and changes in fibrosis related to treatment or disease progression cannot be detected. Thus, standard endpoints for effective response to maintenance therapy in cirrhotic patients are prevention of clinical decompensation (ascites, variceal hemorrhage, and encephalopathy) and stabilization of liver function as measured clinically by Childs-Turcotte-Pugh (CTP) score. However, clinical endpoints and CTP score were known to be insensitive parameters of disease progression. Dual isotope techniques employing distinguishable cholates were used in development of the SHUNT test and used in conjunction with the HALT-C trial. The term “SHUNT test” refers to a previously disclosed QLFT (quantitative liver function test) used as a comprehensive assessment of hepatic blood flow and liver function. The SHUNT test is used to determine plasma clearance of orally and intravenously administered distinguishable cholic acids in subjects with and without chronic liver disease. SHUNT fraction or percent quantifies the spillover of the PO d4-cholate into the systemic circulation from the ratio of the clearance of the intravenously administered 13C-cholate to the clearance of the orally administered d4-cholate. In the SHUNT test, at least 5 blood samples are analyzed which have been drawn from a patient at intervals over a period of at least about 90 minutes after oral and intravenous administration of differentiable cholates. The SHUNT test is disclosed in Everson et al., U.S. Pat. No. 8,613,904, which is incorporated herein by reference. These studies demonstrated reduced clearance of cholate in patients who had either hepatocellular damage or portosystemic shunting. The “SHUNT test value” refers to a number (in %). The term “SHUNT %” represents a quantitative measurement of portal-systemic shunting. SHUNT % is a measurement of the percentage of spillover of the orally administered d4-cholate. The first-pass hepatic elimination of cholate in percent of orally administered cholate is defined as (100%−SHUNT). SHUNT test methods are disclosed in U.S. Pat. Nos. 8,613,904, 9,639,665, 8,778,299, 9,417,230, and 10,215,746, each of which is incorporated herein by reference in its entirety. Analysis of samples for stable isotopically labeled cholates is performed by, e.g., GC-MS, following sample derivitization, or LC-MS, without sample derivitization, or LC-MS/MS, or MS/MS as disclosed herein. The ratio of the AUCs of orally to intravenously administered cholic acid, corrected for administered doses, defines cholate shunt. The cholate shunt can be calculated using the formula: AUCₒᵣₐₗ/AUCᵢᵥ×Doseᵢᵥ/Doseₒᵣₐₗ×100%, wherein AUCₒᵣₐₗ is the area under the curve of the serum concentrations of the orally adminstered cholic acid and AUCᵢᵥ is the area under the curve of the intravenously administered cholic acid.

The SHUNT test allows measurement of first-pass hepatic elimination of bile acids from the portal circulation. Flow-dependent, first pass elimination of bile acids by the liver ranges from about 60% for unconjugated dihydroxy, bile acids to about 95% for glycine-conjugated cholate. Free cholate, used herein has a reported first-pass elimination of approximately 80% which agrees closely with previously observed first pass elimination in healthy controls of about 83%. After uptake by the liver, cholic acid is efficiently conjugated to either glycine or taurine and secreted into bile. Physicochemically cholic acid may be easily separated from other bile acids and bile acid or cholic acid conjugates, using chromatographic methods.

The term “Cholate Elimination Rate”, kₑₗᵢₘ min⁻¹ represents the first phase of elimination of the intravenously administered 13C-cholate, calculation from Ln/linear regression of [13C-cholate] versus time (using only the 5- and 20-minute time points). Intravenously administered 13C-cholate is rapidly delivered to the liver via the hepatic artery. In contrast, the same 13C-cholate slowly transits to the liver via the portal vein due to the capacitance of the splanchnic vascular bed. Thus, the first phase of cholate elimination is more dependent upon clearance from the hepatic artery than from portal vein.

The term “Volume of distribution”, Va., L kg′ represents the body's volume into which cholate is distributed. This is calculated from the intercept on the Y axis of the Ln/linear regression of [13C-cholate] versus time (using only the 5- and 20-min time points).

The acronym “IV” or “iv” refers to intravenous route of administration.

The acronym “PO” refers to per oral route of administration.

The acronym “PHM” refers to perfused hepatic mass.

The acronym “SF” refers to shunt fraction, for example, as in liver SF, or cholate SF.

The acronym “ROC” refers to receiver operating characteristic. The ROC curve is a graphical plot which illustrates performance of a binary classifier system as its discrimination threshold is varied. It is created by plotting the fraction of true positives out of the positives (TPR=true positive rate) vs. the fraction of false positives out of the negatives (FPR=false positive rate), at various threshold settings. Sensitivity is the probability of a positive test result, or of a value above a threshold, among those with disease. Sensitivity is defined as the true positive rate (TPR): TPR=TP/P=TP/(TP+FN). False positive rate (FPR) is FPR=FP/N=FP/(FP+FN). Accuracy (ACC) is defined as ACC=(TP+TN)/(P+N). Specificity is the probability of a negative test result, or a value below a threshold, among those without disease. Specificity (SPC), or true negative rate (TN) is defined as SPC=TN/N=TN/(FP+TN)=1-FPR. Positive prediction value (PPV) is defined as: PPV=TP/(TP+FP). Negative predictive value (NPV) is defined as NPV=TN/(TN+FN).

The c-statistic is the area under the ROC curve, or “AUROC” (area under receiver operating characteristic curve) and ranges from 0.5 (no discrimination) to a theoretical maximum of 1 (perfect discrimination).

The term “sustained virologic response” (SVR) is used to describe a desired response in a patient when, e.g., hepatitis C virus is undetectable in the blood six months after finishing treatment. Conventional treatment using interferon and ribavirin doesn't necessarily eliminate, or clear, the hepatitis C virus. A sustained virologic response is associated with a very low incidence of relapse. SVR is used to evaluate new medicines and compare them with proven therapies.

The term “oral cholate clearance” (Clₒᵣₐₗ) refers to clearance from the body of a subject of an orally administered cholate compound as measured by a blood or serum sample from the subject. Oral cholate clearance is used as a measure of portal blood flow. Orally administered cholic acid is absorbed across the epithelial lining cells of the small intestine, bound to albumin in the portal blood, and transported to the liver via the portal vein. Approximately 80% of cholic acid is extracted from the portal blood in its first pass through the liver. Cholic acid that escapes hepatic extraction exits the liver via hepatic veins that drain into the vena cava back to the heart, and is delivered to the systemic circulation. The area under the curve (AUC) of peripheral venous concentration versus time after oral administration of cholic acid quantifies the fraction of cholic acid escaping hepatic extraction and defines “oral cholate clearance”.

The term “portal hepatic filtration rate”, “portal HFR”, “FLOW test” refers to oral cholate clearance (portal hepatic filtration rate; portal HFR) used as a measure of portal blood flow, or portal circulation, obtained from analysis of concentration of distinguishable cholate compound in at least 5 blood samples drawn from a subject over a period of, for example, about 90 minutes after oral administration of a distinguishable cholate compound, for example, a distinguishable cholate. The units of portal HFR value are typically expressed as mL/min/kg, where kg refers to kg body weight of the subject. “Portal HFR”, mL kg′ may be used to Model independent apparent clearance of orally administered d4-cholate, adjusted for body weight, and calculated from dose/AUC. FLOW test methods are disclosed in U.S. Pat. Nos. 8,778,299, 9,417,230, and 10,215,746, each of which is incorporated herein by reference in its entirety. “Systemic HFR”, mL min⁻¹ kg⁻¹ may be used to Model independent clearance of intravenously injected 13C-cholate, adjusted for body weight, and calculated from dose/AUC.

The term “STAT test” refers to an estimate of portal blood flow by analysis from one patient blood sample drawn at a defined period of time following oral administration of a differentiable cholate. In one aspect, the STAT test refers to analysis of a single blood sample drawn at a specific time point after oral administration of a differentiable cholate. In one specific aspect, the STAT test is a simplified convenient test intended for screening purposes that can reasonably estimate the portal blood flow (estimated flow rate) from a single blood sample taken 60 minutes after orally administered deuterated-cholate. In some embodiments, STAT, is the d4-cholate concentration in the 60 minute blood sample. STAT correlates well with DSI and can be used to estimate DSI. The STAT test value is typically expressed as a concentration, for example, micromolar (uM) concentration. STAT test methods are disclosed in U.S. Pat. Nos. 8,961,925, 10,222,366, each of which is incorporated herein by reference in its entirety. STAT test value may be used to estimate portal HFR, as provided in U.S. Pat. Nos. 8,961,925, 10,222,366. A STAT test value in a patient may be used to estimate a DSI value in a patient, as provided herein.

The term “DSI test” refers to Disease Severity Index test which is derived from one or more liver function test results based on hepatic blood flow. The DSI score is a function of the sum of cholate clearances from systemic and portal circulations adjusted to disease severity ranging from healthy subjects to end stage liver disease. DSI is a score without units representing a quantitative measurement of liver function. A disease severity index (DSI) value may be obtained in a patient by a method comprising (a) obtaining one or more liver function test values in a patient having or at risk of a chronic liver disease, wherein the one or more liver function test values are obtained from one or more liver function tests selected from the group consisting of SHUNT, portal hepatic filtration rate (portal HFR), and systemic hepatic filtration rate (systemic HFR); and (b) employing a disease severity index equation (DSI equation) to obtain a DSI value in the patient, wherein the DSI equation comprises one or more terms and a constant to obtain the DSI value, wherein at least one term of the DSI equation independently represents a liver function test value in the patient, or a mathematically transformed liver function test value in the patient from step; and the at least one term of the DSI equation is multiplied by a coefficient specific to the liver function test. DSI is an index, or score, that encompasses the cholate clearances from both systemic and portal circulations. DSI has a range from 0 (healthy) to 50 (severe end-stage disease) and is calculated from both HFRs. Based on the reproducibility of DSI, the minimum detectable difference indicating a change in liver function in a subject may be about 1.5 points, about 2 points, or about 3 points. DSI test methods and equations are disclosed in U.S. Pat. Nos. 9,091,701, 9,759,731, 10,520,517, each of which is incorporated herein by reference in its entirety. Additional DSI equations have been developed and are provided herein. A method of estimating a DSI value in a patient from a STAT test value is also provided herein.

The term “Hepatic Reserve” refers to percentage of maximum hepatic functional capacity measured by DSI, indexed hepatic reserve may be normalized to the DSI range in subjects of lean body mass.

HR (algebraic) is simply an algebraic conversion of the DSI value in the subject: HR=[100−(2×DSI)].

Indexed HR, is normalized against the results within a cohort of normal lean controls.

One formula for Hepatic Reserve (indexed) begins with the DSI equation:

$$DSI = A \sqrt{{( ln ( \frac{b}{{HFR}_{p}} ) )}^{2} + {( ln ( \frac{c}{{HFR}_{s}} ) )}^{2}} \qquad Eqn . 9$$

But, in contrast to DSI, a minimal limit is placed on the range of normal—the average values for Portal HFR and Systemic HFR minus one SD of the mean for each in healthy controls of lean body mass (y and z, respectively). Thus, the HRindexed formula may be written as:

$$HRindexed = 100 - ( X \sqrt{{( ln ( \frac{y}{{HFR}_{p}} ) )}^{2} + {( ln ( \frac{z}{{HFR}_{s}} ) )}^{2}} ) , \qquad Eqn . 10$$

wherein:
X is a scaling multiplier from 20 to 35 (optionally 29.40578) to yield a range from 100 (normal hepatic reserve) to 0 (no hepatic reserve),
y is the minimum value for Portal HFR determined from the average values for Portal HFR minus one SD of the mean for each in a plurality of healthy controls of lean body mass in a range or 15-40, optionally wherein y=29.1; and
z is the minimum value for Systemic HFR determined from the average values for Systemic HFR minus one SD of the mean for each in the plurality of healthy controls of lean body mass in a range of 4-10, optionally wherein z=6.52. The variables in the HR equation, y, z, HFRₚ, and HFRₛ, are all clearance values with units of mL min⁻¹ kg⁻¹—but, the units drop in the equation due to factoring the variables as ratios.

Based on the range of HFRs in 30 lean controls (Portal HFR 29.10±9.04 mL min⁻¹ kg⁻¹; Systemic HFR 6.52±1.49 mL min⁻¹ kg⁻¹), the minimum values for normal Portal HFR and Systemic HFR were set at:

| | | |
|---|---|---|
| Portal HFR | y = 29.1 | (range 20-40, or 25-35) |
| Systemic HFR | z = 6.52 | (range 4-12, or 5-10) |

Use of range in lean controls, versus all controls, allows detection of changes in HR in overweight and obese subjects for possible underlying fatty liver disease. As HFRₚ and HFRₛ approach y and z, respectively, HR approaches 100—“NORMAL HEPATIC RESERVE”. As HFRs approach 1, HR approaches 0—“NO HEPATIC RESERVE”.

The term “intravenous cholate clearance” (Clᵢᵥ) refers to clearance of an intravenously administered cholate compound. Intravenously administered cholic acid, bound to albumin, distributes systemically and is delivered to the liver via both portal venous and hepatic arterial blood flow. The AUC of peripheral venous concentration versus time after intravenous administration of cholic acid is equivalent to 100% systemic delivery of cholic acid. The ratio of the AUCs of orally to intravenously administered cholic acid, corrected for administered doses, defines cholate shunt.

The term “RCA20” represents the amount of the intravenously administered distinguishable compound, for example, a distinguishable cholate compound such as 13C-CA, that remains in the circulation 20 minutes after the intravenous injection. The formula for RCA20 may be expressed as:

RCA20=(1−([¹³C CA]ₜ₌₀−[¹³C CA]ₜ₌₂₀)/[¹³C CA]ₜ₌₀)×100%, Eqn. 11:

wherein RCA20 represents the amount of the intravenously administered 13C-CA that remains in the circulation 20 minutes after the intravenous injection. [¹³C CA]ₜ₌₀ is determined from Ln/linear regression of [13C-CA] versus time. RCA20 can be compared to R15 for ICG data. The indocyanine green (ICG) clearance test (K) and retention rate at 15 minutes (R15) have been used as one indicator of liver function for example in patients with cirrhosis.

The term “Quantitative Liver Function Test” (QLFT), refers to assays that measure the liver's ability to metabolize or extract test compounds, can identify patients with impaired hepatic function at earlier stages of disease, and possibly define risk for cirrhosis, splenomegaly, and varices. One of these assays is the cholate shunt assay where the clearance of cholate is assessed by analyzing bodily fluid samples after exogenous cholate has been taken up by the body.

The term “Ishak Fibrosis Score” is used in reference to a scoring system that measures the degree of fibrosis (scarring) of the liver, which is caused by chronic necroinflammation. A score of 0 represents no fibrosis, and 6 is established fibrosis. Scores of 1 and 2 indicate mild degrees of portal fibrosis; stages 3 and 4 indicate moderate (bridging) fibrosis. A score of 5 indicates nodular formation and incomplete cirrhosis, and 6 is definite cirrhosis.

The term “Childs-Turcotte-Pugh (CTP) score” or “Child-Pugh score” refers to a classification system used to assess the prognosis of chronic liver disease as provided in Pugh et al., Transection of the oesophagus for bleeding oesophageal varices. Br J Surg 1973; 60:646-649, which is incorporated herein by reference. The CTP score includes five clinical measures of liver disease; each measure is scored 1-3, with 3 being the most severe derangement. The five scores are added to determine the CTP score. The five clinical measures include total bilirubin, serum albumin, prothrombin time international normalized ratio (PT INR), ascites, and hepatic encephalopathy. The CTP score is one scoring system used in stratifying the seriousness of end-stage liver disease. Chronic liver disease is classified into Child-Pugh class A to C, employing the added score. Child-Pugh class A refers to CTP score of 5-6. Child-Pugh class B refers to CTP score of 7-9. Child-Pugh class C refers to CTP score of 10-15. A website calculates post-operative mortality risk in patients with cirrhosis. http://mayoclinic.org/meld/mayomodel9.html

The term “Model for End-Stage Liver Disease (MELD) refers to a scoring system used to assess the severity of chronic liver disease. MELD was developed to predict death within three months of surgery in patients who had undergone a transjugular intrahepatic portosystemic shunt (TIPS) procedure patients for liver transplantation. MELD is also used to determine prognosis and prioritizing for receipt of a liver transplant. The MELD uses a patient's values for serum bilirubin, serum creatinine, and international normalized ratio for prothrombin time (INR) to predict survival. The scoring system is used by the United Network for Organ Sharing (UNOS) and Eurotransplant for prioritizing allocation of liver transplants instead of the older Child-Pugh score. See UNOS (2009 Jan. 28) “MELD/PELD calculator documentation”, which is incorporated herein by reference. For example, in interpreting the MELD score in hospitalized patients, the 3 month mortality is: 71.3% mortality for a MELD score of 40 or more.

The term “standard sample” refers to a sample with a known concentration of an analyte used for comparative purposes when analyzing a sample containing an unknown concentration of analyte.

The term “Chronic Hepatitis C” (CHC) refers to a chronic liver disease caused by viral infection and resulting in liver inflammation, damage to the liver and cirrhosis. Hepatitis C is an infection caused by a blood-borne virus that attacks the liver and leads to inflammation. Many people infected with hepatitis C virus (HCV) do not exhibit symptoms until liver damage appears, sometimes years later, during routine medical tests.

The term “Alcoholic SteatoHepatitis” (ASH) refers to a chronic condition of inflammation of the liver which is caused by excessive drinking. Progressive inflammatory liver injury is associated with long-term heavy intake of ethanol and may progress to cirrhosis.

The term “Non-Alcoholic SteatoHepatitis” (NASH) refers to a serious chronic condition of liver inflammation, progressive from the less serious simple fatty liver condition called steatosis. Simple steatosis (alcoholic fatty liver) is an early and reversible consequence of excessive alcohol consumption. In people that don't drink much alcohol, the cause of fatty liver disease is less clear, but may be associated with factors such as obesity, high blood sugar, insulin resistance, or high levels of blood triglycerides. In certain cases the fat accumulation can be associated with inflammation and scarring in the liver. This more serious form of the disease is termed non-alcoholic steatohepatitis (NASH). NASH is associated with a much higher risk of liver fibrosis and cirrhosis than NAFLD. Patients with NASH have increased risk for hepatocellular carcinoma. NAFLD may progress to NASH with fibrosis cirrhosis and hepatocellular carcinoma.

The term “Non-Alcoholic Fatty Liver Disease” (NAFLD) refers to a common chronic liver disease characterized in part by a fatty liver condition with associated risk factors of obesity, metabolic syndrome, and insulin resistance. Both NAFLD and NASH are often associated with obesity, diabetes mellitus and asymptomatic elevations of serum ALT and gamma-GT. Ultrasound monitoring can suggest the presence of a fatty infiltration of the liver; differentiation between NAFLD and NASH, typically requires a liver biopsy.

The term “Primary Sclerosing Cholangitis” (PSC) refers to a chronic liver disease caused by progressive inflammation and scarring of the bile ducts of the liver. Scarring of the bile ducts can block the flow of bile, causing cholestasis. The inflammation can lead to liver cirrhosis, liver failure and liver cancer. Chronic biliary obstruction causes portal tract fibrosis and ultimately biliary cirrhosis and liver failure. The definitive treatment is liver transplantation. Indications for transplantation include recurrent bacterial cholangitis, jaundice refractory to medical and endoscopic treatment, decompensated cirrhosis and complications of portal hypertension (PHTN). PSC progresses through chronic inflammation, fibrosis/cirrhosis, altered portal circulaton, portal hypertension and portal-systemic shunting to varices-ascites and encephalopathy. Altered portal flow is an indication of clinical complications.

A “quantifier ion” is a single fragment ion selected from each analyte used for quantitation of the analyte. The quantifier ion may be the most intense fragment ion, and additional ions may be qualifier ions.

A “qualifier ion” is an ion selected from the mass spectrum of the target analyte compound. The presence of the qualifier ion in the correct amount relative to the quantifier ion gives evidence of correct target compound identification.

The term “ion ratio monitoring” refers to the ratio of quantifier ion and a selected qualifier ion. For example, the qualifier ion signal >50% that of the quantifier ion, the ion ratio in the patient samples should not change by ±20-30% from that of the mean ratio of the standards.

A “calibration sample” is a sample containing a known concentration of a compound to be quantitated (target analyte compound). It may also be referred to as a calibration standard. If the method used an internal standard that compound may also be in the calibration sample.

An “internal standard” is a distinguishable version of the target analyte compound or an analog of the target analyte compound. For example, the internal standard may be an isotopically labeled analyte compound or an analog of the target analyte compound distinguishable by mass. The internal standard may be added at the beginning of the sample processing, for example, before solid phase extraction. The amount of internal standard may be within the working standard curve, preferably in the lower half, lower third, or lower quarter of the working standard curve.

The term “selected ion monitoring” (SIM) refers to wherein the mass spectrometer is set to scan over a very small mass range, typically one mass unit. The narrower the range, the more specific the SIM data. Only compounds with the selected mass are detected and plotted. However, the intact mass of a compound may not be a unique identifier, so the SIM plot may show a number of peaks without a unique identifier. For example, in a complex sample, many compounds may have the same mass or have the same m/z ratio.

The term “multiple reaction monitoring” (MRM) refers to a method used for analyte quantitation in a tandem mass spectrometry in which an ion of a particular mass is selected in the first stage of a tandem mass spectrometer and an ion product of a fragmentation reaction of the precursor ion is selected in the second mass spectrometer stage for detection. For example, in the first stage of MRM, the sample ionization method may be by EI, ESI, or MALDI to create a precursor ion utilizing m/z separation. The precursor ion is subjected to fragmentation and further m/z separation to create a fragment ion.

The term “lower limit of quantitation” (LLOQ) refers to lowest amount of analyte that can reliably detected and meets laboratory requirements for accuracy and precision. For example, LOQ may meet stated acceptable precision (CV<20%) and accuracy (<15% bias).

Terms not otherwise defined herein may be found in “CLSI. Liquid-chromatography-mass spectrometry methods. Approved guideline, C62-A, Wayne, Pa., Clinical and Laboratory Standards Institute”, 2014; and “CLSI. Mass spectrometry in the clinical laboratory: general principles and guidance. Approved guideline, C50-A, Wayne, Pa., Clinical and Laboratory Standards Institute”, 2007.

The mass spectrometer (MS) may be, for example, any suitable mass spectrometer known in the art. The MS may be a quadrapole mass spectrometer (Q), or a Time of Flight (TOF) mass spectrometer, or an ion trap mass spectrometer. The MS/MS may be a triple quadrapole LC-MS/MS Mass spectrometer wherein Q1 resolves molecular ions, Q2 fragments molecule, Q3 resolves fragments, for example, an API 4000 (AB Sciex Instruments). A given molecule producing a given fragment is a reaction. Multiple Reaction Monitoring (MRM) may be employed measuring several molecules each giving a characteristic fragment or several fragments. The MS, MS/MS, or LC-MS/MS may involve any appropriate ionization technique known in the art. Ionization techniques may be selected from electron ionization (EI), electrospray ionization (ESI), atmospheric pressure chemical ionization (APCI). In some embodiments, methods for introducing samples to the MS or MS/MS instrument without chromatography are employed. For example, matrix-assisted laser desorption ionization (MALDI), may be used to introduce sample without chromatography and also ionize at the same time. Methods to introduce sample to MS or MS/MS may include laser diode thermal desorption (LDTD) such as Phytronix Luxon source. The MS or MS/MS may also involve acoustic ejection mass spectrometry (AEMS), for example, combining open port interface (OPI) with acoustic droplet ejection (ADE) to allow sample analysis directly from plate without LC. One example of ADE is AB Sciex Echo MS.

Chromatography techniques may optionally be used with MS or MS/MS methods for quantitation of distinguishable compound(s) in patient samples. Liquid chromatography (LC) may be employed, for example, in line with MS or MS/MS techniques. LC may be used for chromatographic separation of sample components using any appropriate solid phase matrix. Any appropriate chromatographic method may be employed, including normal phase, reverse phase, ion exchange, hydrophobic interaction, size exclusion, affinity chromatography, and so forth. For example, the LC solid phase may be a C18 or C8 or other reverse phase solid phase matrix. Gas chromatography may be used in line with MS or MS/MS techniques. Chromatographic separation of sample components in a gas phase may be employed. For example, GC using a matrix comprising silica or other solid phase may be employed.

### Computer/Processor

The detection, prognosis and/or diagnosis method employed in the SHUNT, FLOW, STAT, and/or DSI tests can employ the use of a processor/computer system. For example, a general purpose computer system comprising a processor coupled to program memory storing computer program code to implement the method, to working memory, and to interfaces such as a conventional computer screen, keyboard, mouse, and printer, as well as other interfaces, such as a network interface, and software interfaces including a database interface find use one embodiment described herein.

The computer system accepts user input from a data input device, such as a keyboard, input data file, or network interface, or another system, such as the system interpreting, for example, the data such as MS, MS/MS, LC-MS/MS, or GC/MS data, and provides an output to an output device such as a printer, display, network interface, or data storage device. Input device, for example a network interface, receives an input comprising detection of distinguishable cholate compound measured from a processed blood or serum sample described herein and quantification of those compounds. The output device provides an output such as a display, including one or more numbers and/or a graph depicting the detection and/or quantification of the compounds.

Computer system is coupled to a data store, which stores data generated by the methods described herein. This data is stored for each measurement and/or each subject; optionally a plurality of sets of each of these data types is stored corresponding to each subject. One or more computers/processors may be used, for example, as a separate machine, for example, coupled to computer system over a network, or may comprise a separate or integrated program running on computer system. Whichever method is employed these systems receive data and provide data regarding detection/diagnosis in return.

In embodiments, a method for selecting a treatment for a subject is provided, wherein the subject has an abnormal level of one or more distinguishable bile acid compounds in a blood or serum sample drawn at a multiplicity of time points or a single time point following oral administration and/or intravenous administration comprises calculating an output score, using a computing device, by inputting the distinguishable cholate compound level into a function that provides a predictive relationship between cholate level and outcome, for subjects having a liver disease or disorder; and displaying the output score, using a computing device.

In embodiments, the method further comprises determining whether the output score is greater than, or equal to, or less than a cutoff value, using a computing device; and displaying whether the subject is likely to experience a clinical outcome if the output score is greater than, or equal to, or less than a cutoff value.

In embodiments, a computing device, comprises a processing unit; and a system memory connected to the processing unit, the system memory including instructions that, when executed by the processing unit, cause the processing unit to: calculate a level of distinguishable cholate compound from a single blood sample from a subject into a function that provides a predictive relationship between distinguishable cholate level of the subject having a liver disease or dysfunction; and display the output score. In embodiments, the system memory includes instructions that when executed by the processing unit, cause the processing unit to determine whether the output score is greater than or equal to or less than a cutoff value; and displaying whether the subject is likely to experience a clinical outcome if the output score is greater than or equal to the cutoff value.

As utilized herein, the following acronyms apply. DPBS refers to Dulbecco's phosphate-buffered saline. EDTA refers to ethylenediaminetetraacetic acid. GLP refers to Good Laboratory Practice. HPLC refers to high-performance liquid chromatography. LC refers to liquid chromatography. LC-MS/MS refers to liquid chromatography-tandem mass spectrometry. LLOQ refers to lower limit of quantification. m/z refers to mass-to-charge ratio, wherein m is an ion's mass in atomic mass units (amu), and z is the ion's formal charge, where formal charge is typically +1 unless otherwise specified. MRM refers to Multiple Reaction Monitoring. MS refers to Mass spectrometry. MS/MS refers to Tandem mass spectrometry. Q1 refers to Quadrupole 1. Q3 refers to Quadrupole 3. QAU refers to Quality assurance unit. QC refers to Quality control. r refers to Correlation coefficient. RSD % refers to Relative standard deviation in %. StdDev refers to Standard deviation. v/v refers to Volume by volume.

Sample Collection and Processing

Improved methods of sample collection and processing are provided herein for quantitation of a distinguishable bile acid in a patient blood or serum sample.

In prior art employing HPLC-MS or GC-MS, collection of at least 0.5 mL blood or serum sample was required per time point. This is because each sample was subjected to extensive processing prior to analysis.

In order to ensure accurate liver function testing, the labeled cholate test compounds must be isolated and identified from patients' serum samples. Cholate compounds are amphipathic molecules with both hydrophobic and hydrophilic regions. Cholates are also carboxylic acids that can exist in either an uncharged free acid form (cholic acid) or a charged carboxylic acid form (cholate) depending on pH. These properties can be exploited to isolate cholate compounds from serum. The use of HPLC-MS as opposed to GC-MS, allows for analysis of cholate without sample derivitization. Alternatively, GC-MS can be used for sample analysis with derivitization by any technique known in the art, for example, by the method of Everson and Martucci, US 2008/0279766, which is incorporated herein by reference.

Methods for sample processing and quantitation of a distinguishable cholate compound in a blood or serum sample using HPLC-MS are provided in, for example, U.S. Pat. No. 9,091,701, which is incorporated herein by reference. For example, typical sample processing in prior art methods may involve adding an unlabeled cholic acid internal standard to 0.5 mL of a patient's serum. Dilute sodium hydroxide is then added and the sample is centrifuged, then added to solid phase extraction SPE cartridges pre-equilibrated with water and 10% methanol. The cartridges are washed with water and 10% aq. methanol, then the labeled cholate is eluted from SPE cartridge with 90% aq. methanol. The eluted sample is dried to remove methanol, and acidified using 0.2 N HCl prior to convert the cholate compounds to their free acid form. Diethyl ether is added to the acidified sample and then the sample is vortexed to extract the free acid form of the cholate compounds into the ether phase. The upper ether lay is collected, and gently dried to remove the ether, for example, in a fume hood without heating or under N₂ gas. HPLC-MS mobile phase buffer is added to dried samples prior to injecting to HPLC-MS. This laborious prior art method of sample processing was nevertheless an improvement over the previous GC-MS sample processing method, because analyte derivitization was not required in the HPLC-MS method. However, manual multiple steps were still required rendering the process cumbersome and time consuming.

Methods for sample processing and quantitation of a distinguishable cholate compound in a blood or serum sample using HPLC-MS analysis following analyte isolation are known, for example, as provided in U.S. Pat. No. 9,091,701, example 4, which is incorporated herein by reference. For example, an HPLC-MS system Agilent 1100 series Liquid Chromatograph Mass Spectrometer equipped with G1956A multimode source, using Agilent Eclipse XDB C8, 2.1×100 mm 3.5 um liquid chromatograph column may be employed for example, employing a column temperature of 40° C. and conditioned by running mobile phase for 30 min. An isocratic mobile phase buffer may be employed using 60% 10 mM ammonium acetate methanol/40% 10 mM ammonium acetate water. The MS may be run in multimode electrospray (MM-ES) ionization with atmospheric pressure chemical ionization (APCI). Selected ion monitoring (SIM) is performed at 407.30, 408.30 and 411.30 m/z. Three QC samples are assayed with each analytical run. The concentration of the QC samples must fall within 15% accuracy. Peaks are integrated by the system software.

Data from selective ion monitoring of either or both intravenous and oral samples are used to generate individualized oral and intravenous clearance curves for the patient. The curves are integrated along their respective valid time ranges and an area is generated for each. Comparison of intravenous and oral cholate clearance curves allows determination of first-pass hepatic elimination or portal shunt. The liver shunt fraction calculated by the formula:

ShuntFraction=[AUCₒᵣₐₗ/AUC_{IV}]*[Dose_{IV}/Doseₒᵣₐₗ]*100%,

wherein AUC represents area under the curve and Dose represents the amount (in mg) of dose administered.

A comparison of the advantages of the improved methods provided in the present disclosure for use in liver function tests provide a number of advantages compared to prior art LC-MS methods, for example, as disclosed in U.S. Pat. Nos. 8,778,299 and 9,091,701. These are summarize4d in Table 2.

TABLE 2
Comparison of MS, MS/MS, or LC-MS/MS methods
compared to LC-MS Prior Art Methods

| | Reference or mode | | |
|---|---|---|---|
| | U.S. Pat. Nos. 8,778,299 | | |
| | and 9,091,701 | LC-MS/MS | MS or MS/MS |
| Sample size serum | 0.5 mL | 0.05 mL | 0.01 mL |
| Extraction | Manual extraction | Partially or fully | add internal standard, |
| procedure | including adding | automated online | and aq. buffer, mix |
| | dilute aq. NaOH to | extraction including | with organic solvent, |
| | sample, solid phase | adding protein | phase separate and |
| | extraction, | precipitation solution | add upper layer to |
| | acidification, liquid- | to sample, centrifuge, | sample plate OR |
| | liquid extraction, | inject supernatant | sample dilution and |
| | evaporation, | | introduction to MS |
| | reconstitution with | | from microplate- |
| | mobile phase, and | | acoustic droplet |
| | injection | | ejection-no LC |
| | | | required |
| Analyte sample | 60-80% analyte | >90% analyte | >90% analyte |
| extraction | recovery | recovery | recovery |
| recovery | | | |
| Instrumentation | LC-MS selected ion | LC-MS/MS multiple | MS or MS/MS |
| detection mode | monitoring (SIM) | reaction mode | (MRM) |
| | | (MRM) | |
| Quantifies in each | d4-CA, 13C-CA | CA, d4-CA, 13C-CA, | CA, d4-CA, 13C-CA, |
| sample | | | |
| Correction factor | Isotopic contribution | Isotopic contribution | Isotopic contribution |
| required | of d4-CA to d5-CA | of d4-CA to d5-CA | of d4-CA to d5-CA |
| | and 12CA to 13CA | and 12CA to 13CA | and 12CA to 13CA |
| | taken into account | taken into account; | taken into account; |
| | particularly when | correction factor ~5% | correction factor ~5% |
| | endogenous CA too | or less | or less |
| | high; correction | | |
| | factor of ~25% or | | |
| | higher of signal may | | |
| | be required; | | |
| Run time | 7 min | 4.5 min | ~0.3 sec to 12 |
| | | | seconds |
| Throughput; | Lower throughput; | Higher throughput; <1 hr | Very high throughput |
| sample process + | days | (40+ test per week) | (up to ~260,000 |
| run time | (~5 tests per week) | | samples/day) |

Sample Collection

Improved methods for distinguishable bile acid quantitation are provided herein requiring only about 10 microliters or less, 10 microliters or more, 20 microliters or more, 30 microliters or more, 40 microliters or more, or 50 microliters or more of a patient blood or serum sample. This is at least a 10-fold reduction in amount of blood or serum sample compared to prior art methods, as disclosed in for example, U.S. Pat. No. 9,091,701. In contrast, methods provided herein require blood or serum sample size of about 20 microliters or greater, about 50 microliters or greater, or about 50 microliters to about 500 microliters, or about 50 to about 100 microliters.

The blood or serum sample for use in the present methods may be collected from a subject by any known method in the art. For example, see WHO guidelines on drawing blood: best practices in phlebotomy, World Health Organization, 2010, Geneva, Switzerland or BP-EIA: Collecting, processing, and handling venous, capillary, and blood spot samples, PATH, 2005. For example, venipuncture using needle and syringe or indwelling catheter, arterial blood sampling, pediatric or neonatal blood sampling, or capillary sampling may be employed. The choice of site and procedure may depend on the volume of blood needed for the procedure and laboratory test to be done. For example, a venous site, finger-prick or heel-prick, also known as capillary sampling or skin puncture, may be employed.

Whole blood samples may be obtained by venipuncture, collected in anticoagulant-containing vacutainer tubes, and refrigerated during storage and shipment. Blood samples can be further processed into different fractions. For example, after collection of whole blood, the blood may be allowed to clot by leaving it undisturbed at room temperature and then centrifuged. The upper portion is termed serum and does not contain fibrinogen. For example, whole blood may be allowed to stand for about 15-30 min. The resultant clot may be removed by centrifugation, for example, at 1,000-2,000×g for about 10 min in a refrigerated centrifuge. The resulting supernatant is designated serum. From a practical viewpoint, serum may be preferred to whole blood because of the possible rupture of erythrocytes that makes sample handling delicate. However, for blood or serum samples, storage and shipment require refrigerator, freezers, and/or special packaging with dry ice, so logistics may translate into significant costs.

Dried Blood Spots (DBS)

Dried blood spots (DBS) is a form of bio-sampling where blood samples are blotted and dried on filter paper. DBS may typically include the deposition of small volumes of capillary blood or venous blood onto dedicated paper cards. Comparatively to whole blood or plasma samples, their benefits rely in the fact that sample collection is easier and that logistic aspects related to sample storage and shipment can be relatively limited, respectively, without the need of a refrigerator or dry ice. Wagner et al., Mass Spectrometry Reviews, 2016, 35, 361-438.

DBS typically consist in the deposition of a few droplets of capillary blood, obtained by heel- or fingerpricking, onto filter papers in a card format (also known as “Guthrie cards”). Samples are simply allowed to dry, without any other processing. Chemically speaking, analytes are adsorbed with blood components onto a solid, cellulose-based matrix.

Compared to conventional venipuncture, much less volume is required, blood collection is simple, non-invasive and inexpensive, risk of bacterial contamination or hemplysis is minimal, and DBS can be preserved for long periods of time with almost no deterioration of analytes allowing ease of transport due to sample stability.

DBS sampling includes minimal sample volume, about 10-100, 20-80, or 30-70 microliters per spot.

Paper cards dedicated to DBS are commercially available from several manufacturers, and can be categorized in two groups: untreated and chemically treated papers. Untreated papers consist of pure cellulose and may be manufactured from 100% pure cotton linters.

Treated papers include cellulose treated with different proprietary chemicals. These may include Whatman (now part of GE Healthcare) FTA, FTA Elute, FTA DMPK-A, Whatman FTA DMPK-B (Majumdar & Howard, 2011), and Macherey Nagel NucleoCard (Moeller et al., 2012). FTA DMPK-A is impregnated with sodium dodecyl sulfate (SDS, <5%) and tris (hydroxymethyl)aminomethane (<5%), whereas FTA DMPK-B is impregnated with guanidinium thiocyanate (30-50%). Alternatively, untreated paper can be impregnated with chemicals by soaking it in a solution and allowing it to dry before use.

Adsorption and the solid nature of DBS make analytes typically less reactive than in (liquid) blood. One notable advantage of DBS is that analytes often exhibit excellent stability in ambient conditions, at least for several days (and up to several months in some cases), with only few precautions (samples packed in sealed bags with desiccant).

Compared to other blood-based samples, sample processing and short-term storage are greatly facilitated, and circumvent the need for dedicated apparatus such as centrifuges, homogenizers, refrigerators, or freezers. Moreover, DBS may reduce or eliminate biohazard risks. DBS offer significant advantages related to sample collection because DBS collection requires only pricking, it is not difficult to perform and can be learned easily by the medical staff or even by the patients themselves, whereas a phlebotomist is mandatory for venous blood collection. The volume of blood collected may be fairly low (typically a few dozen microliters), whereas standard blood-derived sampling in tubes requires volumes of 0.1 to a few milliliters. DBS are therefore appropriate when the volume of blood collected is limited, for example in newborns, infants, or critically ill patients.

The puncture site may be cleaned with 70% isopropanol. The skin may be pricked with a single-use, sterile lancet, and, after discarding the first drop of blood (to avoid leakage from interstitial fluids), subsequent drops are directly applied to the paper. Optionally, an internal standard may be spiked into the blood sample prior to spotting. The circles printed on the paper (e.g., 12 mm on Whatman 903 or Ahlstrom 226) should be filled completely and homogeneously. Samples are allowed to dry (room temperature, horizontal position, 3-4 hr). DBS samples are shipped to the laboratory within 24 hr and must meet some appearance criteria to be considered as suitable for screening. In particular, DBS that exhibit clotting, layering, super-saturation, insufficient volume, serum rings, visible traces of hemolysis, or contamination may be systematically rejected. Filter cards may be packaged in gas-impermeable zipper bags with desiccant sachets for shipping or storage. Samples may be frozen (−20° C. or lower) for longer term storage, stored at −4° C., or at ambient temperature for up to 14 days.

One method of elution of dried blood spots may be performed at ambient room temperature by punching out one spot with a single-use device from each blood-soaked circle. A circular punch (e.g., 9 mm, 7 mm, or 6 mm diameter) may be used. One or more dried blood spots from a single patient may be transferred to a multi-well plate. The well may be filled with phosphate-buffered saline using 0.05% TWEEN 20 and 0.08% sodium azide. The cell culture plate may be placed on a laboratory shaker allowing the dried blood spots to elute for about 4 hours or overnight. The next day, the spots typically are almost free of blood and hemolytic supernatants have formed. The eluate may be transferred to microfuge tubes and centrifuged to free supernatants from any debris. Supernatants may be transferred to sample vials or multi-well format for LC/MS-MS.

In another method of elution, the DBS punch sample or a VAMS tip may be exposed to an extraction solution to solubilize the analyte. The punch sample of VAMS tip may be optionally presoaked in water. The extraction solution may be, for example, water, acetonitrile, methanol, methanol-acetonitrile, methanol-water-formic acid, methanol-water, (e.g., 90% aq. MeOH; 4:1 v/v), or CHCl₃/MeOH (e.g., 2:1 v/v), for example at a temperature of about 25° C., without stirring for 30 min. or more. Optionally, the punch samples in the extraction solution may be vortexed, sonicated, incubated, and centrifuged. The supernatant may be dried in a lyophilizer. The dried sample may be dissolved in, or extracted using an extraction solution and diluted in, a mobile phase buffer (e.g., acetonitrile-water-formic acid; e.g., 5:95:0.1, v/v) and transferred to sample vials or multi-well format for LC/MS-MS.

A blood collector card, dried blood spot (DBS) technology, or HemaSpot™ device, such as a HemaSpot™-HF device may be employed. For example, a HemaSpot™ HF device uses a finger-stick to collect and dry blood within a protective cartridge. For example, an EBF blood spot collection card, Eastern Business Forms, Inc. Mauldin, S.C., may be employed, for example, a Five Spot blood card, or a Generic mulipart card, wherein each circle holds up to about 75-80 microliters of sample. Once dried, the sample is stable at ambient temperature and can be safely and easily shipped to a laboratory for analysis.

In another method, a capillary device such as a capillary tube is employed to obtain a fixed volume of blood sample.

Volumetric Absorptive Microsampling

Alternatively, a volumetric absorptive microsampling (VAMS™) device may be employed to obtain a blood sample. VAMS™ devices are handheld devices including a hydrophilic polymer tip connected to a plastic handle which wicks up a fixed volume (approximately 10, 20 or 30 microliters) when contacting a blood surface. VAMS effectively results in absorption of a fixed volume of blood, irrespective of the hematocrit.

Volumetric absorptive microsampling may take advantage of small volume sampling. A sample volume as low as 10, 20 or 30 microliters or more of a blood sample may be employed to wick a fixed volume of a capillary, venous blood, or serum sample.

VAMS™ samples may be obtained by dipping VAMS™ tips into appropriate blood or serum sample and drying for a period of time, e.g., about 2 hours of more, before extracting. The dried VAMS™ tip may be removed from the sampler by pulling the tip against the side of the extraction tube and adding 200 microliters of an extraction solution such as methanol, optionally containing an internal standard. The tube may be sealed and mixed on a lateral shaker. An aliquot of the supernatant (e.g., 50 microliters) may be diluted with mobile phase or, for example, of 1:4 methanol water prior to injection to LC-MS/MS system. Other extraction solutions may be employed as described above.

VAMS™ small volume collection devices are commercially available, for example, a Mitra® cartridge (Neoteryx, LLC).

Sample Extraction from Venous Sample

Previous multi-step analyte extraction procedure from a blood or serum sample included a laborious combination of solid phase extraction, liquid-liquid extraction, evaporation, and reconstitution. Methods are provided herein to replace several previous manual sample extraction steps with a simplified and partially automated online extraction procedure. In addition, unlabeled compounds such as unlabeled cholic acid may be quantified in each individual sample rather than only in the baseline samples.

Aliquots of the calibrator, quality control sample, or study sample may be added to a sample vial or deep well 96-well plates. The sample aliquot size may vary, for example from about 20 μL to about 500 μL, about 30 μL to about 400 μL, or about 40 to about 200 μL. A protein precipitation solution is added to each vial or well. The protein precipitation solution may contain a water-miscible organic solvent such as acetonitrile, or an alcoholic solvent such as methanol. The protein precipitation solution may be, for example, acetonitrile or 0.1 M ZnSO₄/methanol 60:40, or methanol. The sample aliquot may be mixed with 3-5 times its volume with the protein precipitation solution. In some embodiments, an acid is not added to the protein precipitation solution. In some embodiments, the protein precipitation solution is not acidified.

The protein precipitation solution may contain an internal standard. For example, if the distinguishable compound is a distinguishable bile acid, the internal standard may be a different distinguishable bile acid. For example, if the analyte is d4-CA or 13C-CA, the internal standard may be d5-CA. After addition of the protein precipitation solution to the sample aliquot, samples are vortexed, centrifuged, and the supernatant may be injected directly to an HPLC system, for example, in a LC-MS/MS system.

The samples may be vortexed for 1-10 min, 2-8 min, or about 5 min, centrifuged (16,000·g, 4° C., 15 min or 4,750·g for 20 min using deep-well 96 well plates), and the supernatant may be transferred into, for example, HPLC sample vials or into 0.5 mL 96 well injection plates.

The process of manual solid phase extraction, liquid-liquid extraction, evaporation, and reconstitution is no longer required when compared to prior art methods.

Sample analyte extraction recovery may be determined by comparing the LC, LC-MS, or LC-MS/MS peak areas and/or peak area ratios of samples prepared in serum to samples prepared in methanol.

Absolute extraction recovery may be assessed in human serum samples following the protocol described by Matuszewski et al. (2003), for example, using the analyte/internal standard ratios in the following samples as follows.

Pre-extraction spike: the human serum samples may be each spiked with distinguishable compound internal standard at the same level as the QC samples 0.25, 0.75, 2.5, and 7.5 μmol/L then extracted and analyzed. For example, human serum contains cholic acid. Before samples are spiked, cholic acid concentrations may be quantified. Cholic acid is spiked on top of the endogenous cholic acid to result in 0.25 (+ endogenous cholic acid) μmol/L, 0.75 (+ endogenous cholic acid) μmol/L, 2.5 (+ endogenous cholic acid) μmol/L, and 7.5 (+ endogenous cholic acid) μmol/L.

Post-extraction spike: The samples may be first extracted and then spiked resulting in the same concentrations as described for the pre-extraction spiked samples above: 0.25, 0.75, 2.5, and 7.5 μmol/L internal standard. For example, cholic acid is spiked on top of the endogenous cholic acid to result in 0.25 (+ endogenous cholic acid) μmol/L, 0.75 (+ endogenous cholic acid) μmol/L, 2.5 (+ endogenous cholic acid) μmol/L, and 7.5 (+ endogenous cholic acid) μmol/L.

Sample Preparation

The present disclosure provides methods comprising simplified patient sample preparation, compared to prior art methods. The sample may be any appropriate patient sample. In some embodiments, the patient sample is a blood or serum sample. Sample preparation may involve off-line or in-line sample preparation. For example, off-line sample preparation may be performed prior to MS or LC-MS or LC-MS/MS. Sample preparation may optionally include protein precipitation with organic solvents such as methanol or acetonitrile or other organic solvents. Liquid-liquid extraction (LLE) may be performed with organic solvents such as ether or hexane or other organic solvents. Solid phase extraction (SPE) may be performed with any appropriate chromatography solid phase media, including normal phase, reverse phase, ion exchange, hydrophobic interaction, size exclusion, affinity chromatography, and so forth. For example, a solid phase matrix such as C18 or C8 or other reverse phase matrix. Liquid chromatography may be performed with a matrix such as C18 or C8 or other reverse phase matrix. Gel electrophoresis by slab 1D or 2D or capillary electrophoresis may be performed. The sample preparation may involve simple sample dilution. Sample preparation may involve in-line, or automated, sample preparation, which may be continuous with MS or LC-MS or LC-MS/MS. In line sample preparation may involve solid phase extraction (SPE) with matrix such as C18 or C8 or other solid phase matrix.

For each corresponding samples pair (pre- and post-extraction spike), the absolute extraction recovery is calculated as follows:

Extraction recovery [%]=analyte/internal standard ratio pre-extraction spike/post-extract spike×100.

Analyte Detection and Quantitation

Improved methods for detecting and quantifying a distinguishable bile acid in a blood or serum sample are provided. Previous methods for quantifying analytes employed LC-MS with selected ion monitoring. In the present disclosure, detection and quantification is based on analyte ion transitions in multiple reaction mode (MS/MS versus MS).

LC-MS and LC-MS/MS are the combination of liquid chromatography (LC) with mass spectrometry (MS). A sample in a liquid form may be injected into the LC system and different chemical components are separated based on differing affinities for the stationary phase inside the column and the mobile phase flowing through a solid phase column. The output of the LC column is directed into the mass spectrometer where it is ionized by e.g., electrospray or chemical ionization.

In single mass spectrometry (MS) only the precursor ion is analyzed as generated in the source, for example, in an ion trap, a single quadrapole, or time of flight MS. In contrast, MS/MS is the combination of two mass analyzers in one instrument. The first mass spectrometer filters for the precursor ion followed by fragmentation of the precursor ion, e.g., with high energy and nitrogen gas. A second mass spectrometer is used to filter for the product ions generated by fragmentation. LC-MS/MS may utilize, for example, a tandem quadrapole (triple quadrapole) mass spectrometer (QQQ) or a quadrapole time-of-flight mass spectrometer (QTOF). The advantages of MS/MS is increased sensitivity, for example in the QQQ, due to reduction in noise, and more structural information can be obtained on the analyte (QTOF) based on fragmentation pattern. LC-MS/MS when used in MRM mode scanning for both precursor and product ion increases specificity in addition to enhanced sensitivity. For example, two compounds of the same molecular weight will produce the same precursor ion, but can be identified and quantified based on different product ions formed after fragmentation. The increase in sensitivity of MS/MS over single MS may be exploited to decrease the required sample volume of the blood or serum sample from the subject. The present methods exhibit about ten-fold increased sensitivity compared to previous LC-MS methods. Increased sensitivity allows for decreased serum sample volume by about 10-fold. For example, methods are provided requiring about 50 microliters of patient serum sample, whereas previous methods may require 0.5 mL (500 microliters) of serum sample, for example, as disclosed in U.S. Pat. No. 9,091,701.

One advantage of multiple reaction monitoring (MRM) is that a unique fragment ion may be monitored and quantified in the midst of a complicated sample matrix. MRM plots may be simple, usually containing a single peak.

The multiple reaction monitoring (MRM) method is accomplished by specifying the parent mass of the analyte compound for MS/MS fragmentation and then specifically monitoring for a simple fragment ion. The specific experiment is known as a “transition” and can be written (parent mass→fragment mass). The MS/MS may be run in positive ionization mode or in negative ionization mode using MRM monitoring. For example, when the MS/MS is run in the negative multiple reaction monitoring mode, cholic acid (CA) may be monitored at, for example, m/z=407.3→343.1 (quantifier ion) and 289.2 (qualifier ion), ¹³C-CA at m/z=408.3→343.1 (quantifier ion) and 289.2 (qualifier ion), D₄-CA at m/z=411.3→347.1 (quantifier ion) and 290.2 (qualifier ion) and the internal standard D₅-CA at m/z=412.3→290.2 (quantifier ion) and 348.1 (qualifier ion). In another example, the MS/MS may be run in negative mode with Q1 mass and Q3 mass for each of 12C-CA (Q1=407.25 Da, Q3=343.1 Da), 13C-CA (Q1=408.25 Da, Q3=343.1 Da), d4-CA (Q1=411.25 Da, Q3=347.2 Da) and d5-CA (Q1=412.25 Da, Q3=348.1 Da) as shown in Table 19. In another example, the MS/MS may be run in positive ionization mode, with Q1 mass and Q3 mass for each of 12C-CA (Q1=409.3 Da, Q3=355.4 Da), 13C-CA (Q1=410.3 Da, Q3=356.3 Da), d4-CA (Q1=413.4 Da, Q3=359.4 Da) and d5-CA (Q1=414.4 Da, Q3=360.4 Da and 245.1 Da) as shown in Table 17.

The supernatant sample vials or 96 well injection plates loaded with processed sample supernatant may be added to an autosampler or manually injected to a separation system.

The separation system may be an in-line separation system coupled with a mass detection system. The separation system may include a preparative component and an analytical component. The separation system may comprise a chromatography system. The chromatography system may include LC (liquid chromatography), HPLC (high performance liquid chromatography), or UPLC (UHPLC, ultra-performance liquid chromatography). The preparative component may be used to pre-purify, isolate, and/or concentrate the one or more distinguishable compounds in the sample, or sample supernatant. The preparative component may include an extraction column. The preparative component may include a solid phase resin. The separation system may include an analytical component. The analytical component may be used to purify, concentrate, and/or assist in separating the distinguishable compounds from each other and from other sample components. The analytical component may include a solid phase component. The preparative solid phase component may include a solid phase. In some embodiments, the solid phase resin of the preparative component and the analytical component are each independently selected from the group consisting of a normal phase resin, reverse phase resin, hydrophobic interaction solid phase resin, hydrophilic interaction solid phase resin, ion-exchange solid phase resin, size-exclusion solid phase resin, and affinity-based solid phase resin.

The solid phase resin in the preparative component and/or analytical component may be selected from, for example, a normal phase resin, for example, a silica gel resin, a reverse phase resin such as a C4, C8, C18, phenyl, propyl, or other hydrophobic interaction solid phase, an ion-exchange solid phase resin, a size-exclusion solid phase resin, and an affinity-based solid phase resin. In some embodiment, the preparative component and the analytical component employ the same solid phase material. In some embodiments, the preparative component and the analytical component comprise different solid phase materials.

The separation system may include an LC-MS/MS system, as described herein.

For example, twenty μL of the samples may injected onto an extraction column. The extraction column may be a reverse phase extraction column. For example, a C84.6-12.5 mm 5 μm extraction column may be employed (e.g., Eclipse XDB C-8, Agilent Technologies, Palo Alto, Calif.). Samples may be washed with a mobile phase buffer (e.g., an isocratic buffer containing 15% methanol with 0.1% formic acid and 85% water with 0.1% formic acid). The flow may be 2-3 mL/min within 0.5 min and the temperature for the extraction column may be set to a temperature selected from between about 30-45° C., or 40° C.

After 0.5 min, or from about 3 to about 8, or about 5 column volumes, the switching valve may be activated and the analytes (one or more distinguishable compounds) may be eluted in the backflush mode from the extraction column onto an analytical column. The analytical column may be a reverse phase analytical column. The analytical column may be, for example, a 150·4.6 mm C8, 5 μm analytical column (e.g., Zorbax XDB C8, Agilent Technologies, Palo Alto, Calif.). An isocratic or gradient elution of the analyte from the analytical column may be performed. For example, a gradient elution may use an A:B mobile phase buffer, e.g., methanol with 0.1% formic acid (solvent B) and 0.1% formic acid in HPLC grade water (solvent A). The following gradient may run: 0 to 0.5 minutes: 60% solvent B, 0.5 to 1.5 minutes: 60% to 98% solvent B, 1.5 to 4 minutes: hold at 98% solvent B, 4 to 4.1 minutes: 98% to 60% solvent B, and stay at 60% solvent B for the next 0.5 min.

The mass detection system may comprise a mass spectrometer. The mass spectrometer may include an ion source system and a mass resolution/detection system. The ion source system may be any appropriate ion source system known in the art. In some embodiments, the ion source system is selected from the group consisting of electrospray ionization (ES), matrix-assisted laser desorption/ionization (MALDI), fast atom bombardment (FAB), chemical ionization (CI), atmospheric pressure chemical ionization (APCI), liquid secondary ionization (LSI), laser diode thermal desorption (LDTD), and surface-enhanced laser desorption/ionization (SELDI). In one aspect, the electrospray ionization system is a turbo electrospray ionization system. The mass resolution/detection system may be any appropriate mass resolution/detection system known in the art. In some embodiments, the mass resolution/detection system is selected from the group consisting of triple quadrupole mass spectrometer (MS/MS); single quadrupole mass spectrometer (MS); Fourier-transform mass spectrometer (FT-MS); and time-of-flight mass spectrometer (TOF-MS).

In one embodiment, the mass detection system includes an ion source comprising an electrospray ionization system. In one embodiment, the mass detection system includes a triple quadrupole mass spectrometer (MS/MS). The MS/MS may be run in the negative multiple reaction monitoring (MRM) mode, where cholic acid may be monitored at m/z=407.3→343.1 and 289.2 (qualifier ion), ¹³C-CA at m/z=408.3→343.1 and 289.2 (qualifier ion), D₄-CA at m/z=411.3→347.1 and 290.2 (qualifier ion) and the internal standard D₅-CA at m/z=412.3→290.2 and 348.1 (qualifier).

The present disclosure provides methods for processing, detecting, and quantifying distinguishable compound sample analytes in patient blood or serum samples. The results may be used for calculating liver function test values in a patient, for example, in SHUNT, FLOW (portal HFR), systemic HFR, STAT and/or DSI tests.

The portal HFR (FLOW), SHUNT, DSI and STAT tests may be used for defining disease severity in patients having chronic liver diseases.

STAT Test

The STAT test is a screening method for estimating portal blood flow and hepatic function. The STAT test is disclosed in Everson et al., U.S. Pat. No. 8,961,925, which is incorporated herein by reference. The STAT test is intended for screening purposes and is used in conjunction with FLOW and SHUNT tests to monitor hepatic blood flow and hepatic function. For example, a patient with a STAT screening test result above a cut-off level may be subjected to the more comprehensive portal HFR, SHUNT or DSI tests to monitor hepatic blood flow and hepatic function in the patient.

The STAT test is different from the SHUNT and FLOW tests in that only a single blood sample is drawn from the patient making the test more economical in terms of requiring less clinical personnel time, instrumentation time, and fewer clinical and laboratory supplies. For example, a single blood draw does not require an indwelling catheter. Preparation of a single sample is also less prone to error than multiple sequential samples. The test is also more comfortable for the patient and requires less time spent at the clinic

Rationale and Study Design for the Application of Liver Function Testing to CLD.

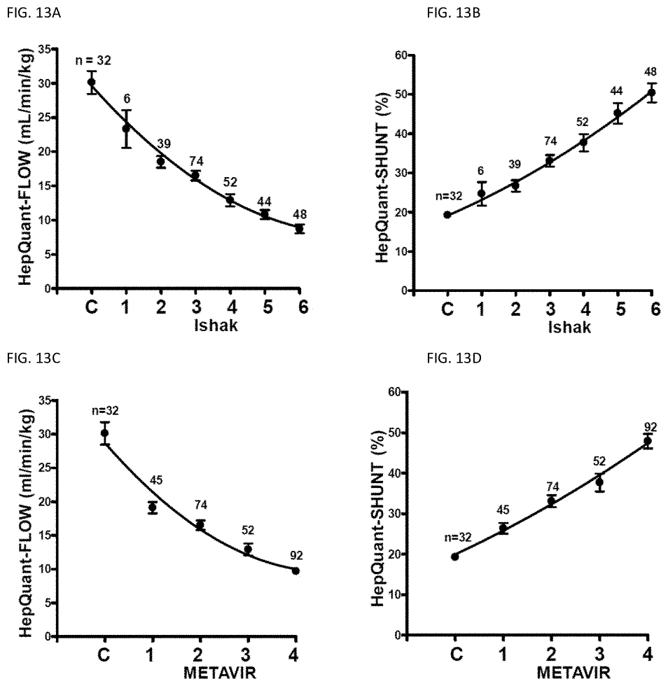

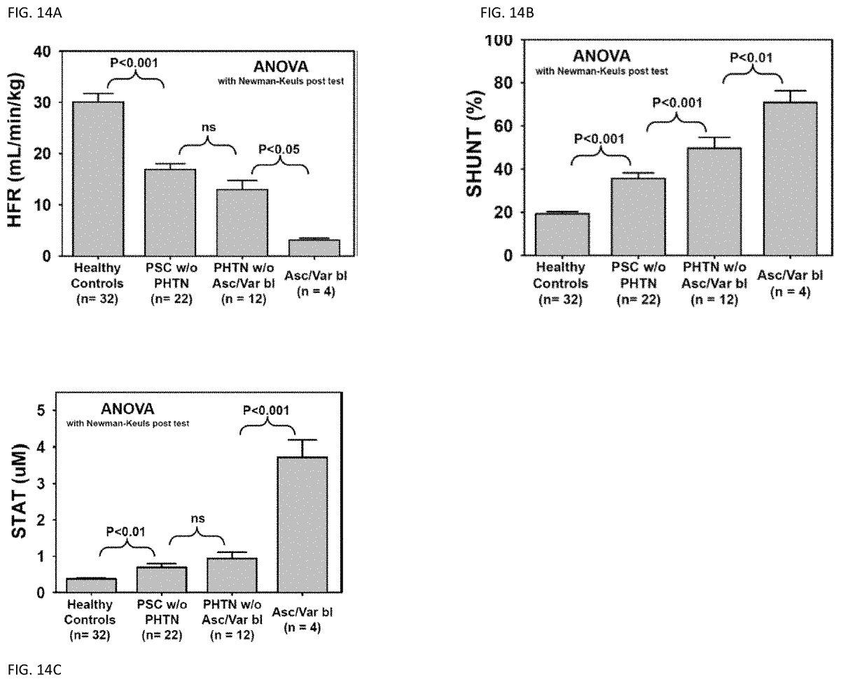

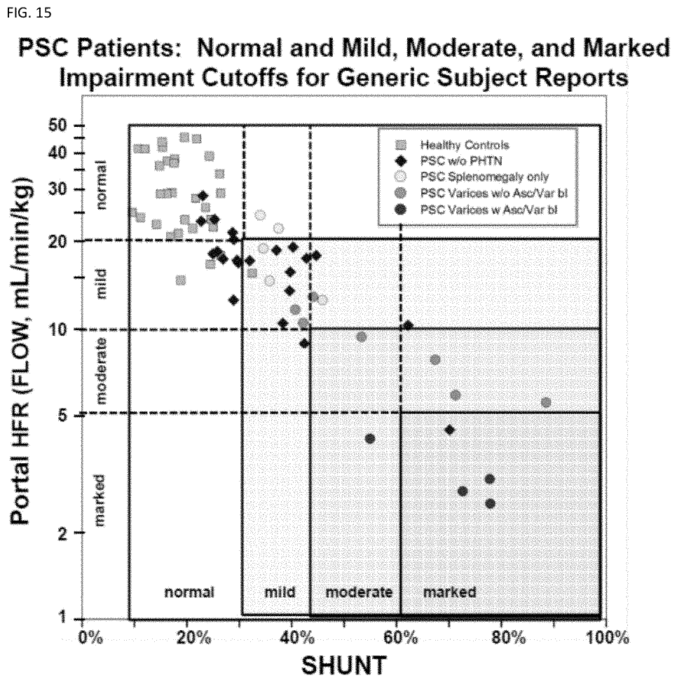

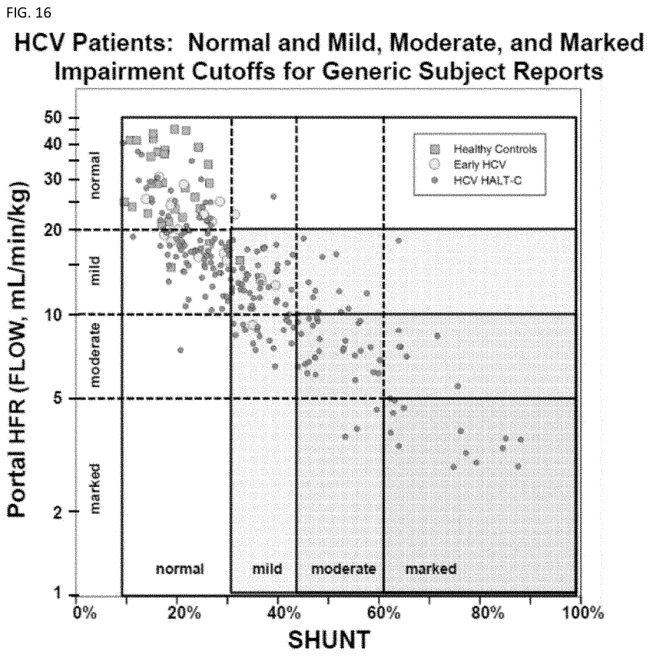

There is an expected similarity in disease progression of chronic liver diseases. For example, when comparing NAFLD to CHC, it is feasible to assess the entire spectrum of NAFLD because the pathophysiological progression is very similar to that of CHC. Progression is typically described by 4 stages of histologically described fibrosis. In both the CHC Metavir system (Group, TFMCS. 1994. Intraobserver and interobserver variations in liver biopsy interpretation in patients with chronic hepatitis C Hepatology. 20: 15-20) and NASH system (Brunt et al., 1999, Nonalcoholic steatohepatitis: A proposal for grading and staging the histological lesions. Am J Gastroenterol. 94: 2467-2474; Kleiner et al., 2005. Design and validation of a histological scoring system for nonalcoholic fatty liver disease. Hepatology. 41: 1313-1321) the absence of observable fibrosis is scored F0. Early stage fibrosis, F1, tends to be more periportal in CHC and can be periportal and/or perisinusoidal in NASH. In both scoring systems, F2 is more extensive periportal and perisinusoidal fibrosis, F3 is bridging fibrosis, and F4 is cirrhosis (Group, TFMCS. 1994; Brunt et al., 1999; Kleiner et al., 2005; Goodman, Z D. 2007. Grading and staging systems for inflammation and fibrosis in chronic liver diseases. J Hepatol. 47: 598-607). Because of this similar pattern of progression, it is expected that the portal flow impairment in NASH patients at stages F1-F4 to be comparable to CHC patients at corresponding Metavir stages F1-F4. Our previous CHC data stratified according to the 6 stage Ishak system can be readily converted (Goodman et al., 2007, infra) to the Metavir system to allow the estimation described below of the expected effect size, the number of subjects required, and approximate power of our proposed study. The correlation between scoring systems for FLOW and Ishak scoring, SHUNT and Ishak scoring, FLOW and Metavir scoring, and SHUNT and Metavir scoring is shown in FIGS. 13A-D, respectively.

Impact of Liver Testing in the Early Stages of Chronic Liver Disease. While most previous test development has focused on detecting advanced fibrosis and cirrhosis, it has been argued that the most serious need in NAFLD is the ability to distinguish early stage NASH from simple steatosis (Wilson and Chalasani, N. 2007. Noninvasive markers of advanced histology in nonalcoholic fatty liver disease: Are we there yet? Gastroenterology. 133: 1377-1378; discussion 1378-1379; and Vuppalanchi and Chalasani 2009. Nonalcoholic fatty liver disease and nonalcoholic steatohepatitis: Selected practical issues in their evaluation and management. Hepatology. 49: 306-317). The FLOW and SHUNT tests could detect the hepatic dysfunction of NASH patents and differentiate them from those with simple steatosis which are expected to have near normal portal flow.

In contrast to the FLOW and SHUNT tests, which require a minimum of 5 blood samples drawn from the patient over a period of 90 minutes or more following distinguishably-labeled cholate administration, it has been surprisingly discovered that results from a test including a single blood sample drawn after oral administration of a distinguishably-labeled cholate compound correlate to the results from FLOW, SHUNT, and DSI tests. The single time point screening test is called the STAT test.

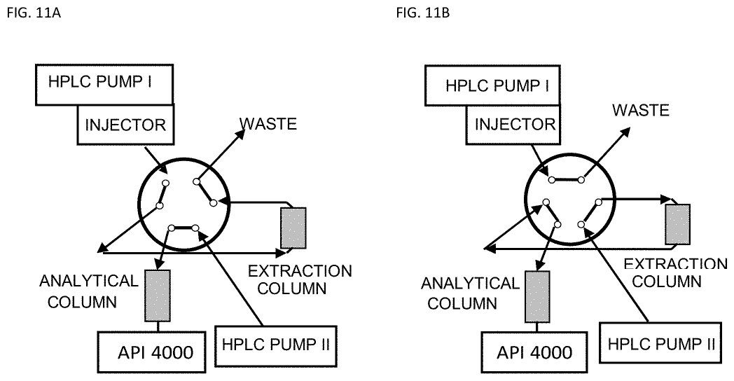

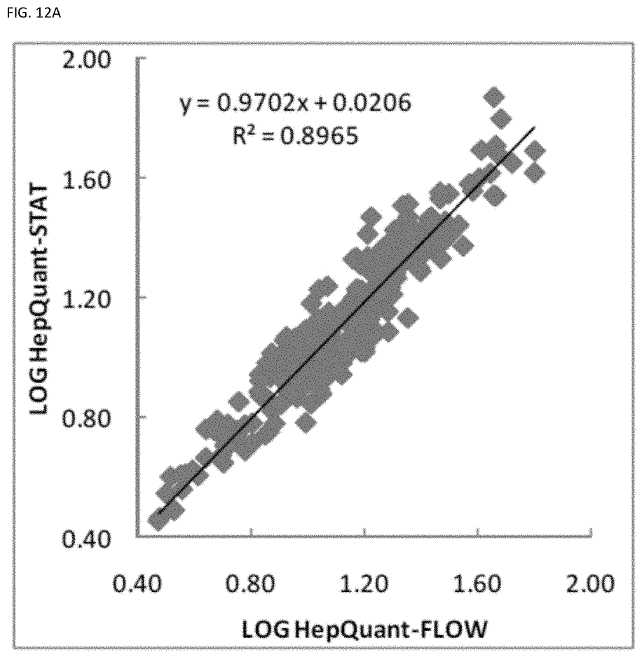

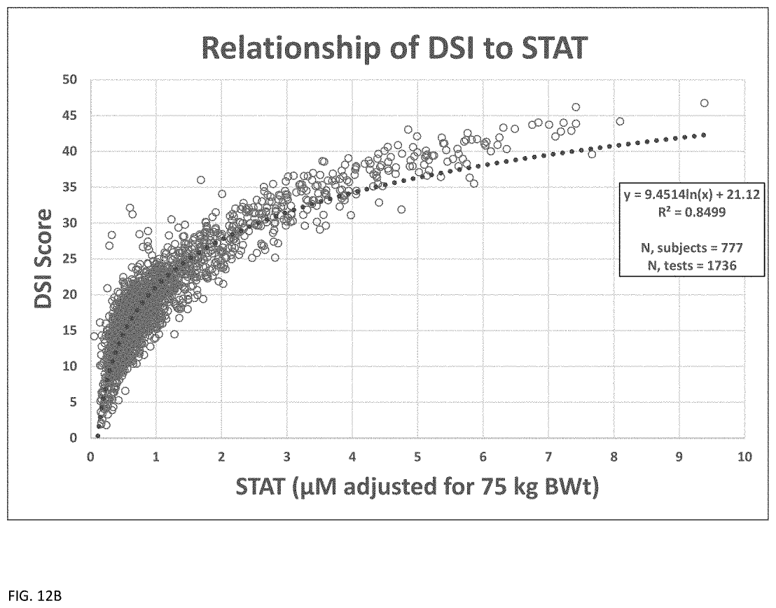

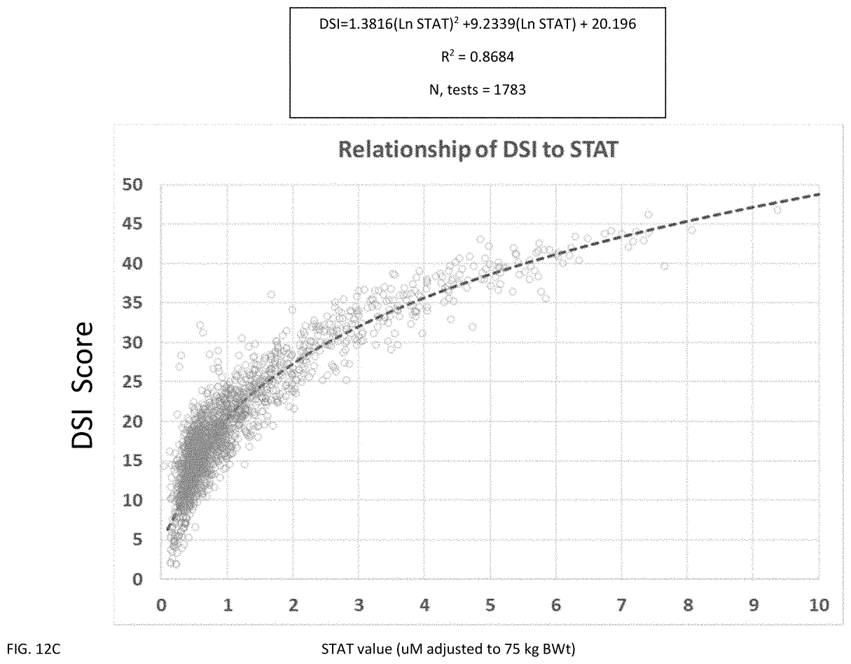

The time point for the STAT test single blood draw from the patient can be selected from, for example, any time point following oral administration of a distinguishable cholate; for example any time point selected from between about 10 and about 180 minutes post-administration. In one aspect, the time point is a single time point selected between about 20 and about 120 minutes post-administration. In another aspect, the time point is a single time point selected between about 30 and about 90 minutes post-administration. In one aspect the blood sample is drawn from the patient at any time point selected from about 30, 35, 40, 45, 50, 55, 60, 65, 70, 75, 80, 85, or 90 minutes, or any time point in between, post oral administration of the distinguishable cholate. In one aspect the time point for the single blood draw is selected from one of about 45, about 60 or about 90 minutes post administration. In one particular aspect, the single blood sample is drawn from the patient at about 45 minutes post administration. See for example, FIG. 5, where the results of the STAT test at 45 minutes post administration, are compared to the FLOW test. In another particular aspect, the single blood sample is drawn from the patient at about 60 minutes post oral administration of a distinguishable cholate. See for example, FIG. 12A, where the results of the STAT test at 60 minutes post administration, are compared to the FLOW test. The cholate concentrations at 60 minutes have been converted by the equation into estimated flow rates (mL/min/kg) and compared to the actual FLOW test results.

In one embodiment, the distinguishable compound for oral administration can be any distinguishable bile acid that is distinguishable analytically from an endogenous bile acid. In one aspect, the distinguishable bile acid is selected from any isotopically labeled bile acid known in the art. Distinguishable bile acids used in any one of these assays might be labeled with either stable (e.g., ¹³C, ²H, ¹⁸O) or radioactive (e.g., ¹⁴C, ³H) isotopes. Distinguishable cholate compounds can be purchased commercially (for example, Sigma-Aldrich, or CDN Isotopes Inc., Quebec, CA). In a preferred aspect, the distinguishable cholate is selected from any known safe, non-radioactive stable isotope of cholic acid. In one specific aspect, the distinguishable cholate compound is 2,2,4,4-²H cholic acid. In another specific aspect, the distinguishable cholate compound is 24-¹³C cholic acid.

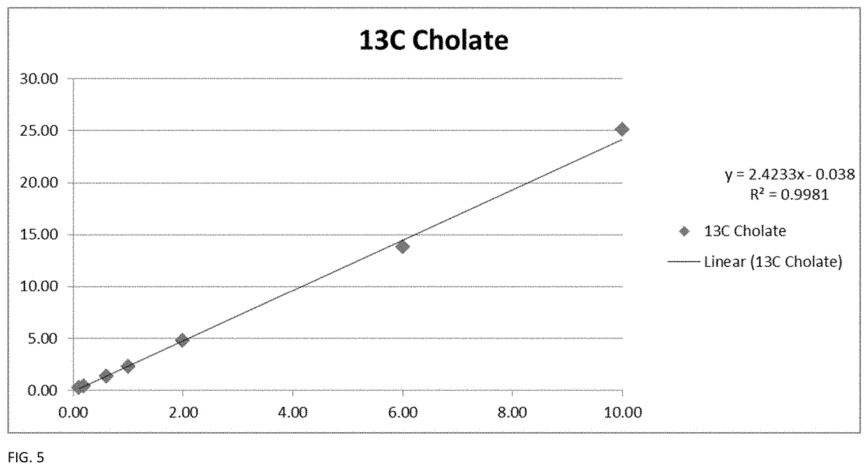

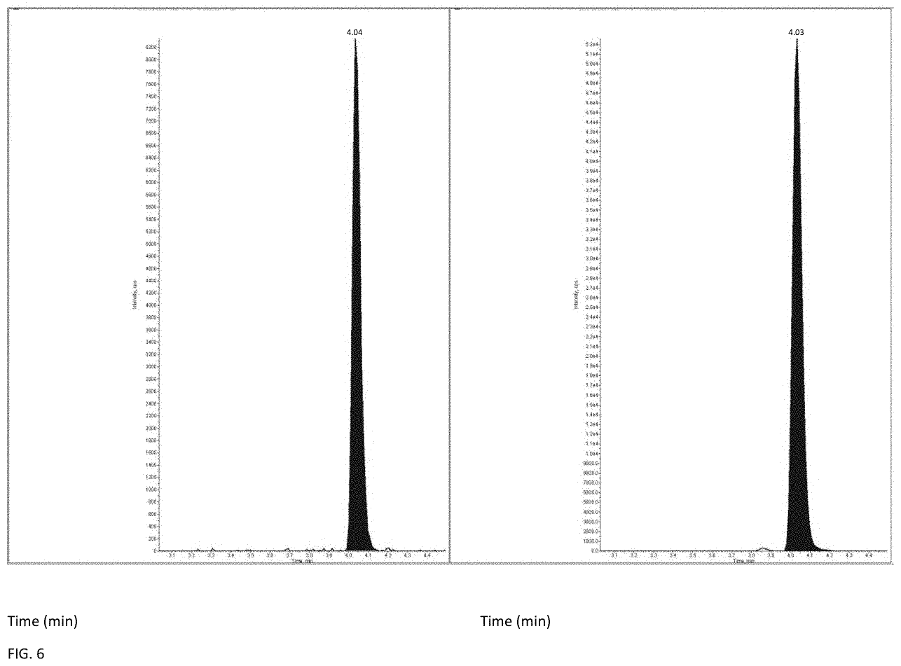

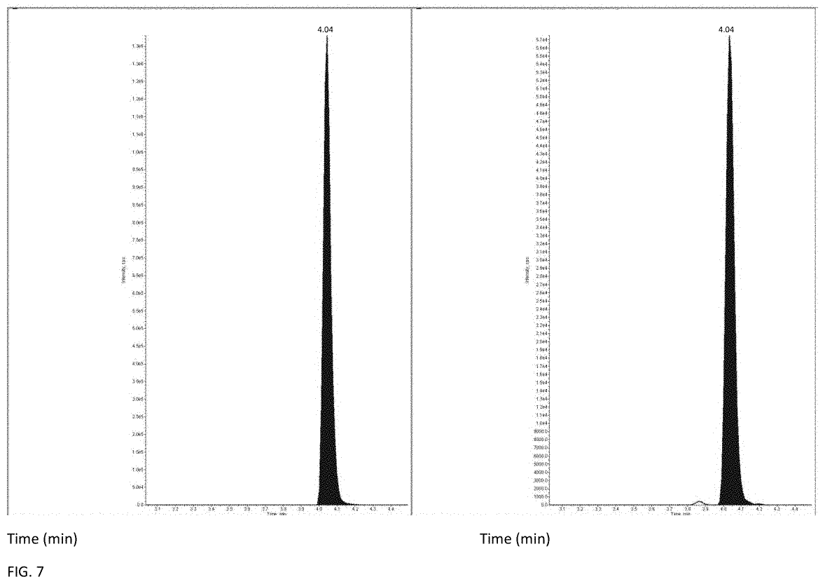

The STAT may be used as a screening test in a patient having, or suspected of having or at risk of any chronic liver disease (CLD). A STAT test result of 0.4±0.1 indicates a healthy patient. For example, the STAT test may be used as a screening test for a patient having, or suspected of having or at risk of NAFLD. Hepatitis can also be caused by excessive drinking as in Alcoholic SteatoHepatitis (ASH), or viral infection, i.e. Chronic Hepatitis C (CHC). All these chronic liver diseases (CLDs) are characterized by a similar patho-physiology with inflammation, cell death, and fibrosis leading to a progressive disruption of the hepatic microvasculature so, in various aspects, the STAT test will work on all CLD. For example, in patients diagnosed with PSC, 0.7±0.5 indicates PSC without PHTN, 1.6±1.5 indicates PSC with PHTN (splenomegaly of varices), 2.2±1.4 indicates PSC with varices, and 3.7±0.9 indicates PSC decompensated (varceal bleed or ascites). In another aspect, a STAT result indicates the patient should be followed with additional tests, such as FLOW, SHUNT, DSI or other diagnostic tests. See, e.g., FIGS. 6 and 7.

In another aspect, the single-point STAT test is used as an in vitro screen for disease progression of any chronic liver disease. For example, an individual patient diagnosed with, e.g., chronic hepatitis C, chronic hepatitis B, cytomegalovirus, Epstein Barr virus, alcoholic liver disease, amiodarone toxicity, methotrexate toxicity, nitrofurantoin toxicity, NAFLD, PSC, haemochromatosis, Wilson's disease, autoimmune chronic hepatitis, primary biliary cirrhosis, primary sclerosing cholangitis, or hepatocellular carcinoma, may be monitored over time using the STAT test.

In another aspect, the STAT test result is an indication of portal blood flow in any patient. The STAT test was initially developed especially to screen large numbers of potential patients. Those with a suspiciously low estimated portal flow would be referred for a FLOW or SHUNT test to more precisely assess hepatic impairment in early stage NASH. Patients with NASH need to be regularly monitored for progression in order to predict the course of their disease (Soderberg et al., 2010, Decreased survival of subjects with elevated liver function tests during a 28-year follow-up. Hepatology. 51: 595-602; Rafiq et al., 2009, Long-term follow-up of patients with nonalcoholic fatty liver. Clin Gastroenterol Hepatol. 7: 234-238). The prognostic utility of biopsy in NAFLD has been questioned (Angulo, P. 2010. Long-term mortality in nonalcoholic fatty liver disease: Is liver histology of any prognostic significance? Hepatology. 51: 373-375). As previously disclosed, FLOW and SHUNT testing was found to be superior to biopsy in predicting outcomes in chronic liver diseases such as CHC and is expected to be superior in NAFLD as well.

In another aspect, the STAT test is used to monitor effectiveness of treatment for a patient with liver disease. In one aspect the treatment is antiviral treatment.

In another aspect, the STAT test may be used to help prioritize patients waiting for a liver transplant. In one aspect, the patients waiting for liver transplant are patients with PSC, NASH, or chronic HCV.

In one embodiment, the STAT test is a non-invasive, in vitro test used to screen patients for liver function or liver disease; monitor liver disease patients undergoing antiviral therapy; monitor disease progression in patients with chronic liver disease; determine stage of disease in a patient diagnosed with HCV or PSC; prioritize liver disease patients for liver transplant; determine selection of patients with chronic hepatitis B who should receive antiviral therapy; assessing the risk of hepatic decompensation in patients with hepatocellular carcinoma (HCC) being evaluated for hepatic resection; identifying a subgroup of patients on waiting list with low MELD (Model for End-stage Liver Disease score) who are at-risk for dying while waiting for an organ donor; as an endpoint in clinical trials; replacing liver biopsy in pediatric populations; tracking of allograft function; measuring return of function in living donors; measuring functional impairment in cholestatic liver disease (PSC, Primary Sclerosing Cholangitis); or, used in combination with ALT to identify early stage F0-F2 HCV patients.

In one embodiment, the STAT test methods are used for the early detection of undiagnosed liver disease. In certain aspects, the STAT test methods disclosed herein are used to detect early stage liver disease and accurately monitor the progression of liver disease. Early detection with a test such as STAT leads to early intervention when it can be most effective and can reduce healthcare costs and greatly lower morbidity and mortality.

In embodiments, a STAT test value is obtained following oral administration of a distinguishable compound to the subject, a single blood or serum sample is drawn at a single specific time point following administration.

In some embodiments, the STAT test value, expressed as concentration of distinguishable cholate compound in the sample may be converted to an estimated portal flow rate (FLOW) value, or estimated portal HFR (FLOW) (expressed as mL/min/kg) in the subject by using an equation. In embodiments, the equation is y=0.9702x+0.0206, with R²=0.8965, where x is the log Hepquant FLOW and y is LOG Hepquant STAT, as shown in FIG. 12A.

In some embodiments, the STAT test value may be used to estimate a DSI value in a subject. For example, FIG. 12B shows the relationship of DSI to STAT values in n=1363 subjects, and n=1736 tests. The equation Y=9.4514 ln(x)+21.12, where x=STAT value (μM adjusted to 75 kg bodyweight), and Y=DSI value. The R2=0.8499. The coefficient of determination is R²=0.8499. R² is the square of the correlation coefficient R. The correlation coefficient formula may be used to indicate the strength of a linear relationship between two variables.

In another aspect, if the STAT test result for a patient is above a threshold value, the patient will undergo the FLOW, SHUNT, and/or DSI tests are used in conjunction with the STAT test. The FLOW and SHUNT tests can be used to accurately track liver disease. Patients attempting to modify their diet and lifestyle can see even small positive effects in a relatively short timeframe encouraging them to persevere. Physicians can track their patients and manage their care more effectively. Rapidly and accurately evaluating the efficacy of new drugs and therapies will greatly accelerate their development.

In one aspect, the STAT test can be administered to any patient, for example a patient having, or suspected of having or at risk of a chronic liver disease. In various specific aspects, the STAT test can be administered to a patient diagnosed, or suspected of having, NAFLD, PSC, hepatitis C, hepatitis B, alcoholic liver disease, and/or cholestatic disorders.

In further aspects, it is contemplated that the methods of the disclosure, can be used in conjunction with FLOW, SHUNT tests (oral cholate clearance and cholate shunt) and or DSI (dual cholate clearance tests) may be useful for a number of clinical applications, for example, selection of patients with chronic hepatitis B who should receive antiviral therapy; assessing the risk of hepatic decompensation in patients with hepatocellular carcinoma (HCC) being evaluated for hepatic resection; identifying a subgroup of patients on waiting list with low MELD (Model for End-stage Liver Disease score) who are at-risk for dying while waiting for an organ donor; as an endpoint in clinical trials; replacing liver biopsy in pediatric populations; tracking of allograft function; measuring return of function in living donors; and measuring functional impairment in cholestatic liver disease (PSC, Primary Sclerosing Cholangitis). The clinical endpoint may be a primary clinical endpoint or a secondary clinical endpoint.

In one embodiment, the herein disclosed STAT screening methods can be used in conjunction with FLOW and SHUNT tests (oral cholate clearance and cholate shunt) or DSI tests (dual cholate clearance tests) to monitor hepatic blood flow and hepatic function in an individual patient. A known population of patients is used to establish various cutoff values for the STAT, single-point screening test at a particular selected time point for drawing the single blood sample following oral administration of the distinguishable cholate.

In another aspect, the STAT test result for an individual patient is compared to the established cutoff values.

In one embodiment, the STAT test may be used in a patient suspected of having liver disease. A STAT test result from a patient falling within the range of about 0 to about 0.6 uM (“A” range) is likely to be predictive that the FLOW test result will also fall within the normal range for portal circulation. The patient with a STAT test result falling within the A range can be followed, for example, by use of an annual STAT test. A STAT test result falling within the range of about 0.6 uM to about 1.50 uM (“B” range) is likely to be predictive that the FLOW test result will fall within a compromised range for portal circulation. The patient with a STAT test result falling within the B range should be further evaluated, for example, with the FLOW, SHUNT and/or tests, for assessment of portal circulation and cholate clearances and shunt, respectively. A STAT test result falling above about 1.50 uM (“C” range) is likely to be predictive of advanced disease. The patient with a STAT test result falling within the C range should be further evaluated, for example, by EGD (upper endoscopy, esophagogastroduodenoscopy) and HCC (hepatocellular carcinoma) screening.

In another aspect, the STAT test may be used to monitor a patient periodically for improvement or liver disease progression. The patient can be monitored periodically for improvement or disease progression. For example, depending on the STAT test result, the patient can be followed for quantitative improvement with annual STAT, FLOW, SHUNT and/or DSI tests.

In another aspect, the STAT test can be used to screen and assess disease severity in a patient diagnosed or suspected of having a chronic liver disease, for example, PSC. STAT showed significant differences between healthy controls and patients with mild disease, and those with PHTN and decompensation (ascites or variceal bleeding), as shown in FIG. 14C. The simple and convenient STAT test can be used as a screen to direct patients to the more elaborate FLOW and SHUNT tests shown in FIGS. 14A and 14B, respectively. The SHUNT test was demonstrated to significantly differentiate between each subgroup, distinguishing PSC patients with mild disease from healthy controls, and also differentiating the cohorts with and without PHTN, and the group with PHTN from the group with a history of ascites or variceal bleeding, as in FIG. 14B.

Disease Severity Index (DSI)

Although various direct cut-offs for the FLOW and SHUNT tests were previously developed for specific conditions, in some cases use of a Disease Severity Index (DSI) more clearly delineates patient categories in chronic liver disease.

The “Disease Severity Index” (DSI) employs a mathematical model designed for adaptation of a bioassay result (liver function test) to the assessment of disease severity of an individual patient. For example, a DSI equation is developed using liver function test results from a defined patient population and healthy controls. In some embodiments, a DSI equation is developed from a specific patient population. The DSI equation has one or more terms selected from SHUNT, Portal HFR, and/or Systemic HFR depending on type or severity of liver disease. In some embodiments, one or more DSI cut-offs are used for DSI comparison, depending on type of disease and severity of disease. In some embodiments, use of the DSI value in a patient requires only a simple table look up.

In some embodiments, a method is provided for determining a disease severity index (DSI) value in a patient having or suspected of having or at risk of a chronic liver disease comprising (a) obtaining one or more liver function test values in the patient, wherein the one or more liver function test values are obtained from one or more liver function tests selected from the group consisting of SHUNT, Portal HFR and Systemic HFR; and (b) employing a disease severity index equation (DSI equation) to obtain the DSI value; where at least one term of the DSI equation independently represents a liver function test value in the patient, or a mathematically transformed liver function test value in the patient; and optionally wherein the at least one term of the DSI equation is multiplied by a coefficient specific to the liver function test. In some embodiments, the mathematically transformed liver function test value in the patient is selected from a log, antilog, natural log, natural antilog, or inverse of the liver function test value in the patient. In some embodiments, each term of the DSI equation represents a liver function test value or a mathematically transformed liver function test value.

DSI is a function of Shunt and Portal HFR and Systemic HFR, so DSI may be determined using a Generic DSI equation 1:

DSI= [symbol] (Shunt,Portal HFR,Systemic HFR)

Specific DSI equations are provided in U.S. Pat. Nos. 9,091,701, 9,759,731, 10,520,517, each of which is incorporated herein by reference in its entirety.

Additional specific DSI equations include the following example equations.

DSI equation 2 employs SHUNT, portal HFR and systemic HFR patient values.

DSI=A(Shunt)+B(Log Portal HFR)+C(log Systemic HFR)+D, DSI Equation 2:

wherein the constants A, B, C, D for use in DSI Equation 2 are shown in Table 3.

TABLE 3
Constants and coefficients for use in DSI Equation 2

| | A | B | C | D |
|---|---|---|---|---|
| DSI 3.1 | 5.75 | 7.22 | 8.45 | 50 |
| DSI 3.2 | 5.75 | 7.22 | 9.28 | 51.27 |
| DSI 3.3 | 5.34 | 6.65 | 8.57 | 44.66 |
| DSI 3.4 | 5.3 | 6.6 | 8.7 | 44.7 |

DSI equation 3 employs portal HFR and systemic HFR patient values.

$$DSI = A \sqrt{\frac{{( B - Log Portal HFR )}^{2} +}{{( C - Log Systemic HFR )}^{2}}} \qquad DSI Equation 3$$

Constants and coefficients for use in Equation 3 are shown in Table 4.

TABLE 4
Constants and Coefficients for use in DSI Equation 3

| | A | B | C |
|---|---|---|---|
| DSI rtuln | 10.86186 | 3.94527 | 2.37168 |

In some embodiments, a SHUNT test value in the patient may be used in the DSI equation, and the SHUNT test value is determined by a method comprising receiving a plurality of blood or serum samples collected from the patient having PSC, following oral administration of a dose of a first distinguishable cholate (doseₒᵣₐₗ) to the patient and simultaneous intravenous co-administration of a dose of a second distinguishable cholate (doseᵢᵥ) to the patient, wherein the samples have been collected over intervals spanning a period of time after administration; quantifying the concentration of the first and the second distinguishable cholates in each sample; generating an individualized oral clearance curve from the concentration of the first distinguishable cholate in each sample comprising using a computer algorithm curve fitting to a model oral distinguishable cholate clearance curve and computing the area under the individualized oral clearance curve (AUCoral); generating an individualized intravenous clearance curve from the concentration of the second distinguishable cholate in each sample by use of a computer algorithm curve fitting to a model intravenous second distinguishable cholate clearance curve and computing the area under the individualized intravenous clearance curve (AUCiv); and calculating the shunt value in the patient using the formula:

AUCₒᵣₐₗ/AUCᵢᵥ×Doseᵢᵥ/Doseₒᵣₐₗ×100%.

In some embodiments, the SHUNT test employs a first distinguishable cholate is a first stable isotope labeled cholic acid and a second distinguishable cholate is a second stable isotope labeled cholic acid. In some embodiments, the first and second stable isotope labeled cholic acids are selected from 2,2,4,4-d4 cholate and 24-¹³C-cholate. In some embodiments, the samples have been collected from the patient over intervals of from two to seven time points after administration. In some embodiments, the samples have been collected from the patient at 5, 20, 45, 60 and 90 minutes after administration. In some embodiments, the samples have been collected over intervals spanning a period of time from the time of administration to a time selected from about 45 minutes to about 180 minutes after administration. In some embodiments, the samples have been collected over intervals spanning a period of time of about 90 minutes or less after administration.

The portal HFR, systemic HFR and/or SHUNT values in the patient may be determined by a method provided herein.

In some embodiments, the portal HFR, systemic HFR and/or SHUNT values in the patient is/are provided by a method comprising measuring concentration of the distinguishable compound in each sample by a method comprising LC-MS/MS with MRM.

In some embodiments, a portal HFR value in the patient may be determined by a method comprising (i) receiving a plurality of blood or serum samples collected from a patient having or at risk of a chronic liver disease, following oral administration of a dose of a distinguishable compound (doseₒᵣₐₗ) to the patient, wherein the samples have been collected from the patient over intervals spanning a period of time of less than 3 hours after administration; (ii) measuring concentration of the distinguishable compound in each sample; (iii) generating an individualized oral clearance curve from the concentration of the distinguishable compound in each sample comprising using a computer algorithm curve fitting to a model distinguishable compound clearance curve; (iv) computing the area under the individualized oral clearance curve (AUC) (mg/mL/min) and dividing the dose (in mg) by AUC of the orally administered distinguishable compound to obtain the oral distinguishable compound clearance in the patient; and (v) dividing the oral distinguishable compound clearance by the weight of the patient in kg to obtain the portal HFR value in the patient (mL/min/kg).

In some embodiments, a systemic HFR value in the patient may be determined by a method comprising (i) receiving a plurality of blood or serum samples collected from a patient having or at risk of a chronic liver disease, following intravenous administration of a dose of a distinguishable compound (closely) to the patient, wherein the samples have been collected from the patient over intervals spanning a period of time of less than 3 hours after administration; (ii) measuring concentration of the distinguishable compound in each sample; (iii) generating an individualized intravenous clearance curve from the concentration of the distinguishable compound in each sample comprising using a computer algorithm curve fitting to a model distinguishable compound clearance curve; (iv) computing the area under the individualized intravenous clearance curve (AUC) (mg/mL/min) and dividing the dose (in mg) by AUC of the intravenously administered distinguishable compound to obtain the intravenous distinguishable compound clearance in the patient; and (v) dividing the intravenous distinguishable compound clearance by the weight of the patient in kg to obtain the systemic HFR value in the patient (mL/min/kg).

In some embodiments, a method is provided for calculating a disease severity index (DSI) value in a patient having or suspected of having or at risk of a chronic liver disease, the method comprising obtaining serum samples from a patient having or suspected of having or at risk of a chronic liver disease, wherein the patient previously received oral administration of a first stable distinguishable compound and simultaneously intravenous administration of a second distinguishable compound, and wherein blood samples had been collected from the patient over an interval of less than 3 hours following administration of the first and second distinguishable compounds; assaying the serum samples to calculate the portal hepatic filtration rate (portal HFR) as mL/min/kg, wherein kg is body weight of the patient, the systemic hepatic filtration rate (systemic HFR) as mL/min/kg wherein kg is body weight of the patient, and SHUNT as %; and calculating a DSI value for the patient by using a DSI equation, as provided herein.

In some embodiments, a peripheral venous catheter is placed in the patient, oral (D4-cholate, 40 mg), and simultaneously, IV (13C-cholate, 20 mg) are administered to the patient. Blood samples are drawn at t=5, 20, 45, 60 and 90 minutes post administration. Optionally, a baseline sample is drawn prior to administration. The samples are processed and the distinguishable bile acids are measured by LC-MS/MS with MRM according to the disclosure to obtain STAT, portal HFR, systemic HFR, SHUNT, cholate elimination rate, RCA20, DSI values, algebraic HR values, and/or indexed HR values in a subject. DSI values may be obtained from portal HFR, systemic HFR, and/or SHUNT values by employing a DSI equation.

In some embodiments, the DSI equation may be selected from:

DSI=f(Shunt,Portal HFR,Systemic HFR) Eqn. 1:

DSI=A(Shunt)+B(Log Portal HFR)+C(log Systemic HFR)+D, Eqn. 2:

wherein the constants and coefficients for use in DSI Equation 2 are shown in Table 3.

$$DSI = A \sqrt{\frac{{( B - Log Portal HFR )}^{2} +}{{( C - Log Systemic HFR )}^{2}}} \qquad Eqn . 3$$

wherein constants and coefficients for use in Equation 3 are shown in Table 4.

DSI=A(SHUNT)+B(logₑ portal HFR)+C(logₑ systemic HFR)+D, Eqn 4:

wherein SHUNT is SHUNT test value in the patient (%); portal HFR is portal hepatic flow rate (HFR) test value in the patient as mL/min/kg, wherein kg is body weight of the patient; systemic HFR is systemic HFR value in the patient as mL/min/kg, wherein kg is body weight of the patient; A is a SHUNT coefficient; B is a Portal HFR coefficient; C is a Systemic HFR coefficient; and D is a constant. In some embodiments, the SHUNT, the portal HFR, and the systemic HFR test values in the patient are obtained on the same day. The constant D may be a positive number from 5 to 125. The SHUNT coefficient A may be a number from 0 to positive 25. The Portal HFR coefficient B may be a number from 0 to negative 25. The Systemic HFR coefficient C may be a number from 0 to negative 25.

For example, the DSI equation may be selected from:

DSI=9.84(SHUNT)−12.36 Logₑ(portal HFR)+50.5; Eqn. 5:

DSI=5.75(SHUNT)−7.22(LogₑPortal HFR)−8.45(Logₑ Systemic HFR)+50; Eqn. 6:

or

DSI=5.34(SHUNT)−6.65(Logₑ Portal HFR)−8.57(Logₑ Systemic HFR)+44.66. Eqn. 7:

The Formula for DSI may be given as a function with 3 coefficients and 2 measured variables. One general equation for calculating DSI is:

DSI=A{square root over ((B−(ln HFRₚ))²+(C−(ln HFRₛ))²)}, Eqn. 8:

where A is a scaling multiplier (10.86186) to yield a range from 0 (no disease) to 50 (end-stage disease), B is the natural logarithm of the maximum value for Portal HFR, b, and C is the natural logarithm of the maximum value for Systemic HFR, c. Thus, the DSI formula can also be written as:

$$DSI = A \sqrt{{( ln ( \frac{b}{{HFR}_{p}} ) )}^{2} + {( ln ( \frac{c}{{HFR}_{s}} ) )}^{2}} \qquad Eqn . 9$$

wherein A=a scaling multiplier from 8 to 12 (optionally 10.86) to yield a range from 0 (no disease) to 50 (end-stage disease), b is the maximum value for Portal HFR in a range from 25-75, and c is the maximum value for Systemic HFR in a range from 5-15.

The variables in the DSI equation, b, c, HFRₚ, and HFRₛ, are all clearance values with units of mL min⁻¹ kg⁻¹—but, the units drop in the equation due to factoring the variables as ratios. HFRₚ is an apparent clearance, dependent upon the amount of orally administered d4-cholate that spills into the systemic compartment where peripheral venous blood is sampled.

Based on the range of HFRs in 50 healthy controls (30 lean, 16 overweight, 4 obese) (Portal HFR 26.57±8.37 mL min⁻¹ kg⁻¹; Systemic HFR 6.09±1.54 mL min⁻¹ kg⁻¹), the maximum values for Portal HFR and Systemic HFR may be set at:

| | | |
|---|---|---|
| Portal HFR | B = 3.94527 | b = 51.69 |
| Systemic HFR | C = 2.37168 | c = 10.72 |

As HFRₚ and HFRₛ approach b and c, respectively, DSI approaches 0—“NO DISEASE”. As HFRs approach 1, DSI approaches 50—“END STAGE DISEASE”.

In some embodiments, the disease severity index equation used to assess chronic liver disease in the patient includes where SHUNT is SHUNT test value in the patient (%) and portal HFR is portal HFR test value in the patient as mL/min/kg, wherein kg is body weight of the patient, wherein the SHUNT and the portal HFR test values in the patient were obtained on the same day.

In some embodiments, at least one term of the DSI equation independently represents a mathematically transformed liver function test value in the patient from step wherein the mathematically transformed liver function test value in the patient is selected from a log, antilog, natural log, natural antilog, or inverse of the liver function test value in the patient.

In some embodiments, each term of the DSI equation independently represents a liver function test value in the patient, or a mathematically transformed liver function test value in the patient, and the at least one term of the DSI equation is multiplied by a coefficient specific to the liver function test.

The constant and coefficient(s) of the DSI equation can vary with liver disease type and/or disease severity. In some embodiments, the constant and coefficients are interrelated so, for example, if all were divided by 10 then the DSI would go from 0-5, rather than 0-50, and healthy would be 1 instead of 10. In some embodiments, the constant is a positive number from 5 to 125. In some embodiments, the SHUNT coefficient is a number from 0 to positive 25. In some embodiments, the Portal HFR coefficient is a number from 0 to negative 25. In some embodiments, the Systemic HFR coefficient is a number from 0 to negative 25.

In some embodiments, the at least one term in the DSI equation is multiplied by a coefficient specific to each type of test, to obtain the DSI. In some embodiments, the DSI in the patient is compared to one or more DSI cut-off values indicative of at least one clinical outcome.

In some embodiments, each term of the DSI equation independently represents a liver function test value in the patient, or a mathematically transformed liver function test value in the patient, and the at least one term of the DSI equation is multiplied by a coefficient specific to the liver function test.

In some embodiments, the DSI value in the patient is used to assess chronic liver disease severity, status, or resolution in the patient selected from chronic hepatitis C, chronic hepatitis B, cytomegalovirus, Epstein Barr virus, alcoholic liver disease, amiodarone toxicity, methotrexate toxicity, nitrofurantoin toxicity, non-alcoholic steatohepatitis (NASH), non-alcoholic fatty liver disease (NAFLD), haemochromatosis, Wilson's disease, autoimmune chronic hepatitis, primary biliary cirrhosis, primary sclerosing cholangitis (PSC), and hepatocellular carcinoma (HCC).

In some embodiments, the DSI value may be used for identifying increased risk for portal hypertension or decompensation in the chronic liver disease patient wherein a DSI≥18 indicates increased risk for portal hypertension (PHTN), and a DSI≥36 indicates an increased risk for decompensation. In some embodiments, the portal hypertension (PHTN) is defined as splemomegaly or varices, and decompensation is defined as ascites or variceal hemorrhage. In some embodiments, the chronic liver disease is primary sclerosing cholangitis.

In some embodiments, the DSI value in a patient suffering from a chronic liver disease may be used for prediction of clinical outcomes in the chronic liver disease patient, wherein a DSI≥25 indicates an increased risk of severe clinical outcome in the patient. In some embodiments, the chronic liver disease is chronic hepatitis C.

In some embodiments, the severe clinical outcome is selected from CTP progression, variceal hemorrhage, ascites, hepatic encephalopathy, ascites+encephalopathy, or liver-related death.

In some embodiments, the DSI value in a patient on the waiting list for liver transplant (LT) may be used for prioritizing the patient on the waiting list for LT, wherein the priority of the patient on the waiting list for LT is increased following an increase in the DSI value over time in the patient, or following a DSI value in the patient of greater than 40.

In some embodiments, the DSI value in a patient having a chronic liver disease may be used for prediction of future clinical outcomes, wherein a DSI>19 indicates an increased risk of clinical outcomes in the patient.

In some embodiments, a DSI equation is provided comprising two or more terms and a constant to obtain the DSI value, wherein at least one term of the DSI equation independently represents a liver function test value in the patient, or a mathematically transformed liver function test value in the patient; wherein the at least one term of the DSI equation is multiplied by a coefficient specific to the liver function test, and the DSI equation optionally comprises one or more additional terms representing values from clinical biochemistry laboratory assays selected from the group consisting of serum albumin, alanine transaminase, aspartate transaminase, alkaline phosphatase, total bilirubin, direct bilirubin, gamma glutamyl transpeptidase, 5′ Nucleotidase, PT-INR (prothrombin time-international normalized ratio), caffeine elimination, antipyrine clearance, galactose elimination capacity, formation of MEGX from lidocaine, methacetin-C13, and methionine-C13; and/or one or more additional terms representing clinical features selected from varices, ascites, or hepatic encephalopathy.

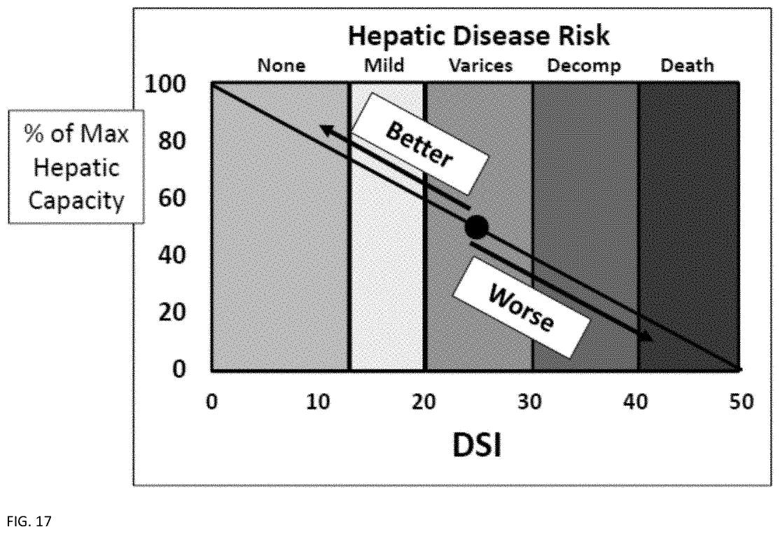

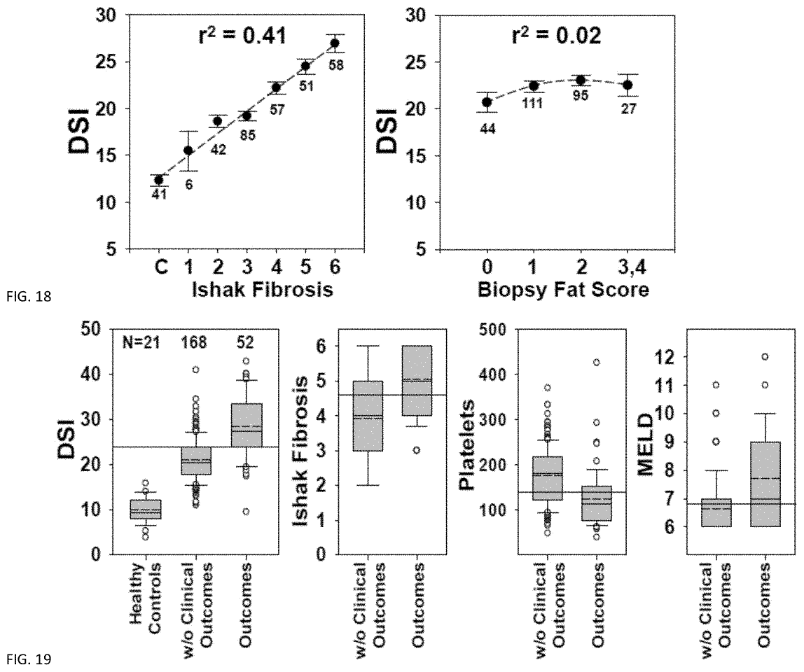

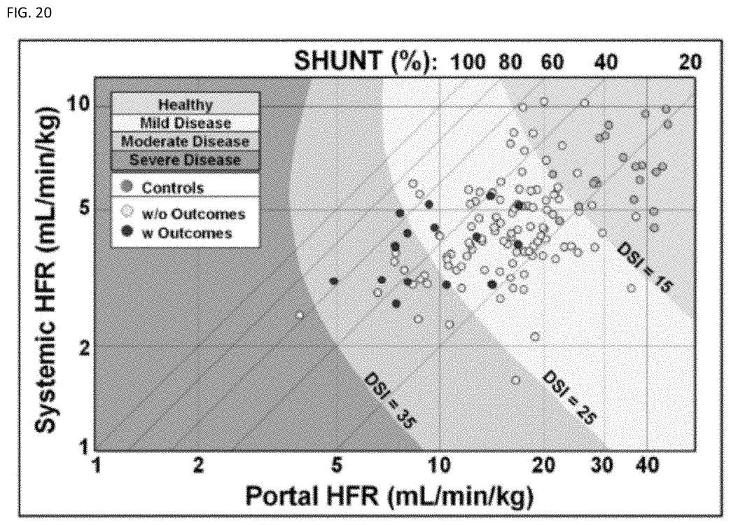

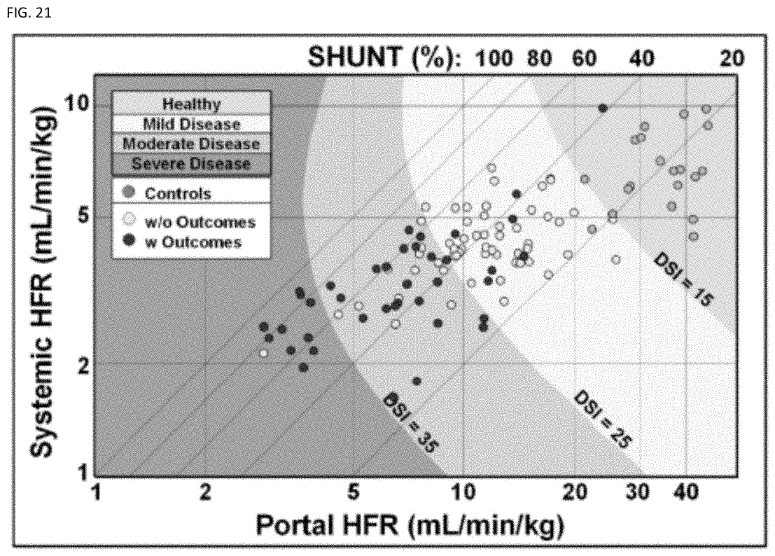

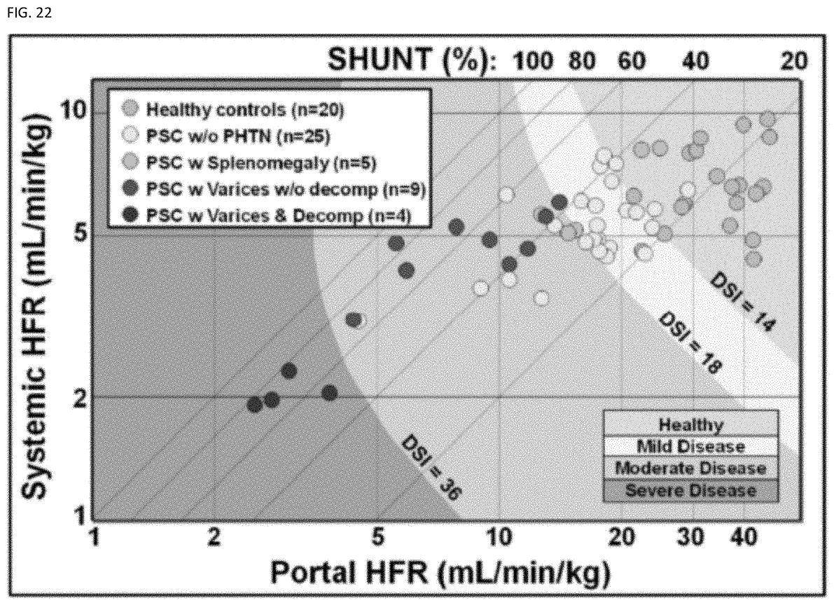

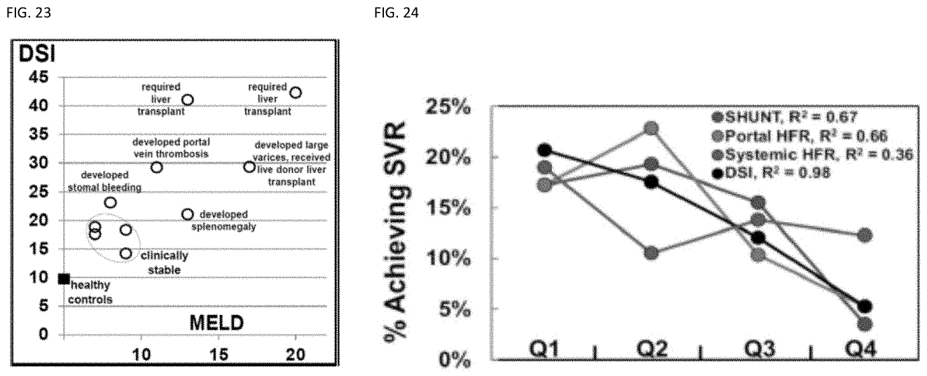

In some embodiments, a DSI value in a patient may be used to determine % of maximum hepatic capacity in a patient. For example, FIG. 17 shows a graph of the relationship of a DSI value in a patient to % of maximum hepatic capacity. A higher DSI value is indicative of a lower % of maximum hepatic capacity and worsening of chronic liver disease severity. Conversely, a lower DSI value is indicative of a higher % of maximum hepatic capacity and decreased chronic liver disease severity. For example, a DSI value of 12 may be indicative of about 75% of maximum hepatic capacity in a patient. A DSI value of 20 may indicate about 60% of maximum hepatic capacity in a patient. A DSI value of 25 may indicate about 50% of maximum hepatic capacity in a patient. A DSI value of 30 may indicate about 30% of maximum hepatic capacity in a patient. A DSI value of 40 may indicate about 20% of maximum hepatic capacity in a patient.

In some embodiments, the method for determining a disease severity index (DSI) value in a patient further comprises comparing the DSI value in the patient to one or more DSI cut-off values, one or more normal healthy controls, or one or more DSI values within the patient over time. In some embodiments, the comparing the DSI value in the patient to one or more DSI cut-off values is indicative of at least one clinical outcome. In some embodiments, the clinical outcome is selected from the group consisting of Child-Turcotte-Pugh (CTP) increase, varices, encephalopathy, ascites, and liver related death.

In some embodiments, comparing the DSI value within the patient over time is used to monitor the effectiveness of a treatment of chronic liver disease in the patient, wherein a decrease in the DSI value in the patient over time is indicative of treatment effectiveness.

In some embodiments, comparing the DSI value in the patient over time is used to monitor the need for treatment of chronic liver disease in the patient, wherein an increase in the DSI value in the patient over time is indicative of a need for treatment in the patient.

In some embodiments, the DSI value in the patient is used to monitor the need for, or the effectiveness of, a treatment of chronic liver disease in the patient wherein the treatment is selected from the group consisting of antiviral treatment, antifibrotic treatment, antibiotics, immunosuppressive treatments, anti-cancer treatments, ursodeoxycholic acid, insulin sensitizing agents, interventional treatment, liver transplant, lifestyle changes, and dietary restrictions, low glycemic index diet, antioxidants, vitamin supplements, transjugular intrahepatic portosystemic shunt (TIPS), catheter-directed thrombolysis, balloon dilation and stent placement, balloon-dilation and drainage, weight loss, exercise, and avoidance of alcohol.

In some embodiments, the DSI value in the patient is used to assess severity of chronic liver disease in the patient, for example, wherein the CLD is chronic hepatitis C, non-alcoholic fatty liver disease or primary sclerosing cholangitis. A DSI value may be used for identifying increased risk for portal hypertension or decompensation in a patient having a the chronic liver disease wherein a cut-off of DSI≥18 indicates increased risk for portal hypertension (PHTN), and a cut-off of DSI≥36 indicates an increased risk for decompensation. DSI cut-off values of 15, 25 and >35 may indicate mild disease, moderate disease and severe chronic liver disease, respectively. A DSI value may be used to predict a response to treatment, such as % of patients with CHC who will achieve SVR following treatment with an antiviral drug, such as PEG/RBV, for example, wherein a DSI cut-off of 30, may indicate limited or no ability to achieve SVR.

In some embodiments, comparing a DSI value within a patient over time may be used to monitor chronic liver disease status or disease progression of a chronic liver disease in the patient, wherein change in DSI value within the patient over time is used to inform the patient of status of the disease and risk for future clinical outcomes, wherein an increase in the DSI value within the patient over time is indicative of a worse prognosis, and a decrease in the DSI value within the patient over time is indicative of a better prognosis.

In some embodiments, kits are provided for determining one or more of STAT, portal HFR, systemic HFR, SHUNT, cholate elimination rate, RCA20, DSI values, algebraic HR values, and/or indexed HR values in a subject by the methods described herein. One or more distinguishable compound(s) may be provided in a kit may be employed to assess liver function in a health facility and/or a home kit depending on format. Kits may thus comprise, a suitable container means, and an oral dose of distinguishable compound. The oral dose of distinguishable compound may be administered to a subject inside or outside of a health facility such as a clinic, test center, or hospital. In addition, a second IV dose of a distinguishable compound may be administered in a health facility. Sample tubes for collection and/or shipping of the samples such as blood collection may also be included. In one example, a kit may comprise an oral and an IV dose of one or more distinguishable compounds and sample tubes for collection of samples over a period of less than 3 hours after administration of the distinguishable compounds. In another example, a kit may comprise an oral dose of one or more distinguishable compounds and sample tubes for collection of samples over a period of less than 3 hours, after administration of the distinguishable compounds. In another example, a kit may comprise components necessary for a test period of 90 minutes post administration of one or more distinguishable compounds. In a further example, a kit may comprises components necessary for a test period of 30 minutes post administration of distinguishable compounds.

The sample may be serum, whole blood, venous blood, or capillary blood. For example, the sample may be whole blood or serum collected by venipuncture, or may be capillary blood, for example, collected by fingerstick or heelstick.

In a further example, the kit may further include lancet(s), capillary tube(s), filter paper cards for dried blood spot sample collection, and/or volumetric absorptive microsampling devices.

The distinguishable compounds may be administered to the subject and samples collected in the same health facility. Samples may be analyzed within the same facility, or may shipped to a reference lab, hospital lab, or test center for analysis.

The kit may include a point of care, lateral-flow test cassette. The blood, serum or capillary blood sample may be processed and applied to the lateral flow device. When the distinguishable compound is present in the sample at a concentration above one or more threshold value(s), the distinguishable compound may be visualized in the test.

Further suitable reagents for use in the present kits may include a distinguishable agent diluent, intravenous distinguishable compound diluent, serum albumin for the intravenous sample, protein precipitation solution, oral distinguishable compound diluent. The kits may further comprise a suitably aliquoted composition of one or more distinguishable compounds, whether labeled or unlabeled, which as may be used as internal control(s) or external control(s), for example, to prepare a standard curve for a detection assay.

The container means of the kits will generally include at least one vial, test tube, flask, bottle, syringe or other container means, into which the distinguishable agent may be placed, and preferably, suitably aliquoted. The kits of the present invention will also typically include a means for containing the distinguishable compound(s) and any other reagent containers in close confinement for commercial sale. Such containers may include injection or blow-molded plastic containers into which the desired vials are retained. In addition, the kits may contain a product or diluent for diluting the oral distinguishable compound such as a fruit juice or other liquid. The juice may be a non-citrus juice.

The distinguishable compound may be provided in a kit may be employed in an in vitro test to assess liver function in a health facility and/or a home kit format. For example, a patient suspected of having a disease or condition can be tested with the STAT test after undergoing a History or Physical Exam or standard lab tests. A low test result (“A” range) will suggest the patient be followed with a yearly exam. An intermediate result (“B” range) will indicate the patient should be tested with either the FLOW, SHUNT, and/or DSI test. A high result (“C” range) indicates the patient should be suspected of having an advanced stage of disease and should undergo further screeing, e.g. undergo esophagogastroduodenoscopy (EGD) or hepatocellular carcinoma (HCC) screening.

The distinguishable compound may be used as a hepatic blood flow assessing agent and may comprise a suitable container means, an oral dose of distinguishable cholate to possibly be administered in an outpatient facility, within a hospital setting, or outside of a hospital environment. Sample tubes, dried blood spot filter papers, volumetric absorptive sample devices, lancets, capillary tubes, and/or lateral flow devices may be included. In one example, a kit may comprise an oral dose of the distinguishable cholate and sample tubes for collection of a single sample following a period of, for example, selected from a specific time point from about 10 to about 200 minutes after oral administration of the distinguishable cholate. In a specific example, one blood sample is collected at a time point of about 45 minutes after administration of the distinguishable cholate. In another specific example one blood sample is collected at a time period of about 60 minutes after administration of the distinguishable cholate. In a further example, a kit may comprise components necessary for a test period of 30 minutes post administration of distinguishable agents. The kits may further comprise a suitably aliquoted composition of the specific agent such as cholate, or a diagnostic pharmaceutical composition comprising a distinguishable cholate, whether labeled or unlabeled, as may be used to prepare a standard curve for a detection assay. The diagnostic pharmaceutical composition may include a distinguishable compound and optionally additional pharmaceutically acceptable excipients, diluents, buffer compounds, pH adjusting agents, colorings, flavorings, and/or vehicles as known in the art.

The container means of the kits will generally include at least one vial, test tube, flask, bottle, syringe or other container means, into which the distinguishable agent may be placed, and preferably, suitably aliquoted. The kits of the present invention will also typically include a means for containing the distinguishable agent and any other reagent containers in close confinement for commercial sale. Such containers may include injection or blow-molded plastic containers into which the desired vials are retained. In addition, the kits may contain a product for diluting the distinguishable oral agent.

In embodiments, the kit may further include instructions for comparing the amount of distinguishable cholate compound to a cutoff value or cutoffs of values to determine the state of portal blood flow and/or hepatic function in the patient.

Preparation of Quality Control Samples for Kits.

The FDA provides guidance as to acceptable levels of accuracy and precision of analytical methods. See, for example, Bioanalytical Method Validation, May 2001, Section VI. Application of Validated Method to Routine Drug Analysis. Once the analytical method has been validated for routine use, its accuracy and precision should be monitored regularly to ensure that the method continues to perform satisfactorily. To achieve this objective, a number of QC samples are prepared separately and should be analyzed with processed test samples at intervals based on the total number of samples. The QC samples may be run in duplicate at three concentrations (one near the lower limit of quantification (LLOQ) (i.e., 3×LLOQ), one in midrange, and one close to the high end of the range) and should be incorporated in each assay run. The number of QC samples (in multiples of three) will depend on the total number of samples in the run. The results of the QC samples provide the basis of accepting or rejecting the run. At least four of every six QC samples should be within 15% of their respective nominal value. Two of the six QC samples may be outside the 15% of their respective nominal value, but not both at the same concentration.

The QC samples must include the high, middle, and low ranges of both standard curves. The QC samples are designed to closely simulate the actual concentrations of labeled compounds found in patient serum over the time course of the testing. For example, when the intravenously administered distinguishable compound is [24-²³C]-CA, its sample concentration is very high at the early time point and falls exponentially to medium and low concentrations. when the orally administered distinguishable compound is [2,2,4,4-²H]-CA concentration its sample concentration is very low at the early time point, rises to its highest value in the middle time points and then falls to a medium concentration.

In some embodiments, a kit of components is provided for estimation of portal blood flow and/or hepatic function in a subject; the kit comprising a first component comprising one or more vials, each vial comprising the single oral dose of the distinguishable compound; and a second component comprising one or more sets of labeled sterile blood-serum sample collection tubes. The kit may further comprise one or more sets of labeled transport vials. Optionally, each transport vial contains an internal distinguishable compound standard.

The kit may further comprises a single box for both shipping the vials to a health care practitioner and shipping the samples from the health care practitioner to the reference laboratory for analysis.

The distinguishable compound may be a distinguishable bile acid, bile acid conjugate, bile acid analog, or FXR agonist as provided herein. The distinguishable compound may be in a powder form or in a solution form. The first and second distinguishable compounds may be stable isotope labeled distinguishable bile acids. In some embodiments, the first and second stable isotope labeled distinguishable bile acids may be selected from 2,2,4,4-²H-cholic acid and 24-¹³C cholic acid.

The first composition and/or the second composition may further independently further comprise one or more components selected from the group consisting of pharmaceutically acceptable excipients, diluents, colorings, flavorings, buffer compounds, pH adjusting agents, and vehicles. For example, the diluent may be selected from water, sodium bicarbonate solution, non-citrus juice, or normal saline (NS). The first and/or second composition may comprise sodium bicarbonate. The first composition and the second composition may independently be in a form selected from a powder form or a solution form. In some embodiments, the first and second compositions may both be in a solution form.

In some embodiments, the first composition may comprise a first distinguishable bile acid and sodium bicarbonate, optionally, wherein the first distinguishable bile acid is 2,2,4,4-²H-cholic acid. In some embodiments, the second composition comprises a second distinguishable bile acid and sodium bicarbonate, optionally, wherein the second distinguishable bile acid is 24-¹³C-cholic acid.

The kit may include container means selected from one or more of the group consisting of plastic containers, reagent containers, vials, tubes, flasks, and bottles. The kit may include shipping box, labels, instructions, package inserts, lancets, capillary tubes, syringes, indwelling catheter, 3-way stopcock, timer, and transfer pipets. For example, the kit may include a the shipping box comprising a single box for both shipping the vials to a health care practitioner and shipping the samples from the health care practitioner to a reference lab for analysis.

### EXAMPLES

### Example 1. Development of LC-MS/MS Assay

Previously, the present inventors developed and partially validated an LC-MS assay to quantify D₄-CA and ¹³C-CA that included a multi-step extraction procedure of the two analytes from human serum and detection of the analytes using selected ion monitoring, for example, as disclosed in U.S. Pat. No. 8,778,299.

Improved methods of sample extraction, analyte detection and quantification are provided herein employing LC-MS/MS for use in liver function assays in order to improve sample extraction efficiency, assay throughput, and to validate the assay for evaluation of analytical assay performance in support of pre-market device approval.

The previous multi-step extraction procedure including a combination of solid phase extraction, liquid-liquid extraction, evaporation and reconstitution was replaced by the automated online extraction procedure as described herein. Moreover, the assay was further streamlined and now quantified unlabeled cholic acid in each individual sample rather than only in the baseline samples.

Assay development followed the procedures and principles as set forth in CLSI. Liquid-chromatography-mass spectrometry methods. Approved guideline, C62-A, Wayne, Pa., Clinical and Laboratory Standards Institute, 2014; and CLSI. Mass spectrometry in the clinical laboratory: general principles and guidance. Approved guideline, C50-A, Wayne, Pa., Clinical and Laboratory Standards Institute, 2007.

However, the final method developed differed slightly from that described in the original and fully executed study plan. For example, one difference in comparison to previous assays was that instead of detecting the analytes in single ion mode (LC-MS), the analytes are now detected in the negative multi-reaction mode (MRM, LC-MS/MS).

Reference Materials:

Cholic acid-24²³C (¹³C-CA) and cholic-acid-2,2,4,4-d₄ (D₄-CA) were from Sigma Aldrich (St. Louis, Mo.). Cholic acid and the internal standard cholic acid-2,2,3,4,4-d₅ (D₅-CA) was from Sigma Aldrich (St. Louis, Mo.). All reference materials were in the free acid forms and had valid certificates of analysis.

Study Samples:

Human serum for assay validation was purchased from BioreclamationIVT (Westbury, N.Y.) and Gemini Bio Products (West Sacramento, Calif.). One hundred study samples were supplied by the Sponsor. Samples these had been kept at −70° C. or below in an appropriately qualified and monitored freezer and aliquots were transferred to Laboratory 1. After receipt, samples were stored at −70° C. or below in an appropriately qualified and monitored freezer.

Preparation of Calibration- and Quality Control Samples

Preparation of Stock and Working Solutions

Stock solutions of cholic acid (CA), ¹³C-CA, D₄-CA and the internal standard D₅-CA were based on three independent weightings of each compound. Stock solutions (10 nmol/L) were prepared in 1.0 mol/L NaHCO₃ buffer and stored at −70° C. or below. Five hundred μL of the 10 nmol/L stocks were diluted in 4500 μL of LC-MS grade water resulting in a working solution of 1 nmol/L. Working solutions for quality control samples and standard curves were prepared by dilution of the stock solutions with methanol/0.1 mol/L NaHCO₃ buffer (80/20, v/v).

Preparation of Internal Standard Solution:

The internal standard solution was aliquoted and transferred into 1.5 mL conical polypropylene tubes with snap-on lids and was stored at −70° C. or below. To prepare the protein precipitation solution containing 0.5 μmol/L D₅-CA, 50 μL of the internal standard solution were added to 99.95 mL of protein precipitation solution (in this case LC-MS grade methanol). Volumes were scaled according to the volumes needed. Protein precipitation solution was stored at +4° C. for up to 1 week.

Preparation of Calibrators and Quality Control (QC) Samples:

Serum quality control samples and calibration control samples as well as “blank” samples were prepared in bulk using human serum. In the following, “blank” samples were defined as samples from individuals who have neither received ¹³C-CA nor D₄-CA. As human serum contains cholate, samples for the cholic acid calibrators, but not for any of the other analytes, were diluted using Dulbecco's Phosphate Buffer Solution (DPBS) containing 5% human serum albumin: 1:10 (v/v) for the 0.1, 0.2, 0.6 and 1.0 μmol/L calibrators and 1:5 (v/v) for the 2.0, 6.0 and 10.0 μmol/L calibrators. Cholic acid was added to result in the calibrator concentrations. Two sets of calibrators were prepared to yield the concentrations below: one set with cholic acid (CA) and the other set containing both ¹³C-CA and D₄-CA.

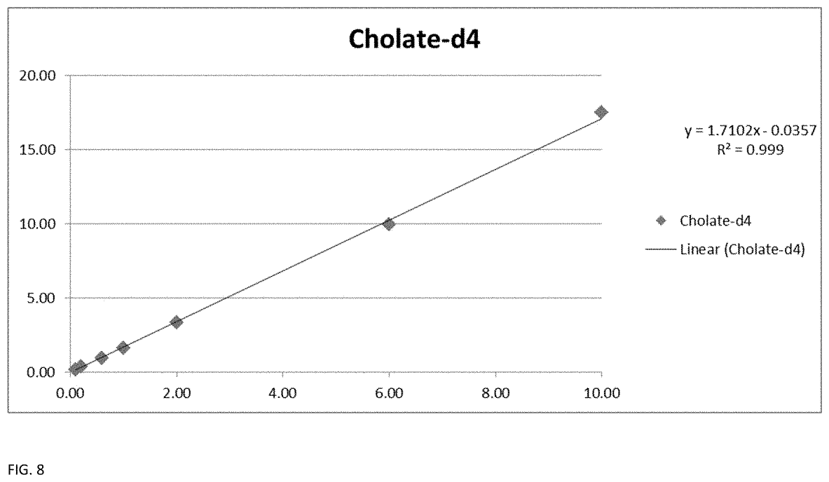

The serum calibration curves (0.1, 0.2, 0.6, 1.0, 2.0, 6.0, and 10 μmol/L) were constructed by plotting nominal concentration versus analyte area, after appropriate correction of areas for cross ion interferences. For example, see FIG. 2B for representative cholate calibration curve, FIG. 5 for 13C-cholate representative calibration curve, and FIG. 8 for representative d4-cholate calibration curve. A regression equation with 1/x weighting was used. Concentrations were calculated as μmon serum.

The QC samples for D₄-CA and ¹³C-CA (1-4) were:
1. 0.25 μmon D₄-CA and 7.5 μmol/L¹³C-CA
2. 0.75 μmon D₄-CA and 2.5 μmol/L¹³C-CA
3. 2.5 μmon D₄-CA and 0.75 μmol/L¹³C-CA
4. 7.5 μmon D₄-CA and 0.25 μmol/L¹³C-CA

The QC samples for CA (1-4) were:
1. Endogenous level QC (human serum)
2. Human serum enriched with 0.75 μmon CA
3. Human serum enriched with 2.5 μmon CA
4. Human serum enriched with 7.5 μmon CA

Calibrators and quality control were prepared fresh each day of analysis by spiking the appropriate working solutions into human serum or diluted serum for Cholic Acid calibrator samples.

Method of Sample Processing

Aliquots of 50 μL of the study serum sample, calibrator, or quality control sample were transferred into 1.5 mL deep-well 96 well plates. Two hundred μL of the protein precipitation solution containing the internal standard D₅-CA (0.5 μmon) were added to each well.

Samples were vortexed for 5 min, centrifuged (16,000·g, 4° C., 15 min or 4,750·g for 20 min using deep-well 96 well plates) and the supernatant was transferred into HPLC vials (or into 0.5 mL 96 well injection plates).

LC-MS/MS Equipment:

The Analytical system AB Sciex API 4000 LC-MS/MS was employed using a turbo electrospray interface, and negative multiple reaction monitoring (MRM) mode. The HPLC system included two G1312A binary pumps, two G1379A vacuum degassers and a G1316A thermostatted column compartment (all Agilent 1100 series, Agilent Technologies, Palo Alto, Calif.) with integrated 6-port Rheodyne column switching valve (Rheodyne, Cotati, Calif.). The connections of the switching valve are shown in FIG. 11A-B. The system included a Leap CTC PAL autosampler with cooling stack (Leap Technologies, Carrboro, N.C.). The analytical column was 150·4.6 mm C8, 5 μm (Zorbax Eclipse XDB C8, Agilent Technologies). The online extraction column was 12.5·4.6 mm C8, 5 μm (Zorbax Eclipse XDB C8, Agilent Technologies)

Mobile phase buffer was HPLC grade water+0.1% formic acid/HPLC grade methanol+0.1% formic acid. The flow rate was 2-3 mL/min during online extraction, and 1 mL/min for analytical column. The Autosampler temperature was 4° C. The Extraction column temperature was 40° C.

The Analytical column temperature was 40° C. The Injection volume of 20 is employed, unless otherwise specified. The Assay run time was 4.5 min.

Connections and positions of the column switching valve in the LC-MS/MS system are shown in FIG. 11A-B. As shown in FIG. 11A, in mode 1, the HPLC Pump I flows through the injector so the sample is injected to the extraction column. As shown in FIG. 11B, in mode 2, HPLC Pump II back flushes the extraction column onto the analytical column which is eluted to the API 4000 MS/MS system where MRM monitoring is employed.

Method of Analysis:

Twenty μL of the samples were injected onto a 4.6 12.5 mm 5 μm extraction column (Eclipse XDB C-8, Agilent Technologies, Palo Alto, Calif.). Following injection, sample extraction column was washed with a mobile phase of 15% methanol with 0.1% formic acid and 85% water with 0.1% formic acid. The flow was 2-3 mL/min within 0.5 min and the temperature for the extraction column was set to 40° C., as shown in FIG. 11A. After 0.5 min, the switching valve was activated and the analytes were eluted in the backflush mode from the extraction column onto a 150·4.6 mm C8, 5 μm analytical column (Zorbax XDB C8, Agilent Technologies, Palo Alto, Calif.), as shown in FIG. 11B. The mobile phase consisted of methanol with 0.1% formic acid (solvent B) and 0.1% formic acid in HPLC grade water (solvent A). The following gradient was run: 0 to 0.5 minutes: 60% solvent B, 0.5 to 1.5 minutes: 60% to 98% solvent B, 1.5 to 4 minutes: hold at 98% solvent B, 4 to 4.1 minutes: 98% to 60% solvent B, and stay at 60% solvent B for the next 0.5 min. Detection of eluate was performed by MS/MS run in the negative multiple reaction monitoring (MRM) mode. Cholic acid was monitored at m/z=407.3→343.1 and 289.2 (qualifier ion), ¹³C-CA at m/z=408.3→343.1 and 289.2 (qualifier ion), D₄-CA at m/z=411.3→347.1 and 290.2 (qualifier ion) and the internal standard D₅-CA at m/z=412.3→290.2 and 348.1 (qualifier).

After the analysis was completed, peaks were integrated (linear fit with 1/x weighting, generated in Excel) and the results were printed. The concentrations of cholic acid, ¹³C-cholic acid and cholic acid-D₄ were quantified using the calibration curves based on analyte/internal standard ratios that were included in each batch. Signal integrations were carried out by the Applied Biosystems Analyst Software (version 1.6.2 or higher), quantification was carried out using Microsoft Excel with semi-validated spread sheet.

Data Processing:

After the analysis was completed, peaks were integrated and the results were printed (AB SCIEX Analyst Software, version 1.6.2). Before concentrations were reported, the analyte and internal standard signals were corrected for analyte isotope interferences. Importantly, the natural abundance of ¹³C in cholic acid (m/z=407.3) interfered with the molecular ion of ¹³C-CA (m/z=408.3) and that of D₄-CA (m/z=411.3) interfered with the molecular ion of the internal standard D₅-CA (m/z=412.3) and that of ¹³C-CA (m/z=408.3) interfered with the molecular ion of D₄-CA (m/z=411.3). The calculations to correct for these isotope interferences were based on the following:

(A) blank samples (to monitor for potential matrix interferences);
(B) the unlabeled cholic acid at m/z=408.3→343.1 signal (from cholic acid calibrators if necessary);
(C) the D₅-CA signal at m/z=412.3→348.1 (D₅-CA suitability sample, f[13C]);
(D) the ¹³C-CA signal at m/z=408.3→343.1 (from ¹³C-CA calibrators if necessary); and
(E) the D₄-CA signal at m/z=411.3347.1 (from CA and D₄-CA suitability and/or CA calibrators).

After the ¹³C-CA signal was corrected for the cholic acid isotope interference and the D₅-CA signal for the D₄-CA isotope interference in each individual sample, the resulting analyte/internal standard ratios were calculated and used to quantify the analytes based on the calibration curves that were included in each batch.

Quality control levels for CA were calculated against the mean signal response of the QCendo based on the mean during each run or based on an individual samples QCendo signal response, depending on the validation experiment. The QC signals (for QC 1-4) were calculated by subtracting the signal response of the endogenous QC from the signal of the enriched QC levels (nominal CA QC signal response=enriched QC signal response−endogenous QC signal response).

Isotope Cross-Talk Factors:

For ¹³C-Cholic Acid (f[¹³C)]=¹³C-CA signal (peak area)/CA signal (peak area);
For Cholic Acid-D₄ (f[d₄])=CA-D₄ signal (peak area)/CA-D₅ signal (peak area); and
For Cholic Acid-D₅ (f[d₅])=CA-D₅ signal (peak area)/CA-D₄ signal (peak area).

These correction factors were determined within either each run or, if no suitable factor was able to be determined, based on a set of predetermined factors:

f[¹³C] from a CA suitability sample (50 μL of 10 μmol/L CA in methanol+200 μL of methanol+internal standard) and was predetermined at 0.01331 (1.33% cross talk);
f[D₄] from a CA suitability sample (50 μL of 10 μmon CA in methanol+200 μL of methanol+internal standard) and was predetermined at 0.04603 (4.60% cross talk); and
f[D₅] from a d₅ suitability sample (50 μL of 5 μmon CA-d₄ in methanol+200 μL of methanol without internal standard) and was predetermined at 0.04342 (4.34% cross talk).

Cross-Talk correction for analyzed signals were as follows. The corrected signal for ¹³C-Cholic Acid signal (cPeak Area[¹³C])=Cholic Acid signal (Peak Area)−Cholic Acid signal (peak area)*f[¹³C]. The corrected signal for Cholic Acid-D₄ (cPeak Area[D₄])=Cholic Acid-D₄ signal (Peak Area)−Cholic Acid-d5 signal (Peak Area)*f[D₄]. The corrected signal for Cholic Acid-D₅ (cPeak Area[D₅])=Cholic Acid-D₅ signal (Peak Area)−Cholic Acid-D₄ signal (Peak Area)*f[D₅]. The Cholic Acid signals do not require any correction factors.

Response signals were used for generating 1/x weighted linear calibration curves, correction for CA quality control signal responses and all quantification:

Cholic Acid Response signal=Cholic Acid signal (Peak area)/Corrected Cholic Acid signal (cPeak Area).

¹³C-Cholic Acid Response signal=Corrected ¹³C-Cholic Acid signal (cPeak area)/Corrected Cholic Acid signal (cPeak Area).

Cholic Acid-D₄ Response signal=Corrected Cholic Acid-D₄ signal (cPeak area)/Corrected Cholic Acid signal (cPeak Area).

Validation Procedures:

The re-developed assay was validated to the extent considered fit for an FDA device (PMA) application. The validation followed applicable FDA and Clinical Laboratory Standards Institute guidelines as closely as reasonable.

The results of this validation met the predefined acceptance criteria and the assay was considered fit for analyzing clinical samples and study samples from regulatory clinical trials. For acceptance of an analytical run, the following minimum criteria were met: (i) the assay was under control and all corrective actions, if applicable, were successfully resolved and documented; (ii) acceptable system/instrument suitability test; (iii) linearity of the calibration curve has to be better than r=0.99; (iv) low calibration sample signal to noise ratio of at least 8:1; (v) no significant interference in blank samples; (vi) no significant carry-over; (vii) trueness of ¾ of calibration samples (=75%) were better than ±15% from nominal, except for the LLOQ sample, which were better than ±20%; and (viii) ¾ quality control samples in an analytical batch were within 15% of the nominal concentration.

Blanks were serum samples from individuals that had neither received ¹³C-CA nor D₄-CA. Since an endogenous compound, human serum samples always contained cholic acid and CA concentrations were measured in these “blank” samples. For CA, “no significant interference in blank sample” was assumed if the cholic acid concentration in the “blank” samples were not higher by more than 15% of the in initially measured concentrations.

Sample Storage:

Study samples, calibrators, stock solutions and quality control samples were stored at −70° C. or below unless they are being utilized for testing. Samples were kept for possible further analysis at −70° C. or below.

The analysis sequences for the samples was: Methanol, CA-d4 system suitability testing samples, methanol, blank sample (=blank serum+methanol), zero sample in duplicates (=blank serum+internal standard), calibrators for 13C-CA and CA-d4 in serum; methanol (carry-over control); CA system suitability testing samples, methanol, blank sample (=1:10 diluted serum+methanol), zero sample in duplicates (=1:10 diluted serum+internal standard), calibrators for CA in 1:10 diluted serum (1:5 for 2, 6 and 10 μmol/L calibrators); methanol (carry-over control); quality controls for 13C-CA and CA-d4; methanol (carry-over control); endogenous quality control; quality control for CA; methanol (carry-over control); validation study samples (maximum 100 samples), methanol, blank sample, zero sample, calibrators; methanol (carry-over control); quality controls; methanol.

One of the most important changes in comparison to previous assay protocols was that instead of detecting the analytes in single ion mode (LC-MS), the analytes were now detected in the negative multi-reaction mode (MRM, LC-MS/MS). The re-developed assay was then validated to the extent considered fit for an FDA pre-market device (PMA) application. The validation followed applicable FDA and Clinical Laboratory Standards Institute guidelines as closely as reasonable.

The assay development and validation included the following elements: lower limit of quantification; range of reliable response; intra-day trueness; intra-day imprecision; inter-day trueness (20 days); inter-day imprecision (20 days); exclude carry-over; dilution linearity; matrix interferences and ion suppression/enhancement; absolute extraction recovery; incurred sample re-analysis; robustness; and stability testing. The validation procedures are described in more detail in Examples 1 a) to 1m) as follows.

### Example 1 a) Lower Limit of Quantification (LLOQ)

Previously, using the old LC-MS method according to U.S. Pat. No. 8,778,299, standard curve samples prepared in matrix of normal human serum for 13C-CA ranged from the lower limit of quantification (LLOQ) of 0.1 μM to the upper limit of quantification (ULOQ) of 10.0 μM, and standard curve for 4D-CA ranges from the LLOQ of 0.1 μM to the ULOQ of 5.0 μM.

Based on previous validation of the LC-MS assay, the target LLOQ for each of the analytes in the present LC-MS/MS assay was 0.1 μmol/L, which is also the lowest calibrator. Thus, it was the goal to verify this LLOQ for the herein developed and validated version of the assay. Determination of the LLOQ followed the procedures and principles as set forth in CLSI EP17-A2. Accordingly, 24 samples with concentrations of 0.1 μmol/L were analyzed. The acceptance target followed applicable FDA recommendations [FDA, 2013] of ±20% trueness (compared to the nominal concentration). Based on Table 4 in CLSI EP17-A2, with n=20 observations, 85% of the results (17/20 samples) must fall within the acceptance target of 80-120% of the nominal concentration. In addition, imprecision (CV %) at the LLOQ for each of the analytes in all samples had to be 20% or less [FDA, 2013].

Results for Lower limit of quantification (LLOQ). For cholic acid, ¹³C-cholic acid and cholic acid-D₄, the LLOQs were 0.1 μmol/L in serum (for cholic acid in a 1:10 [v/v] serum dilution in 5% albumin in Dulbecco's phosphate-buffered saline (DPBS) as cholic acid-free serum is not available) A representative cholic acid calibration curve is shown in FIG. 2.

In summary, Trueness and imprecisions (CV %) at the LLOQ were (n=24):

TABLE 5
Summary of mean accuracies and imprecisions at the LLOQ

| Analyte | Trueness [%] | CV % |
|---|---|---|
| Cholic acid | 99.6 | 4.39 |
| ¹³C Cholic acid | 92.6 | 5.05 |
| Cholic acid D₄ | 95.0 | 6.22 |

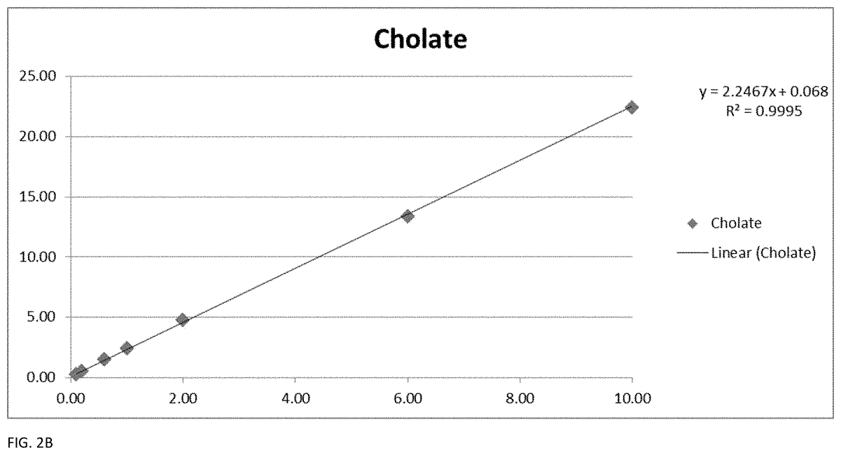

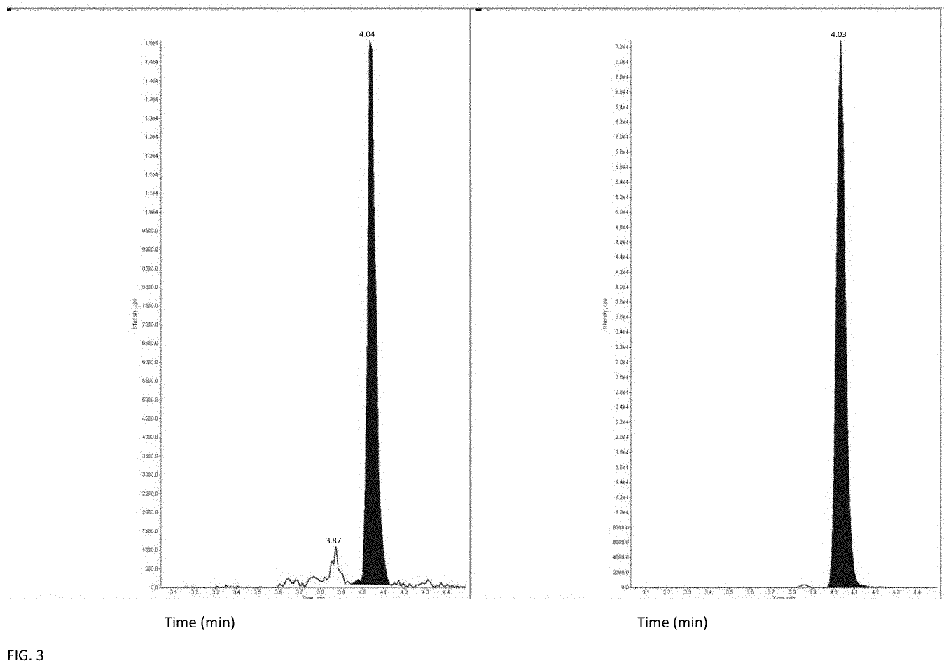

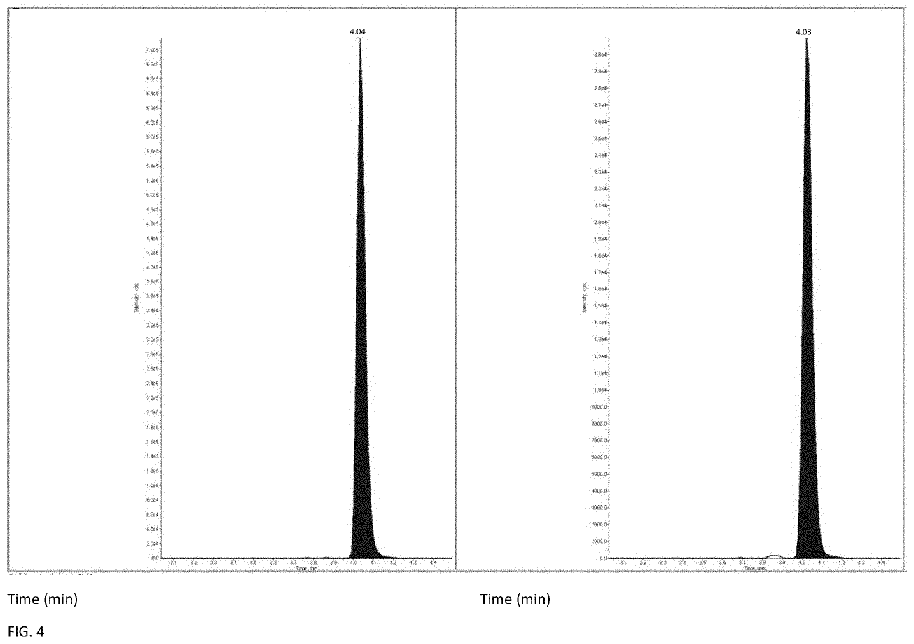

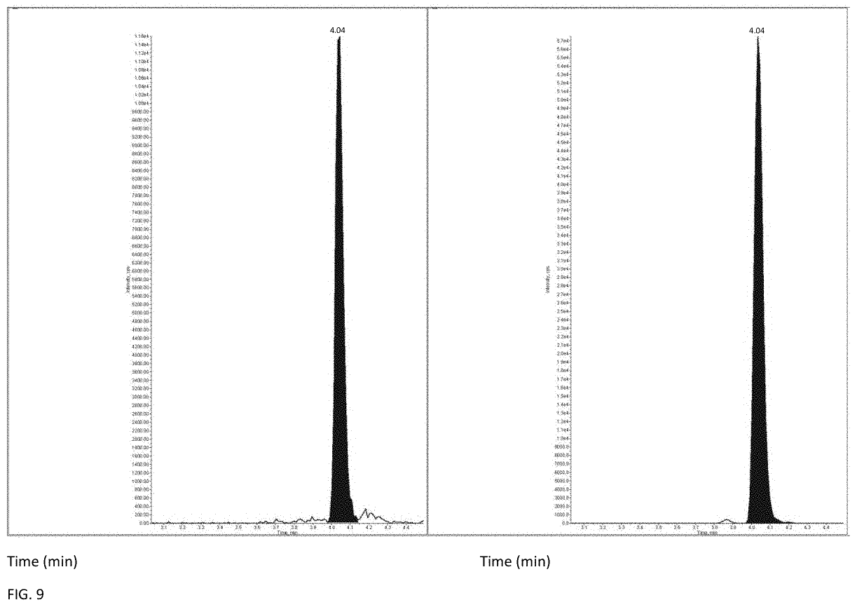

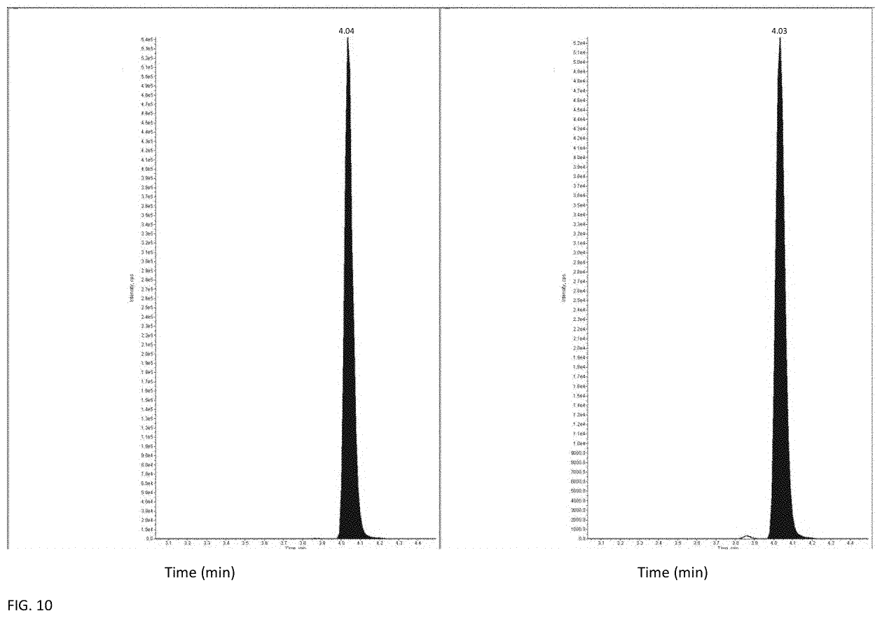

For all three analytes in Table 5, the measured concentrations of 24 of 24 samples (100%) were within 80-120% of the nominal concentrations and all imprecisions (CV %) were ≤20%. Thus, the concentration of 0.1 μmol/L met LLOQ acceptance criteria for all three analytes cholic acid, ¹³C-cholic acid and cholic acid-D₄ in human serum. Representative ion chromatograms of cholic acid, ¹³C-cholic acid and cholic acid-D₄ at the LLOQ of 0.1 μmol/L are shown in FIGS. 3, 6 and 9, respectively, compared to internal standard cholic acid-D₅.

### Example 1 b) Range of Reliable Response (Analytical Measuring Range)

The analytical measuring range is determined by the LLOQ and the upper limit of quantification (ULOQ). The ULOQ is the highest amount of analyte in a sample without dilution that can be quantitatively determined with acceptable imprecision and trueness. Based on previous validation of the LC-MS, the target ULOQ for each analyte is 10 μmol/L, which is also the highest calibrator. The ULOQ was confirmed following the procedures and principles as set forth in CLSI EP17-A. As required by applicable FDA guidelines (FDA, 2001 and 2013), the acceptance target was ±15% trueness (compared to the nominal concentration). Again, based on CLSI EP17-A2, with n=20 observations 85% of the results (17/20 samples) had to fall within the acceptance target. In addition, imprecision (CV %) for each of the analytes in all ULOQ samples had to be 15% or less (FDA, 2013).

To further confirm the analytical measuring range (CLSI EP06-A), 40 sets of calibrators for each of the analytes were assessed. These calibration curves were also used to confirm the most appropriate fit (CLSI C62-A). Acceptance criteria were met if the calibration curves consistently had a correlation coefficient of r>0.99 and at least 75% of the non-zero calibrators met acceptance criteria. To meet acceptance criteria, the measured concentration of a calibrator had to fall with 85-115% of the nominal concentration, except for the lowest calibrator (LLOQ, 0.1 μmol/L), which had to fall within 80-120% of the nominal concentration.

Range of reliable response was linear from 0.1-10 μmol/L for all three analytes cholic acid, ¹³C-cholic acid and cholic acid-D₄ in human serum. The correlation coefficients of the calibration curves were consistently r>0.99 (40 calibration curves measured on 20 different days). Acceptance criteria were met because the calibration curves consistently had a correlation coefficient of r>0.99 and at least 75% of the non-zero calibrators met acceptance criteria. To meet acceptance criteria, the measured concentration of a calibrator had to fall with 85-115%, except for the lowest calibrator (LLOQ, 0.1 μmol/L), which had to fall within 80-120% of the nominal concentration. As shown in Table 6 below, all calibrator levels for all three analytes met said acceptance criteria. The concentrations at the LLOQ met the more stringent acceptance criteria of 85-115% as well.

TABLE 6
% of Calibrators falling within the acceptance
limits of 85-115% of nominal concentrations

% within 85-115% acceptance criteria

| Calibrator | Cholic Acid | ¹³C-Cholic Acid | Cholic Acid-D₄ |
|---|---|---|---|
| [μmol/L] | [%] | [%] | [%] |
| 0.1 | 97.5 | 85.0 | 90.0 |
| 0.2 | 100.0 | 100.0 | 100.0 |
| 0.6 | 100.0 | 100.0 | 97.5 |
| 1.0 | 100.0 | 100.0 | 100.0 |
| 2.0 | 85.0 | 97.5 | 97.5 |
| 6.0 | 100.0 | 100.0 | 100.0 |
| 10 | 100.0 | 100.0 | 100.0 |

Representative individual calibration curves are shown in FIGS. 2, 5 and 8 for cholic acid, ¹³C-cholic acid and cholic acid-D₄, respectively. Representative ion chromatograms of cholic acid, ¹³C-cholic acid and cholic acid-D₄ at the ULOQ of 10 μmol/L are shown in FIGS. 3, 6 and 9, respectively,

### Examples 1 c and d) Intra-Day Trueness and Imprecision

According to applicable FDA guidance (FDA 2001, 2013), intra-day trueness and imprecision need to be established. For this purpose, 10 sets of QCs were extracted and analyzed in the same run together with two sets of calibrators positioned before and after the QC samples. For each analyte and concentration level the trueness (% of nominal concentration) and imprecision (CV %) were calculated. Intra-day trueness was considered acceptable if 75% of the concentrations fell within the acceptance limit of 85-115% and the CV % was ≤15%. Intraday trueness and precision are shown in Table 7.

### Examples 1 e and f) Inter-Day Trueness and Imprecision

To establish inter-day trueness and imprecision (FDA 2001, 2013, CLSI C62-A), the “20×2×2” protocol recommended by CLSI EP05-A3 for single site evaluation studies was followed (please also see ISO 5725-5). Said protocol uses a nested components-of-variance design with 20 testing days, two runs per testing days and two replicate measurements for each sample. A single reagent lot and a single calibration lot were used and testing was performed on a single instrument. As described in more detail in CSI EP05-A3 (sections 3.4. and 3.6.), inter-day imprecision was estimated using a balanced nested linear components analysis of variance (ANOVA, n=80) model involving 2 factors: “drug” and “run” with “run” nested within “day”. An inter-day imprecision of ≤15% was considered acceptable. Trueness was calculated for each individual study day and distribution statistics for the 20 test days were calculated. Acceptance limits for trueness were 85-115% of the nominal concentration. Intraday and interday trueness and precision are shown in Table 7.

TABLE 7
Summary of intra-day and inter-day imprecisions and Trueness

| | | intra-day | | inter-day | |
|---|---|---|---|---|---|
| | QC | Trueness | Imprecision | Trueness | Imprecision |
| Compound | [μmol/L] | [%] | CV % | [%] | CV % |
| Cholic acid | endogenous | N/A | 3.1 | N/A | 38.6 |
| | 0.25 | 84.2 | 5.2 | 98.5 | 11.8 |
| | 0.75 | 100.5 | 4.1 | 99.0 | 9.1 |
| | 2.5 | 104.1 | 4.9 | 100.0 | 7.8 |
| | 7.5 | 105.9 | 3.2 | 102.0 | 8.2 |
| | 10.0 | 108.1 | 1.9 | 98.9 | 8.4 |
| ¹³C-cholic acid | 0.1 | 106.6 | 4.0 | 98.6 | 14.9 |
| | 0.25 | 102.3 | 6.4 | 100.7 | 10.0 |
| | 0.75 | 95.1 | 2.5 | 98.8 | 11.8 |
| | 2.5 | 94.1 | 2.8 | 101.3 | 10.3 |
| | 7.5 | 95.9 | 3.0 | 99.0 | 7.3 |
| | 10.0 | 95.3 | 3.6 | 99.7 | 7.5 |
| Cholic acid-D₄ | 0.1 | 93.1 | 5.8 | 96.9 | 16.1 |
| | 0.25 | 95.2 | 3.9 | 99.2 | 8.5 |
| | 0.75 | 96.8 | 2.8 | 100.7 | 9.1 |
| | 2.5 | 94.7 | 1.7 | 101.4 | 11.3 |
| | 7.5 | 96.9 | 5.8 | 101.4 | 8.1 |
| | 10.0 | 94.4 | 5.7 | 99.1 | 6.8 |

Results for Intra-day and inter-day imprecisions and trueness. The assay met acceptance criteria for intra-day imprecision (CV %≤15%) and trueness (at least 80% of the samples within 85-115% of nominal concentration) at the six QC levels with the exception of the intra-day trueness of the 0.25 μmon QC level for cholic acid, which failed with only 50% of the samples meeting trueness acceptance criteria. For inter-day imprecision, ≤15% was considered acceptable. Trueness was calculated for each individual study day and distribution statistics for the 20 test days were calculated. Acceptance limits for trueness were 85-115% of the nominal concentration. All three analytes met the acceptance criteria for trueness and imprecision except for cholic acid at the endogenous compound level in serum, for which inter-day imprecision was 38.6%.

### Example 1 g) Exclude Carry-Over

Carry-over was assessed as required by FDA and CLSI guidelines (FDA 2001, 2013, CLSI EP-10-A3) based on the methanol samples injected after the highest calibrators during the testing of inter-day trueness and imprecision. During these experiments, 2 sets of each of the calibrators were run on each day on 20 different days. This means that a total of 40 carry-over blank methanol samples for each set of calibrators (1: cholic acid, 2: ¹³C-CA and D₄-CA) were included in the analysis. A significant carry-over effect was excluded if the signal for any of the three analytes (cholic acid, ¹³C-CA and D₄-CA) and the internal standards in 67% of the methanol samples was less than 20% of the signal of the corresponding lowest calibrator (=LLOQ). Results: Consistently, less than 0.25% carry-over was found, and less than 67% of the signals in the methanol samples were more than 20% of the signal of the corresponding lowest calibrator (=LLOQ), which was considered acceptable.

### Example 1 h) Dilution Linearity

Allowable dilutions that yield accurate results within and outside the measuring range require validation. Due to the critical influence of specimen matrix on LC-MS/MS separation and ionization chemistry, chosen diluents should be matrix-appropriate (CLSI C62-A). ¹³C-CA and D₄-CA are dosed to result in concentrations that fall into the linear range of the assay. For the dilution experiments, human serum samples spiked with 20 μmon (twice above the ULOQ) and at the ULOQ (10 μmon) were used. Samples were diluted 1:1 and 1:5 (n=12 per concentration level and dilution). Dilution linearity was assumed if 75% of the diluted samples fell with 85-115% of the nominal concentrations [CLSI C62-A]. Imprecision for each dilution had to be ≤15%.

### Example 1 i) Matrix Interferences and Matrix Effects (Ion Suppression/Ion Enhancement)

Matrix interferences were tested following CLSI EP07-A3. Accordingly, the impact of potential interferences on imprecision (CLSI EP05-A3) and trueness (CLSI EP09-A3) were evaluated.

### Matrix Interferences by Endogenous Compounds in Human Serum.

To assess if any compounds physiologically contained in human serum interfered with the quantification of the analytes, samples from 12 different, diverse individuals were used. These samples were gender balanced, included samples from African Americans and Hispanics and from patients with cholestasis. Blank samples were extracted (in triplicate) and analyzed using LC-MS/MS. No significant interference was assumed if in all samples there was no signal in the retention time window of the analytes and the internal standard higher than 20% of the signal of ¹³C-CA and D₄-CA and the internal standard at the LLOQ or an increase of the endogenous cholic acid signal of not more than 20%.

### Matrix Interferences by Endogenous Compounds (Cholesterol, Triglycerides and Bilirubin)

A potential interference by cholesterol, triglycerides and bilirubin was assessed in blank, zero and the QC samples. These samples were spiked to result in the following concentrations in these samples: Bilirubin: 0, 30 and 60 μmon; Triglycerides: 0, 150 and 300 μmon; and Cholesterol: 0, 250 and 500 μmon. The number of observations for each of the QC levels and spiked endogenous concentrations was n=8. No interference was assumed if: (i) in the blank samples (n=24) there was no signal in the corresponding retention time window of higher than 20% for the signals of ¹³C-CA and D₄-CA at the LLOQ; and (ii) in the spiked samples, the imprecision (CV %) did not exceed 15% and trueness was acceptable (75% of the measured concentrations fell within 85-115% of the nominal concentrations).

### Matrix Interferences in Hemolytic Blood.

A potential interference with hemoglobin was tested in hemolytic human serum samples. Hemolytic serum collected from 3 different individuals and serum samples spiked with lysed blood cells as well as serum spiked with hemoglobin were tested. Blank samples, zero samples and samples spiked at the same concentration level as the QCs (0.25, 0.75, 2.5, and 7.5 μmon) were prepared, extracted and analyzed. No interference was assumed if: (i) in the blank and zero samples there was no signal in the corresponding retention time window of higher than 20% for the signal of ¹³C-CA, D₄-CA and D₅-CA (blanks only) at the LLOQ or an increase of the endogenous cholic acid signal of not more than 20% and (ii) in the spiked samples, the imprecision (CV %) did not exceed 15% and trueness was acceptable (75% of the measured concentrations fell within 85-115% of the nominal concentrations).

Interferences with Drugs

Interference with 136 drugs (100 ng/mL), drugs of abuse and their key metabolites were studied. Potential interferences were assessed in blank, zero and the QC samples. These samples were spiked at 3 different concentration levels with the test drugs and their selected key metabolites. The number of observations for each of the QC levels and spiked endogenous concentrations was n=6. No interference was assumed if: (i) in the blank and zero samples there was no signal in the corresponding retention time window of higher than 20% for the signal of ¹³C-CA, D₄-CA and D₅-CA (blanks only) at the LLOQ or an increase of the endogenous cholic acid signal of not more than 20%; and (ii) in the spiked samples, the imprecision (CV %) did not exceed 15% and trueness was acceptable (75% of the measured concentrations fell within 85-115% of the nominal concentrations).

### Isotope Interferences

Stability of the ¹³C-signal of unlabeled cholic acid and of the ¹³C-signal of D₄-CA are essential for the correct calculations of the analyte concentrations. Hence, it was important to also study potential interferences with the ¹³C-signal of unlabeled cholic acid and of the ¹³C-signal of D₄-CA. This was achieved by using the same approach as described above for matrix interferences, interference by endogenous compounds (cholesterol, triglycerides, and bilirubin), interferences in hemolyzed blood and interference by 136 drugs and selected key metabolites. However, the samples were spiked only with cholic acid and D₄-CA at the QC levels. The numbers of observations were the same as for the corresponding interference tests described above. The potential effects on the ¹³C signals of cholic acid and D₄-CA (m/z=408.3 and m/z=412.3) were analyzed. No internal standard was added to these samples.

For cholic acid, the m/z=408.3/407.3 signal ratio and for D₄-CA the m/z=412.3/411.3 signal ratio was calculated. Based on CLSI C62-A (section 7.4.) and CLSI C50-A, no significant isotope interference can be assumed if the ion ratios for each QC level do not change by more than ±20%.

### Matrix Effects (Ion Suppression/Ion Enhancement)

The matrix components of a biological sample that are to be assayed by mass spectrometry generally include salts, lipids, proteins, peptides, and organic small molecules. It is well-known that any matrix component can interfere with or enhance the ionization of the analyte of interest in the mass spectrometric experiment. The most important matrix components that alter the ionization efficiency of the analyte of interest are salts and lipids, most specifically phospholipids [CLSI C50-A, CLSI C62-A and CLSI EP14-02). In general, the magnitude of the matrix effect should be evaluated in the context of total allowable error (TEa) limits required for the method, where TEa is partitioned into imprecision, bias and interference components (CLSI EP07-A3 and CLSI EP21-A).

Based on CLSI C62-A the following was considered best practice: Evaluate matrix effects in at least 5 different native matrix samples by comparing the signal obtained from samples that were spiked with analyte post-extraction versus the signal obtained by spiking analyte into neat solutions according to the procedure described in CLSI C-50A and Matuszewski et al. (2003). We used samples from 12 different, diverse individuals. These samples were gender balanced, included samples from African Americans and Hispanics and from patients that either had cholestasis, liver fibrosis and sustaining and/or non-sustaining liver cirrhosis. Five calibration curves were prepared in methanol/0.1 mmol/L NaHCO₃ (80/20, v/v). These calibrators were injected to obtain mean signals at each calibration level from the neat solutions. These signals were compared with the mean signal obtained from 12 calibration curves prepared by spiking the analyte post extraction in 12 different lots of matrix (from 12 different individuals). This was achieved by extracting the 12 different lots of blank matrix and then spiking the post extract before injection into the instrument.

In addition, potential matrix effects were assessed using post-column infusion experiments as described by Mueller et al. (2002).

Acceptance Criteria:

The % Matrix Effect (% ME) was converted into % Matrix Bias by the following equation:

% Matrix Bias=100−% ME (absolute matrix effect).

To assess if the internal standard effectively compensated any potential matrix effects the relative matrix effects based on the analyte/internal standard ratios were also calculated. The % Matrix Bias was evaluated in the context of total allowable error [CLSI EP7-A3] which was set to ±15%. The % CV of the peak areas were also evaluated to determine the magnitude for which the matrix contributed to assay imprecision. Results were acceptable if imprecision did not exceed 15%.

Matrix effects (ion suppression/ion enhancement) test results. Matrix effects were tested in serum collected from 12 different individuals using two different approaches: (1) the procedure described in CLSI C-50A and by Matuszewski et al. (2003); and (2) post-column infusion experiments as described by Müller et al. (2002).

The method described by Matuszewski et al. (2003) is based on the comparison of the MS/MS signals of samples spiked after extraction with the MS/MS signals after injection of neat solutions of the analytes at the corresponding concentrations. The results are summarized in Table 8. The absolute matrix effect compares the signals of the analytes, the relative matrix effects the analyte/internal standard ratios. The data showed ion suppression of cholic acid of an average of −19.7% (matrix bias), whereas the MS/MS signals of ¹³C-cholic acid, cholic acid-D₄ and the internal standard cholic acid D₅ were depressed by an average of −34.0%, −34.9% and −41.3%, respectively. Accordingly, when the relative ion suppression effects were assessed, the internal standard overcompensated for the ion suppression of cholic acid resulting in a relative average ion enhancement of +40.4% (relative matrix bias). The internal standard compensated for the ion suppression of ¹³C-cholic acid (average+12.7%) and cholic acid-D₄ (average+11.1% matrix bias).

TABLE 8
Absolute and relative matrix effects as estimated based
on the method described by Matuszewski et al. (2003)

| | Absolute matrix effect | | | | Relative matrix effect | | |
|---|---|---|---|---|---|---|---|
| | Cholic | ¹³C-Cholic | Cholic | Cholic | Cholic | ¹³C-Cholic | Cholic |
| Calibrator | Acid | acid | Acid-D₄ | Acid-D₅ | Acid | acid | Acid-D₄ |
| [μmol/L] | [%] | [%] | [%] | [%] | [%] | [%] | [%] |
| 0.1 | 97.3 | 58.0 | 56.6 | 49.3 | 201.6 | 117.5 | 115.0 |
| 0.2 | 90.5 | 65.3 | 61.3 | 55.7 | 165.1 | 117.6 | 110.6 |
| 0.6 | 73.1 | 63.6 | 63.6 | 56.2 | 131.5 | 113.3 | 113.4 |
| 1.0 | 79.0 | 66.7 | 64.1 | 58.1 | 134.9 | 114.8 | 110.4 |
| 2.0 | 78.9 | 70.3 | 68.8 | 62.1 | 128.0 | 113.6 | 111.0 |
| 6.0 | 77.4 | 74.0 | 76.4 | 68.8 | 112.9 | 107.2 | 110.6 |
| 10.0 | 66.1 | 63.9 | 64.7 | 61.0 | 108.7 | 105.2 | 106.5 |

Overall, the matrix effects did not affect assay trueness and imprecision as shown in Table 8 with the only potential exception of low-concentration cholic acid, where it may explain the high imprecision of 38.9% at endogenous concentration levels in serum.

The post-column infusion following the protocol described by Müller et al. (2002) was based on the continuous infusion of a mixture of cholic acid, ¹³C-cholic acid, cholic acid-D₄, and cholic acid D₅ (1 μmon of each compound in methanol) at an infusion rate of 25 μmon. To test for ion suppression/ion enhancement, 20 μL of the blank extracted matrix samples (from different individuals, no internal standard added during extraction) were injected. A dip of the MS/MS signal at the retention time of the analytes indicates ion suppression, a peak ion enhancement. There was a dip in the MS/MS signal at the analytes's retention times, which is consistent with the ion suppression also detected in the matrix effect experiments following the protocol described by Matuszewski et al. (2003) described above. In summary: Absolute matrix effects resulted in negative matrix bias (ion suppression) of −19.7 to −34.9%. Ion suppression was compensated by the internal standard cholic acid-D₄, albeit was overcompensated in the case of cholic acid (+40.4% relative matrix bias). Nevertheless, as shown in the Table 8 above, matrix effects did not have a relevant negative effect on trueness and imprecision of the assay.

Interference testing results. After addition of the highest concentrations of interfering compounds (60 μmon bilirubin, 300 μmon triglycerides, 500 μg/L cholesterol and 100 ng/mL drugs), the LC-MS/MS signals within retention time window and at the ion transitions of analytes in blank samples were (n=24): (i) ¹³C cholic acid: 0.23%±0.42% (range 0%-1.31%) of the average signal at the LLOQ; (ii) Cholic acid-D₄: 2.40%±1.73% (range 0%-7.28%) of the average signal at the LLOQ; (iii) for hemolyzed serum, the LC-MS/MS signals within retention time window and at the ion transitions of analytes in blank samples were (n=8): ¹³C cholic acid: 2.29%±1.06% (range 0.85%-3.65%) of the average signal at the LLOQ, and Cholic acid-D₄: 4.70%±2.78% (range 1.95%-9.23%) of the average signal at the LLOQ.

In both cases, this was lower than the acceptance limit of less than 20% of the average signal at the LLOQ so that there was no evidence for interference by the tested endogenous compounds, drugs and hemolysis with the assay.

Interference testing in samples spiked with the analytes at the QC concentrations gave the results summarized in Table 9. As results in Table 9 show, all tested interference levels at each of the QC levels met acceptance criteria. This again provided evidence that there was no interference by the tested endogenous compounds, drugs and hemolysis with the assay.

TABLE 9
% of interference testing samples within the acceptance
limits of 85-115% of the nominal concentrations

| | % of the measured concentrations within 85-115% | | | | | | | |
|---|---|---|---|---|---|---|---|---|
| | Drug and endogenous interferences | | | | | | | |
| QC | ¹³C-cholic acid | | | cholic acid-D₄ | | | Hemolysis | |
| [μmol/L] | None | medium | high | none | medium | high | ¹³C-cholic acid | cholic acid-D₄ |
| 0.25 | 100 | 100 | 100 | 100 | 83.3 | 83.3 | 87.5 | 87.5 |
| 0.75 | 100 | 100 | 100 | 83.3 | 83.3 | 100 | 100 | 100 |
| 2.5 | 100 | 100 | 100 | 100 | 83.3 | 100 | 87.5 | 87.5 |
| 7.5 | 100 | 100 | 100 | 100 | 83.3 | 100 | 100 | 87.5 |

Specificity/Selectivity. In pooled and serum samples collected from 12 different individuals, the response at the ion transitions and retention times of cholic acid, ¹³C cholic acid and cholic acid-D₄ was less than 20% of the detector response of that of the lowest calibrators for the analytes (LLOQ) and their internal standards. Based on these results, the assay was considered specific. Results of the serum samples collected from 12 different individuals after addition of 60 μmol/L bilirubin, 300 μmol/L triglycerides, 500 μg/L cholesterol and 100 ng/mL drugs were measured in duplicate. (data not shown).

### Example 1 j) Absolute Extraction Recovery

Absolute extraction recovery was assessed in the human serum samples collected from 12 different diverse individuals as also used for the study of matrix interferences and matrix effects. Following the protocol described by Matuszewski et al. (2003), the analyte/internal standard ratios in the following samples were compared as follows.

Pre-extraction spike: The 12 samples were each spiked at the same level as the QC samples 0.25, 0.75, 2.5, and 7.5 μmon then extracted and analyzed. Human serum contains cholic acid. Before samples were spiked, cholic acid concentrations were quantified. Cholic acid was spiked on top of the endogenous cholic acid to result in 0.25 (+ endogenous cholic acid) μmon, 0.75 (+ endogenous cholic acid) μmon, 2.5 (+ endogenous cholic acid) μmon, and 7.5 (+ endogenous cholic acid) μmon.

Post-extraction spike: The samples from the 12 individuals were first extracted and then spiked resulting in the same concentrations as described for the pre-extraction spiked samples above: 0.25, 0.75, 2.5, and 7.5 μmon. Cholic acid was spiked on top of the endogenous cholic acid to result in 0.25 (+ endogenous cholic acid) μmon, 0.75 (+ endogenous cholic acid) μmon, 2.5 (+ endogenous cholic acid) μmon, and 7.5 (+ endogenous cholic acid) μmon.

For each corresponding samples pair (pre- and post-extraction spike), the absolute extraction recovery was calculated as follows:

Extraction recovery [%]=analyte/internal standard ratio pre-extraction spike/post-extract spike×100.

Distribution statistics for each concentration level were calculated.

Extraction recovery. The mean recoveries of cholic acid, ¹³C-cholic acid and cholic acid-D₄ extracted from the serum of 12 different individuals and 1 individual with cholestasis (¹³C-cholic acid and cholic acid-D₄ only) are summarized in Table 10.

TABLE 10
Summary of extraction recovery results.

| | QC | Average Recovery |
|---|---|---|
| | Nominal concentration | [%] |
| Analyte | [μmol/L] | Mean ± StdDev |
| Cholic acid | Endogenous | 90.6 ± 6.4 |
| | 0.25 | 97.9 ± 6.5 |
| | 2.5 | 98.4 ± 4.4 |
| | 7.5 | 95.9 ± 4.4 |
| ¹³C-cholic acid | 0.1 | 110.2 ± 9.4 |
| | 0.25 | 107.9 ± 6.5 |
| | 2.5 | 104.0 ± 7.2 |
| | 7.5 | 106.6 ± 7.0 |
| Cholic acid-D₄ | 0.1 | 106.1 ± 8.2 |
| | 0.25 | 104.4 ± 6.9 |
| | 2.5 | 106.3 ± 7.3 |
| | 7.5 | 111.0 ± 7.7 |

There was no obvious difference in extraction recovery between the subject with cholestasis and the subjects without.

Results of Extraction recovery: Depending on the concentration tested, the mean recoveries of cholic acid ranged from 90.6% to 95.9%, that of ¹³C-cholic acid from 104.0% to 110.2% and that of cholic acid-D₄ from 104.4% to 111.0%, respectively.

### Example 1 k) Incurred Sample Re-Analysis

One-hundred patient samples were each analyzed on two different days. Acceptance followed the criteria as set forth in the 2013 FDA draft guideline: Two-thirds (67%) of the repeated sample results must fall within 20% difference (first versus second day). The percentage difference of the results was determined using the following equation:

Percent difference=(first day result−re-run result)·100/mean

### Example 1 l) Robustness

The robustness of the method was considered during validation to assess the impact of small changes such as temperature or humidity fluctuation, preparation of calibrator materials by different operators, instrument cleanliness, and incubation time [CLSI EP09-A3; CLSI C62-A]. Robustness was tested based on the patient samples analyzed during the incurred sample re-analysis, and the cross-validation study. These samples were run by different analysts using independently prepared calibrators, samples in a different laboratory environment and using different instrumentation on different days. For acceptance, at least for 67% of the samples, the difference between analyses of the same sample had to be 20% or less. Furthermore, assay robustness was assessed based upon QC sample reproducibility (trueness and imprecision) over 20 days. For acceptance, at least 67% of each of the QCs had to fall within 85-115% of the nominal (trueness) and CV % must be 15% or less (imprecision). Only if all of these acceptance criteria were met, sufficient robustness was assumed.

### Example 1 m) Stability Testing

Short-term stabilities were examined under the following condition: Sample benchtop stability (1 day), Sample storage stability at 4° C., −20° C. and −70° C. or below for 48 hours, 3 days and 1 week, Protein precipitation solution stability (benchtop for 24 hours, +4° C. for 1 week), Extracted sample/autosampler stability for up to 72 hours. Samples were placed in the autosampler and reinjected at baseline, after 12 hours, 24 hours, 48 hours and 72 hours. This also tested re-injection reproducibility [FDA, 2013].

Long-term stabilities were tested under the following conditions: Initial assessment for 1 (±3 days) month (at −20° C. and −70° C. or below), Long-term stability for 2 (±3 days), 3 (±7 days), 6 (±14 days), 12 months (±14 days) (at −20° C. and −70° C. or below), and Freeze thaw cycles (−70° C. or below), for 3 cycles.

For stability testing (except for protein precipitation solution stability), 5 sets of QCs were analyzed at each testing time point. Stability was assumed if the results fell within 85-115% of the nominal concentrations.

The results showed that cholic acid, ¹³C-cholic acid and cholic acid-D₄ were stable for 1 week at −80° C., during three freeze/thaw cycles (−80° C./room temperature), for 1 week at −20° C., for 1 week at +4° C., for 24 hours and 48 hours in the autosampler (at +4° C., extracted sample stability. Long-term stability testing is still ongoing.

Manual re-integration. Manual integration was allowed to minimize errors made by the integration software, which may occur in the case of blanks and low-concentration samples, when integration software tends to include obvious baseline noise into the integrated peak. However, it was critical to avoid investigator/analyst bias. Therefore, manual integration and its documentation strictly followed the rules, procedures, checks and balances as set forth in iC42 Clinical Research and Development standard operation procedure iC42-WP-303 “Manual Integration of Chromatograms” and was closely reviewed by quality assurance personnel. Any manual integration of ion chromatograms was justified in writing (with signature and date) and was listed together with values from the automatic integration. Copies of both the original integration and all versions of the modified integration were printed, dated, signed and filed together with the study information. Only the approved results after manual integration were included in the Validation Report.

Conclusions. The re-developed assay was validated to the extent considered fit for an FDA device (PMA) application. The validation followed applicable FDA and Clinical Laboratory Standards Institute guidelines as closely as reasonable. The results of this validation met the predefined acceptance criteria and the assay was considered fit for analyzing clinical samples and study samples from regulatory clinical trials. For acceptance of an analytical run, the following minimum criteria were met. The assay was under control and all corrective actions, if applicable, were successfully resolved and documented. Acceptable system/instrument suitability test. Linearity of the calibration curve was better than r=0.99. Low calibration sample signal to noise ratio was at least 8:1. No significant interference was found in blank samples. No significant carry-over was found. Trueness of ¾ of calibration samples (=75%) were better than ±15% from nominal, except for the LLOQ sample, which were better than ±20%. Three-fourths of the quality control samples in an analytical batch were within 15% of the nominal concentration.

### Example 2. Cross Validation Study

After the assays described in Example 1 were completed in Laboratory 1 employing LC-MS/MS with negative mode MRM, the assay was transferred to a different laboratory (Laboratory 2) using different instrumentation. For this purpose, the following was run in parallel on the Laboratory 1 LC-MS/MS system on which the assay was validated and the Laboratory 2 LC-MS system. It is noteworthy that Laboratory 2 had to use selected ion monitoring (LC-MS SIM) due to the lack of an LC-MS/MS instrument. Based on CLSI C-62A a cross-evaluation between instruments should constitute of testing LLOQ, trueness, precision and analytical measurement range. Thus, the following samples were prepared and split to be analyzed in both laboratories in parallel:
Six sets of calibrators;
Six sets of QCs; and
One hundred patient serum samples provided by sponsor HepQuant.

The number of 100 patient samples exceeded the minimum number of n=40 required by CLSI EP09-A3. The results of the patient samples were compared using Deming regression, Passing-Bablok regression and Bland-Altman plots. Data acquisition, integration of peak signals and raw data export was done using AB Sciex Analyst Software (Version 1.6.2). Quantification of raw data and distribution statistics were calculated using EXCEL (Version 2010 and up, Microsoft, Redmond, Wash.). SigmaPlot 13.0. (SyStat, Richmond, Calif.) was used for graphic analysis and MedCalc Statistical Software version 18.0 (MedCalc Software, Ostend, Belgium) was used for assay comparison (Deming regression, Passing-Bablok analysis and Bland-Altman plots).

Acceptance followed the criteria as set forth in the 2013 FDA draft guideline: two-thirds (67%) of the repeated sample results fell within 20% difference (Laboratory 1 versus Laboratory 2). The percentage difference of the results was determined using the following equation:

Percent difference=(HepQuant result−iC42 Clinical Research and Development result)·100/mean

In addition, the calibrators and QC samples met acceptance criteria in both laboratories as described above. The results of this validation met the predefined acceptance criteria and the LC-MS/MS assay was considered fit for analyzing clinical samples and study samples from regulatory clinical trials.

### Example 3. Estimating Portal Flow from a Single Blood Draw

The individual time point serum cholate concentrations from the FLOW and SHUNT tests in HALT-C and Early CHC studies were carefully analyzed and differences in serum cholate concentration at 45, 60, and 90 minutes were found to be highly significant (p<0.005) as disclosed in U.S. Pat. No. 8,961,925 comprising measuring distinguishable bile acid by HPLC-MS. The concentration at 60 minutes had the best correlation (r²=0.8) with the portal flow. An equation was derived that could transform the serum cholate concentration (uM) at 60 min into an estimated portal flow (mL/min/kg) with 85% accuracy of the 5 point FLOW method. This led to the development of the STAT test, in which, in one embodiment, the patient drinks an oral dose of distinguishable cholate compound, e.g., deuterated-cholate, and gives a single blood sample after 1 hour. The accuracy of the STAT test relative to the FLOW test is shown in FIG. 12A. FIG. 12A shows the accuracy and correlation (R²=0.8965) of the 60 minute STAT test relative to the FLOW test from early CHC patients and the equation for interconverting the log STAT and log FLOW values to obtain an estimated flow rate:

y=0.9702x+0.0206, wherein;
x=LOG HepQuant FLOW value, and
y=LOG HepQuant STAT value.

### Example 4. Efficacy of STAT (Estimated Portal Flow) in Detecting Hepatic Dysfunction

In an Early CHC study as disclosed in U.S. Pat. No. 8,961,925 comprising measuring distinguishable bile acid by HPLC-MS, healthy controls had a portal flow of 34±14 ml/min/kg (mean±SD). Hepatic dysfunction was defined as a portal flow more than 1 SD below the control mean, a flow <20 ml/min/kg. In the early CHC group, about ½ the patients exhibited hepatic dysfunction. The estimated portal flows in the early CHC patients were calculated from the equation shown in FIG. 8 using their 60 min serum cholate level. The estimated flow could detect hepatic dysfunction with a sensitivity of 90%, a specificity of 85%, a positive predictive value (PPV) of 82%, and a negative predictive value (NPV) of 92%. These preliminary results demonstrate that a single blood sample after an oral cholate dose could be used to detect hepatic dysfunction in early stage CLD.

Furthermore, in the Early CHC study we analyzed the potential impact of STAT if used as a screening test. Currently adults are screened for liver disease in the primary care setting by ALT. In our analysis of the Early CHC study we found that addition of STAT to ALT could improve detection of patients with chronic hepatitis C. In early stage patients, ALT was abnormal in only 34%, STAT was abnormal in 48%, and 65% of the patients had either abnormal ALT or STAT. Screening with combination of ALT and STAT would double the detection rate for patients with liver disease due to chronic hepatitis C. Of course, when used in such a strategy, STAT would also detect patients with liver diseases other than chronic hepatitis C as well.

STAT also has test cutoffs that correlate with advanced liver disease. In patients with chronic hepatitis C and in patients with the chronic cholestatic liver disease, primary sclerosing cholangitis, STAT result with estimated FLOW of <10 mL/(kg min) correlated with risk for liver decompensation or clinical complications. In this situation, STAT would reflex to either FLOW or SHUNT to provide precise quantification of the portal circulation.

### Example 5. Procedure for Performance of an Exemplary STAT Test

The STAT test was previously disclosed in U.S. Pat. No. 8,961,925 comprising measuring distinguishable bile acid by HPLC-MS SIM. In the present disclosure, a similar procedure is followed, except the method comprises measuring distinguishable bile acid by LC-MS/MS with MRM.

PO (Per Oral) Test Compounds:

²H4-Cholate ([2,2,4,4-²H]-Cholic Acid, 40 mg) (e.g. CDN Isotopes).
Sodium bicarbonate (e.g. 600 mg).

### Patient Testing Supplies:

Serum/plasma transfer tubes and labels.
10 cc syringe for drawing blood sample.
7 cc red top and 7 cc gray top vacutainer tubes for serum sample collection.
Needle discard bucket

A drinking substance such as apple or grape juice for diluting oral test compounds.

### Exemplary Test Compound Preparation

One exemplary solution of an oral composition may contain 2,2,4,4-²H-Cholate, and Sodium bicarbonate (e.g. 40 mg, and 600 mg, respectively). In one exemplary method, the day before the test, water can be added to about the 10 cc mark on a tube containing the oral test compounds to obtain the Oral Test Solution. Cap tube tightly and shake to mix. Swirl contents to get all the powder granules down into the water.

On the test day pour dissolved Oral Test Solution into a container such as a urine cup. Rinse tube into urine cup with about 10 mls water. Prior to beginning the test, add a diluting liquid such as grape or apple juice (not citrus juice) to about the 40 ml mark on the urine cup containing the Oral Test Solution. Swirl gently to mix; do not shake or stir, or mixture may foam out of container. Have extra juice on hand for rinse.

### Testing Procedure

In one exemplary method the following procedure will be used. Optionally collect baseline serum sample (see Sample Collection) before test compound is administered.

Administration of Test Compounds.

Start timer. Record T=0.0—have patient drink oral solution of cholate and juice. Rinse cup with a little more juice and have patient drink rinse. Record timer time.

Blood Sample Collection

Collect an intravenous blood sample from the patient at 60 minutes post cholate administration. Record timer time.

Process blood samples to serum, and further by the procedure according to Example 1, and perform sample analysis by LC-MS/MS to determine the concentration of distinguishable cholate in the blood sample. The sample test result for a given patient at a specific date/time point can be compared to cutoff values established from, e.g., a control group, or alternatively each patient may serve as his/her own control over time.

### Example 6. Procedure for Performance of SHUNT and FLOW Assays with Analysis by LC-MS/MS

Performance of FLOW (Oral Cholate Clearance Test) and SHUNT (Cholate Shunt Test) assays comprising measuring distinguishable cholate compounds by GC-MS or HPLC-MS with SIM are disclosed in U.S. Pat. Nos. 8,613,904 and 8,778,299, each of which is incorporated herein by reference. In the present disclosure, a similar procedure is followed, except the method comprises measuring distinguishable bile acid by LC-MS/MS with MRM.

Collection and Processing of Samples.

Reagents and Supplies.

The following reagents and supplies may be utilized in the Cholate Shunt and Cholate Clearance Test procedures. If the patient is undergoing only the oral cholate clearance test, the IV Solution and 25% Human Albumin for injection are omitted.

IV Solution—20 mg 24-¹³C-Cholic Acid in 5 cc 1 mEq/ml Sodium Bicarbonate
PO test compounds 2,2,4,4-²H (40 mg) and Sodium Bicarbonate (600 mg)
25% Human Albumin for injection (5 ml) to be added to 24-¹³C-Cholic Acid solution.
IV supplies, including 250 mls NS, indwelling catheter, 3-way stopcock.
10 cc syringes for administering IV test compounds
7 cc red top tubes for sample collection
3 ml crovials for serum storage
Needle discard bucket
Apple or Grape (non-citrus) juice for oral test compounds

### Timer

### Centrifuge

Transfer pipets

Patient Preparation.

It is ascertained that the patient has no allergic reaction to latex. It is further ascertained that the patient has had nothing to eat or drink (NPO), except water, since midnight the night before the test day. The patient height and weight are measured and recorded. The patient is fitted with an IV with a three-way stopcock and normal saline to keep open (NS TKO) is placed before the test begins.

Cholate Compound Stock Solutions.

Test Compound Preparation.

The Oral Solution may be utilized for either or both of the oral cholate clearance test and/or the cholate shunt assay. An oral solution including 2,2,4,4-²H-Cholic acid (40 mg) and Sodium Bicarbonate (600 mg) is dissolved in about 10 cc water 24 hours prior to testing by mixing vigorously. The solution is stored in either the refrigerator or at room temperature. Just prior to administration, grape or apple (non-citrus) juice is added to the mixture. The juice solution is mixed well and poured into cup for patient to drink. The cup is rinsed with extra juice which is administered to the patient.

The IV Solution is utilized for either or both of the IV cholate clearance test and/or the cholate shunt assay. A formulation of 20 mg Cholic Acid-24-¹³C in 5 cc 1 mEq/ml Sodium Bicarbonate is prepared by pharmacy staff. The Test dose is 20 mg Cholic Acid-24-¹³C in 10 cc diluent. If vial is frozen, it is allowed to thaw completely. Just prior to beginning the test, the Cholic Acid-24-¹³C solution is mixed with albumin as follows (this method prevents loss of test compound during mixing process). Draw up all of 24-²³C-Cholic Acid solution (about 5 cc) in a 10 cc syringe. Draw up 5 cc albumin in another 10 cc syringe. Detach needle from the 24-²³C-cholate syringe and attach a 3-way stopcock. Detach needle from albumin syringe and inject albumin through stopcock into 24-¹³C Cholate syringe. Draw a little air into the bile acid/albumin syringe and mix solutions gently by inverting syringe several times. Expel air.

Test Compound Administration.

Baseline samples are before test compounds are given. The time these specimens are collected should be recorded on sample collection record sheet. Administration of test compounds is performed as follows. Start timer. Record 24 hour clock time as T=0. Record time. At T=1-3 minutes administer oral compounds. Have the patient drink the oral solution and juice. Rinse cup with more juice and have patient drink rinse. Record timer time. At T=4-5 minutes-using the 3-way stopcock administer the IV push of 20 mgs ¹³C Cholic acid in 5 mls 25% Human Albumin. Record timer time. Return line to NS through 3-way stopcock.

Specimen Collection.

Collect all samples via the 3-way stopcock, optionally with 0.5 ml discard before each sample to prevent dilution or cross-contamination of samples. Collect blood samples (at least 0.1 mL each, or at least 0.05 mL each) at the following times. (T=timer time).
1. T=10 minutes, collect 5 minute, record timer time;
2. T=25 minutes, collect 20 minute, record timer time;
3. T=50 minutes, collect 45 minute, record timer time;
4. T=65 minutes, collect 60 minute, record timer time; and
5. T=95 minutes, collect 90 minute, record timer time.

Specimen Handling.

The samples are allowed to clot at room temperature for at least 30 minutes. The samples are spun for 10 minutes at 3000 rpm. Serum is removed to properly labeled vials or 9-well plates and frozen at −20° C. until samples are transported.

Preparation of Cholate Compound Stock Solutions.

Accurate determination of cholate clearances and shunt is dependent on accurate calibration standards prepared as shown in Example 1. Concentrations of cholic acid compounds in stock solutions must be accurate and reproducible. Very accurate (error <0.5%) portions of the cholic acid powders are weighed and glass weighing funnels and washes of 1 M NaHCO₃ are used to ensure quantitative transfer of the powder to the flask. Volumetric flasks are used to ensure accurate volumes so that the final concentrations of the primary stock solutions are accurate. Calibrated air displacement pipettes are used to dispense accurate volumes of the primary stock solutions that are brought to full volume in volumetric flasks to prepare secondary stock solutions that are also very accurate. Secondary stock solutions are used to prepare the standard curve samples, accuracy and precision samples, recovery samples, quality control samples, selectivity samples, and stability samples.

Preparation of Internal Standard Solution

The internal standard solution was aliquoted and transferred into 1.5 mL conical polypropylene tubes with snap-on lids and was stored at −70° C. or below. To prepare the protein precipitation solution containing 0.5 μmon D₅-CA, 50 μL of the internal standard solution were added to 99.95 mL of protein precipitation solution (in this case LC-MS grade methanol). Volumes were scaled according to the volumes needed. Protein precipitation solution was stored at +4° C. for up to 1 week.

Method of Extraction

Aliquots of 50 μL of the study serum sample, calibrator, or quality control sample were transferred into 1.5 mL deep-well 96 well plates. Two hundred μL of the protein precipitation solution (methanol) containing the internal standard D₅-CA (0.5 μmol/L) were added to each well.

Samples were vortexed for 5 min, centrifuged (16,000·g, 4° C., 15 min or 4,750·g for 20 min using deep-well 96 well plates) and the supernatant was transferred into HPLC vials, or into 0.5 mL 96 well injection plates.

LC-MS/MS Equipment

The Analytical system AB Sciex API 4000 LC-MS/MS was employed using a turbo electrospray interface, and negative multiple reaction monitoring (MRM) mode. The HPLC system included two G1312A binary pumps, two G1379A vacuum degassers and a G1316A thermostatted column compartment (all Agilent 1100 series, Agilent Technologies, Palo Alto, Calif.) with integrated 6-port Rheodyne column switching valve (Rheodyne, Cotati, Calif.). The connections of the switching valve are shown in FIG. 11A-B. The system included a Leap CTC PAL autosampler with cooling stack (Leap Technologies, Carrboro, N.C.). The analytical column was 150·4.6 mm C8, 5 μm (Zorbax Eclipse XDB C8, Agilent Technologies). The online extraction column was 12.5·4.6 mm C8, 5 μm (Zorbax Eclipse XDB C8, Agilent Technologies)

Mobile phase buffer was HPLC grade water+0.1% formic acid/HPLC grade methanol+0.1% formic acid. The flow rate was 2-3 mL/min during online extraction, and 1 mL/min for analytical column. The Autosampler temperature was 4° C. The Extraction column temperature was 40° C.

The Analytical column temperature was 40° C. The Injection volume 20 μL, unless otherwise specified. The Assay run time was 4.5 min.

Connections and positions of the column switching valve in the LC-MS/MS system are shown in FIG. 11A-B. As shown in FIG. 11A, in mode 1, the HPLC Pump I flows through the injector so the sample is injected to the extraction column. As shown in FIG. 11B, in mode 2, HPLC Pump II back flushes the extraction column onto the analytical column which is eluted to the API 4000 MS/MS system where MRM monitoring is employed.

Method of Analysis

Twenty μL of the samples were injected onto a 4.6·12.5 mm 5 μm extraction column (Eclipse XDB C-8, Agilent Technologies, Palo Alto, Calif.). Following injection, sample extraction column was washed with a mobile phase of 15% methanol with 0.1% formic acid and 85% water with 0.1% formic acid. The flow was 2-3 mL/min within 0.5 min and the temperature for the extraction column was set to 40° C., as shown in FIG. 11A. After 0.5 min, the switching valve was activated and the analytes were eluted in the backflush mode from the extraction column onto a 150·4.6 mm C8, 5 μm analytical column (Zorbax XDB C8, Agilent Technologies, Palo Alto, Calif.), as shown in FIG. 11B. The mobile phase consisted of methanol with 0.1% formic acid (solvent B) and 0.1% formic acid in HPLC grade water (solvent A). The following gradient was run: 0 to 0.5 minutes: 60% solvent B, 0.5 to 1.5 minutes: 60% to 98% solvent B, 1.5 to 4 minutes: hold at 98% solvent B, 4 to 4.1 minutes: 98% to 60% solvent B, and stay at 60% solvent B for the next 0.5 min. Detection of eluate was performed by MS/MS run in the negative multiple reaction monitoring (MRM) mode. Cholic acid was monitored at m/z=407.3→343.1 and 289.2 (qualifier ion), ¹³C-CA at m/z=408.3→343.1 and 289.2 (qualifier ion), D₄-CA at m/z=411.3→347.1 and 290.2 (qualifier ion) and the internal standard D₅-CA at m/z=412.3→290.2 and 348.1 (qualifier).

After the analysis was completed, peaks were integrated (linear fit with 1/x weighting, generated in Excel) and the results were printed. The concentrations of cholic acid, ¹³C-cholic acid and cholic acid-D₄ were quantified using the calibration curves based on analyte/internal standard ratios that were included in each batch. Signal integrations were carried out by the Applied Biosystems Analyst Software (version 1.6.2 or higher), quantification was carried out using Microsoft Excel with semi-validated spread sheet.

Data from MRM of distinguishable cholate compounds in samples are used to generate individualized oral and intravenous clearance curves for the patient. The curves are integrated along their respective valid time ranges and an area is generated for each. Comparison of intravenous and oral cholate clearance curves allows determination of first-pass hepatic elimination or portal shunt. The liver shunt fraction calculated by the formula: ShuntFraction=[AUCₒᵣₐₗ/AUC_{IV}]*[Dose_{IV}/Doseₒᵣₐₗ ]*100%, wherein AUC represents area under the curve and Dose represents the amount (in mg) of dose administered.

### Example 7. Use of LC-MS/MS Data to Determine DSI, STAT Values and Estimate DSI

A DSI value in a patient may be calculated using oral and intravenous clearance of distinguishable compounds, as described above, and/or according to methods in U.S. Pat. Nos. 9,091,701, 9,759,731 and 10,520,517, each of which is incorporated herein by reference. The DSI value in a patient, or DSI value over time in the patient, may be used to help determine liver function, disease progression, and treatment efficacy in an individual patient.

In addition, the STAT test value may be used to estimate DSI value in a patient, as provided herein.

The STAT test was administered to patients having or suspected of having a chronic liver disease, and healthy controls.

A STAT test was performed on n=1363 subjects, for example, according the method of Example 5. Briefly, patients were orally administered a composition containing 2,2,4,4-²H-Cholate, and Sodium bicarbonate (e.g. 40 mg, and 600 mg, respectively) at t=0. Blood samples were drawn at t=60 min post administration and samples were sent to laboratory 1 and/or laboratory 2 for sample processing and analysis by HPLC-MS using SIM and/or LC-MS/MS using negative ion MRM, respectively. FIG. 12B shows a graph of the relationship of DSI to STAT values in n=1363 subjects, and n=1736 tests. The relationship of DSI to STAT was used to derive the equation:

y=9.4514 ln(x)+21.12,

where x=STAT value (μM adjusted to 75 kg bodyweight), y=estimated DSI value, and R²=0.8499. Therefore, STAT value may be used to estimate DSI value in a patient.

### Example 8. Baseline and Serial Estimated DSI from STAT Values: Incident HCC in the HALT-C QLFT Ancillary Study

Each year up to 1 million individuals are diagnosed with hepatocellular carcinoma (HCC) worldwide.

Only 40% of patients with hepatocellular carcinoma (HCC) are diagnosed at an early stage; suggesting breakdowns in the surveillance process.

The Hepatitis C Antiviral Long-term Treatment against Cirrhosis (HALT-C) trial was designed to determine whether maintenance interferon therapy could slow disease progression in patients who had failed to eradicate hepatitis C virus (HCV) during prior interferon treatment. (nonresponders). Ten clinical sites, a virological testing center, and a data coordinating center were selected to collaborate in the design and implementation of final protocol. If patients failed to achieve clearance of virus from blood after a 20 week lead-in therapy of pegylated interferon plus ribavirin they were entered into main trial at week 24 and randomized to receive a low dose of pegylated interferon weekly alone or no further therapy for an additional 3½ yrs. Lee W M et al, Controlled Clinical Trials 2004; 25: 472-492.

Primary trial outcomes in HALT-C included increase in Ishak fibrosis score by ≥2 points at 2- or 4-year biopsies; death from any cause; development of hepatocellular carcinoma; Child-Turcotte-Pugh score of ≥7 at two consecutive study visits; variceal hemorrhage; ascites; spontaneous bacterial peritonitis; and hepatic encephalopathy.

Over a mean patient follow-up of 6.1 years, 692 (68.9%) of 1,005 patients had consistent surveillance. Consistent surveillance was defined as having an ultrasound and alpha-fetoprotein every 12 months. 83 patients developed HCC; and 23 (27.7%) were detected beyond Milan criteria (advanced HCC). Definite HCC was defined by (a) imaging demonstrating a mass with AFP levels >1,000 ng/ml or (b) histological confirmation.

A study was performed to assess the reasons behind surveillance process failures among patients in the HALT-C Trial, which prospectively collected HCC surveillance data on a large cohort of patients. Singal A G, et al. Am J Gastroenterol 2013; 108:425-432.

Surveillance failures among patients who developed. ICC were classified into one of three categories: absence of screening, absence of follow-up, or absence of detection.

Of 83 patients with HCC, 23 (27.7%) were detected beyond an criteria (advanced HCC). Three (13%) had late-stage HCC due to the absence of screening, 4 (17%) due to the absence of follow-up, and 16 (70%) due to the absence of detection.

Surveillance process failures, including absence of screening or follow-up, are common and potentially contribute to late-stage tumors in one-third of cases. However, the most common reason for finding HCC at a late stage was determined to be an absence of detection, suggesting better surveillance strategies are needed. Singal et al. 2013.

An ancillary study was performed to determine incident HCC in the HALT-C trial. Baseline and serial DSI values after 2 yrs, 4 yrs, or more were determined by the present inventors from 220 patient samples from the HALT-C study. Twelve of 113 patients having a baseline DSI value >18.3 were diagnosed with HCC during the years of follow-up period. In contrast, only 1 of 107 patients having a baseline DSI of <18.3 was diagnosed with HCC during follow-up period. In this case, DSI had increased to ˜23 by year 2, prior to his diagnosis of HCC at year 4.

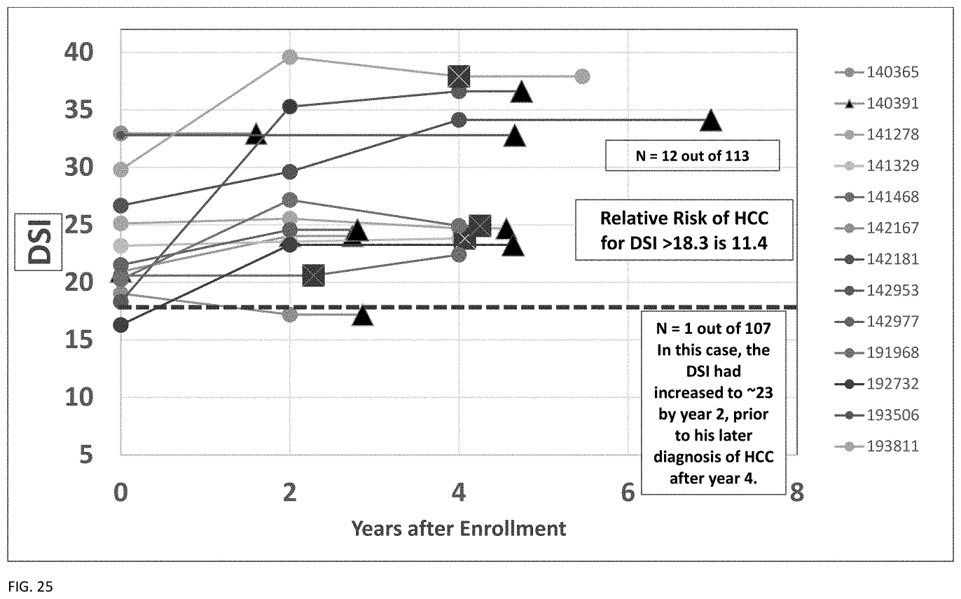

A graph of DSI baseline and serial DSI values in 13 patients eventually diagnosed with HCC is shown in FIG. 25. The Dashed Horizontal Line near bottom of the graph is DSI 18.3; this DSI cutoff is based upon large varices, but is also a cutoff for clinical outcomes (ascites, variceal hemorrhage, encephalopathy, SBP, and liver-related death); and, now, as shown to the left, it may also be a cutoff for risk for HCC. Twelve of thirteen HCC cases diagnosed during follow-up period had baseline DSI>18.3. The relative risk of HCC for DSI>18.3 is 11.4.

In a further analysis of the HALT-C ancillary study, STAT values were used to obtain baseline and serial estimated DSI values similar to example 7, except according to the equation:

Est DSI=9.9013 ln(STAT)+19.53

R²=0.8752

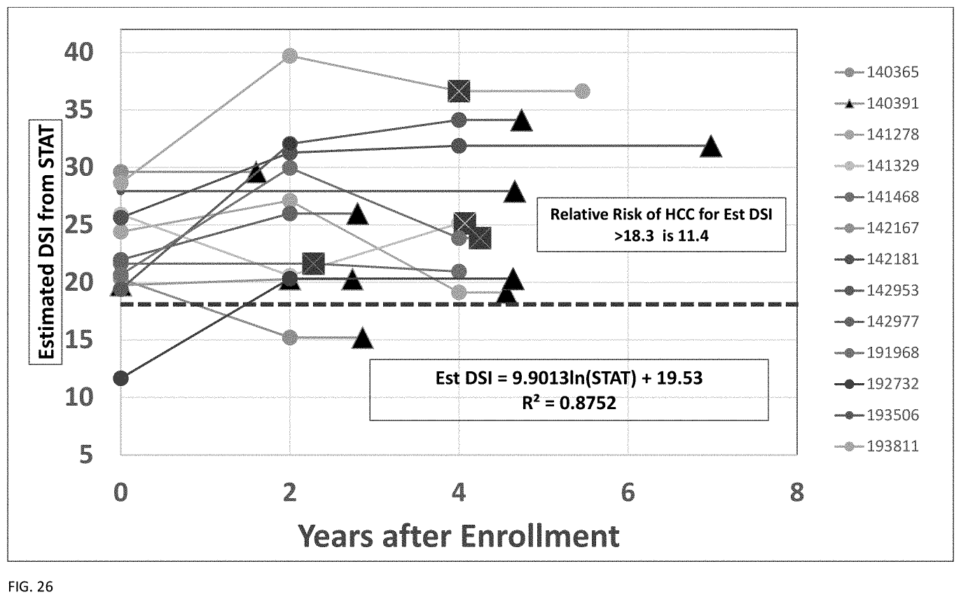

A graph of estimated DSI from STAT at baseline and over years of enrollment for 13 patients eventually diagnosed with HCC is shown in FIG. 26. Twelve of thirteen HCC cases had baseline estimated DSI values >18.3. The relative risk of HCC for estimated DSI>18.3 is 11.4. This example supports use of DSI values and estimated DSI values from STAT values in surveillance of patients at risk of developing HCC.

### Example 9. Predict Risk for Clinical Outcome Using Baseline DSI Values from HALT-C

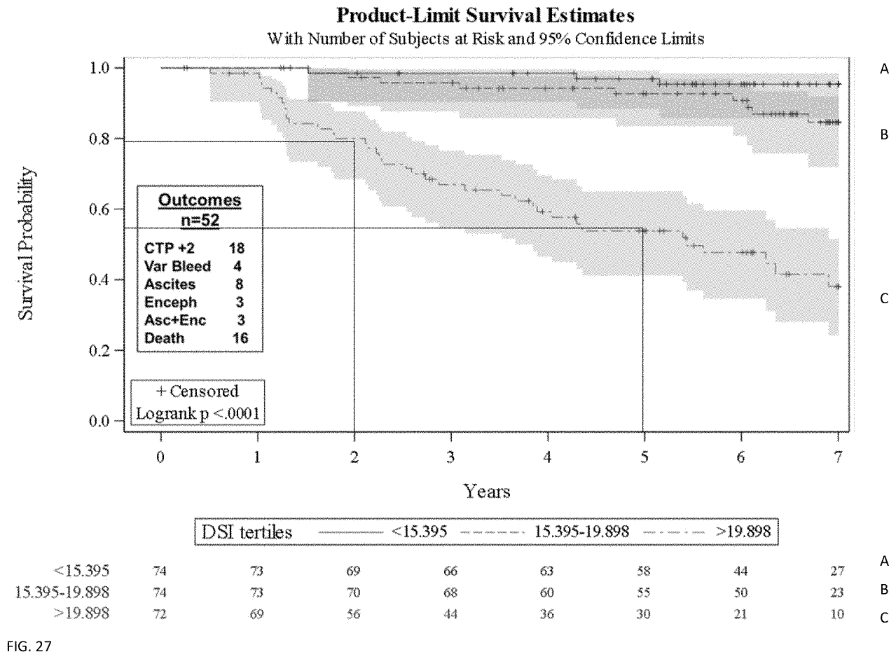

Baseline DSI values were calculated from data obtained from 220 patients infected with hepatitis C virus participating in the HALT-C study. Among the 220 patients for which an baseline DSI value was calculated, a total of 52 patients experienced severe clinical outcomes over the course of the study. Clinical outcomes included CTP progression (CTP+2; n=18), variceal hemorrhage (Var Bleed; n=4), ascites (n=8), hepatic encephalopathy (n=3), ascites+encephalopathy (n=3), and death (n=16). Baseline DSI values were used to divide 220 HALT-C patients into tertiles: patients having (A) DSI value <15.395, (B) DSI value from 15.395-19.898, and (C) DSI value >19.898, as shown in FIG. 27. FIG. 27 shows a graph of survival probability per DSI tertile vs. study years with number of subjects shown under the graph. Hazard ratios, upper and lower confidence intervals, and p-values are shown in Table 11. In survival analysis, the hazard ratio is the ratio of the hazard rates corresponding to the conditions described by two levels of an explanatory variable. An baseline DSI value >19.898 was found to significantly indicate risk for decreased survival probability (p<0.001). The other variables tested including ISHAK fibrosis stage (4 vs. 3, 5 vs. 3, or 6 vs. 3), baseline DSI value between 15.395-19.898, platelets per unit, age per year, gender, or race were not significant.

TABLE 11
Hazard Ratios for Survival Probability

| | | Lower 95% | Upper 95% | |
|---|---|---|---|---|
| Variable | Hazard Ratio | CI | CI | p-value |
| DSI tertile | 1.97 | 0.52 | 7.49 | 0.319 |
| 15.395-19.898 | | | | |
| DSI | 14.77 | 3.91 | 55.73 | <0.001 |
| tertile >19.898 | | | | |
| Fibrosis | . | . | . | . |
| ISHAK 2 vs 3 | | | | |
| Fibrosis | 2.31 | 0.76 | 7.05 | 0.140 |
| ISHAK 4 vs 3 | | | | |
| Fibrosis | 1.11 | 0.34 | 3.64 | 0.866 |
| ISHAK 5 vs 3 | | | | |
| Fibrosis | 1.73 | 0.57 | 5.23 | 0.334 |
| ISHAK 6 vs 3 | | | | |
| Platelets per unit | 1.00 | 0.99 | 1.00 | 0.202 |
| Age per year | 0.98 | 0.94 | 1.02 | 0.238 |
| Gender Male vs | 1.53 | 0.76 | 3.08 | 0.230 |
| Female | | | | |
| Race Black vs | 0.48 | 0.18 | 1.28 | 0.143 |
| Non-Hispanic, | | | | |
| White | | | | |
| Race | 0.87 | 0.42 | 1.80 | 0.713 |
| Hispanic/other vs | | | | |
| Non-Hispanic, | | | | |
| White | | | | |

The baseline DSI value may be used in a method for amplifying clinical data into relatable risk for clinical outcome such as survival probability in a chronic liver disease patient.

### Example 10. Analysis of Clinical Event Rate as a Function of DSI

In this example, regression models were developed using baseline DSI value, subsequent DSI value, and/or change in DSI value over time in HALT-C patients to determine relatable risk for clinical outcome in chronic liver disease patients.

1. The HALT-C data set includes 220 subjects with baseline DSI. There are a total of 52 clinical events. Thirty-two subjects are missing the 24-month DSI measurement. Those 32 subjects experienced 7 events. The models in this example are restricted to subjects who are not missing 24-month DSI; thus, the analyses have 188 subjects and 45 clinical events.

2. Models Poisson regression is used to estimate the event rate (events per person-year of observation) as a function of baseline DSI and 24-month DSI. Conceptually the models all have the form:

Y=β₀+β₁X₁+β₂X₂+β₃X₃

where Y=0 if the subject did not have an event and Y=1 if the subject experienced a clinical event. The explanatory variables are denoted by X1, X2, and X3 and the regression coefficients are denoted by β0(intercept)-β3. Poisson regression estimates the log of the event rate, and so the coefficients are interpreted as the log of the event rate for a 1-unit change in the explanatory variable.

Four Poisson regression models were developed. Model A employs the baseline DSI value alone, model B employs both the baseline DSI value and the 24 month DSI value, model C employs baseline DSI value, 24 month DSI value, and interaction of DSI value to 24 month DSI value, and model D employs both baseline DSI value and the 24 month change in DSI value.

Model A: Relationship with baseline DSI (denoted by dsi0):

Y=β₀+β₁dsi0

Model B: Relationship with baseline DSI and 24-month DSI (denoted by dsi24):

Y=β₀+β₁dsi0+β₂dsi24

Model C: Interaction between DSIO and DSI24 (i.e., does contribution of 24-month DSI depend on the level of baseline DSI).

Y=β₀+β₁dsi0+β₂dsi24+β₃(dsi0*dsi24)

Model D: Relationship with baseline DSI and 24-month change DSI (denoted by dltaDSI). Note that this model is essentially the same as model B:

$$\begin{matrix}Y = {β}_{0} + \\ = {β}_{0} + \\ = β\end{matrix} {β}_{1} dsi 0 + {β}_{1} dltaDSI$$

3. Model results: Table 12 shows the fitted coefficients for each of the models described in the previous section. For example in model B: Y=β0+β1dsi0+β2dsi24, and the fitted values are β0=−7.201, β1=0.033, and β2=0.139. The p-value is from a test of whether or not the coefficient equals 0.

TABLE 12
Model Coefficients for Regression Models A-D using DSI values

| | Coef- | Coef- | | |
|---|---|---|---|---|
| Model | ficient | ficient | (95% CI) | P-value |
| Model A:(Intercept) | β0 | −6.2997 | (−7.359, −5.279) | 0.00000 |
| ModelA:dsi0 | β1 | 0.1498 | (0.107, 0.191) | 0.00000 |
| Model B:(Intercept) | β0 | −7.2008 | (−8.417, −6.057) | 0.00000 |
| Model B:dsi0 | β1 | 0.0331 | (−0.025, 0.091) | 0.26515 |
| Model B:dsi24 | β2 | 0.1395 | (0.092, 0.185) | 0.00000 |
| Model C:(Intercept) | β0 | −9.4069 | (−13.971, −5.291) | 0.00002 |
| Model C:dsi0 | β1 | 0.1373 | (−0.061, 0.341) | 0.18193 |
| Model C:dsi24 | β2 | 0.2119 | (0.073, 0.362) | 0.00396 |
| Model C:dsi0:dsi24 | β3 | −0.0033 | (−0.010, 0.003) | 0.29520 |
| ModelD:(Intercept) | β0 | −7.2008 | (−8.417, −6.057) | 0.00000 |
| ModelD:dsi0 | β1 | 0.1726 | (0.127, 0.217) | 0.00000 |
| ModelD:dltaDSI | β2 | 0.1395 | (0.092, 0.185) | 0.00000 |

As shown in Table 12, Model A employs the baseline DSI value alone (dsi0), which is shown to be significant for correlation to a clinical event.

Model B employs both the baseline DSI value (dsi0) and the 24 month DSI value (dsi24). In model B, all significance is found at the 24 month DSI value, and baseline DSI drops out.

Model C employs baseline DSI value (dsi0), 24 month DSI value (dsi24), and interaction of DSI value to 24 month DSI value (dsi0:dsi24). The significance is found at the 24 month DSI value.

Model D employs both baseline DSI value (dsi0) and the 24 month change in DSI value (dlta DSI). In this model, both baseline DSI and dlta DSI values are highly significant.

For each of model A, B, C, and D equations, Y=1 year predicted clinical event rate(ln(rate)); β0, β1, β2, and β3 are coefficients, found in Table 6; dsi0=baseline DSI value in a patient, dsi24=repeat dsi value (at 24 months).

4. Model interpretation: The above coefficients can be used to calculate the event rate for each individual according to the DSI measurements at baseline and month 24. Examples of using the above coefficient table are shown below.

### Example A

The first subject (id=140175) has baseline DSI=23.50 and 24-month DSI=26.20. The results from model B predict that the log of the event rate is: ln(rate)=−7.201+0.033×23.50+0.139×26.20=−2.7837, and so their predicted event rate is exp(−2.7837)=0.0618. That is, if followed for 1-year then this person would have about a 6.2% risk of experiencing an event, or if 100 equivalent subjects were followed for 1-year then we would predict that 6 of them would experience an event.

### Example B

If the first subject (id=140175) had an intervention that reduced the 24-month DSI from 26.20 to 24.20, then their predicted event rate would be reduced to: ln(rate)=−7.201+0.033×23.50+0.139×24.20=−3.0617, and so their predicted event rate is exp(−3.0617)=0.0468. That is, the event rate is reduced by 24% (0.0468/0.0618=0.76).

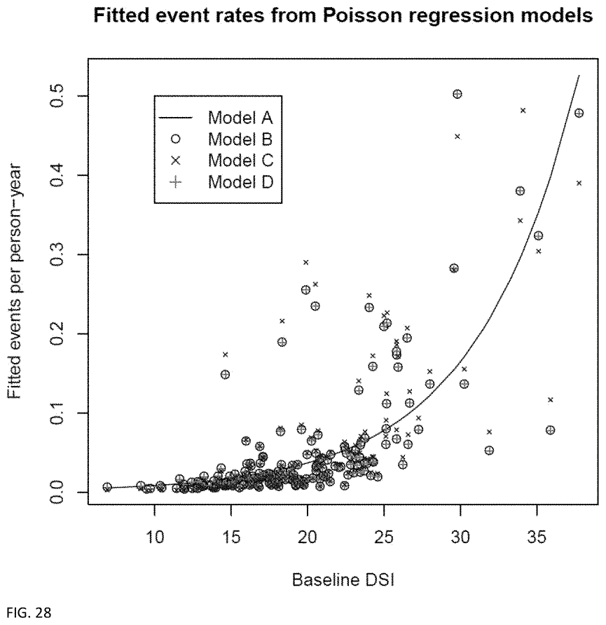

Predicted event rates for each of the regression models are shown in FIG. 28. FIG. 28 shows the predicted event rate for each of the 4 models for all 188 subjects as a function of their baseline DSI. Notice that B and D have the same predicted values. This is expected as shown in the equations in the description of Model D.

Conclusions:

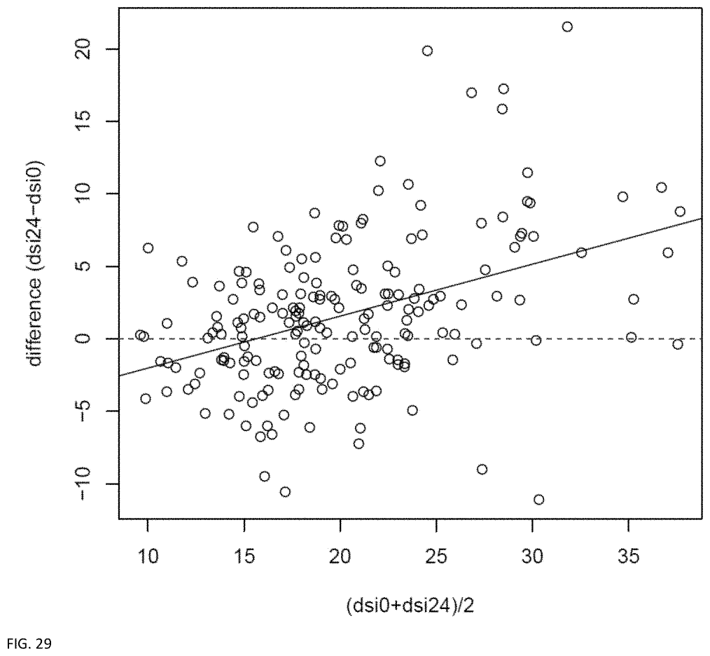

Model results: There is no significant interaction between baseline and 24-month DSI for predicting event rates. Therefore, it may be optional to include an interaction term. As shown in the FIG. 28 models B and D are the same, but model D evaluates the contribution of change separately from baseline. Relationship between baseline and 24-month DSI. The relationship between baseline and 24-month DSI may be explored. With baseline follow-up measurements in many other models, it is usually expected to see regression to the mean (i.e., lower baseline DSI gets larger at 24-months and higher base-line gets lower at 24-months). It was surprising to see that the opposite occurred: those with lower baseline DSI tended to have smaller DSI at 24-months and those with higher baseline DSI tended to have higher DSI 24-months. FIG. 29 shows this phenomenon.

Tables 13-16 are provided to facilitate additional calculations based on the models A, B, C, D using DSI values from Example A and Example B.

TABLE 13
Model A: Calculation for Risk of event
in one year from baseline DSI

| | | | Risk of | | |
|---|---|---|---|---|---|
| | | Base | event in | | |
| | | DSI | 1 yr | LL CI | UL CI |
| | Ex A | 23.5 | 6.2% | 0.8% | 44.9% |
| | Ex B | 23.5 | 6.2% | 0.8% | 44.9% |
| From DSI Table by | | | | | |
| stage of fibrosis | | | | | |
| | F2 | 16.92 | 2.3% | 0.4% | 12.8% |
| | F3 Out | 20.66 | 4.1% | 0.6% | 26.1% |
| | F3 No Out | 15.84 | 2.0% | 0.3% | 10.4% |
| | F4 Out | 22.18 | 5.1% | 0.7% | 34.9% |
| | F4 No Out | 17.96 | 2.7% | 0.4% | 15.6% |
| | F5 Out | 23.91 | 6.6% | 0.8% | 48.5% |
| | F5 No Out | 19.07 | 3.2% | 0.5% | 19.3% |
| | F6 Out | 25.53 | 8.4% | 1.0% | 66.1% |
| | F6 No Out | 21.62 | 4.7% | 0.6% | 31.4% |

TABLE 14
Model B: Calculation for Risk of event in
one year from baseline DSI and 24-month DSI

| | | | Risk of | | |
|---|---|---|---|---|---|
| | Base | M24 | event in | | |
| | DSI | DSI | 1 yr | LL CI | UL CI |
| Ex A | 23.5 | 26.2 | 6.3% | 0.1% | 255.7% |
| Ex B | 23.5 | 24.2 | 4.8% | 0.1% | 176.6% |
| From DSI Table by | | | | | |
| stage of fibrosis | | | | | |
| F2 | 16.92 | 17.01 | 1.4% | 0.1% | 25.6% |
| F3 Out | 20.66 | 25.6 | 5.3% | 0.1% | 176.5% |
| F3 No Out | 15.84 | 17.49 | 1.4% | 0.1% | 25.3% |
| F4 Out | 22.18 | 28.25 | 8.0% | 0.2% | 331.1% |
| F4 No Out | 17.96 | 18.74 | 1.8% | 0.1% | 38.8% |
| F5 Out | 23.91 | 29.69 | 10.4% | 0.2% | 506.2% |
| F5 No Out | 19.07 | 17.95 | 1.7% | 0.1% | 37.1% |
| F6 Out | 25.53 | 29.7 | 11.0% | 0.2% | 588.1% |
| F6 No Out | 21.62 | 22.45 | 3.5% | 0.1% | 107.6% |

TABLE 15
Model C: Calculation for Risk of event in one year from baseline DSI, 24-
month DSI, and interaction of DSI value to 24 month DSI value(dsi0:dsi24)

| | | | | Risk of | | |
|---|---|---|---|---|---|---|
| | Base | M24 | Base * | event in | | |
| | DSI | DSI | M24 | 1 yr | LL CI | UL CI |
| Ex A | 23.5 | 26.2 | 615.7 | 7.1% | 0.0% | 103323929.0% |
| Ex B | 23.5 | 24.2 | 568.7 | 5.4% | 0.0% | 44230206.0% |
| From DSI Table by | | | | | | |
| stage of fibrosis | | | | | | |
| F2 | 16.92 | 17.01 | 287.8 | 1.2% | 0.0% | 164529.0% |
| F3 Out | 20.66 | 25.6 | 528.9 | 5.6% | 0.0% | 25043698.7% |
| F3 No Out | 15.84 | 17.49 | 277.0 | 1.2% | 0.0% | 131555.1% |
| F4 Out | 22.18 | 28.25 | 626.6 | 8.8% | 0.0% | 142286601.7% |
| F4 No Out | 17.96 | 18.74 | 336.6 | 1.7% | 0.0% | 499514.3% |
| F5 Out | 23.91 | 29.69 | 709.9 | 11.5% | 0.0% | 539552539.4% |
| F5 No Out | 19.07 | 17.95 | 342.3 | 1.6% | 0.0% | 556691.4% |
| F6 Out | 25.53 | 29.7 | 758.2 | 12.2% | 0.0% | 1070474981.2% |
| F6 No Out | 21.62 | 22.45 | 485.4 | 3.8% | 0.0% | 9905028.6% |

TABLE 16
Model D: Calculation for Risk of event in one year from
baseline DSI, and change in 24 month DSI from baseline

| | | | | Risk of | | |
|---|---|---|---|---|---|---|
| | Base | | | event in | | |
| | DSI | M24 | ΔDSI | 1 yr | LL CI | UL CI |
| Ex A | 23.5 | 26.2 | 2.7 | 6.3% | 0.6% | 63.4% |
| Ex B | 23.5 | 24.2 | 0.7 | 4.8% | 0.5% | 43.8% |
| From DSI Table by | | | | | | |
| stage of fibrosis | | | | | | |
| F2 | 16.92 | 17.01 | 0.09 | 1.4% | 0.2% | 9.4% |
| F3 Out | 20.66 | 25.6 | 4.94 | 5.3% | 0.5% | 51.8% |
| F3 No Out | 15.84 | 17.49 | 1.65 | 1.4% | 0.2% | 9.9% |
| F4 Out | 22.18 | 28.25 | 6.07 | 8.0% | 0.6% | 88.7% |
| F4 No Out | 17.96 | 18.74 | 0.78 | 1.8% | 0.2% | 13.3% |
| F5 Out | 23.91 | 29.69 | 5.78 | 10.4% | 0.8% | 122.4% |
| F5 No Out | 19.07 | 17.95 | −1.12 | 1.7% | 0.2% | 11.9% |
| F6 Out | 25.53 | 29.7 | 4.17 | 11.0% | 0.8% | 129.2% |
| F6 No Out | 21.62 | 22.45 | 0.83 | 3.5% | 0.4% | 29.8% |

### Example 11. RISK-ACE: Using DSI and/or ΔDSI to Determine an Individual's Risk for a Clinical Event within 1 Year

DSI values and change in DSI over time (ΔDSI) may also be used to determine individual risk for a clinical event within 1 year according to models A, B, C or D from Example 9, using coefficients shown in Tables 7-10. Determination of risk for a clinical event within 1 year (RISK ACE) for an individual patient is shown in examples 10.1 to 10.4.

### Example 11.1 Calculating RISK-ACE for Low Risk Patient from Baseline DSI Value

The estimated risk of experiencing a clinical event in 1 year (RISK-ACE) was calculated for an individual patient having a baseline DSI value (dsi0)=16.9 using model A equation.

Model A: Y=β0+β1dsi0

Using model A coefficients from Table 6: β0=−6.2997, β1=0.1498, for a patient having a baseline DSI value (dsi0)=16.9, the results from model A predict that the log of the event rate is:

ln(rate)=−6.2997+0.1498 (16.9)=−3.768, and so their predicted event rate is exp(−3.768)=0.0231. The RISK-ACE (model A) for the patient is 2.3%.

### Example 11.2. Calculating RISK-ACE for Low Risk Patient from Baseline DSI and ΔDSI Values

The estimated risk of experiencing a clinical event in 1 year (RISK-ACE) was calculated for an individual patient having a baseline DSI value (dsi0) of 16.9 and a repeat DSI value (dsi24) of 17.0 using model D equation.

Model D:
Y=β0+β1dsi0+β2(Δdsi)
Y=β0+β1dsi0+β2(dsi24−dsi0)
   Using Model D coefficients from Table 6: β0=−7.2008, β1=0.1726, β2=0.1395, for a patient having a baseline DSI value (dsi0)=16.9, and a repeat DSI value (dsi24) of 17.0, the results from model D predict that the log of the event rate is:

ln(rate)=−7.2008+0.1726×16.9+0.1395 (17.0−16.9), and so the predicted event rate is exp(−4.269)=0.01398. The RISK-ACE (model D) for the patient is 1.4%. Surprisingly, even though the repeat DSI value had increased over baseline DSI, the risk of experiencing a clinical event in 1 year (RISK-ACE) decreased from 2.3% to 1.4%.

### Example 11.3 Calculating RISK-ACE for High Risk Patient from Baseline DSI Value

The estimated risk of experiencing a clinical event in 1 year (RISK-ACE) may be calculated for an individual patient having a baseline DSI value of 25.5 model A equation.

Model A: Y=β0+β1dsi0

Using model A coefficients from Table 6: β0=−6.2997, β1=0.1498, for a patient having a baseline DSI value (dsi0)=25.5, the results from model A predict that the log of the event rate is:

ln(rate)=−6.2997+0.1498 (25.5)=−2.4798, and so their predicted event rate is exp(−2.4798)=0.08376. The RISK-ACE (model A) for the patient is 8.4%.

### Example 11.4. Calculating RISK-ACE for High Risk Patient from Baseline DSI and ΔDSI Values

The estimated risk of experiencing a clinical event in 1 year (RISK-ACE) may be calculated for an individual patient having a baseline DSI value of 25.5 and a repeat DSI value of 29.7 (24 months) using model D equation.

Model D:
Y=β0+β1dsi0+β2(Δdsi)
Y=β0+β1dsi0+β2(dsi24−dsi0)
   Using Model D coefficients from Table 6: β0=−7.2008, β1=0.1726, β2=0.1395, for a patient having a baseline DSI value (dsi0)=25.5, and a repeat DSI value (dsi24) of 29.7, the results from model D predict that the log of the event rate is:

ln(rate)=−7.2008+0.1726×25.5+0.1395 (29.7−25.5)=−2.2136, and so the predicted event rate is exp(−2.2136)=0.1093. The RISK-ACE (model D) for the patient is 11.0%.

The RISK-ACE may be used to inform patient of their risk of experiencing a clinical event within one year of their most recent DSI test. The clinical event, or clinical outcome, may be CTP score progression (CTP+2), variceal hemorrhage, ascites, encephalopathy, or death.

### Example 12. Quantification of 12C-CA, 13C-CA, d4-CA in Serum Using MS/MS without Chromatography

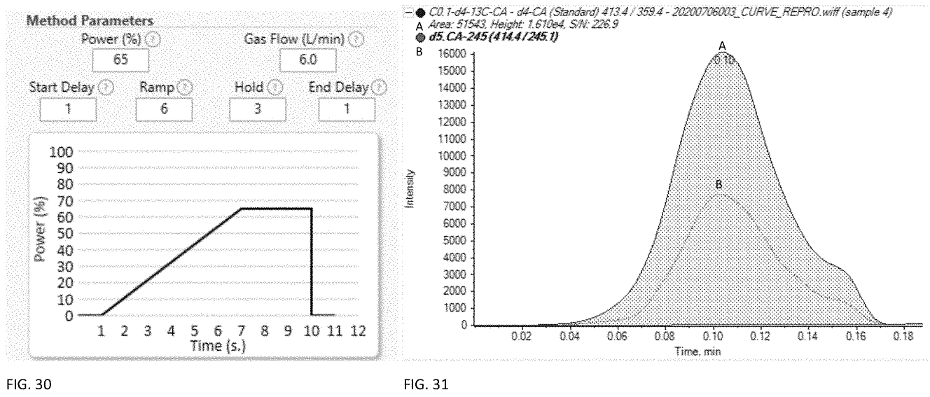

Standard and QC human serum samples (10 uL) were evaluated for 12C-CA, 13C-CA, d4-CA with d5-CA internal standard using MS/MS without chromatography. A LUXON® ion source (Phytronix Technologies, Inc.) laser diode thermal desorption (LDTD) process was used to generate solvent free atmospheric pressure chemical ionization (APCI). The gas composition allows protonation in positive mode which is typically not employed in ESI or LC-APCI. Isotopic contribution of d4-CA to d5-CA and 12C-CA to 13C-CA was taken into account. A LUXON® S-960 ion source was employed with a Q-trap 5500, Sciex triple-quadrapole mass spectrometer. Samples were prepared using an Automation liquid handler (Phytronix) with LLE (liquid-liquid extraction) as follows. A 10 uL serum sample was mixed with 10 uL internal standard of d5-CA at 10 uM in a bicarbonate mixture. In this case, the d5-CA internal standard (IS) level was raised to 10 uM from typical concentration of 2.5 uM in IS to limit the d4-CA contribution. 20 uL KH₂PO₄ (1M in water) was added and diluted sample was mixed. 100 uL hexane/ethyl acetate (1:1 v/v) was added, and samples were mixed and phase separation by gravity was allowed. 4 uL upper layer was added to a LazWell™-DEC (desorption enhancing coating) plate (Phytronix) followed by fast evaporation of solvent. LUXON-MS/MS analysis was performed. LUXON® method parameters are shown in FIG. 30, including Ionization mode: Positive. Flow: 6 L/min. Gaz: Air. Q1 mass and Q3 mass for each of 12C-CA, 13C-CA, d4-CA and d5-CA are shown in Table 17.

TABLE 17
MS/MS Q1 Masses and Q3 Masses for 12C-CA, 13C-
CA, d4-CA and d5-CA in Human Serum Samples

| | Q1 Mass (Da) | Q3 Mass (Da) | Time (msec) | ID |
|---|---|---|---|---|
| 1 | 409.300 | 355.400 | 25.0 | 12-CA |
| 2 | 410.300 | 356.300 | 25.0 | 13-CA |
| 3 | 413.400 | 359.400 | 25.0 | d4-CA |
| 4 | 414.400 | 360.400 | 25.0 | d5-CA |
| 5 | 414.400 | 245.100 | 25.0 | d5-CA-245 |

FIG. 31 shows an exemplary desorption peak for C 0.1 d4-CA standard 413.4/359.4 (large peak) with internal standard d5-CA-245 (414.4/245.1) (inset peak).

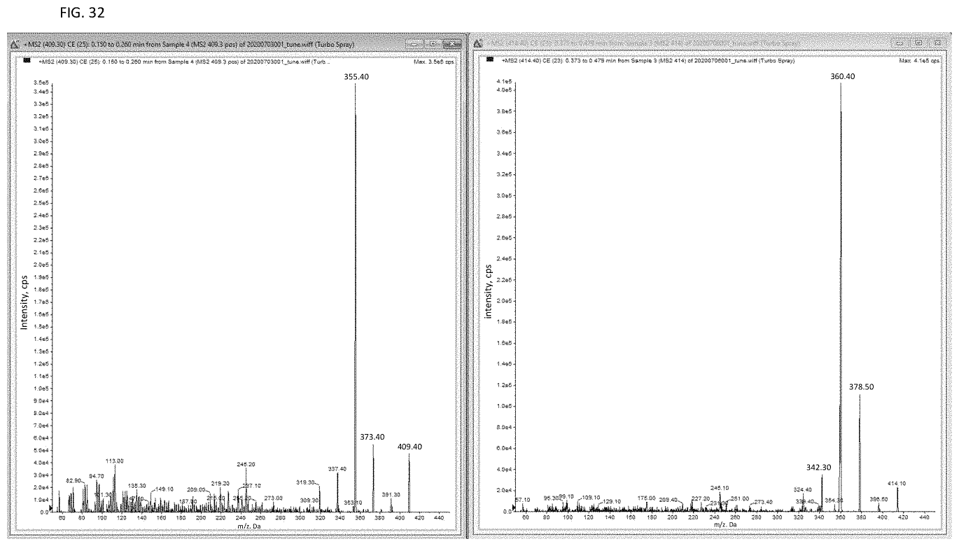

FIG. 32 shows product ion (MS2) positive mode 12CA at 355.40 m/z, Da (left panel) and product ion (MS2) for d5-DA at 360.40 m/z, Da (right panel).

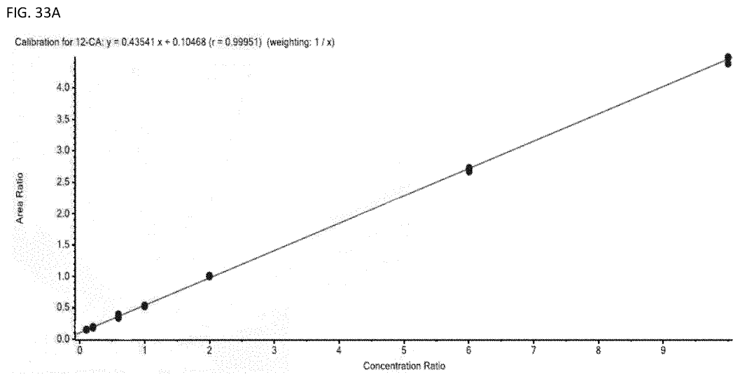

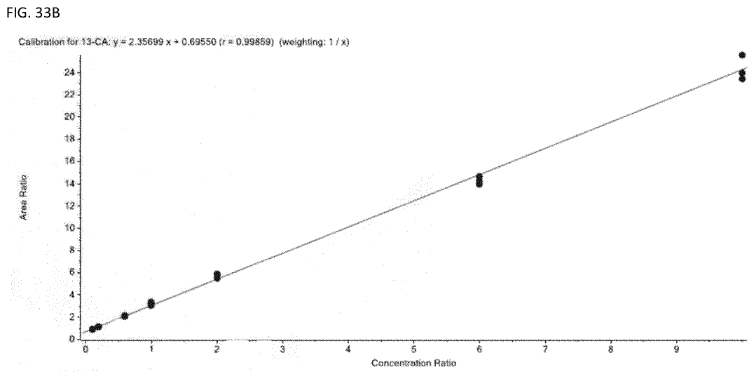

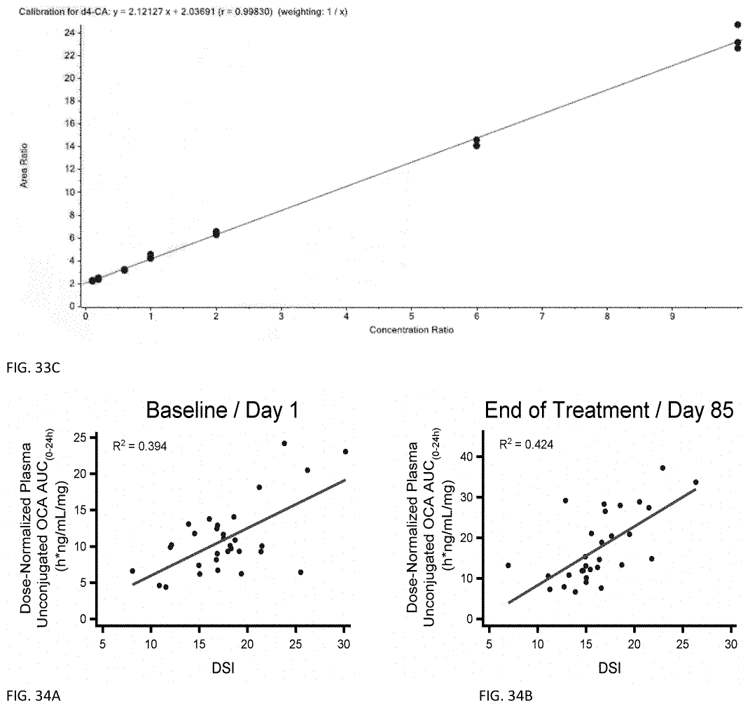

FIGS. 33A, B and C show graphs of concentration ratio vs. area ratio for standard samples of 12C-CA, 13C-CA, and d4-CA, respectively, at 0.1 uM, 0.2 uM, 0.6 uM, 1 uM, 2 uM, 6 uM, and 10 um (n=3 each). Calibration for 12C-CA was y=0.43541x+0.10468 (linearity r=0.99951)(weighing 1/x), as shown in FIG. 33A. Calibration for 13C-CA was y=2.35699 x+0.69550 (linearity r=0.99859)(weighing 1/x) as shown in FIG. 33B. Calibration for d4-CA was y=2.12127 x+2.03691 (linearity r=0.99830)(weighing 1/x) as shown in FIG. 33C.

Four QC serum samples Q1, Q2, Q3, Q4 were subjected to MS/MS. Tables 18A-C show results of quantitation.

TABLE 18A
Quantitation by MS/MS for 12C-CA in QC samples

| Sample | N | Mean (μM) | % RSD |
|---|---|---|---|
| QC1 | 3 | 0.8 | 6.8 |
| QC2 | 3 | 0.8 | 2.5 |
| QC3 | 3 | 2 | 5.5 |
| QC4 | 3 | 4.3 | 2.2 |

TABLE 18B
Quantitation by MS/MS for 13C-CA in QC samples

| Sample | N | Mean (μM) | % RSD |
|---|---|---|---|
| QC1 | 3 | 8.3 | 4.8 |
| QC2 | 3 | 2.8 | 2.1 |
| QC3 | 3 | 1.5 | 3.1 |
| QC4 | 3 | 2.2 | 6.5 |

TABLE 18C
Quantitation by MS/MS for d4-CA in QC samples

| Sample | N | Mean (μM) | % RSD |
|---|---|---|---|
| QC1 | 3 | 0.27 | 2.7 |
| QC2 | 3 | 0.61 | 3.8 |
| QC3 | 3 | 2.27 | 2.5 |
| QC4 | 3 | 6.50 | 4.6 |

Linearity was greater than 0.99 for standard samples of 12C-CA, 13C-CA, and d4-CA. Precision and accuracy were within the acceptance criteria. Sample to sample analysis time was 12 sec. Care was taken to account for isotopic contribution of adjacent mass. This example shows that distinguishable agents may be quantified in serum samples by MS/MS without using chromatography, and using automated sample preparation.

### Example 13. Quantification of 12C-CA, 13C-CA, d4-CA in Serum Using Acoustic Ejection MS without Chromatography

An Acoustic Ejection Mass Spectrometer system was used to perform analysis of 12C-cholic acid (CA), 13C-CA, d4-CA and d5-CA concentration from standard samples and four QC human serum samples. Echo® MS technology (Sciex) includes open port interface (OPI) for direct liquid transferring, acoustic droplet ejection (ADE) for low volume sampling, and MS with ESI ionization using triple Quad 6500+ mass spectrometer. Sample dilution and introduction to conventional MS/MS with electrospray ionization was performed directly from plate, without liquid chromatography.

Serum samples were prepared off line by dilution in methanol 1:5, mixing for 5 min, followed by a 20 min centrifugation, and supernatant collection. Samples from crashed serum 1:5 dilution scheme (20% concentration from patient sample) were employed in analysis. Processed samples were subjected to Echo® MS technology with four transitions monitored in MRM for each analyte using Q1 mass and Q3 mass as shown in Table 19. Total scan time per sample was 0.120 sec.

TABLE 19
MS/MS Q1 Masses and Q3 Masses for 12C-CA, 13C-
CA, d4-CA and d5-CA in Human Serum Samples

| | | Q1 Mass | Q3 Mass | Dwell Time |
|---|---|---|---|---|
| | ID | (Da) | (Da) | (msec) |
| 1 | 12-CA-1 | 407.250 | 343.100 | 25.0 |
| 2 | 13-CA-1 | 408.250 | 343.100 | 25.0 |
| 3 | d4-CA-1 | 411.250 | 347.200 | 25.0 |
| 4 | d5-CA-1 | 412.250 | 348.100 | 25.0 |

A droplet ladder using ejection volumes of 2.5 nL to 50 nL was run using ejection volumes 2.5, 5, 10, 15, 20 and 50 nL. A 1:1 water:sample of 10 uM sample was employed using carrier methanol, a 1:1 water:sample of 0.6, 1.6, and 6 uM and pure 6 uM sample were run using 1 mM NH₄OH in methanol, and a 1:1 water:sample of 0.6, 1.6, and 6 uM and pure 6 uM sample were run in carrier 1 mM NH4F in methanol. The carrier solvent was selected as 1 mM NH4F in 98% methanol. The ejection volume was 25 nL. MRM with four transitions for each analyte were monitored. Calibration curves were run for each analyte at 0.10, 0.20, 0.60, 1.00, 2.00, 6.00 and 10.00 uM. An entire 290 sample run of standards, QC samples and cross talk samples was run in less than 11 minutes. Calibration curves with and without internal standard were run for each analyte. QC samples Q1, Q2, Q3 and Q4 were analyzed for 12C-CA and 12C-CA concentration. Results are shown in Tables 20A and 20B

TABLE 20A
Quantitation by MS/MS for 12C-CA in QC samples

| | | Num. | Mean | Std. | % |
|---|---|---|---|---|---|
| Analyte | Sample | values | (μM) | Dev. | CV |
| 12C-CA | QC1 | 10 of 10 | 0.30821 | 0.06798 | 22.06 |
| 12C-CA | QC2 | 10 of 10 | 0.69979 | 0.11134 | 15.91 |
| 12C-CA | QC3 | 10 of 10 | 2.29634 | 0.12316 | 5.36 |
| 12C-CA | QC4 | 10 of 10 | 7.06786 | 0.89498 | 12.66 |

TABLE 20B
Quantitation by MS/MS for 13C-CA in QC samples

| | | Num. | Mean | Std. | Percent |
|---|---|---|---|---|---|
| Analyte | Sample | values | (μM) | Dev. | CV |
| 13C-CA | QC1 | 10 of 10 | 7.59879 | 0.38415 | 5.06 |
| 13C-CA | QC2 | 10 of 10 | 2.77981 | 0.45354 | 16.32 |
| 13C-CA | QC3 | 10 of 10 | 0.84441 | 0.16347 | 19.36 |
| 13C-CA | QC4 | 10 of 10 | 0.40565 | 0.06211 | 15.31 |

The Echo® MS cholate assay demonstrates linearity over a biologically relevant range from 0.2 uM to 10 uM. LOD of 0.1 uM visible. Sample preparation techniques may be used to increase LOD/LLOQ. The Echo® MS allowed quantitation of four analytes (2 compounds of interest) independently or with matching internal standard (IS). Further optimization of carrier solvent and ejection volumes may be used to increase linearity and dynamic range depending on sample preparation techniques.

### Example 14. DSI Correlates with Childs-Turcotte-Pugh Class and Score

Childs-Turcotte-Pugh (CTP, CP) score has been used as a main clinical score by the pharmaceutical industry, the Food and Drug Administration (FDA), and others in the baseline stratification of underlying liver disease—both in treatment clinical trials and in hepatic impairment studies.

The present inventors compared DSI scores to CTP scores in several clinical studies. The additional granularity provided by DSI over Child-Pugh class and score is shown in Table 21.

TABLE 21
Comparison of CP class, CP score, and DSI values

| CP Class | CP Score | DSI (continuous), |
|---|---|---|
| (categorical) | (categorical) | 0 (healthy) to 50 (very sick) |
| A | 5 | 19.9 ± 3.5 |
| | | (HALT-C, N = 52)* |
| | 6 | 25.6 ± 7.0 |
| | | (HALT-C, N = 41) |
| B | 7-9 | 35.0 ± 1.9 |
| | | (Clinical Trial T, CPB, N = 8) |
| C | 10-15 | 38.5 ± 4.7 |
| | | (Clinical Trial S, CP 10-12**, N = 6) |

* DSI was 16.7 ± 3.7 for non-cirrhotic subjects in HALT-C for CP A5
**The CP C group in Clinical Trial S did not include CP scores from 13-15

Within each class and score of the Child-Pugh classification, DSI provided a continuous spectrum of functional severity of the underlying liver disease. The data in Table 21 demonstrates that DSI values correlate with both CP class and CP score, but also provide unique quantitative data within each CP class and score. DSI scores may be a useful alternative to CTP class and score for use in baseline stratification of underlying liver disease, as a possible endpoint in treatment clinical trials, and in hepatic impairment studies.

### Example 15. DSI as a Determinant of Drug Pharmacokinetics

Hepatic impairment studies assessing drug pharmacokinetics (PK) have typically used Childs-Turcotte-Pugh (CTP, CP) class or score to rate the severity of liver disease in study subjects. Using CP class, subjects are rated categorically as having mild (CP class A), moderate (CP class B), or severe (CP class C) hepatic impairment.

The Dual Cholate Clearance Test DSI is a continuous functional score that quantifies global liver function and physiology and parallels Child-Pugh Class and Score. Review of results from QLFT Ancillary Study from the HALT-C trial suggested that DSI may be a better predictor of drug PK compared to Child Pugh classification.

The present inventors analyzed the relationship of DSI to pharmacokinetic parameters of various classes of drugs. Six drugs were assessed based on PK measurement and compartment/metabolic pathway, as shown in Table 22.

TABLE 22
Types of drugs and PK assessments

| | PK | Compartment |
|---|---|---|
| Drug | Measurement | (pathway) |
| Unconjugated | Clearance | Cytosol (transport/ |
| OCA | (h*ng mL mg) | BA conjugation) |
| Galactose* | Clearance | Cytosol |
| | (mg min−1 kg−1) | (transport/ |
| | | galactokinase) |
| Antipyrine* | Clearance | Microsome |
| | (mg min−1) | (CYP 1A2, 2B6, |
| | | 2C8, 2C9, 2C18, 3A4) |
| Caffeine | Elimination | Microsome |
| | rate (h−1) | (CYP 1A1, 1A2, |
| | | 2A6, 2E1, 3A) |
| Lidocaine* | Metabolite MEGX | Microsome |
| | (ng mL−1) | (CYP 3A4, 1A2) |
| ¹³C-methionine | Metabolite ¹³C—CO₂ (score) | Mitochondria |
| | | (oxidation) |

Obeticholic acid (OCA) is a Farnesoid X Receptor (FXR) agonist, bile acid analog of chenodeoxycholic acid that is FDA approved for treatment of primary biliary cholangitis, and is being studied for use in treating other hepatic diseases and conditions. Certain results from a clinical trial for use of OCA in NASH treatment in patients with fibrosis F1 to compensated F4, primarily F2 and F3, showing DSI value v OCA PK are shown in FIGS. 34A and B. Graphs of DSI v Dose-normalized plasma unconjugated OCA AUC(0-24 h)(h*ng/mL/mg) at baseline (day 1) and end of treatment (day 85), are shown in FIGS. 34A and 34B, respectively. The circles are observed data; red lines are linear regression fit. OCA=Obeticholic acid. Obeticholic acid plasma exposure was related to DSI measurement. DSI correlates with OCA PK both at baseline (day 1) and at day 85.

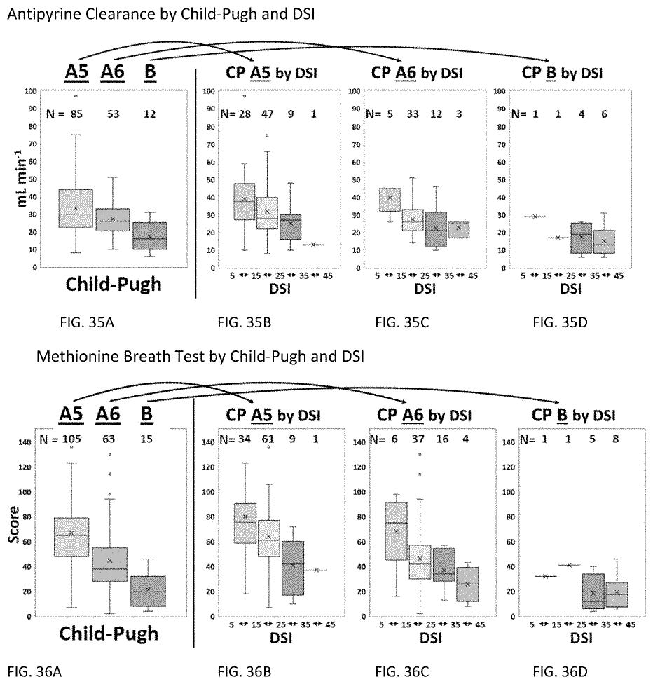

Antipyrine is an analgesic and antipyretic. Antipyrine is an exogenous/xenobiotic substrate that exhibits low first pass hepatic metabolism and may be used for measuring hepatic metabolism. Antipyrine may be used in testing the effects of other drugs or diseases on drug-metabolizing enzymes in the liver, such as various cytochrome p450 (CYP) enzymes, for example, as shown in Table 22. Antipyrine clearance by Child-Pugh and DSI are shown in FIG. 35A-D. CP A5 (n=85) subjects are shown in FIG. 35A. The CP A5 subjects in FIG. 35A were sub-divided into 4 functional groups based on DSI score, and these four groups each exhibited different average antipyrine clearance values as shown in FIG. 35B. The CP A6 (n=53) subjects shown in FIG. 35A were sub-divided into 4 functional groups based on DSI score, and these four groups each exhibited different average antipyrine clearance values as shown in FIG. 35C. The CP B class (n=12) subjects shown in FIG. 35A were sub-divided into 4 functional groups based on DSI score, and these four groups each exhibited different average antipyrine clearance values as shown in FIG. 35D. In general, patients in higher DSI score groups exhibited reduced average antipyrine clearance, regardless of CP class.

Methionine is an essential amino acid in humans, and is the substrate for other amino acids such as cysteine or taurine. Methionine also may be used to prevent liver toxicity in acetaminophen (Tylenol) poisoning, to increase acidity of urine, treat liver disorders, or improve wound healing. Methionine is an endogenous substrate that exhibits relatively low first pass hepatic extraction, and may be employed as a substrate for measuring hepatic metabolism. The methionine breath test may involve orally administered ¹³C-methionine to detect hepatic mitochondrial dysfunction by evaluating level of ¹³CO₂ exhalation. ¹³C methionine breath test may be used to monitor hepatic mitochondrial oxidation and drug-related mitochondrial toxicity in vivo, for example, to detect antiretroviral drug-related mitochondrial toxicity. Milazzo et al., 2005, J Antimicrobial Chemother, 55(1), pp. 84-89. The breath test with 13C-labelled methionine is non-invasive, non-radiolabelled technique that has been used to investigate drug-related acute liver toxicity, ethanol-induced liver oxidative stress, and impaired hepatic mitochondrial oxidation in liver steatosis and cirrhosis. Methionine breath test results for groups of patients divided by Child-Pugh scores A5, A6 and B are shown in FIG. 36A. The same three CP groups of patients were further sub-divided by DSI score groups as shown in FIG. 36B-D. Child-Pugh A5 patients (N=105) were sub-divided into 4 DSI score groups as shown in FIG. 36B. Child-Pugh A6 patients (N=63) were sub-divided into 4 DSI score groups as shown in FIG. 36C. Child-Pugh class B patients (N=15) were sub-divided into 4 DSI score groups as shown in FIG. 36D. In general, patients in higher DSI score groups exhibited reduced average ¹³CO₂ exhalation score, regardless of CP class.

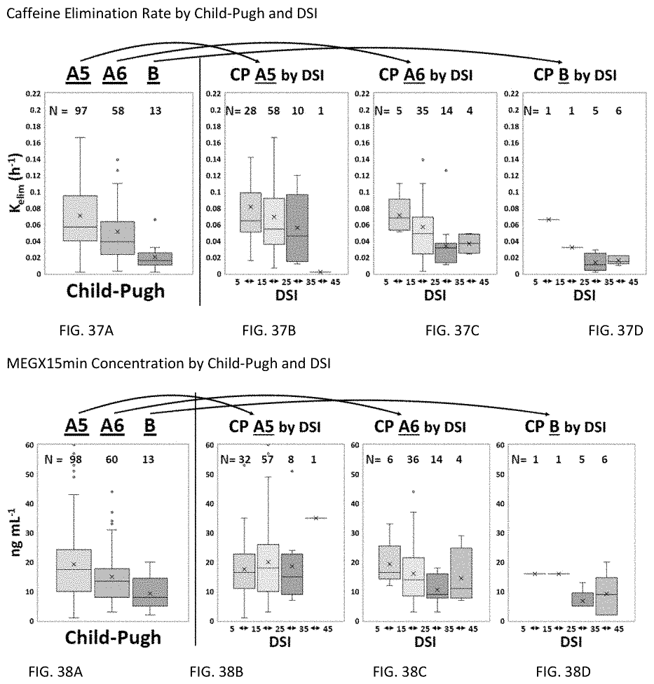

Caffeine is a natural substrate that exhibits relatively low first pass hepatic extraction and is a common substrate for measuring hepatic metabolism. Other common substrates for measuring hepatic metabolism include phenylalanine, aminopyrine, phenacetin, methacetin, anyipyrine, diazepam, erythromycin, methionine, and α-ketoisocaproic acid. Helmke et al., 2015, Curr Opin Gastroenterol 31(3):199-208. Average caffeine elimination rate for groups of patients divided by Child-Pugh scores A5, A6 and B are shown in FIG. 37A. The CP groups of patients in FIG. 37A were further sub-divided by DSI score groups as shown in FIG. 37B-D. Child-Pugh A5 patients (N=97) were sub-divided into 4 DSI score groups as shown in FIG. 37B. Child-Pugh A6 patients (N=58) were sub-divided into 4 DSI score groups as shown in FIG. 37C. Child-Pugh class B patients (N=13) were sub-divided into 4 DSI score groups as shown in FIG. 37D. In general, patients in higher DSI score groups exhibited reduced caffeine elimination rate, regardless of CP class.

Lidocaine is an exogenous/xenobiotic substrate that exhibits relatively high first pass hepatic extraction. Lidocaine elimination and MEGX (monoethylglycinexylidide) formation after oral lidocaine administration may be used as a quantitative assessment of liver function. FIG. 38A shows Child-Pugh score v MEGX 15 min concentration for three groups CP A5, CP A6, and CP B. The three CP groups of patients in FIG. 38A were further sub-divided by DSI score groups as shown in FIG. 38B-D. Child-Pugh A5 patients (N=98) were sub-divided into 4 DSI score groups as shown in FIG. 38B. Child-Pugh A6 patients (N=60) were sub-divided into 4 DSI score groups as shown in FIG. 38C. Child-Pugh class B patients (N=13) were sub-divided into 4 DSI score groups as shown in FIG. 38D. In general, patients in different DSI score groups exhibited different avg. MEGX 15 minute concentrations.

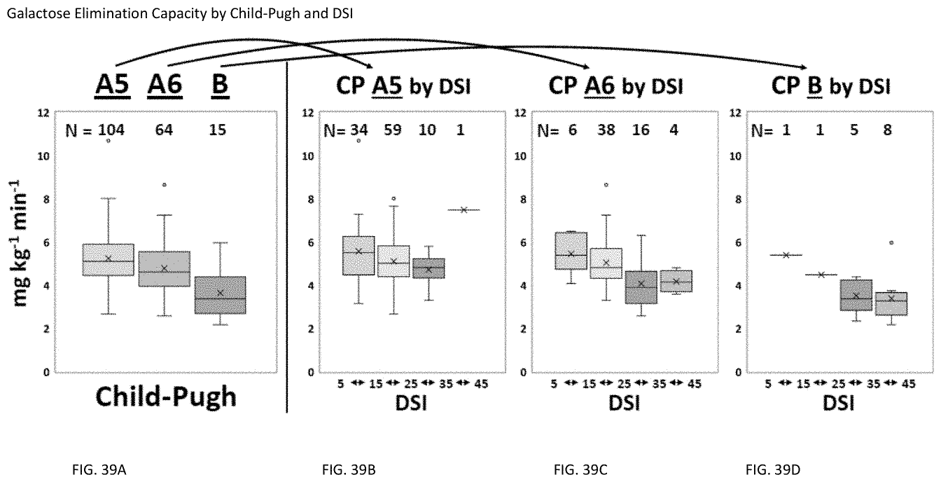

Galactose is a natural substrate that exhibits relatively high first pass hepatic extraction. FIG. 39A shows avg. galactose elimination capacity v Child-Pugh score for three groups: CP A5, CP A6, and CP B. The three CP groups of patients in FIG. 39A were further sub-divided by DSI score groups as shown in FIG. 39B-D. Child-Pugh A5 patients (N=104) were sub-divided into 4 DSI score groups as shown in FIG. 39B. Child-Pugh A6 patients (N=64) were sub-divided into 4 DSI score groups as shown in FIG. 39C. Child-Pugh class B patients (N=15) were sub-divided into 4 DSI score groups as shown in FIG. 39D. In general, patients in higher DSI score groups exhibited reduced galactose elimination capacities, regardless of CP class and score.

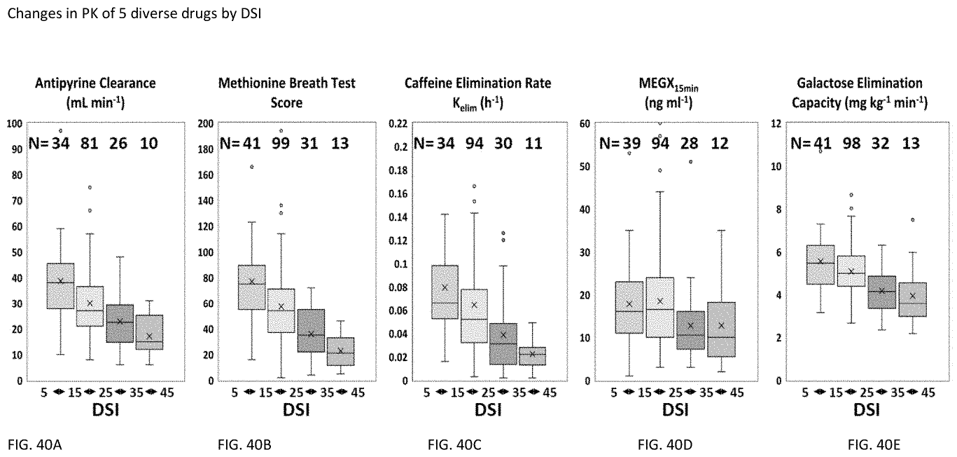

A summary of the changes in PK of 5 diverse drugs is shown in FIG. 40A-D comparing DSI score groups of DSI 5-15, DSI 15-25, DSI 25-35, DSI 35-45 in each of antipyrine clearance, methionine breath test, caffeine elimination rate, lidocaine MEGX15 min concentration, and Galactose elimination capacity. Patients exhibiting the highest DSI scores (35-45), exhibited the lowest PK average values for each of antipyrine clearance, methionine breath test, caffeine elimination, lidocaine MEGX15 min concentration, and galactose elimination capacity. The dual cholate clearance test DSI may provide an improved understanding and distinction in changes in PK for diverse classes of drugs within and across Child-Pugh class and score. As evidenced in FIG. 40A to FIG. 40 E, the DSI value in a subject may be used to independently predict drug pharmacokinetic data PK for a drug in the subject, regardless of Child-Pugh class or score, for example, wherein the PK is selected from a drug clearance, metabolite formation, or drug elimination rate.

### Example 16. Hepatic Reserve and Indexed Hepatic Reserve

Patients who undergo the Fontan operation as children for a complex heart defect may be at risk for developing progressive liver fibrosis. Fontan surgery for single ventricle congenital heart disease leads to Fontan-associated liver disease (FALD). Routine imaging, transient elastography, and serum biomarkers may be unable to accurately determine clinical severity of FALD. A pilot study determining SHUNT and DSI in Fontan patients (N=14) has been published, and most Fontan patients exhibited hepatic impairment detected by abnormal DSI, with a smaller number having elevated SHUNT values >49% suggesting intrinsic liver disease. Lemmer, Alexander et al., Congenital Heart Disease, 2019:00:1-9, DOI: 10.1111/chd.12831.

In the present study, Fontan patients (N=18) were evaluated for STAT, SHUNT, DSI, Hepatic Reserve, and indexed Hepatic Reserve. Patients received simultaneous oral (d4-CA) and intravenous (13C-CA) distinguishable cholic acids. Patient blood samples were obtained at baseline (0 minutes), 5, 20, 45, 60, and 90 minutes, and serum concentration of the distinguishable cholates was determined as described herein. Liver function values were calculated for Fontan patients including HR (algebraic), HR (indexed avg lean), STAT, portal HFR, systemic HFR, SHUNT, and DSI.

The calculation of Hepatic Reserve (HR) was simply an algebraic conversion of the DSI value: HR (algebraic)=[100−(2×DSI). HR (algebraic) is expressed as % since it is simply a conversion of DSI to a percentage scale, because given range of DSI is from 0 to 50.

Indexed HR is essentially a normalization of DSI by indexing the result in a given patient in reference to the mean of lean controls. HR (indexed avg lean) is a separately calculated index based on the change of both Systemic and Portal HFRs indexed against their respective values in lean controls. Since the scale is 0 to 100, one can consider the indexed HR in terms of % reduction in liver function from healthy lean controls.

Systemic HFR and portal HFR values for Fontan patients were indexed to lean controls by use of a generic HR equation:

Indexed HR=100−(X×SQRT((Y−LN(Syst HFR))²+(Z−LN(Port HFR))²)),

wherein X is a scaling multiplier, and Y and Z are the mean values in lean controls.

In the present example, mean values for lean controls were determined and systemic HFR and portal HFR were indexed by 6.52 and 29.1 mL min-1 kg-1—the averages for the lean controls. The resultant equation for indexed HFR is shown below:

Indexed HR=100−(25.92638×SQRT((LN 6.52−LN(Systemic HFR))²+(LN 29.1−LN(Portal HFR))²)).

Table 23A and Table 23 B show liver function values for Fontan patients including HR (algebraic), HR (indexed avg lean), STAT, portal HFR, systemic HFR, SHUNT, and DSI.

TABLE 23A
Liver Function Values and Indexed Hepatic Reserve in Fontan Patients

| | | Patient ID | | | | | | | | |
|---|---|---|---|---|---|---|---|---|---|---|
| | unit | 6 | 10 | 12 | 13 | 16 | 17 | 14 | 15 | 9 |
| Fasting | μM | BLOQ | BLOQ | BLOQ | BLOQ | BLOQ | BLOQ | BLOQ | 0.171 | 0.472 |
| Cholate | | | | | | | | | | |
| STAT | μM | 0.59 | 0.88 | 0.79 | 0.88 | 0.14 | 1.94 | 0.54 | 0.64 | 0.56 |
| Portal | mL/min/kg | 11.61 | 12.14 | 12.82 | 12.63 | 43.85 | 5.20 | 11.09 | 16.23 | 10.92 |
| HFR | | | | | | | | | | |
| Systemic | mL/min/kg | 4.28 | 2.43 | 2.70 | 3.25 | 7.18 | 2.78 | 3.25 | 3.87 | 3.55 |
| HFR | | | | | | | | | | |
| SHUNT | (%) | 36.9% | 20.0% | 21.1% | 25.7% | 16.4% | 53.5% | 29.3% | 23.8% | 32.5% |
| DSI | (0 to 50) | 19.04 | 22.53 | 21.30 | 20.06 | 4.70 | 28.93 | 21.15 | 16.75 | 20.72 |
| HR | (%) | 61.9% | 54.9% | 57.4% | 59.9% | 90.6% | 42.1% | 57.7% | 66.5% | 58.6% |
| (algebraic) | | | | | | | | | | |
| HR | (0 to 100) | 73.8 | 65.8 | 68.8 | 71.8 | 100.0 | 50.2 | 69.2 | 79.7 | 70.1 |
| (indexed | | | | | | | | | | |
| avg lean) | | | | | | | | | | |

TABLE 23B
Liver Function Values and Indexed Hepatic Reserve in Fontan Patients

| | | Patient ID | | | | | | | | |
|---|---|---|---|---|---|---|---|---|---|---|
| | unit | 18 | 20 | 19 | 24 | 25 | 27 | 28 | 31 | 22 |
| Fasting | μM | 1.113 | BLOQ | 0.153 | BLOQ | 0.116 | 0.257 | 1.473 | BLOQ | BLOQ |
| Cholate | | | | | | | | | | |
| STAT | μM | 0.69 | 1.95 | 1.07 | 0.50 | 1.09 | 0.41 | 1.81 | 0.39 | 0.37 |
| Portal | mL/min/kg | 12.34 | 5.42 | 15.90 | 25.50 | 17.83 | 32.53 | 6.69 | 26.17 | 13.64 |
| HFR | | | | | | | | | | |
| Systemic | mL/min/kg | 3.90 | 2.84 | 3.81 | 2.07 | 4.56 | 5.05 | 5.08 | 4.18 | 4.01 |
| HFR | | | | | | | | | | |
| SHUNT | (%) | 31.6% | 52.4% | 24.0% | 8.1% | 25.6% | 15.5% | 75.9% | 16.0% | 29.4% |
| DSI | (0 to 50) | 19.04 | 28.43 | 17.03 | 19.44 | 14.82 | 9.60 | 23.64 | 12.62 | 17.98 |
| HR | (%) | 61.9% | 43.1% | 65.9% | 61.1% | 70.4% | 80.8% | 52.7% | 74.8% | 64.0% |
| (algebraic) | | | | | | | | | | |
| HR | (0 to 100) | 74.1 | 51.4 | 79.0 | 70.1 | 84.3 | 93.4 | 61.3 | 88.2 | 76.7 |
| (indexed | | | | | | | | | | |
| avg lean) | | | | | | | | | | |

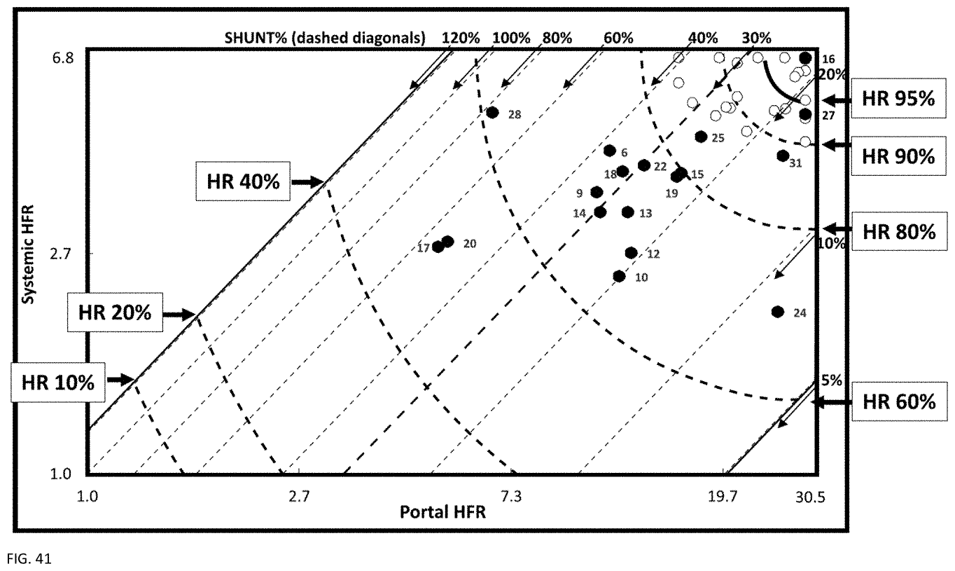

Indexed hepatic reserve (HR) may be displayed in a function map. An indexed HR function map is similar to a DSI function map. The main difference is the reference point. For DSI calculation and function maps, the upper limit of normal for Systemic and Portal HFRs were the means of all controls (N=50)+3 SDs. For Indexed HR calculation and function maps, the upper limit of normal for Systemic and Portal HFRs are the means of lean controls (N=30). A function map of Indexed Hepatic Reserve in Fontan patients versus lean controls is shown in FIG. 41. Solid circles indicate HR values in individual Fontan patients (n=18), open circles show values in individual lean controls. Numbers near data points indicate Fontan patient ID, as shown in Tables 23A and 23B. Two Fontan patients 17 and 20 exhibited indexed HR of about 50% and 51% respectively, compared to lean healthy controls. The same two patients exhibited a SHUNT value >50%, indicative of advanced liver disease severity. In contrast, Fontan patient 16 exhibits an indexed HR of about 100% compared to lean controls.

### REFERENCES CITED

Everson G T, Shiffman M L, Hoefs J C, Morgan T R, Sterling R K, Wagner D A, Desanto J L, Curto T_{M}, Wright E C; HALT-C Trial Group. Quantitative tests of liver function measure hepatic improvement after sustained virological response: results from the HALT-C trial. Aliment Pharmacol Ther. 2009; 29: 589-601.
Everson G T, Shiffman M L, Hoefs J C, Morgan T R, Sterling R K, Wagner D A, Lauriski S, Curto T_{M}, Stoddard A, Wright E C; HALT-C Trial Group. Quantitative liver function tests improve the prediction of clinical outcomes in chronic hepatitis C: results from the Hepatitis C Antiviral Long-term Treatment Against Cirrhosis Trial. Hepatology 2012; 55: 1019-29.
Everson G T, Martucci M A, Shiffman M L, Sterling R K, Morgan T R, Hoefs J C; HALT-C trial group. Portal-systemic shunting in patients with fibrosis or cirrhosis due to chronic hepatitis C: the minimal model for measuring cholate clearances and shunt. Aliment Pharmacol Ther. 2007; 26: 401-10.
Everson G T, Shiffman M L, Morgan T R, Hoefs J C, Sterling R K, Wagner D A, Kugelmas M, Curto T, Wright E C, and the HALT-C Trial Group (2008). The Spectrum of Hepatic Functional Impairment in Patients with Fibrosis and Compensated Cirrhosis Due to Chronic Hepatitis C: Results from the HALT-C Trial. Aliment Pharmacol Ther. 27(9):798-809.
Matuszewski B K, Constanzer M L, Chavez-Eng C M. Strategies for the assessment of matrix effect in quantitative bioanalytical methods based on HPLC-MS/MS. Anal Chem. 2003; 75: 3019-30.
Müller C, Schaefer P, Stortzel M, et al. Ion suppression effects in liquid chromatography-electrospray-ionisation transport-region collision induced dissociation mass spectrometry with different serum extraction methods for systematic toxicological analysis with mass spectra libraries. J Chromatogr B Analyt Technol Biomed Life Sci. 2002; 773: 47-52.
U.S. Department of Health and Human Services, Food and Drug Administration, Center for Drug Evaluation and Research and Center for Veterinary Medicine. (2001) Guidance for the Industry. Bioanalytical Method Validation. Version May 2001. http://www.fda.gov/downloads/Drugs/GuidanceComplianceRegulatoryInformation/Guidances/UCM070107.pdf
U.S. Department of Health and Human Services, Food and Drug Administration. Bioanalytical Method Validation. Draft Guidance, 2013.http://www.fda.gov/downloads/drugsiguidancecomplianceregulatoryinformation/guidances/ucm368107.pdf
Lan K, Su M, Xie G, et al. Key Role for the 12-Hydroxy Group in the Negative Ion Fragmentation of Unconjugated C24 Bile Acids. Anal Chem. 2016; 88(14):7041-7048. doi:10.1021/acs.analchem.6b00573
Lee W M, et al, Evolution of the HALT-C Trial: pegylated interferon as maintenance therapy for chronic hepatitis C in previous interferon nonresponders. Controlled Clinical Trials 2004; 25: 472-492.
Singal A G, et al. Detection of Hepatocellular Carcinomaat Advanced Stages Among Patients in the HALT-C Trial: Where did Surveillance Fail? Am J Gastroenterol 2013; 108:425-432.
Dameron, 2018, Microsampling takes blood collection out of the lab, Dark Daily Clinical Laboratory and Pathology.
Lemmer, Alexander et al., Assessing hepatic impairment in Fontan-associated liver disease using the HepQuant SHUNT test. Congenital Heart Disease, 2019:00:1-9, DOI: 10.1111/chd.12831.
Gregory T. Everson, John C. Hoefs, Claus U. Niemann, Kim M. Olthoff, Robert Dupuis, Shannon Lauriski, Andrea Herman, Norah Milne, Brenda W. Gillespie, Nathan P.
Goodrich, and James E. Everhart. Functional Elements Associated With Hepatic Regeneration in Living Donors After Right Hepatic Lobectomy. Liver Transpl 2013; 19:292-304.
Helmke S, Colmenero J, Everson G T. Noninvasive assessment of liver function. Curr Opin Gastroenterol. 2015 May; 31(3):199-208.
Maarouf A. Hoteit, Andrezj Wojcieszynski, Brian Currie, Matthew H. Levine, Kimberly A. Forde, Kim A. Reiss, Greg Nadolski, Michael C. Soulen3, Steve Helmke, Gregory T. Everson, Edgar Ben-Josef. Deterioration in liver function after liver-directed therapy for hepatocellular carcinoma measured by cholate clearance. GastroHep. 2020; 00:1-8. https://doi.org/10.1002/ygh2.421.
Burton, James R. et al. The within-individual reproducibility of the disease severity index from the HepQuant SHUNT test of liver function and physiology. Translational Research, 2021 Jan. 2:S1931-5244(20)30321-2. doi: 10.1016/j.trs1.2020.12.010. Online ahead of print. PMID: 33400995.
Fallahzadeh M A, Hansen D J, Trotter J F, Everson G T, Saracino G, Rahimi R S, Helmke S, Boutte J, Asrani S K. Predicting clinical decompensation in patients with cirrhosis using the HepQuant SHUNT Test. Alimentary Pharmacology and Therapeutics. 2021. Doi: 10.1111/apt.16252
Maan R, Sonneveld M J Editorial: stratifying risk of adverse outcomes in cirrhosis: the Hepquant SHUNT test. Alimentary Pharmacology and Therapeutics. 2021. In press
Asrani S, Everson G T. Authors' Response to “Editorial: stratifying risk of adverse outcomes in cirrhosis: the Hepquant SHUNT test”. Alimentary Pharmacology and Therapeutics. 2021. In press
Wieland A, Etzion O, Ali R O, Levy E, Kleiner D, Helmke S M, Heller T, Everson G T. HepQuant SHUNT Detects Portal Hypertension in Early Stages of Clinically Compensated Chronic Liver Disease. Submitted to Clinical Gastroenterology and Hepatology. 2021.

1. A method for quantifying one or more distinguishable compounds in a blood or serum sample from a subject, the method comprising:
receiving a blood or serum sample obtained from a subject having or suspected of having or developing a chronic liver disease or hepatic disorder, wherein the sample was collected from the subject less than 3 hours after oral and/or intravenous administration of the one or more distinguishable compounds to the subject;
processing the blood or serum sample to form a processed sample;
injecting the processed sample onto a mass detection system;
measuring the concentration of the one or more distinguishable compounds in the processed sample comprising mass detection; and
quantifying the concentration of the one or more distinguishable compounds in the blood or serum sample.

2. The method of claim 1, wherein the processed sample is a supernatant or an eluate.

3. The method of claim 2, wherein the processing of the blood or serum sample comprises forming a supernatant.

4. The method of claim 3, further comprising injecting the supernatant onto a separation system comprising a preparative component, and/or an analytical component to form an eluate.

5. The method of claim 4, wherein the separation system comprises a chromatography system.

6. The method of claim 5, wherein the chromatography system includes a liquid chromatography (LC) system, optionally wherein the LC system is selected from the group consisting of an HPLC and a UPLC system.

7. The method of claim 1, wherein the mass detection system comprises a mass spectrometer.

8. The method of claim 7, wherein the mass spectrometer comprises an ion source system and a mass resolution/detection system.

9. The method of claim 8, wherein the ion source system is selected from the group consisting of electrospray ionization (ES), matrix-assisted laser desorption/ionization (MALDI), fast atom bombardment (FAB), chemical ionization (CI), atmospheric pressure chemical ionization (APCI), liquid secondary ionization (LSI), laser diode thermal desorption (LDTD), and surface-enhanced laser desorption/ionization (SELDI).

10. The method of claim 9, wherein the mass resolution/detection system is selected from the group consisting of triple quadrupole mass spectrometer (MS/MS); single quadrupole mass spectrometer (MS); Fourier-transform mass spectrometer (FT-MS); and time-of-flight mass spectrometer (TOF-MS).

11. The method of claim 10, wherein the triple quadrupole mass spectrometer (MS/MS) is run in a multiple reaction mode (MRM).

12. The method of claim 4, wherein the processing comprises:
injecting the supernatant to the preparative component; and
eluting the preparative component onto the analytical component to form the eluate.

13. The method of claim 12, wherein the preparative component comprises a solid phase resin and the analytical component each comprise a solid phase resin.

14. The method of claim 13, wherein the solid phase resin of the preparative and analytical components are each independently selected from the group consisting of a normal phase resin, reverse phase resin, hydrophobic interaction solid phase resin, hydrophilic interaction solid phase resin, ion-exchange solid phase resin, size-exclusion solid phase resin, and affinity-based solid phase resin.

15. The method of claim 1, wherein the subject had received the oral and intravenous one or more distinguishable compounds less than 3 hours prior to collecting the sample from the subject.

16. The method of claim 3, wherein the processing comprises
adding a protein precipitation solution to the sample to form a protein precipitate and the supernatant.

17. The method of claim 16, wherein the protein precipitation solution comprises a water miscible organic solvent.

18. The method of claim 17, wherein the water miscible organic solvent is selected from the group consisting of methanol, ethanol, isopropanol, acetonitrile, and acetone.

19. The method of claim 1, further comprising adding an internal standard distinguishable compound to the blood or serum sample.

20. The method of claim 1, wherein the volume of the blood or serum sample is 10 μL or more, 20 μL or more, 30 μL or more, 40 μL or more, 50 μL or more, no more than 500 μL, no more than 400 μL, no more than 300 μL, no more than 200 μL, no more than 100 μL, or from 10-500 μL, from 20-400 μL, from 30-300 μL, from 30-200 μL, or from 40-100 μL.

21. The method of claim 20, wherein the blood or serum sample is obtained in the form of a dried blood spot sample, capillary blood sample, or a dried volumetric absorptive microsampling device sample.

22. The method of claim 21, wherein the processing comprises exposing the sample to an extraction solution to form the supernatant, and optionally diluting the supernatant prior to the injecting.

23. The method of claim 1, wherein the at least one distinguishable compound(s) is capable of exhibiting high hepatic extraction of at least 50%, 60%, or 70% in first pass through the liver of a healthy subject following oral administration.

24. The method of claim 1, wherein the at least one distinguishable compound(s) is a distinguishable bile acid, bile acid conjugate, or bile acid analog.

25. The method of claim 24, wherein the distinguishable bile acid, bile acid conjugate, or bile acid analog is selected from the group consisting of a distinguishable cholic acid (CA), dehydrolithocholic acid (dehydroLCA), lithocholic acid (LCA), isodeoxycholic acid (isoDCA), isolithocholic acid (isoLCA), allolithocholic acid (alloLCA), glycolithocholic acid (GLCA), deoxycholic acid (DCA), chenodeoxycholic acid (CDCA), taurolithocholic acid (TLCA), apocholic acid (apoCA), 23-nordeoxycholic acid (nor-DCA), 12-ketolithocholic acid (12-ketoLCA), 7-ketolithocholic acid (7-ketoLCA), 6,7-diketolithocholic acid (6,7-diketoLCA), glycodeoxycholic acid (GDCA), 6-keto-lithocholic acid (6-ketoLCA), glycochenodeoxycholic acid (GCDCA), hyodeoxycholic acid (HDCA), ursodeoxycholic acid (UDCA), cholic acid (CA), taurodeoxycholic acid (TDCA), allocholic acid (ACA), beta-hyodeoxycholic acid (beta-HDCA), murocholic acid (muroCA), hyocholic acid (HCA), 12-dehydrocholic acid (12-DHCA), beta-muricholic acid (beta-MCA), norcholic acid (norCA), 7-ketodeoxycholic acid (7-ketoDCA), glycocholic acid (GCA), alpha-muricholic acid (alpha-MCA), glycohyodeoxycholic acid (GHDCA), 3beta-cholic acid (betaCA), glycoursodeoxycholic acid (GHCA), omega-muricholic acid (omegaMCA), taurocholic acid (TCA), glycohyocholic acid (GHCA), taurohyodeoxycholic acid (THDCA), 7,12-diketolithocholic acid (7,12-diketoLCA), dehydrocholic acid (DHCA), ursocholic acid (UCA), taurohyocholic acid (THCA), tauro beta-muricholic acid (TbetaMCA), tauro alpha-muricholic acid (TalphaMCA), glycodehydrocholic acid (GDHCA), tauro omega-murichlic acid (TomegaMCA), taurohydrocholic acid (TDHCA), ursodeoxycholic acid (UDCA), hyodeoxycholic acid (HDCA), (3α,6α-dihydroxy-5β-cholan-24-oic acid), deoxycholic acid, all beta cholic acid, lithocholic acid 3-hemisuccinate, epideoxycholic acid, ursodeoxycholic acid methyl ester, ursocholanic acid, obeticholic acid (2alpha-ethyl-chenodeoxycholic acid), cholic acid methyl ester, cholic alcohol, epilithocholic acid, or an isotopically labeled derivative, or analog or epimer thereof.

26. The method of claim 24, wherein the distinguishable bile acid, bile acid conjugate or cholic acid analog is an isotopically labeled bile acid, bile acid conjugate, or bile acid analog.

27. The method of claim 26, wherein the isotopically labeled bile acid, bile acid conjugate, or bile acid analog is a stable isotope labeled bile acid, bile acid conjugate, or bile acid analog.

28. The method of claim 27, wherein the stable isotope labeled bile acid, bile acid conjugate, or bile acid analog, is selected from the group consisting of 2,2,4,4-d4-cholic acid (D4-CA; CA-D4), 24-¹³C-cholic acid (¹³C-CA), 2,2,3,4,4-d₅ cholic acid (D₅-CA), 3,6,6,7,8,11,11,12-d8 cholic acid (D8-CA), lithocholic acid-2,2,4,4-D4 (LCA-D4), ursodeoxycholic acid-2,2,4,4-D4 (UDCA-D4), ursodeoxycholic acid (24-13C-UDCA), deoxycholic acid-2,2,4,4-D4 (DCA-D4), glycochenodeoxycholic acid-2,2,4,4-D4 (GCDCA-D4), glycochenodeoxycholic acid (glycine-2,2,3,4,4,6,6,7,8-D9-CDCA), glycodeoxycholic acid-2,2,4,4-D4 (GDCA-D4), glycocholic acid-2,2,4,4-D4 (GCA-D4), glycocholic acid (glycine-1-13C-CA), deoxycholic acid-24-13C (DCA-24-13C), deoxycholic acid (2,2,4,4,11,11-D6-DCA), alpha-muricholic acid (2,2,3,4,4-D5-αMCA), beta-muricholic acid (2,2,3,4,4-D5-βMCA), chenodeoxycholic acid (2,2,3,4,4,6,6,7,8-D9-CDCA), chenodeoxycholic acid (2,2,3,4,4-D5-CDCA), chenodeoxycholic acid (2,2,4,4-D4-CDCA), chenodeoxycholic acid (24-13C-CDCA), gamma-muricholic acid (2,2,3,4,4-D5-γMCA), omega-muricholic acid (2,2,3,4,4-D5-ωMCA), taurochenodeoxycholic acid, sodium salt (taurine-2,2,3,4,4,6,6,7,8-D9-CDCA); taurochenodeoxycholic acid, sodium salt (taurine-2,2,4,4-D4-CDCA); taurocholic acid, sodium salt (taurine-13C2-CA); taurocholic acid, sodium salt (taurine-2,2,4,4-D4-CA); taurodeoxycholic acid, sodium salt (taurine-2,2,4,4,11,11-D6-DCA); taurodeoxycholic acid, sodium salt (taurine-2,2,4,4-D4-DCA); tauroursodeoxycholic acid, sodium salt (taurine-2,2,4,4-D4-UDCA); tauroursodeoxycholic acid, sodium salt (taurine-13C2-UDCA), glycolithocholic acid (glycine-2,2,4,4-D4-LCA), 11,12-dideuterated chenodeoxycholic acid (D2-chenodeoxycholic acid, D2-CA), glycoursodeoxycholic acid (glycine-2,2,4,4-D4-UDCA), and glycoursodeoxycholic acid (glycine-13C2-UDCA).

29. The method of claim 22, wherein the processing extraction recovery of the distinguishable compound from the blood or serum sample is >80%, >90%, or >95%.

30. A method for screening for or monitoring of liver function, liver disease, or a hepatic disorder in a subject comprising:
obtaining a blood or serum sample from a subject having or suspected of having or at risk of a chronic liver disease, following oral administration of a composition comprising a distinguishable compound to the subject, wherein the blood or serum sample was collected from the subject less than 3 hours after oral administration of the distinguishable compound to the subject, optionally wherein the blood or serum sample consists of a single blood or serum sample;
measuring the concentration of the orally administered distinguishable compound in the blood or serum sample from the subject, comprising quantifying the concentration of the distinguishable compound in the sample according to claim 1 to obtain a STAT value; and
optionally comparing the concentration of distinguishable compound in the blood or serum sample (STAT value) to (i) a distinguishable compound concentration cutoff value or cutoffs of values established from a known patient population, and/or to (ii) the concentration of the distinguishable compound in one or more earlier samples from the same subject over time.

31. The method of claim 30, wherein the comparing of the concentration of the orally administered distinguishable compound in the single sample from the patient over time is used to monitor the effectiveness of a treatment of chronic liver disease in the patient, wherein a decrease in concentration of the distinguishable compound over time is indicative of treatment effectiveness.

32.-34. (canceled)

35. The method of claim 30, wherein the blood or serum samples were collected at one or more time points after the oral administration of the distinguishable compound, selected from the group consisting of baseline, 5, 10, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, and 180 minutes, or any time point in between; optionally wherein the blood or serum sample is a single blood or serum sample collected at a single specific time point.

36. The method of claim 35, wherein the single blood or serum sample was collected at one time point selected from about 45, about 60 or about 90 minutes after oral administration of the distinguishable compound.

37. The method of claim 35, wherein the concentration of distinguishable compound in the single blood or serum sample (STAT value) is used in a method to estimate portal hepatic filtration rate (portal HFR) (FLOW) in the subject.

38. The method of claim 37, wherein the method for estimation of portal HFR (FLOW) in the subject further comprises:
converting the concentration of the distinguishable compound in the single blood or serum sample (STAT value) by using an equation into an estimated portal HFR (FLOW) (mL/min/kg) in the subject; and
comparing the estimated portal HFR in the subject to a portal HFR (FLOW) cutoff value or cutoffs of values established from a known patient population or within the subject over time.

39. The method of claim 38, wherein the comparing of the estimated portal HFR (FLOW) (mL/min/kg) values in the subject over time is used to monitor the effectiveness of a treatment of chronic liver disease in the subject, wherein an increase in estimated portal HFR (FLOW) (mL/min/kg) value over time is indicative of treatment effectiveness.

40. The method of claim 38, wherein the equation for converting the STAT value into an estimated portal HFR (FLOW)(mL/min/kg) value in the subject is:
y=A(x)+C, wherein
x=LOG estimated portal HFR (FLOW) value (mL/min/kg) in the subject;
y=LOG STAT value (μM adjusted to 75 kg bodyweight) in the subject;
A=slope coefficient from 0.9 to 1.1; and
C=a constant from −0.05 to 0.05.

41. The method of claim 39, wherein the equation for converting the STAT value into an estimated portal HFR (FLOW)(mL/min/kg) value in the subject is
y=0.9702x+0.0206.

42. The method of claim 38, wherein the equation for converting the STAT value into an estimated portal HFR (FLOW)(mL/min/kg) value in the subject is:
Ln(x)=1.031×Ln(y)−0.0212, wherein;
x=estimated portal HFR value (mL/min/kg) in the subject; and
y=STAT value (μM adjusted to 75 kg bodyweight) in the subject.

43. A method for estimating a DSI value in a subject comprising obtaining a concentration of a distinguishable compound in a single blood or serum sample (STAT value) according to the method of claim 30.

44. The method of claim 43, wherein the method for estimation of a DSI value in the subject further comprises:
converting the concentration of the distinguishable compound in the sample by using an equation into a DSI value in the subject; and
comparing the estimated DSI value in the subject to a DSI value cutoff value or cutoffs of values established from a known patient population or within the subject over time.

45. The method of claim 44, wherein the comparing of the estimated DSI values in the subject over time is used to monitor the effectiveness of a treatment of chronic liver disease in the subject, wherein a decrease in estimated DSI value over time is indicative of treatment effectiveness, optionally wherein the decrease in estimated DSI value over time is at least about −1.5 points, at least about −2 points, or at least about −3 points.

46. The method of claim 44, wherein the equation for converting the concentration of the distinguishable compound in the single specific sample (STAT value) into an estimated DSI value in the subject is:
y=A ln(x)+C, wherein;
A=slope value from 8.5 to 10.5;
C=constant from 18 to 22;
x=STAT value (in μM adjusted to 75 kg bodyweight); and
y=estimated DSI value in the subject.

47. The method of claim 46, wherein the equation for converting the concentration of the distinguishable compound in the single specific sample into an estimated DSI value in the subject is:
y=9.4514 ln(x)+21.12.

48. The method of claim 44, wherein the equation for converting the concentration of the distinguishable compound in the single specific sample (STAT value) into an estimated DSI value in the subject is:
y=A(Ln x)²+B(Ln x)+C, wherein;
y=estimated DSI value in the subject;
x=STAT value in the subject;
A=coefficient from 1 to 1.5;
B=coefficient from 9 to 10; and
C=constant from 19.5 to 22.

49. The method of claim 48, wherein the equation for converting the concentration of the distinguishable compound in the single specific sample (STAT value) into an estimated DSI value in the subject is:
y=1.3816(Ln x)²+9.2339(Ln x)+20.196, wherein;
y=estimated DSI value, and
x=STAT value in the subject.

50. The method of claim 30, wherein the hepatic disorder or liver disease in the subject is selected from the group consisting of chronic hepatitis C, chronic hepatitis B, cytomegalovirus, Epstein Barr virus, alcoholic liver disease, drug-induced liver disease, portal hypertension, cryptogenic cirrhosis, alpha 1-antitrypsin disease, hemochromatosis, nodular regenerative hyperplasia, idiopathic liver disease, congenital liver diseases, non-alcoholic steatohepatitis (NASH), non-alcoholic fatty liver disease (NAFLD), haemochromatosis, Wilson's disease, autoimmune chronic hepatitis, primary biliary cirrhosis, primary sclerosing cholangitis (PSC), and hepatocellular carcinoma (HCC).

51. The method of claim 50, wherein the estimated portal HFR value or estimated DSI value in the subject is used to screen patients for liver function or liver disease; monitor liver disease patients undergoing antiviral therapy; monitor disease progression in patients with chronic liver disease; determine stage of liver disease in a patient; prioritize liver disease patients for liver transplant; determine selection of patients with chronic hepatitis B who should receive antiviral therapy; assessing the risk of hepatic decompensation in patients with hepatocellular carcinoma (HCC) being evaluated for hepatic resection; identifying a subgroup of patients on waiting list with low MELD (Model for End-stage Liver Disease score) who are at-risk for dying while waiting for an organ donor; as an endpoint in a clinical trial; replacing liver biopsy in pediatric populations; tracking of allograft function; measuring return of liver function in living donors; measuring functional impairment in cholestatic liver disease in a subject; for instituting a treatment or intervention in a patient; distinguish fibrosis stages; or, identify early stage F0-F2 HCV patients.

52. A method for assessment of hepatic shunt/or relative hepatic function in a subject having or suspected of having or at risk of a hepatic disorder or chronic liver disease, comprising the steps of:
(a) obtaining a multiplicity of blood or serum samples collected from a subject over intervals for a period of less than 3 hours after the subject had been orally administered a first distinguishable compound and simultaneously intravenously administered a second distinguishable compound;
(b) quantifying the first and the second distinguishable compounds in the samples comprising the method according to claim 1;
(c) calculating the hepatic shunt in the subject using the formula:
AUCoral/AUCiv×Doseiv/Doseoral×100%;
wherein AUCoral is the area under the curve of the serum concentrations of the first distinguishable compound and AUCiv is the area under the curve of the second distinguishable compound; and
(d) comparing the hepatic shunt in the subject to a shunt cutoff value or cutoffs of values established from a known patient population wherein the hepatic shunt in the subject compared to shunt cutoff value or cutoffs of values is an indicator of relative hepatic function of the subject.

53. The method of claim 52, wherein the samples comprise blood or serum samples collected from the subject at 2 or more, 3 or more, 3 or more, 4 or more, 5 or more, 6 or more, 7 or more, 8 or more, 9 or more, 10 or more, 11 or more, 12 or more, 13 or more or 15 or more time points, preferably collected over intervals spanning a period of time of about 90 minutes or less after administration, preferably collected at about 5, 20, 45, 60 and 90 minutes after the administration of the distinguishable compounds.

54. A method for determining a portal hepatic filtration rate (portal HFR) value in a patient having or suspected of having or at risk of a chronic liver disease, comprising:
(i) receiving a plurality of blood or serum samples collected from a patient having or at risk of a chronic liver disease, following oral administration of a dose of a distinguishable compound (doseₒᵣₐₗ) to the patient, wherein the samples have been collected from the patient over intervals spanning a period of time of less than 3 hours after administration;
(ii) measuring concentration of the distinguishable compound in each sample comprising the method according to claim 1;
(iii) generating an individualized oral clearance curve from the concentration of the distinguishable compound in each sample comprising using a computer algorithm curve fitting to a model distinguishable compound clearance curve;
(iv) computing the area under the individualized oral clearance curve (AUC) (mg/mL/min) and dividing the dose (in mg) by AUC of the orally administered distinguishable compound to obtain the oral distinguishable compound clearance in the patient; and
(v) dividing the oral distinguishable compound clearance by the weight of the patient in kg to obtain the portal HFR value in the patient (mL/min/kg).

55. A method for determining a systemic hepatic filtration rate (systemic HFR) value in a patient having or suspected of having or at risk of a chronic liver disease, comprising:
(i) receiving a plurality of blood or serum samples collected from a patient having or at risk of a chronic liver disease, following intravenous administration of a dose of a distinguishable compound (doseᵢᵥ) to the patient, wherein the samples have been collected from the patient over intervals spanning a period of time of less than 3 hours after administration;
(ii) measuring concentration of the distinguishable compound in each sample comprising the method according to claim 1;
(iii) generating an individualized intravenous clearance curve from the concentration of the distinguishable compound in each sample comprising using a computer algorithm curve fitting to a model distinguishable compound clearance curve;
(iv) computing the area under the individualized intravenous clearance curve (AUC) (mg/mL/min) and dividing the dose (in mg) by AUC of the intravenously administered distinguishable compound to obtain the intravenous distinguishable compound clearance in the patient; and
(v) dividing the intravenous distinguishable compound clearance by the weight of the patient in kg to obtain the systemic HFR value in the patient (mL/min/kg).

56. A method for determining a disease severity index (DSI) value in a patient, the method comprising:
(a) obtaining one or more liver function test values in a patient having or at risk of a chronic liver disease, wherein the one or more liver function test values are obtained from one or more liver function tests selected from the group consisting of SHUNT, portal hepatic filtration rate (portal HFR), and systemic hepatic filtration rate (systemic HFR), wherein the liver function tests comprise measuring a distinguishable compound in a blood or serum sample comprising the method according to claim 1; and
(b) employing a disease severity index equation (DSI equation) to obtain a DSI value in the patient, wherein the DSI equation comprises one or more terms and a constant to obtain the DSI value, wherein
at least one term of the DSI equation independently represents a liver function test value in the patient from step (a) or a mathematically transformed liver function test value in the patient from step (a); and
the at least one term of the DSI equation is multiplied by a coefficient specific to the liver function test.

57. The method of claim 56, wherein the DSI equation is selected from the group consisting of:
(I) DSI=f(Shunt, Portal HFR, Systemic HFR), wherein Shunt is a shunt value in the subject, portal HFR is a portal HFR value in the subject, and systemic HFR is an systemic HFR value in the subject;
(II) DSI=A (Shunt)+B (Log Portal HFR)+C (log Systemic HFR)+D, wherein A=a number from 5 to 6; B=a number from 6 to 8; C=a number from 8 to 10; D=a number from 40 to 60;
$${D S I = A \sqrt{( B - Log} H Γ R )}^{2} + {( C - Log H Γ R )}^{2} ,$$
wherein A=a number from 8 to 12; B=a number from 3 to 5; C=a number from 1.5 to 3.5;
(IV)
DSI=A{square root over ((B−(ln HFRₚ))²+(C−(ln HFRₛ)²)};
wherein A=a scaling multiplier from 8 to 12 (optionally 10.86) to yield a range from 0 (no disease) to 50 (end-stage disease), B is the natural logarithm of the maximum value for Portal HFR, b, and C is the natural logarithm of the maximum value for Systemic HFR, c; and
$$D S J = A ↦ {( l n ( \frac{b}{H F {R}_{P}} ) )}^{2} + {( l n ( \frac{c}{H F {R}_{s}} ) )}^{2} ,$$
wherein A=a scaling multiplier from 8 to 12 (optionally 10.86) to yield a range from 0 (no disease) to 50 (end-stage disease), b is the maximum value for Portal HFR in a range from 25-75, and c is the maximum value for Systemic HFR in a range from 5-15.

58. The method of claim 56, further comprising:
(a) comparing the DSI value in the patient to one or more DSI cut-off values, one or more normal healthy controls, or one or more DSI values within the patient over time.

59. The method of claim 58, wherein the comparing the DSI value in the patient to one or more DSI cut-off values is indicative of at least one clinical outcome.

60. The method of claim 59, wherein the clinical outcome is selected from the group consisting of Child-Turcotte-Pugh (CTP) increase, varices, encephalopathy, ascites, and liver related death.

61. The method of claim 58, wherein the comparing the DSI value within the patient over time is used to monitor the effectiveness of a treatment of chronic liver disease in the patient, wherein a decrease in the DSI value within the patient over time is indicative of treatment effectiveness, optionally wherein the decrease in DSI value within the patient over time is at least about −1.5 points, at least about −2 points, or at least about −3 points.

62. The method of claim 58, wherein the comparing the DSI value in the patient over time is used to monitor the need for treatment of chronic liver disease in the patient, wherein an increase in the DSI value within the patient over time is indicative of a need for treatment in the patient.

63.-66. (canceled)

67. A method for estimating a clinical event rate for a patient having a chronic liver disease, the method comprising:
obtaining a baseline DSI value (dsi0) for the patient according to the method of claim 56;
optionally, obtaining a repeat DSI value (dsi_{T}) for the patient, wherein T=months between collection of baseline and repeat DSI samples; and
calculating estimated events per person-year of observation as a function of baseline DSI value, and optionally the repeat DSI value.

68. The method of claim 67, wherein the calculating comprises a Poisson regression model equation:
Y=β0+β1X1+β2X2+β3X3, wherein:
Y=log of the event rat (ln(rate));
X1, X2, and X3 are variables selected from the group consisting of dsi0, dsi_{T}, (dsi_{T}−dsi0), and (dsi_{T}*dsi0); and
β0(intercept), β1, β2, β3 are regression coefficients.

69. The method of claim 68, wherein the regression coefficients are obtained from a clinical study of a multiplicity of patients having a chronic liver disease, and having a defined rate of clinical events over time.

70. The method of claim 67, wherein the clinical events are selected from the group consisting of Childs-Turcotte-Pugh 2 point score progression (CTP+2), variceal hemorrhage, ascites, encephalopathy, and death.

71.-74. (canceled)

75. A kit of components for determining one or more of STAT, portal HFR, systemic HFR, SHUNT, cholate elimination rate, RCA20, DSI values, algebraic HR values, or indexed HR values in a subject comprising
quantifying one or more distinguishable compounds in a blood or serum sample from a subject according to the method of claim 1, the subject having, or suspected of having or developing, a hepatic disorder; the kit comprising:
a first component comprising one or more vials, each vial comprising a first composition comprising a single oral dose of a first distinguishable compound.

76.-94. (canceled)

95. A method for determining an algebraic hepatic reserve (HRₐ) value in a subject, the method comprising:
receiving a plurality of blood or serum samples obtained from a subject having or suspected of having or developing a chronic liver disease or hepatic disorder, wherein the sample was collected from the subject less than 3 hours after simultaneous oral administration of a first distinguishable compound and intravenous administration of a second distinguishable compound to the subject;
processing the blood or serum sample to form a processed sample;
measuring the concentration of the first and second distinguishable compounds in the processed sample comprising mass detection, optionally wherein the measuring comprises injecting the processed sample onto a mass detection system;
quantifying the concentration of the first and second distinguishable compounds in the blood or serum sample;
determining a DSI value in the subject from the concentration of the first and second distinguishable compounds in the blood or serum samples; and
converting the DSI value into an algebraic Hepatic Reserve (HRₐ) value for the subject, wherein:
HRₐ=[100−(2×DSI)].

96. The method according to claim 95, wherein the determining of the DSI value in the subject comprises:
determining a HFRₚ value and a HFRₛ value in the subject from the concentration of the first and second distinguishable compounds in the blood or serum samples, respectively.

97. A method for determining an indexed hepatic reserve (HRindexed) value in a subject, the method comprising:
receiving a plurality of blood or serum samples obtained from a subject having or suspected of having or developing a chronic liver disease or hepatic disorder, wherein the sample was collected from the subject less than 3 hours after simultaneous oral administration of a first distinguishable agent and intravenous administration of a second distinguishable compound to the subject;
processing the blood or serum sample to form a processed sample;
measuring the concentration of the first and second distinguishable compounds in the processed sample comprising mass detection, optionally wherein the measuring comprises injecting the processed sample onto a mass detection system;
quantifying the concentration of the first and second distinguishable compounds in the blood or serum sample;
determining a portal HFR (HFRₚ) value and a systemic HFR (HFRₛ) value in the subject from the concentration of the first and second distinguishable compounds in the blood or serum samples, respectively; and
converting the HFRp and HFRs values into an indexed Hepatic Reserve (HRindexed, HRi) value, wherein
$$= H R = 100 - ( X ↦ {( l n ( \frac{y}{H F {R}_{p}} ) )}^{2} + {( l n ( \frac{Z}{H F {R}_{s}} ) )}^{2} )$$
wherein:
X is a scaling multiplier from 20 to 35 (optionally 29.40578) to yield a range from 100 (normal hepatic reserve) to 0 (no hepatic reserve),
y is the minimum value for Portal HFR determined from the average values for Portal HFR minus one SD of the mean for each in a plurality of healthy controls of lean body mass in a range or 15-40, optionally wherein y=29.1; and
z is the minimum value for Systemic HFR determined from the average values for Systemic HFR minus one SD of the mean for each in the plurality of healthy controls of lean body mass in a range of 4-10, optionally wherein z=6.52.

98. The method of claim 97, wherein the Portal HFR (HFRp), mL min⁻¹ kg⁻¹, is calculated from first distinguishable compound dose/AUC, adjusted for body weight of the subject in kg, and the Systemic HFR (HFRs), mL min⁻¹ kg⁻¹, is calculated from second distinguishable compound dose/AUC, adjusted for body weight of the subject in kg.

99.-102. (canceled)

103. A method for determining an estimated algebraic hepatic reserve (estimated HRₐ) value in a subject from an estimated DSI value in the subject obtained by the method of claim 43, the method comprising:
converting the estimated DSI value into an estimated algebraic Hepatic Reserve (HRₐ) value for the subject, wherein:
estimated HRₐ=[100−(2×estimated DSI)].

104. The method of claim 97, wherein a second or subsequent HRa, estimated HRa, or HRindexed value is determined in the subject after a predetermined interval of time, wherein:
an increase in the second or subsequent HRa, estimated HRa, or HRindexed value is indicative of improved liver function in the subject, optionally wherein the increase in HRa or HRindexed is at least about 2 percent, at least about 3 percent, at least about 4 percent, or at least about 5 percent, or higher.

105. The method of claim 104, wherein a decrease in the second or subsequent HRa, estimated HRa, or HRindexed value is indicative of worsened liver function in the subject.

106. A method of predicting pharmacokinetics (PK) of a drug in a subject having or suspected of having hepatic impairment comprising:
obtaining a single or a plurality of blood or serum samples collected at a single time point, or at intervals of time from the subject within 3 hours, within 90 minutes, within 60 minutes, within 45 minutes, or within 20 minutes after oral administration of a distinguishable compound, and optionally simultaneous intravenous administration of a second distinguishable compound to the subject;
determining a DSI value according to claim 56 or an estimated DSI value in the subject from the concentration of the first and optionally the second distinguishable compound in the sample(s); and
wherein a DSI value or estimated DSI value greater than about 15, greater than about 25, or greater than about 35 is predictive of a lower blood clearance or lower liver metabolism of the drug in the subject, compared to subjects having a DSI value or estimated DSI value of less than 15, or less than 10.

107.-108. (canceled)

109. A method for determining an RCA20 value in a subject having or suspected of having or developing a chronic liver disease or hepatic disorder, comprising
quantifying a distinguishable compound in a blood or serum sample from the subject, according to the method of claim 1, wherein
the blood or serum sample was obtained from the subject about 20 minutes after intravenous administration of the distinguishable compound.
